SEQUENCE LISTING (395 sequences)

SEQ ID NO: 1 - protein, 1539 aa, unidentified
MSKILGLDLGTNSIGWAVVDDTNNKILGIGSRIFPEGVKADTIGKGDKEESKNASRRTSRQQRRQFYRKRLRKIKLLRTLIDLKMCPLTHLELDVWAKWEKTKGKAGRKAPDSPNYEEWHRLNPYVLREKALYEDLTLFQMGRVLYHLIQRRGFLSNRKGKDDGAIYKGKPGMVGIKDTQKQISEKTLGSFLHSIFPKEGEPFKLITDENGNELRVRARYTLRDMYVEEFDQIWNRQAKALGLDDKNATKTKIQFLNGSLKNNRNKKKIENLQNKYGVENVKIEELKDGKSKKTLYKVTTTTNVPLKIFLGGKIEHDDEGNVKHKSQDSVLFWQRPLRSQKGLLAKCRFEPDFKDKNGKYLQRGKTPCHLSHPLYEEFRAYQYINNIEYGKKQKLEETQRLQVLELINSKDSNFNFSEIPKKLKMEYEKWNYSDDQKVAANSTTKHLSTLFSKEIYEANKEEIWHCFHFFEDSDNLIEKLKKDFGLAEKDIEKVDKINLKEGYSNVSLKAIKNILPYLQQGLQMSDATILGGVRNVFGKRWDYFKDAHEQLENDILKINHEKNNKEGEAIQKIKNYLADPENKFGFLKDDKSFIKLYHHSQEIEEKQLQDRLDAVENLRNPIVQQGINETRRLVNQLIGEYGKFDRIQVELGREVKNSKKGRQEQTRRIYDNTTKNDEARKLLTEYGLKHDRENIQKVLLYKEMQEKGVTTVCPYTNKSINISDVLGRENKIQIEHIIPKSVSLDDGFANKTLCDAKFNGLKGNLTPYQFYLKNSDSKLWGGATSWEDVEQRAYKVLPYPKAKRFTSKIKVEDADVKSSFIERQLNDMRYISRKTKEIMSQVCEDVRVLPGGLTAELRHLWGLNNILQPVMLVDLPDYKLEAGKSIPHYVVMDENNKPVLTLPIYNDKPKLGENETTITGKVEKGVFKTSEQFVKLEVKTPELVNGEYWLNLKLSEPKQIIRIFRERPETSENEIVLRGKVEKEKFKNEGIGLVTAKGFDNGSYWAKFTITNKKFEAPEKDKQPKKSGKQVLLFGEVKEGFFKSYIYSCETNELDGKYWLLLDIDTENASFERAINERPIVSDAKIIVEGSVNDEGIFVSEIDAQHQFKTNEKPGKYWFVFDILNELTDFNAIEQPKPLLQEKQTLVEGNIWVDKYTGEIKFDPKKNRDDHRHHAIDALVIALSKQSYFQQLSAYNAARDAKKKLNSFEKEQLDFPDPWNGFHTDAKKEAEKILISHKQNRKVLTEVRKKITKNGKTFTSVGNAVRGQLHKENVYGKRQAPFETTKGYHIRKKVVDLKDNQLDKIVDLEIRKIIINARKEEVTIKKHIDVLEKQKKKVHSDEEETALNNQIQDLKKQIDLLYTLKNKNGERVPIKKVRIREEMSNAQQVKEINQFVNPRNNHHVLIYKKENGDLDKDVVSFWTAVERIKQNNEIVQLPSDGVEVVETFQENDMFLLFLEKDRNIEDLNQKEIFSHLFKVQKIAGADYFMEICFRRHTDSKISTEADYVYIKGFREGKTGWYSFNPIKVQITPTGKIEKI

SEQ ID NO: 2 - protein, 1529 aa, Bizionia saleffrena
MAKILGLDLGTNSIGWAVVYDENNEIAGMGSRIFPEGVVAKTIGTGEKEESKNAARRNSRQQRRQFYRKRLRKIKLLRTLIDLNMCPLSHAELDIWAKWDKSKGKIGRIAPNSKSYIEWHIQNPYYLRNKALHKDLTLFEMGRVLYHIIQRRGFLSNRKGNDEGKIYKGKEGMVGIDQTQKSIVGSTLGSFLNSIYPVEGEPFKLITDENGNELRVRARYTLRDMYVEEFDKIWTIQAKQLGLLGKVGEKNKLTFLKGNLSSNRNKKKIEKLYKKYGEENVNIEEIKNGKSEKSTFKIVTKSILPLKEFFGGEIEYDEEGKVRYKSEDSLLFWQRPLRSQKGLLSKCRFEPTVEKGGVYLQKGKSPCHLSHPTYEEFRAYQYINNIEYGKKQKLNGIQRLIVLDLINCKDSNFDFSEIPKKLKMPYEKWNYSDDQKVPGNYTTKHLAGLFPKEIWENHKNKIWHCFYDFKDNDILVKKLISDFGLDEKLYDKVKKINLKEGYSNVSLKAVKNILPFLREGYRMSDATILGGVMNAFGNLISYFKDSEDVLVKDIIKIIRDKSNKEGEAIEKIKIYLVENKFGFVNDDKRFIKLYHHSQDIERKSLKEKLDPVENLRNPIVQQGINETRRLVNQLIKEHGQFDRIQVELGRNIKNSKKGRQEQSNRINENTVKNDAARELLTEYGLKHNRNNIQKVLLYKEMQDRGVVTVCPYTNTSINISDVLGAENKIQIEHIIPRSISLNDSFANKTLCDSKFNTLKGKLTPYQFYQKNNDPKLWGGAERWEDIEQRAYKLLPYYKAKCFTSKIKIEDSDVQSSFIERQLNDMRYISKKTKEIMSQVCDDVRVLPGSLTAELRHLWGLNNILQPVMDLDITNNNIKEDRVYPHYVVMDEDNKAVSTIVLYNKKPALKPRETTITGIINKDVFKTIDDYVNLTYDTRALNNGKYWLKLKLSQPTKMVRIFKERPESTEKELVLRGKIDREKFKNNSVGMINAKNFDNGSYWAKLAIKDKKFVLPEIEKQPKKLRNQVLLFGEVKDNYFTSFIYECETNKIDGKYWLLLDVDLDNANFNRAINEMPAFDLNQIRIEGTLNSEGVFVSEVDTDHRFKSKEKAGKYWVVFDIVEKPNEFNAIENPKPILEDNQTLIEGNIWVDKYTGEIKFDPKKNRGDHRHHAIDALVVALSKQSYFQQLSDYNAQREAKYKGLPYEKEQLNFPEPWTDFHTTAKNKAEKILVSHKQNRKVLTQVRKRIIKNGQTYTSVGDAVRGQLHKENIYGQRQAPFEISKGYHIRKKVSDLKDNQLKKIVDLEIRKIIVNARVEEVKMQKQITILLTQKKKSRTDEEELIIANQIESFQKDIQQLYTLKNKNGEPVPIKKVRVREEMSNAQKVKELNQYVNPRNNHHILIYKDENGELKECINSFWKVVERQKQKQPIYQLPNTYNKGRIINTLEENDMFLLGLTDEEYNDSKMNYDFLTKHLFRVQKISEGDYSFRHHLASTLENKKEQVRIGSLKKWKEMNPIKVKLTEIGSLI

SEQ ID NO: 3 - protein, 1546 aa, unidentified
MAKILGLDLGTNSIGWAVVDDEKKQILGTGIRIFPEGVVAKTIGTGDREVSKNAARRESRQSRRGFYRHRLRRIKLLETLIEFKMCPLTGKELKQWKQYDKKKGQAGKTFPSSEEFNKWLKLNPYKLRAKALNNDISLFELGRIFYHLIQRRGFLSNRKGNEEGKIFSGKDNMTGIDETNKQIKDKTLGTYLNSVIPKEYEPYKNIIDKNGKELRARGRYTLRDMYVDEFEKIWQRQSKQLGLNSIQKEVKKESIISGSIYNKRNKHRIKNLQKSKGKENVEIKGNILTIKEHIHLKEYLAGKIYYDDEGNLKHKSQDSVLFYQRPLRSQKGLLAKCSLEGRKFYDKKDKRWRTAGPTPCPLSHPEFEEFRALQFINNIEYGIKKRLDENQRLILLNLFNRKDTNFDFKLIPKELKLTYETFNYADDLKAPGNYTNAKLSKLFNPDIWEKHKENIWHCFYFYSDNDKLINKLKTDFGLEEKNEEKAAKIKLKDDYANVSLKAIRNITPFLKKGYKLSDAVLLGGVRNAFKTTDTENKPYDRWENFNNEHEKIEKDIIRINKDKKNKEGDAVKKIKEYLHNNCGFEKDDKNFKKLYHHSQEIEQKKIQDKLDKIENLRNPIVQQGLNELRRLVNTLIDEHGKFDKIKVELGRNLKMGKKQRQETEFKIRDNNKKNEEAREKLSEYGLAHSRENIQKYLLWKEIEDRSGTAKCPYTGKTINISDLLGKENKFQIEHIFPRSTSLDDSFANKTFCDAKFNGLKGNKTPYNFYKKNNDAKLWGGAKSWDEIKQRAFKLLPYKKAKRFTAEKVDEKAGGFIERQLNDTRYMSKKAKEILSQICPKDNIRVMPGSVTSELRHLWGLNNILQPVEILDFKEAKINIDKLEKHWVVFDEKGEVLSFYPIHNEKPNLKPNEITISGYLNNKGEFSSKYIALKSVYEELKGDNFKGEYWKKLKLSNKPLQTVKIFTDKPAVDDDEIVLRGKVAKKYFENDTIGKKIKTNFEDGYYWAKFKVNNVKFEKPIPKKQPKKKSGQILLYGTVEDAIFKSYIFECQTGEADGNYWAIIDLDFDTVDFSNTMNPKPETDDNKILIQGSINETGLFSSDIDNKHNFETTLPQGKYWTIFEIEGEDAKLYPQINLEPKLQDKQKVVEGTVWIDKYTGEIKFDPKKNRDDHRHHAIDAITIALTEQSYLQKLSTYNAQLDEKRRGKAKKPTFEMPWDNFYNDAKKAASEMLISHKANNKVLTKINKVIHKNGRTYTSKGYAAGGQLHKENVYGKRQTPGSKKPYYHIRKSVESLTTDKQIDKIVDGKIRAIILHGKAQEKRIKKEIDELNKQFKKAKTDFEKAEIQKQIKQKQQDIKMLFSLPNKNGERVPIKKVRVKENLGNMMQLKDKKVFAEKKGKEVELNQYVNPRNNHHVAIYKNTEGELFEKVVTFWEAVERKKEGLSVIDKNPTEGSVFVTSLEINDMFLLGINEDNINWENPNYSDLTKYIYRVQKVSSSYYTFRHHLASKLDNSKQEISLQSFKAWQEVNPIKVNIDILGRISKVD

SEQ ID NO: 4 - protein, 1554 aa, Flavobacter branchio
MAKILGLDLGTNSIGWAVVDDTKNKIIDAGVRIFPEGVKSDTIGKGDKEESKNAKRRTSRQTRRGFYRKRLRKIKLLRTLINFNMCPLKHEELDVWSKWDKIKKKEGRMAPELFLPDNHPYCLWLKQNPYELRNKALIEDVTLYEYGRVLYHLIQRRGFLSNRKGKDDGKIYKGKEGIKGIEDTQKEIQNSTLGKYLFDILPKEGEPFKIIQDEKGNELRVRSRYTLRDMYIAEFEAIWNRQAPQLGLDKIDYLNEKKILLKGGLKNKRNNNKIEYFKKSKGIDNVEIIEIKDELSNTDKEKFILKVISKIPLKEFLGGRIEKDEDENIKFKSNDSVLFWQRPLRSQKNLLSKCRFEPDLKDDNGKFIQKGKTPCHLSHLLFEEFRAYGYINNIEYGKKQKLEETQREQVLELINSKDSNFKFSEVKKKLKLEYETFNFDDDAPVNGNYTTKHLNSLFPKEILEKEYVIKVNNIDIVEYGYERIWHLFHYCDDNDVLIEKLQKEYGLEEKNIEKVSKTNLKEGYSNVSLKAIKNILPYLKEGRRLSDAVILGGVRNAFGKKWELYKEDHKELEKDLLKINQDKSNKEGEAIEKIKHYLIENKYGFVENDIRFKKLYHHSQEVIEVKQKDKIEDIDNLRNPIVQQGVNEVKRLVNLLIEEHGKFDQIRVELGRDLKNNKMKRQELTYKISQNTAKNENAKQLLTEYALRHSRENIQKVLLYQELQERGTIAVCPYTNKTINISDVLGRANKIQIEHIIPKSISLDDSFANKTLCDAKFNGLKGERTPYEFYQINKDVNLWGGAKTWEEIERRVFTVLPYPKAKRFTSKTKFDKNDFIERQLNDTRYISKKTAEILSQICGDVRVMPGSLTAELRRLWGLNNILQPAFTIDIPNLHIDENKHIPHYVVLDKDKNPIASQKIYNEKPKLKPNETTLFGKVDKGIFKSKEQYIKFQVDTPELINGEYWAKLLLSNPKSIVRVFKEKPENTEDEIVVRGKIEKEKFKHEGLNTINAQGKDNGVYWAKLTISNKEFKKPIKEDQPKKNGKQILLFGEVKEGVFSSYVYQCDAEKVEDGKYWLVLDIDFEKVDFEKALNDKSTVKENQIVIQGITNDDNVFVSDMDNQHQFTMNEGKGKYYVLFDIISEVDDFYPVKTEIPKIEEGQALVEGTVWVDKYSGEIKFDPKKNREDHRHHAIDALVIALTKETYFQQLSKHNASSENKKRGIEYDNDQLDFAEPWNGFHTDAQKTINSILISHKQNRNILTKITKKITKNGKTYKSEGMAVRGQLHKEFVFGFRTPPNGDEGYHIRKSVESLVTDKQINKIVDTSIREIIIKARRAEIPLYKEIENLSKQLKNPRLSEIEEKNINDKISQLRNDIKQLYTLPNKKGDPIPIKKVRVAEEMGKAQKLKSSINQFVNPRNNHHIVIYKDEEGQLKDTVTNFWKVVERVKQKEEIYRIPSPVVGQSVPKELILTFQENDMFILGLSEDEYNDNINNSSFLSNYLYRVQKISDGDYSFRHHLASTVTNKKEEIRIASMKKFQEMNPIKIVVNILGKNK

SEQ ID NO: 5 - protein, 1618 aa, Flavobacterium ajazii
MAKILGLDLGTNSIGWAVVDDMDKKIIDSGVRIFPEGVVAKTIGTGDKEESKNASRRNSRQQRRGFYRKRLRKIKLLRTLINLDMCPLKNEELDIWSKWDKQKKKEGRMAPEIHLPESHSYCLWLKQNPYLLRDKSLNEDLTLQEFGRILYHLIQRRGFLSNRKGKDDGKIYKGKDGVKGIEETQKELQNSTLGKYLFNILPKDGEPFKLIQDENGNELRVRSRYTLRDMYVAEFEAIWNRQACNLGLETIKCSNEKKVLLKGGLKSKRNNSKIEHLKKIKGEENIEVIELNDKLSKNDKQKYLLKIKTKLSLKEFLAGRIDKDEEGVVKFKSNESVLFWQRALRSQKGLLAKCRFEPDLKDENGKFIQKGKTPCHISHPLYEEYRALQYINNIEYGKKQRLEESQRLQVLELINSKDKKFDFIEIKKKLKLEYETFNYEDDSLVVGNYTTKYLNALFPKSILEKEFLIIVNKEEKLEYGYERIWHLFHFCEDNDKLICSLEKLIKETKIDYILNKAIIEKIEKVNLKEDYSSVSLKAIKNILPYLKKGLRLSDAVILAGVRNAFGKKWELYKNDHIELEKDLLKINHEKSNKEGEAIEKIKVYLIENKYGFVEEDVRFKKLYHHSQEIIQVKQKDKIDTVQNLRNPIVQQGVNETKRIVNELIAQKGKFDQIRIELGRDLKNSKTKREELGYKIRENDKKNEKAKELLIEFGLRQSRENIQKVLLYQEMPISICPYTNKTININDLLGRGNKFQIEHIIPKSISLDDSFANKTLCDSKFNGLKGNRTPHDFYEINKDPNLWGGAKSWEEIERRVFKVLPYPKAKRFVSKTKFETNDFIQRQLNDTRYISKKTSEILSQVCSDVRVMPGSLTAELRRLWGLNNILQPIITVDIPNLHIEENKHIPHYVVLDNDKKPVLSQRIYYDRPTTNKNETLLSGNIDKGVFKSKEQYVKFQVDAPDLENGDYWAKLVLSRPKNIVQVFKDKPSTLENEIVLRGKIERDKFKNDGLNAINATGKENGIYWATFTIEKKEFKKPAKEEQPKKNGKQILLFGEVKDGLFSSYIYECETEIEDGKYWLIIDVDFEKVDFEKALNERPLVRENQILIQGTTNDENIFVSDMDNQHHFEMEEGKGKYYVLFDIVSEVTEFSPVKTAKPVLEEGQTIVEGTTWIDKYTGEIKFDPIKNREDHRHHAIDALVIALTKESFFQRLSKQNASTENKKRGNEHEKEHLDFPEPWNGFHNDAKNVINGILISHKQNKNILTKITKKITKNGQTFKSEGMAVRGQLHETTFYGKNLHCKEKEIIQRLPISKLKFAPAKGGGASIYDIIDDNIRKLIISSVTNSFKQILQKYNDVSTTFEEIKELKKKYNKGKIDEAIEKLGVIEKMETDSNRLQKKSEIDKNQKEINRIKSTISETIESFLKDETFYLPNEGKRFKRLNKIDVGKERNPVPIKKVRIKKVMKNAETINQKIGQYVNPGNNHHILIYKDENEELKDDITNFWKVVERVKQKDEIYKLPEIEIDKPAPKELILTFQENDMFLLGISNDEYNDNKLDHTFLSNYLYRVQKISDGDYSFRHHIASTVTNKMEEIRITSMKKYHEMNPIKVEISVLGKISKM

SEQ ID NO: 6 - protein, 1558 aa, Flavobacterium alvei
MAKILGLDLGTNSIGWAIVDDVDKKIIDSGVRIFPEGVVAKTIGTGDKEESKNASRRNSRQQRRGFYRKRLRKIKLLRTLINLNMCPLKNEELDIWSKWDKQKKKEVRMAPEIHLPENHSYCLWLKQNPYLLRDKALNEDLTLQEFGRVLYHLIQRRGFLSNRKGKDDGKIYKGKDGVKGIEDTQKELQNSTLGKYLFDILPKDGEPFKVIQDENGNELRVRSRYTLRDMYVAEFETIWNRQAANLGLETIEYLNEKKVLLKGGLKNKRNNSKIEHLKKIKGENNVEVVELNDELSKNDKQKYLLKIKTKLPLKEFLAGRIDKDEEGVLKFKSNDSVLFWQRSLRSQKGLLAKCRFEPDLKDENGKFIQKGKTPCHISHPLYEEYRALQYINNIEYGKKQRLEESQRLQVLELINSRDKKFDFIEIKKKLKLEYETFNYEDDAPVIGNYTTKHLNALFPKSILEKEFSITVNEEEKTEYGYERIWHLFHFCEDNDKLIRGIEKLIKESKIDYVLNEAIIEKIEKVNIKEDYSSVSLKAIKNILPYLKMGLRLSDAVILAGVRNAFGKKWELYKNDHIELEKDLLKINHQKSNKEGEAIEKIKVYLIENKYGFIEDDVRFKKLYHHSQEVVQVKLKDKIDTVQNLRNPIVQQGVNETKRLVNELIAQQGKFDQIRVELGRDLKNSKTKREELGYKIRENDKKNEKAKELLIEFGLRQSRENIQRVLLYQEMPISICPYTNKTINISDLLGGKNKFQIEHIIPKSISLDDSFANKTLCDSEFNGLKGNRTPHDFYEINKDPNLWGGAKSWEEIERRVFKVLPYPKAKRFVSKTKFETNDFIQRQLNDTRYISKKTSEILSQVCSDVRVMPGSLTAELRRLWGLNNILQPIMTVDIPNLHIEENKHIQHYVVLDNDKKPVLSQRIYYDRPTTNENETLLSGSIEKGVFKSKEQYVKFQVDAPDLANGDYWAKLVLSNPKNILQVFRDKPSTLENEIVLRGKIEKDKFKNDGLNAINTTGKENGTYWATFSITNKDFKKPVKEEQPKKNGKQILLFGEVKEGLFSSYIYECEAELEDGKYWLIIDVDFEKVSFEKALNERPLVKENQILIQGTTNDENTFVSDMDNQHHFEMEEGKGKYYVLFDIVSEVTEFSPVKTTKPVLEEGQTIVEGTTWVDKYTGEIKFDPIKNREDHRHHAIDALVIALTKESFFQRLSKQNASTENKKRGNEHEKEHLDFPEPWNGFHTDAKNVINSILISHKQNKNILTKITKKITKNGQTFKSVGMAVRGQLHKENLYGNRTPPNGDSGFHIRKSIDSLKTDKQIEKIVDIKVKEIIIKARKAEIPLYKEIEQLSKQLKSPKNTEIEEKELKENIEALHTKIKLLYTLPNKNGEPVPIKKVRVSEEMSNAQQLKSKVKQYVNPRNNHHIVIYKDENEELKDDITNFWKVVERVKQKDEIYKLPEIEIGKPAPKELVLTFQENDMFLLGLSNDEFNDNKQDFTFLSNYLYRVQKISDGDYSFRHHLASTVTNKMEEVRIASMKKYQEMNPIKVRISFTGKIEKL

SEQ ID NO: 7 - protein, 1551 aa, Flavobacterium orientale
MGKILGLDLGTNSIGWALIDDIENKIIDSGVRIFPEGVVAKTIGQGEKEESKNASRRNSRQQRRGFYRKRLRKIKLLRTLINLKMCPLSHSELDIWSKWDKKKKKDGRMAPEIHLPENHPYCIWLKQNPYLLRDRALNEDISLQEFGRVLYHLIQRRGFLSNRKGKEDGKIYKGKDGVKGIEDTQKELQNSTLGKYLFDILPKDSEPFKVIQDENGNELRVRSRYTLRDMYIAEFEAIWNRQAYNLGLDIIEYTNEKKILLKGGLKNKRNNSKIEHLKKIKGENNVEVIELIDELSINDNQKYLLKIKTKLPLKDFLAGKIEKNEEGELKFKSNESVLFWQRPLRSQKGLLAKCRFEPDLKDENGKFIQKGKTPCPLSHPLYEEYRTLQYINNIEYGKKQRLEESQRLQVLELINSKEKKFDFIEIKRKLKLEYETFNYADDSPVIGNYTTKQLNVLFPKSILEKEYSITVNDQETIEYGYERIWHLFHYCDDNDILLEKLQTIYGLTDTDKEKISKINLKEDYSSVSLKAIKNILPYLKKGLRLSDAVILGGVRNAFGKKWELYKNDHFELEKDLLKINHEKSNKEGEAIEKIKDYLIENKYGFAENDIRFKKLYHHSQEVIKVEQKDKIDTVENLRNPIVQQGVNETKRLVNELITLHGKFDQIKVELGRDLKNSKTKREELGYKIRENDKKNEKAKELLIEFGLRQSRDNIQKVLLYQEMPISVCPYTNKTINISDVLGRENKFQIEHIIPKSISLDDSFANKTLCDSKFNGLKGNRTPYDFYEINKDPILWGGAKSWEEIEIRAFKVLPYPKAKRFVSKTKFETNDFIQRQLNDTRYISKKTSEILSQVCSDVRVMPGSLTAELRRLWGLNNILQPIMTVDIPNLHIEENKHIPHYVVLDNEKKPILSQRIYFDKPITKENETLLSGSIEKGVFKSKEQYVKFQVNAPELANGDYWAKLVLSNPKNIVQVFKDKPNALENEIVLRGKIEKEKFKNDGLNAINAIGKDNGTYWATFSIINRDFKKPVKEEQPKKNGKQILLFGEVKEGLFSSYIYDCEAEIEDGKYWLIIDVNFENVSFERALNEKPLVKENQILIQGTTNDENIFVSDMDNQHHFEIEEGKGKYYVLFDIVSEVTEFSPVKTTIPVLEEGQSLVEGTTWVDKYTGEIKFDPIKNREDHRHHAIDALVIALTKESFFQRLSKQNASNENKKRGIEHEKEHLDFPEPWNGFHNEAKNVINSILISHKQNKKILTKITKKITKNGQTFKSNGMAVRGQLHKENLYGYRTPPIGEAGYHIRKSVESLKTDKQLEKIVDVKIKEIIIKARKAEIPLYKEVEQLSKQLKSPKNTEIEEKEIKEKIVALHTKIKSLYTLPNKNGEPVPIKKVRVSEEMGKAQQLKPSIKQYVNPRNNHHIVIFKDENEELKDDITNFWKVVERVKQKDEIYKLPDLEIGKPAPKELVLTFQENDMFLLGLSNEEYNDNKQDLVFLSNYLYRVQKISDGDYSFRYHLASTVTNKKEEVRITSMKKYQEMNPIKVNISVLGRIEKL

SEQ ID NO: 8 - protein, 1532 aa, unidentified
MAKVLGLDLGTNSIGWAVVDDDKQKIFGMGSRIFPEGVVAKTIGKGEKEESKNAARRNSRQQRRQYYRKRLRKIKLLRTLIDLDMCPLTHKELDLWAKWDKYRGKAGRIAPSSKVYIEWHRQNPYHLRDKALNEDLSLHEMGRVLYHLIQRRGFLSNRKGKDDGKIYKGKEGMVGIDQTKNSIDNGTLGSFLNSIYPPEGEPFQLITDENGNELRVRARYTLRDMYVEEFDKIWERQAKPLGLLEKTGEKKKLTFLKGNLDANRNKKKIEKLYQKYGEPNVTITEIENKDTRKSIFKVVVKSIVPLKEFFGGEIEYDKEGTIKHKSQDSVLFWQRPLRSQKGLLAKCRFEPTVTKNGKYLQRGKSPCHLSHPVYEEFRAYQFINNIEYGRKQKLNEYQRLQVLALMNGKDSNFNFSEIPKKLKMEYEKWNYADDQKVPGNYTTKHLSSLFSKEVWEKYKHEIWHCFMDFEDNDNLIEKLISDFDLNEKLIDKVERINLKEGYSNVSLKAIKNILPFLREGFQMSDATIFGGVMNAFGDQINYFKDSKEQLKKDVLNIVRDKTNKEGDAIEKIKSYLVENDFGFTPDDKRFIKLYHHSQDIETKELKDKLDAIENLRNPIVQQGLNETRRLVNHLIDEYGKFDRIQVELGRDVKNSKQGRQNQSNRIKENTTKNDAARKLLTEYGLRHTRVNLQKVLLYKEMQDRGVVTVCPYTNISINISDVLGRENKIQIEHIMPKSTSLNDSFGNKTLCDSKFNGLKGNLTPYQFYLKNNDPKIWGGAETWEDIEQRIYRLLPYPKAKRFTSKIKIEDADVQNSFIERQLNDTRYISKKTKEIMSQVCEDVRVLPGGLTSELRHLWGLNNILQPVMNLDINLKNMEEDRVYPHYVVMDENNNLISSLPIYNEKPTLEANQTTITGKVNKGVFKTLNNYVNITVSVPELTNGNYWLKLNISEPKKVVRIFKERPESTENTIVLRGNVEREKFKNNSIGLVSAKAIDNGSYWAKLPVIDKKFIVPEKEKQPKKTGKQILLFGEVKDNYFKSYIYECETNTTDGKYWLLLDLDTEHPVYERAINEKPSFASNQICIEGTVNDQGVFVSEIDAEHQFATNEKEGKYWVVYDIVNEPNEFNAIENQKPPIEKNQSLIEGNIWVDKYTGEIKFDPKKNRDDHRHHAIDALVIALSKQSYFQQLSTYNAQRVAKYKGLPFEKDQLNFPVPWLNFHTSAKKEAEKILVSHKQNRKVLTQVRKRITKNGKTFTSVGDAVRGQLHKENIYGQRQAPAEVSKGYHMRKKVSDLKDNQLKKIVDPEIRRIITKARIEEVKINKEIALLEKQKKQKGVSDEEELVIVDQINSLKNDIQQLYTLKNKNGERVPIKKVRLREEMSNAQKLKEANQYVNPRNNHHIIIYEDKMGELKETIVSFWEVVERQKQNQEIYQLPEFDKHGKIINTLQENDMFVLGLDDEEFNENKLDSKFLIQYLYRVQKISAGDYSFRHHLASTLRNKEEEVRVTSLKRWNEINPIKVNLTETGSIIEA

SEQ ID NO: 9 - protein, 1558 aa, Kaistella flava
MAKILGLDLGTNSIGWAIVDDLENEILDVGVRIFPEGVISKTIGYGDKEESKNAKRRNSRQLRKQYYRKRIRKIKLLRTLINFEMCPLEHNELDVWSKWDKQKKKEGRMAPELFLPKSHSYNLWLKQDPYKLRNRALREDLTLLEFGRVLYHLIQRRGFLSNRKGKDDGAIYKGKENVKGIDATLKELQDSTLGNYLFHILPKDGEPYKIIQDEDGNELRVRSRYTLRDMYVAEFEAIWNRQSKHLGLETVECLSEKTRFLNGSIENKRNAKKIEHLQLTKGTENVEIKYISDELSKDDKKKILLKVTTKLPLKEFLAGKIEKDEEENLKFKSNESVLFWQRPLKSQKKLLAKCRFEPDLKDEFGKFIQKGKTPCHLSHPLFEQFRAYKFINNIEYGKKQRLEESQRLEVLELINSKDTNFKFSEIQKKLKLEYETFNYDKDSSVSGSYTTKNLNSVFPKSILEKEFVVKVNGEDVIEYGIDRIWHLFHYCEDNDILIKILQNDYNLEEDKIDKIAKINLKEGYSSFSLKAIKNILPYLKEGKRESDAVILGGVRNAFGKRWKYFANEYEELKNNILKINYDKTNKEGDSIEKIKRYLIDKKYGFVESDISFKKLYHHSQEVEIKKQFNKIGDIENLRNPIVQQGVNEVKRIVNELIVQHGKFDQIRVELGRDLKNGKTKRQELTNRISQNTTKNENAKQLLTEYGLSHGRENIQKVLLYQELQEKGILPVCPYTNKTINISDVLGRGNKIQIEHIIPRSVSLDDSFANKTLCDSKFNGLKGERTPYEFYQLNKDVNLWGGAKSWEEIERRAFKVLPYQKAKRFVSQTKFDKNTFIERQLNDTRYISKKTAGILSQVCDDVRVMPGSLTAELRRLWGLNNILQPAFTIDIPNLYIDAERHIPHYVVFDKDKNPISSQRIFNAKPDLLQNETTLFGNVDKGLFKSKEQYVKFQIETPDLLHGEYWTKLKLSNPKNIVRVFKEKPETSEKEIVVRGKIEKEKFKTEGLNMINAVGNENGTYWAKLTILNKTFKKPTKEDQPKKNGKQILLYGEVKEKVFSSYVYQCDTNQEDGKYWLIIDIDVENVLFDKAMNEKPLANDYQIVIQGTTNNENVFVSDMDSQHHFNMDEGQGKYYVLFDIISKVEGFYPVKTQEQKLEDGQTLVEGTVWVDKYTGEIKFDPKKNREDHRHHAIDAIVIALTKEKYFQELSKHNASSENKKRGNEYEKEHLNFIEPWTGFHTDAQKVVNGILISFKQNKNILTEVRKKIVKNGKTYLSVGDSVRGQLHMENVYGKRTPPEGKEGFHRRESVESLVSEKQINKIVDTTIRKIIINARKEEIPIYKEIESLAKQLKSSKLNDFEEVSLNEKIIELRLQIKQLYTLPNKKGEPVPIKKVRVSQEIGNAQQLKPTIKQYVNPRKNHHIVIYKDENGELKDSITNFWNVVERVKHKDKIYKLPEDKYVEPELKELVLTLQENDMFLLGLSNEEYNDNFENSSFLSSYLYRVQKISDGDFSFRHHLASTVTNKNEEIRVASMKKYQEINPIKVKIDLQGKIQITGKD

SEQ ID NO: 10 - protein, 1519 aa, Pontiella sulfatireligans
MSKRILGLDLGTNSIGWAVIDEVQEKIVDAGVRIFPEGVVSKTIGSGDKEETKNATRRKNRQTRRQFYRKRMRKAKLLEVLIEQGMCPLSLKELDLWRRWDKEKKSQGRKFPASDEFMKWIRLSPYELRARAVAGTVSAHELGRVFYHMIQRRGFLSSRKGEAKDSETLFEKGKPGVLPINTTVSEMAKTGSQSFGQYLNAVVPKTGKPYAKSCDSEGNEIRARGRYATRQMYIEEFEQIWKAQADKLGLDGRTVVYRKVRKLQGGEDSARNQKKAEKLRKKFGADRVCFERKGKHTFLVSTQDMPFKELLAGKIWEEDGRLKFKSNESVLFWQRDLRSQKGTLGNCTFEDGLPVIQLDGTVRRGKDGKPVTYSKKPCPVSHPAFELFRAHQFINNIKYGNGQRLTREQRATVLELMNSKDRDFDFKEVVKKLELFSEQFNYEKDFKVPANSTICKLSKLDKGKEWQSVLEEHLPEIWHCFYTYTDSDMLCEKIARTYGVRLDLGKVDKVRFKDDYSRVSLKAIGNILPFLKKGLFYSTSVILGGVKNAFGRRWDDYLTDETKEKIQADVLEVLKEKNGEGDVIEKIKAYLSDPANRYAFSENAPAFGKLYHHSQDVERRNSNAAKLSPVKDVRNPIVQQALHEMRMVVNALMAKYREDDPNFRFEFIHVEMGRDLKNGKKRRRELSRVIDENKKKNDEAREWLAEYGLRPSRENLQKMLLFMELEKRAGRAQSPYTGKTIPLSNLLGANNTIQIEHIMPYSKSLNDSFGNKTLCESEINGMKGDETPYEFYLRNSDPGLWGASSWDDVARRAFALLPYEKARRFASKEPVDAEKFVVRQLNDTRYIAKKAVEVLSEICRDVHVMPGQLTSELRHLWGLNNVLESSIGADRLLGDLAASDVGDYYLVLDQDGQAISLSKRKNNQPDTDANEILGAGGVEKRKFDAKHLVLKLDAPEEADGKYWARLVLKPNLKLTPKYLERPDEGEDYIVFKGMIRKGKFNHDSIPVVLDAEQDDGSCWVKLFLKSKRPVDAKSNPRTTKKEIKLFGQVKDGVFECYIYKCKTDAPDGKYWLILEPDFDAPQFVRAVNPPPEHADSEIIMQGTVDESGIFVSDIDRDFVAKTDKVPGKYYAVAELFSHTPRFHLMENPVPDIAKGQRVVEGTVWIDKYSAEIKFDPKKNREDQRHHAVDAITIALTKRSYLNALSRFRSKEHPEEYKPVFSAPWEGFENDVKSAVEKILVSYRKDERVVSKGKKGTAVRGQLHKDNVFGEYVNLTDGSKSYHRRKALTALETHKQAEKIVDETIKKLVLDHLRDNCGIDIKSKKGVKIPADAFFKDGKPRLFLPPNPKKNGSPVPIKKVRLEETINNARQWKKGKNQYVNPRNNHHVLVYKDSEGELKEQIVSFWDAVQRTMKKQDVYELPDDADEQVLVLEKNDMVVLGAGDIEFGEISCDPALLSQKLYRVQKLYSGDDGFEYVFRYHLASTLNNKNEEVMIKSFKKFRGVNPIKIGISELGEIARC

SEQ ID NO: 11 - protein, 1509 aa, unidentified
MITLGLDIGVSSLGTAIVDTQKNQILHTGVYVFPAGKEAFGTNKEKPKNETRRVARQGRRQNYRRRLRRSQLLKFLYQLSELNSPEGDRYVPLTFEEIDAWLSWDKEGKTAARQFPDSDAFRAWMAMNPYEIRSRAVEGSITRAEFGRLLYHIIQRRGFQSSRKMKADEGAMAKGKDDIIGYTQTKSEIGEQTLGQYLYQISAKEGEPFTQREEKPRGRYTLRSMYVEEMERIWDQQAKRLGLEELEIEGTPVKELLIGQISHDLESGELLYKSNDSVLFWQRPLRSQKHLINKCSLESRKMYDPVKQRHYIQGPRVAAVSHPTSEFHRALQVLSNVRVEGRTLYDMGLFQTSLDFLCSKNSKTIKVADLKRHLSLVSKLNYDDDQSLPGCPTIASITDLFEKGELSYHYGETDTIYIDIWQKLLFYDDERLLEKNIADYAKGRGLKLKADFAKRLDGINLVDDYGSISIHAMDNIIPFLLQGRSHTEAIFLGGVKNAFGLRYYYFEKEEQMWQDLLQIINEPASKGEKIDRLIEFLSNPDEQTGLDEKRLRMKLYHPTQSVQKIEIQNRLGEVDNLRNPIVEQALWAVRRQVNALIDQMIEQTGNPNFRFDRITIELSRDLKSSLDRRLKIRQEQEANRAENEEAVIELRARGVRVTRDNILKYRLHKELQQQDGIARCPYTGKNINPSYLYDGNNFFQIEHIIPRSISLDDSFGNKTLCESNFNREKGNMTPYEFYSVNSSKTLWGAEDWDEITHRVRRVLPARKASRFLSKRSAQEQLEELPSKMLNDTAYMSVKAREYLTTICGDVRSVTGHITADLRRMWQLNKILGETYECAPGAVNEELIARGDTDVYALLDSESQVVNRVAPKLNLIPNPTADEIRIPVVVKGTKLYRDGKEYPKPAHLDLSGVTGEALIAYHAVGGSPHCQPLFNPKPSAKDMLVVPATINSKNQLTSDYIEALRKVKRSSYPTEPGKYWVTLQLKGEPIWELPSNKKRVAANAVRVSGRLNGNAFWSPLFETEVDSSVVNMEEHRNRTAYLNLDYDTAVFTKMLRPAPEVGEGELLLTGVANHGSLLLDQCDGKTIDVQHLNLQEQTKYYVSLRYGETLSPEERVTIYPLYAKAPTPRGGEVLLEGKLFLLDKSQGLYALEPIKNRDDHRHHAVDALVIALAKQNYVTELSTYNRQDDSYRQGEALKPRFDLPWEGFREEAEQSINDILVVHKKNRVDPLSKVTRKITKNGKVYTSEGYAVRGALHKATIYGQRKAPGQTEMGYHYRKSITEVTKMSQLNKVVDAAIRKEMLRILKEELGLDISNPKLVFPKNAFIDEDGKTKVYLPNRNGGEPVPVKKVRLCDKFNNVVRLKEANQYVDPQNNHHVLIYEALDGSLQESVVTMWDVVNRRNRGEEIFQLPPDGKRILFKLMRDRMYLIGITKEEVLLHLSDTAYLSKYLYRVQKLSSKDYFFRKATASTTNYDREMIRVSSLSFFEPEKDNVHSVEISLTGKVTLKDD

SEQ ID NO: 12 - protein, 1511 aa, unidentified
MITLGLDIGVSSLGTAIVDTQKNQILHTGVYVFPAGKEAFGTNKEKPKNETRRVARQGRRQNYRRRLRRSQLLKFLYQLSELNSPEGDRYVPLTFEEIDAWLSWDKEGKTAARQFPDSEAFRAWMAMNPYEIRSRAVEGSITRAEFGRLLYHIIQRRGFQSSRKMKADEGAMAKGKDDIIGYTQTKSEIGEQTLGQYLYQISAKEGEPFTQREEKPRGRYTLRSMYVEEMERIWGQQSKRLGLEGLEIEGTPVKELLIGQISHDPESGELLYKSNDSVLFWQRPLRSQKHLINKCSLESRKMYDPVKQRHYIQGPRVAAVSHPTSEFHRALQVLSNVRVEGRTLYDMGLFQTSLDFLCSKNSKTIKVADLKRHLSLVSKLNYDDDQTLPGCPTIASITDLFEKGALSYHYGETDTIYIDIWQKLLFYDDERLLEKNIADYAQSKGLKLKADFAKRLEGINLVDDYGSISIHAMDNIIPFLLQGRSHTEAIFLGGVKNAFGPRYYYFEKEEQMWQELLQIINEPASKGEKIDRLIQFLSNPDEQTGLDEKRLRMKLYHPTQSVQKIEIQNRLGEVDNLRNPIVEQALWAVRRQVNALIDQMIEQTGNPNFRFDRITIELSRDLKSSKERRSKIQQEQEANRAENEEAVMWLKALDLPYTGDNILKYRLHKELRLKGGGVARCPYTGKDIRLAQLYDGNNLFQIEHIIPRSISLDNSFGNKTLCESNFNREKGNMTPYEFYSVNSSKTLWGAEDWDEIIHRVRQVLPARKASRFLSKRSAQEQREELPSKMLNDTAYMSVKAREYLTSICSDVRTVSGSITANLRRLWGLNKILGETYECLPGTVSKELAAGEEKEVYAILDTKSNVITRVVPKFNPIPYRTADEIRIPVVVKGTKLFCDGKEYPKPAHLDLNGVSGEALLAYHAVGGIPHCQPLFNPKPSNKGVLVVTATRSGKSNLISNDIDVLDKIKKSRYPTEPGKYWVTLQLKGEPIWEFPSNKKRVAANAVRVPGRLKGNTFVSSFFETEEENSVVNMEEHRNRTAYLNLDYDTAEFTKMLRPEPAVGEGELLLTGVVNHGSLLLDQCDGKTIDVQHLNLKEQTRHYVLLQYGKNLSSKEQVTIYPIYNKAPKSRKDEVLLEGKFFALDKDKSLYALEPVKNRDDHRHHAVDALVIALAKQNYVTELSTYNRQDDSYRQGGRLKPRFDLPWDGFREEAEQSINDILVVHKKNRVDPLSKVTRKITKNGKVYTSEGYAVRGALHMDTIYGQRKAPGQAEVGYHSRKSITEVTNMSQLNKVVDAAIRKEMLRILKEELGLDTSNSSLTFPKNAFIDEDGKTKVYLPNRNGGEPVPVKKVRLRENFSSPRCLKNQNQFVKTENNHHMLVYKTKKGKFNESIVTFWDVVERLNRGEAIFQLPEDGEKIIFKLMANDYFIIGLKKQEIISNLSNPSLLSKYLFRVTSLSSRDYRFRLVAASTDKNKHENRRIRTPNFFDPTKENIHAVEVDLNGNIRLKEDD

SEQ ID NO: 13 - protein, 1564 aa, unidentified
MRKEQYILGLDLGTSSLGWAVIVREGASATSILNAGSTVFPSGKAMVNGVEKTNNEERRNSRQARRQLARRRLRKQHLLRLLIEKDMCPLTQTELDGWVKWDKSRPSEERRRFPESPRFREWLALDPYRLRAEAVDTPLEDDADGGFTAREKLGRILYSIIQHRGFLSSRSGKEDGSIYTSTMEKDGKVGIDETRAALGDRTLGQYLYSIAPKEGDTLSDQAERVRSRYTLREMYVEEFDRIWTRQAPLLGLDREQVETVHYRYFTGKSDSYEARRRLERYRTKDPSAVIETLSGDEHRFVYKTTTSLKEHLAGKIWYDKDGTIRFVSKESTLFWQRPLRSQKSMLDKCSLESHKVKDRGTNRFITQGPTVVSASHPYYELYRVLQTLLNMRIRDRKLDLMMLQSMLDKLLTYGKNTKVSKLKKDLRLTLDLNFPDDYELPGCPTISGITKLLDSKKMPSRKGDASPVDRLVIPFDLDLYTRIWEKLFFYDDNHLLAQGLRGIEGAAFVEDLEDKLAKLHLSEDYGNLSLHALRNILPYLLRGEELYSAVLLGGIRNAVRDDALLDDDALRLWIHTQGPLARGEMIDRIVALLQDPDGPFQLSTPELKLRQRLYHPSQGTAKHRLMDQLPPVENLRNPMVEQALWAMRRLVNYLLNNYRSTIRPDFTFDEIHVELARDLKLSTQKRAEMRKRQKKAEEDNDMARDYLRSQGLSTSRENLQKYKLYMELAGRSMGVVTCPYTGKVIRGLSAALDGNSKFQIEHIVPRTMTYDDSFANKTLCDADFNRKKGNLTPWQFYQQDPSPKLWGADSWEEVEHRAFRLLPYRKATRFVSKTIEDQLSGDQLVDTAYISRKAKEYLSYICPQDHIRLYPGRVTARLRQLWGLNSILSQGPTITKGQLPAGLSLSGDQIPAIVIGKKKSGVLSRILPKYTSRPEREERDVVLLVRVQKGKVYSARYSELPLSWDKATSLADGTYYIVLRSTEVLRVERMFADEPRLSEDQLRARVTIELNRDGQPKLSSREMDVRILKDTPLPTAPGNYWLTIPLISVPEVTPEHPRAKVAPDTIKVTGSVEQGVFISKVYRQGAVAMADGVAQGIPAPMLSEASFTKIKLDAPVITSGEVVLTGAIDRSDLFVCDQDDEFRVHIDSFDEPGKYYAVFSFDPMASYILSYDQTPNLSSDEVVLEGELIYNDKDDRIHFSPAKNRDDHRHHALDAIVIACTGQKVLTAMSTYNAHADDYERGAGSRPTFASPWEGFRDDVREAIEAVLVKHKQSTKILNKVTKQITKDGRTHRSKGLAVRGKLHKETFYGQRQSPEEPAPTMHYRVPITSITKSKQLNQVADPTIRALMIRRLQDMGVDTTQEDYAIPGNAFVDKAGNYTILVPNKRGVPVPVKKVRRSRVVNNTVRLDGYNKEVETSNNDYVLIYEDREGNYCEEVVTFWDSADRLLHGQDRVQLPMDVKRVVMTLRINDMFVIGMSEDELRSAVGSSDYGVISDHLYRVQKLSSMHYVFRLHRASTLNHDEELYTIRSFSALKESTPIKVIIDTDGTISIAARDDQANNHHQ

SEQ ID NO: 14 - protein, 1513 aa, unidentified
MGKVLGLDLGTNSIGWAIVEEERDEIIGSGVRIFPSAADNIGTSKESLKNAVRRNARQIRRQFFRRKLRKIKLLEVLIKYNMCPLSTELLNGWKKRVKGEIQKFPQSEEFNRWLKLNPLFLRDKATKEQLSLQEFGRVLYNLIQKRGFISNRKTLNDKGAMYKGKPEERILGIAQTEKELEKKATLGSFLYNIFKKDGEKYKFSVERVRGRYTLRQWYIDEFNVLWDKQTPNLGLDKKNEIYKKRSYIGNINSSRARKKIEILKKQGKTFNIENGYIIVEEHIALRDLIGDPNFGILFFQRPLRSQKHLLSLCSFESRKILINNNGEKIEKAVGKRICSASHPDFEKYRAYKFINSIKYGADLSLNESERKLLAEFMLSKDKAFDFKKISEFLDMRSEKFNYADDFKVFGSPYTATVKKSVSEDLWNNKKKYEELWNVFYFYKDGDKLKNKLKKDFNINDEKILNKLEGMKLSEDYSNLSKKAIVNILEFLVKGYQEHIAILLGGVKNAIGKDKWSSYNKEKIKKIEDEVYRMAKEKMKSGDLIKNIVNLVYEYEKIPINKIYEKIYHHSEEVIQNKTISHKLGEIENLRNPIVQQSLNEMKKLVNKLIDKYLDEDENFKSINIELARELKLPKSKRQLIYKENSDREKINRSACDILDEYGLAHSRENIHKYLLYKELETENGIAICPYTGKTIGISDILGDSNEFQVEHIIPYSISLDDSLKNKTLCESTENNKKGNRTPYQFYKNNPVHWEKVRERAYKLLPYEKAKKFTSQQNFQQDDFIERQLNDTRYITKKALEYLKTICVDVFSLPGSLTADLRNKWGLNNIVRNPSLIDTNSIKISGYGKYWAEYGDSENPNKITKLYPKYNKKPIPDSNEIIITGEIANNEFTPDSNFELKLSKIKLSQKYEDGKHWASVKFDPNLQSIQPVKKPYPIKDKDNLILIGNMIKNQFINKQFKFTVIPSAFQDDKERPSYCKLTIDINNISFKQIKPKTTENTIILPGKVSSNHFFVFPDIFSKKVELPDGRYYAEIPYFPEKTEISPLLNIKPSIENKQILLYGMVRKNAFVPNSNPNLKINLKDIISDGQYWAVFNLTGEIGKFVRKENIPPKVENQTKLEEGEIRYNSESEEYYFYSGKDRTDHRHHAIDAITIALAKRGHLQKLSAFNAKKIERKRNINKINEKLRFETPWDNFTNDAKKSIENILISHKYHNPTVKKVIKNFKKNGKSFRGVGLSVRGALHKDTVYGLHKRPVISYDGKFTFNNGKIVTENENYYHVRKKLSDLKDRKQIEKIVDYRIKEIIYRLIEQSGGFIKDNIPSGALFEIDEDAKSLKSKVFLPNKKGEPIPIYKVRIRENISNARCVKPSYKVDDIEFNQYVNPRNNHHIMIYKNKNGDLSEDVVQFWDVIQRKMNNQPIYQLPEDGVEIVTTMEINDMFILGIRGTKEDALKLSKETLSLHLYRVQKLSSNYYTFRSHLDSTTEQITEMRIQSFSKWIEENPIKVKISEIGEIS

SEQ ID NO: 15 - protein, 1492 aa, unidentified
METTLGIDLGTNSIGIALTDQKQARILYSGIRIFPEGITKDTIGQGEKEESRNATRRMKRQMRRQYFRKRLRKIKLLEILVACNMCPLQPEDIRRWKNWDKHLKSTARQFPDSPAFREWLKLNPYELRKKGVTENISRQELGRILYHFIQRRGFLSSRKGKEDGKIFTGKDQMTGIDETRKNLQNRTLGTYLYEIAPKKGEKYRFRTERVRARYTLRDMYVREFETLWNRQAAHLELDNKQMPQKRHIYLKGTSTNNRNRKIITHLQQKYGTENISIENSRITVSYLLPLKELLGGKIEQQDGNLKFKCNESVLFWQRSLRTQKSLLSKCVFEGRRFYDSIHRKWITAGPTPAPLSHPEFEEFRAYQFINNITYGKGEHLNATQREDLFKLMCSESKEFNFDKIPKHLKLYEKFNFDKQTKVPACTTISQLQKLFPPETWQQHREEIWHCFYFYDDNELLFQKLKKDYNLQLNESEKLKKIRLAEGYGHVSLKAIRRINPYLQKGYAFSTAVLLGGIRNAFGKRFEYFKESETEIEQAVCRILKERNTEGGTILKIKDYLTKNQYGFTEHDRAFQKLYHHSQPVNAKTLQEKLPATENLRNPIVQQALNELRRTVNKLLSDCRQKYGPEFQFDHIHVEMGRDLRSSKTEREKQTRFIRENEKKNEAARQKLREFGLQPNRENIQKYLLYKEIEDKAGIVSCPYTGKTIKISDALGSDGCVQIEHIIPYSISLDDSFANKTLCEATFNREKGERTPYEFYEKNPSSSLWGDVHCWEDVEERAFRLLPYPKAQRFIRRKNPDTNEFIRRQLNDTRYMGKKAVEYLSSICKDVKAFPGQLTAELRHLWGLNNILQSVPDITFATPVPVGMRKNYYLITNEQYQVTQILPQEADRPQTENNEVFITGTVKHNSFKYRGMQDYPTERNDGKYGARLKLSAPVAWHPLFAQKPNSSEEQLVLKGKIDKGIFVCDYLKQKIKTNLPDGSYWVSLPVRNRSFQDSESVNRPKLTPQQVQLYGTIHEGRFHCDTYQCQTTGGDGKFWCILDVDTEQPEFTRIKNEFPALEERQIILTGSIDEKGIFCADDDLNYKVSTTLSKGKYYGIFPWESQQPELSEIEFTAPKINKDEKLIEGQIWVDEHTGEIKFDPKKNREDQRHHAIDAIVIALSSQSLFQRLSTFNAQRENKKRGINSTEHFPLPWSGFIGDVSQSVKNILVSYKQNLRTRCKISKTLIKDGRQIHSVGNAVRGQLHKDSIYGQRLAPGNTEKTYHIRKDIRDLKTKKHIEKVVDPTIRQMLLTHLQENCHINTNQEFDIPSDAFFKDDKYRIFLPNKYGEPVPVKKVRIKEKMENAEQLKNNVNQFVNPRNNHHVMLYIDREGNLKEEIVSFWTVIERQNQHQPVFQLPADGKAPICTLQINDTFIIGLKDEELEVYRNDRSTLSKYLYRVQKLSSMYYTFRHHLASTIKNEKEEVHIRSLEAWKQANPVKVYIDEIGNLTF

SEQ ID NO: 16 - protein, 1517 aa, unidentified
MTKVLGLDLGTNSIGWAVVDKEGEKILDAGVRIFPEGVEPTTIGKGDNEQSKNATRREKRQLRRHFYRKRLRKIKLLEVLIGQKMCPLRIEDLRKWKNWNSKEKAEGRRFPSTIEFDRWIQMNPYELRDKALREQVTLFEFGRIMYHLIQRRGFLSSRKGEQDPKTLFEKGKPDENILPVNETKEKIGNTSLGSYLNSIAYKENTPYKTRTDVKGNEIRVRGRYTVRKMYVEEFMKIWDKQNEYYDLDVIKLKTRKIRELRGSLNGARNRNKLEYLVSKYGEENVIIEQKSNNITQVITISEILLKKYLGGEIYYVENEEGEKKLKFRSNESVLFWQRPLKSQKSLLTDCRFESNLPVIMSNGEIRKDKEGKFVLRSKKPCPISHPEFELFRAYQFVNNIRFGRNTNLTNEQKDTVLDLINSKDASFYFSDIRKKLELTYEKFNYDDKFKVVGNPTIKKLSSLFDKKIWEENYQDIWHCFYFYDDNQKLFEKLKKDYDYSKGIDLLVKVKLKDGYSNVSLKAIRNILPFLKKGYQYDKAVILGGVKNAFGKRWEYFKDSHYKIENEIISILSEDNAEGDAICKIKEHLSSPVFNYGFSKDDNWFAHLYHHSQEIEKASKFSDKLPIPENLRNPIVQQGINEMRRLVNSLMEKYQNQYGDDFRFDAIKVEMGRDLRNSKSARQELTFRIRDNESKNEEARARLAEYGLQQSRSNIHKYLMWKEIEEKAGAAKCPYTGKTISISDLLGTDNSVQIEHIIPYSVSLDDGFGNKTICESHFNNLKGELTPYQFYQKRSDKDLWGVNSWDAVVERAFQILPYNKAKRFTSKKDFEKSSFIERQLNDSRYIAKKSIELLSNICENVRVMPGQLTAELRHLWGINNVLQKPVVGINDHVLKLAKEETVKCYLVVDEENNVIDFVKKQNDRPNTKEGEILIPVSVNKNKLISNLFSFDIDVDDLQDGKYWIKVNLSDDFTVIPKHIDKANVDDDRIVFRGKIEKQVFKNDTSGNVNKTGKEDGVYWANFGIKDKKFVDLKTNKERPKTKSKKQIVLFGNVANDEFKCYIYNCQTNLPDGKYWLILDIDYNDVSFEKAINEKPELRNDQVCLSATVDQNSELILDVDKTYKKQVGLAPGKYYSVYQIVDYTKELCPIENQKPKVANEQKIIEGSIWVDKYTGEAKFDPKKNRDDHRHHAIDAIVIALTEQSYLQHLSTYNAKQKARQRGKIDSTEKFSLPWTGFINDVHKKADEILISYKKQNKVLTKNKKGFSVRGQLHKENVFGKRQAPNQEQGYHRRTKVTDLENNKHIAKVVDDSVRKIILDHLRYNCAVDISNPKGFKVPKDAFVKDGEWQLFLPNKRGDKVPIKKVRIKESIGNAAQLKSNINQFVNPRNNHHVVIYEDFDGNLQENVVQFWTVVERQMNGEEIYKLPHDGKNIITTLEINDMFILGLSDEEFEDNKSNYAFLSKYLYRVQKVSSMYYTFRYHLASTVTNNEQEFSIRSMKSFVEANPIKLKINEHGKLV

SEQ ID NO: 17 - protein, 1538 aa, unidentified
MAKVLGLDLGTNSIGWAVVEDEQNMILGMGSRIFPEGVKADTIGKGDKEESKNAARRNSRQQRRQYYRKRLRKIKLLRTLIDLDMCPLTHKELDGWAKWDKKKGKAGRKAPSSKQYEAWHRQNPYYLRNKALHQDLTLQEFGRVLYHLIQRRGFLSNRKGKDDGAIYKGKPGMTGITETQKQISEETLGSYLYSIYPKEGEPFKMITDENGNELRVRARYTLRDMYVEEFDKIWKRQASVLGLDNKTASKTKTQFLRGSLESNRNKKKVEKLKEKYGNENVVIEKIQDKESNKTLHKITTTTTVSLKEFLGGEIQYDEDGTLKYKSQDSVLFWQRPLRSQKGLLAKCRFEPDVKKDGKYLQKGKTPCHLSHPLYEEFRALQFINNIGYGRKQKLDETQRVQVLEIINSKDSNFNFSEIPKKLKMTYEKWNYADDQKVPGNYTTKHLSLLFESDVWESHKEEIWHSFHFFEDNDLLIEKLSNDFGLANKDFEKVENINLKEGYSNVSLKAIKNITLYLRKGYQMSDATILGGVRNAFGNRWDYFTDSHQQIEKDVLEINRTKGNQEGEAIEKIKKYLANPNNNFGFTENDKSFIRLYHHSQEIEEKPLQDKLDEVENLRNPIVQQGLNETRRIVNTLIDKYGKFDRIQVELGRDVKNSKTGRQQLANRIKENTSKNDAARQLLTEFGLKHSRENVQKVLLYKEMQERGVVTVCPYTNKSINIKDVLGRENKIQIEHIIPKSVSLDDGFANKTLCDSAFNGLKGNLTPYQFYLKNSDASLWGGATSWEEIEQRAYRLLPYPKAKRFTSKIKVEDADVKSDFIERQLNDTRYISKKTKEILSQVCKDVRVLPGALTSELRHLWGLNNILQPVMNLNIQVNELDKDKVYPHYVVLNGNQEAVNAVPIYNQKPKLGTNETTIAGKIDKGVFKTTEKYMKLKVDAPELPNGDYWLKLKLSEPKNTVRIFKERPVVDENQIVLRGKIERSKFKNDGIGTVNANRFDNGSYWATFNVLKKHFEKPQKDKQPKKTSKQLLLFGEVKGNRFKSFIYECETNESDGKYWLLLDVDVENVNFDKAISEKPEVNQNSILIEGSINDEGLFVSEIDPEHQFETQEKEGKYWVVFDIEQTPNEFSAIENLKPTLEENQTLIEGAIWVDKFTGEIKFDPKKNREDHRHHAIDALVVALSKQSYFQQLSRYNAARVAKKKLNPFDKEQLDFPEPWDGFHTAAKKKAEKILVSHKQNRKVLTEVRKRITKNGKTYTSVGDAVRGQLHKENVYGQRQAPFETTKGYHIRKNVADLKDNQLKKIVDLEIRKIIINARKEEAILKKQIDALEKQKKKVRTDEEENTLSEQINTLKNDIINLYTLKNKNGAPVPIKKVRIREKMTNAEKVKDENQWVNPQNNHHILIYRNHKGELDKDVVNFWTAVERMKQNIDLVQLPSDGIEVIETFQENDMFLLFLEDNVEISTLDDNQIFNHLYKVQKIAGADYFMEICFRQHTDSKPSTEADYVYIKGFGNGKTGWNRFNPIKVKLSETGKFEKI

SEQ ID NO: 18 - protein, 1565 aa, unidentified
MCKKQYTLGLDLGTSSLGWAVTVREGVSVGSILNAGVTVFPSGKAMVNGVEKTNNEERRNSRQARRQLARRRLRKQHLLRLLIENDMCPLTQTELDGWMKWDKSKPTEERRRFPDSPRFREWLALDPYRLRAEAVDTPLEDDPDGGFTAREKLGRILYSIIQHRGFLSSRSGKEDGSIYTSTMEKNGKVGIGETRAALGDRTLGQYLYSIAPKEGDPFSDQTDRVRSRYTLREMYVEEFDRIWTRQAPLLGLDREQVETIHYRYFTGKSDSYEARRRLERYRAKDPSAVIETLSGDEHRFVYKTTTSLKEHLAGKIWYDKDGTIRFVSKESTLFWQRPLRSQKGMLDKCSLESHKVKDRGTNRFITQGPTVVSASHPYYELYRVLQTLLNMRVRGQKLDLMMLQSMLDKLLTYGKNTKVSKLKNDLRLTADLNFPDDFELPGCPTISGITKLLDSKKMPSRKGDASPSDRLVIPFDLNLYTRIWEKLFFYDDNHLLAQGLKKIEGAVFVEDLEDKLAKLHLSEDYGNLSLHALRNILPYLLRGEELYDAVLLGGIRNAVRDVALVDDHDDALRSWIHSQGPLERGEMIDRIVALLQDPDGPFHLDTPELKLRQRLYHPSQGTAKHQLMDQLPPVENLRNPMVEQALWSMRRLVNYLLNNYRSTIRPDFTFDEIHVELARDLKLSTQKRAEMRQRQKKAEEDNDMARDYLRSQGLSTSRENLQKYKLYMEIAGRSMGVVTCPYTGKVIRGLSAALDGNSNFQIEHIVPRTVTYDDSFANKTLCDADFNRKKGNLTPWQFYQQDPSPKLWGADSWEEVERRAFRLLPYRKALRFVSKTVEDQLSGDQLVDTAYISRKAKEYLSYICPQDHIRLYPGRVTARLRQLWGLNSILSQGPTVTKDQLPTECALSTDQIPAVVIGNKKSGELSCILPKYTSRPEREEGDVVLLVRVQKGKIYSARYSELPLSWDKATSLADGTYYIVLRSTEVLRVERMFADEPRLSEDQLRARVTIELNRDGQPKLSSREMDVRILKDMPLPAAPGNYWLTIPLVSVPEVTPEHPRAKVAPDTIKVTGSVEQGEFISKVYRQGGVAMADGVAQVILTPMLSEASFTKIKLDAPVITSGEVVLTGAVDTSDLFVCDQDDELRVQIDSFDEPGKYYAVFSFDPMASYILSYEQAPKLSSDEVVLDGELIYNDKDDRIHFSPAKNRDDHRHHALDAIVIACTGQKVLTAMSTYNAHADDYERGTGNRPTFSSPWQGFRDDVRVAIESVLVKHKQSTKILNKVTKKITKDGRTHRSQGLAVRGKLHNQTFFGLRQSPQESFLSLHKRESIQNIESYSSLSRVADVTIRDLMISRLRDIGIDTTRKEYSIPKEAHAFVDEEGNYTIHLPNKRKGGAPIPVKKVRLCERSGSPIQIAGYNKWVKPENNEYLMVYTNYDNKIQYLCVPFFEEVNNKIRGVDGWKKLKDLKEVLLVLRINDMVLWGLNTNEARSILQSGDYREASKYLYRVNAISAGEFELRHHSDARTDKSSRTRITPGTLRKRGLPIKVEVSTMGELSVVRIAESDD

SEQ ID NO: 19 - protein, 1566 aa, unidentified
MCKKQYILGLDLGTSSLGWAVIVREGDSARSILNTGSSVFPSGKTMVGSKERSNNEERRNFRQARRQLARRRLRKQHLLRLLIEKDMCPLTQKELDGWVKWDKSKPSEERRRFPQSPRFREWLALDPYRLRAEAVDTPLEDDPDGGFTAREKLGRILYSIIQHRGFLSSRSGKEDGSIYTSTMEKDGKVGIDETRAALGDRTLGQYLYSIAPKEGDPFSDQTERVRSRYTLREMYVEEFDRIWTRQAPVLGLDREQVETVHYRYFTGKSDSYEARRRLERYRTKDPSAVIETLSGDEHRFVYKTTTSLKEHLAGKIWYDKDGTIRFVSKESTLFWQRPLRSQKGMLDKCSLESHKVKDRGTNRFITQGPTVVSASHPYYELYRVLQTLLNMRIRDRKLDLMRLQSMLDKLLTYGKNTKVSKLKKDLRLTLDLNFPDDYELPGCPTISGITKLLDSKKMPSRKGDASPVDRLVIPFDLDLYTRIWEKLFFYDDNHLLAQGLRGIEGAAFVEDLEDKLAKLHLSEDYGNLSLHALRNILPYLLRGEELYSAVLLGGIRNAVRDDALVDDHDDALRSWIHTQGPLARGEMIDRIVALLQDPDGPFHLSTPELKLRQRLYHPSQGTAKHRLMDQLPPVENLRNPMVEQALWAMRRLVNYLLNNYRSTIRPDFTFDEIHVELARDLKLSTQKRAEVRTRQKKAEEDNDMARDYLRSQGLSTSRENLQKYKLYMELAGRSMGVVTCPYTGKVIRGLSAALDGNSMFQIEHIVPRTMTYDDSFANKTLCDADFNRKKGNLTPWQFYQQDPSPKLWGADSWEEVEHRAFRLLPYRKATRFVSKTIEDQLSGDQLVDTAYISRKAKEYLSYICPQDHIRLYPGRVTARLRQLWGLNSILSQGPTITKGQLPAGLALSGDQIPAIVIGKKKSGVLSRILPKYTSRPEREERDVVLLVRVQKGKIYSARYSELPLFWDKAASLADGTYYFVLRSSEVVRVERMFTDQPRLREDQLRARVTIELNKDGQPKLSSREMDVRILKDVPLPTAPGNYWLTIPLVSVPEVTPEHPRAKVATDTIKVSGSVEQGVFVSKVYRQGGVAMEDGLAQVILTPDLSEVSFTKIKLDAPAITSGEVVLTGAIDRSDLFVCDQDDELRVQIDSFDEPGKYYAVFSFDPMASYILSYDQTPNLSSDEVVLEGDLIYNDKDDRIHFSPSKNRDDHRHHALDAIVIACTGQGVLTAMSTYNAHADDYERGTGTKPTFASPWEGFRDDVREAIEAVLVKHKQSTKILNKVTKQITKDGRTHRSKGLAVRGKLHKETFYGQRQSPEEPAPTMHYRVPITSITKSKQLNQVADPTIRALMIRRLQDMGVDTTQEDYAIPGNAFVDKAGNYTILLPNKRGVPVPVKKVRRRRVVNNTVQLDGYNKEVETSNNDFALIYEDQEGNYCEEVVTFWDSADRLLHGQDRVQLPMDAKRVVMTLRINDMFVIGMSEDELRSAIGSSDYGGISDHLYRVQKLSSMHYVFRLHRASTLNHDEELYTIRSFSALKESTPIKVIIDTDGTISIATRDDQANNHHQ

SEQ ID NO: 20 - protein, 1492 aa, unidentified
METTLGIDLGTNSIGFAIIDQDQTKIVNCGVRIFPEGVNKDTIGQGHKEESRNATRRMKRQIRRQYFRKKLRKIKLLEVLVQYDMCPLKSEEVRQWKNWDKHQKSATREFPDTPELREWLKLNPYELRQKAITDDITRQELGRIFYHLIQRRGFLSSRKGKEDGKIFTGKDQMTGIDETRKNLQHRTLGAYLYEISQKKGEKYKFRSERVRARYTLRDMYIREFELIWNRQAPHLGLSNEQTTQKRNIFLKGSCCNIRNRKIIDHLKLKYGKENVSIKNSRVTVLSQVPLKELLGGKIEYNGENLKFKCNESVLFWQRPLRSQKSLLSKCVFEGKHFYDYRHKKWITIGPTPAPLSHPEFEEFRAFQFINNITYGKSEHLNAAQREKVFQLMCKESKDFSFGKLQKHLNLFEKFNFDEGTKVPVCTTISQLQKLFPEEIWQQQREEIWHCFYFYDDNELLFLKLKQDYNLQIDDVEKIKKIRLAEGYGRVSLKAIRRINPYLQKGYTYSTAVILGGIRNAFGSRFEYFRDSEPEIEQDVCQILKEKNTEGEAMQKIKKYLTHHSYGFTENDRAFQRLYHHSQPLEMKELEEKLPATENLRNPIVQQALNELRRTVNKLLSDCRRKYGESFQFDHIHIELGRELRNSKSEREKQTRSIRDNEKKNEAAKLKLQEFGLQPSRDNIQKYLLYREIEEKNGIVCCPYTGKTINISDVLGSEGCIQIEHIIPYSISLDDSFANKTLCDATFNRNKGELSPYDFYHKNPSPEAWGLHCWEDIEERAFRLLPYSKAKRFIRRKNPDRNEFISRQLNDTRYMGKKAVEYLSTICKDVKAFPGQLTAELRHLWGLNNMLQSVPEISLTLTDPIPGKRKNYYVVTNDQNQVVRLSPKEADKPQTEKNELLLTGSVEGKIFKYKGIREFPTELSDGRYRLRLKLSAPSSWHLLFAEKPVSLENQLVIKGKIEKGIFVCDRLKQKIKTDLPNGSYWISLPVTKRFFVEGESVINSKLTSRQIQLFGTTREGIFQCSTYRCRTTGADGKFWCTLEVDTDHPEFTKIKNEPPYIEDDQIVLRGSVDEQGTFYADDDPHYQLTTSLPKGKYYGIFVIEHRYPELLELEYTAPKIGKNENLIEGHIWVDEHTGEIRFDPKKNREDQRHHAIDAIVIALSSLRLFQCLSTYNAKRENKKRGINSTSSFPLPWPGFIQDVSQSVEEILISYKQNLRTLCKISKTIVKEGRKIQSVGNAVRGQLHKDTVYGQRQAPGSLEKAFHIRKDIRELKTNKHIEKVVDLTIRQLIVKHLQENYHIDTTKEYTVPADAFFKDGFYRIFLPNKHGEPVPVKKIRMKEKMGNAEQLKNSLNQYVNPQNNHHVMLYTDINGNLKEEVVTFWTVIERQNQGQPIFQLPLDGQETICTLQINDTFVLGLKNEELEVYRNDRAVLSKYLYRVQKLSGMDYTFRHHLASTLKNEKDEIRIASLDAWKRANPVKVHIDEVGNLTF

SEQ ID NO: 21 - protein, 1520 aa, unidentified
MAKILGLDLGTNSIGWAVVNAEIENGKVKEYNGIDDSGVRIFPEGVVEKGTSKEISKNATRTEKRQTRRQFYRKRRRKIKLLQVLIDQNMCPLTHEELDVWKKWDSVKKVEGRKFPSTEKFKAWLSLNPYELRAKGVKEELSLEEFGRVLYHLVQRRGFLSGRKVKEDGTIFSKGKPDENILPIYATQEKIKDSTLGAYLYSILPQENKPYTTIKDENGVEIRVRGRYTVRDMYVWEFEKIWQCQNKYRNFNEVLVNYSKSRKLRGNISSSKNQKKLEKLKQKYGEQNVHIKKDTLVSYEKIPLKDLLVGNIWFEDNENGEKTLKFKSSESSLFWQRPLRSQKQYLSDCRFENNMPVIKKNGQFLMNPKGEVQTNGKKPCPISHPDFELFRAYQFINNISYGKNNRLTQEQKELVLDLINANENNFDFSKIPQKLKLTYEKFNYDDKHKVPGNYTHKKVKPLFSKEIWEKHYNEIWHCFHFYDDSDMLLEKLKKNYDFSGSIEDVKKIVLKDDYSNVSLKAIRNILPYLEKGYMYDRAVILGGVKNAFASRWDRFLTIENFEQNLERDIIAILKEKNKEGEAINKIKKYVIENQYGFVENDPFFSRLYHHSHEIEAKEIQEKVPLLENLRNPIVQQALHETRRLVNTLIKKYSEQNGVTFRFDKIHIEMGRDLRQNKSQRLDATFRNNRNNAKNNEARERLAEYGLRPSRENIQKYLLYKEIEERAGTVQCPYTGKTISVSSLLGSNNAIQIEHIIPYSVSLDDGFANKTLCETHFNKLKSEKTPYQFYQENSDSKLWGASSWDEVKDRAFKILPYAKAKRFVSEKDFESNDFIERQLNDSRYIAKKAVELLSAVCEDVRAFPGQVTSELRHLWGVNNVLSPIKNLSKAHFDVDEERRVPYYVIVNNETNEILSIQRKLNEKPKTKPQEILLPVYINKGKIESQKYSFSNEVAHLEDGKYWAHVVVENTVSFIPKYISKPIVDENAIILKGLVEKGSFKNDTLKSSFKTVEKDGYYWGTFAVTNKSFELPQKDKQPAPKTNQIVLFGNVINGVFTCYIYSCKTDFPDGKCWIILDIDPNSIVYTRANNDEPQIHENQLLLTAVVDDTAKAIVDVDKDAVFETDKQPGKYYAVFTVKSVDDFSKIENALPTCNKGERVIEGDVWLDKYTGEIQFDPKKNREDHRHHAIDAIVIALTQQNFLQRLSTEHAKMKDKERGIMNTTEKFPLPWNSFVSDVKHIAEGMLVSYKQNTKILTKNKKGVSVRGALHKETVYGKRKAPQSQQRFHIRKKITGLSNEKDIAKIVDSSIVKLIENHLRDTCGVDISKSYTIPKDAFYKDGEWRLFLPNKNGEPVPIKKIRLKEVIGNAVQLKSELNQYVNPRNNHHVIIFKDEEGNLHEQVVQFWEVVERLRNNEKVYQLPVSEYGKSTPKEIVTTLQENDMFILGLSDEEFSDNRHNHAFLSRYLYRVQKISSGDYSFRFHLASSINNKQEEVRIASIKAWEKNNPIKAVITPLGKIEPIE

SEQ ID NO: 22 - protein, 1535 aa, unidentified
MTKILGLDLGTNSIGWAVVDKEANKIIDAGVRIFTEGIVKETIGKGDKEESKNAVRRQFRQARRQHYRKRLRKIKLLQVLIAHDMCPLKEENLNKWKNWDKKTKTAGKKFPDTVEFIDWLKLNPYILRNKAINGEITRHEFGRILYHFIQRRGFLSSRKGSEDGTIYKGNDKMDGISETKSLLKDNSTLGENLYKVYPTENKPFKRTLIDNKEIRIRARYTLREMYVHEFEQIWKRQAEPLQLNNLKVSVNKDINLETSAQSPKSKQMIEKLQNKFGESNIEIKDKRVTIRKQVPFKEFLAGKIEQTDEGINFKSNESVLFWQRPLRSQKGLLGKCTFENHKFFDKAKNKWITVGSNPCPISHPDFELFRAYQFINNIRFGRSTKLNDEQKQKVLNLINKSDTSIEFGKIPKELKLTYENFNYDEKLKIPGNPTHKRIKGFFTDKQWENHYEDIWHCFYFYEDANLLKQKLINSYGLSEGEAEKASKIRLADGYGSVSLKAIRNILYFLRLGYRYSTAVVLGGVKNAFGKERWNRFETFHSEIILKVVQLVENELHKEYEVVEKLKLMLADPENQFGFVENDRSFQKLYHHSQAIEKKTVVKRLSEIENMRNPIVQKGLNEMRRLVNELLDNYSNNPEYGPSFSFDKIHVEMGRDIKNSKKMRQEMVFKIREGENENEDARLRLAEFGLKPSRENITKYRLFKEIQSKFGSVVCPYTRKTVKITDLLGRDNLYQIEHIIPFSVSLDDSFGNKTICESNFNRDKGELTPYEFYQKNSDPDLWGAESWEEVEQRAFALLPYRKAKKFVTKKRDEDSGFIARQLNDMRYISKKAAEILSEICQDVRVLPGQLTAELRRLWGLNNIIQPVLPLDLPGYNVDADLKLPHYLVLDKYGKPTEIKPILASRPEASANQILIPGFITEKKQFIADRNYRHLKFELQTDGLKAGKYWAKLNVGKPIVLTRIFTERPPINPETVVYRGKIEKGYFTHDSLGRKLKANEPDGIYWLKLQVKGKEFIEPEKDKQPKAGKNQILLFGTVQNGLFTSYIYQCETEMLPGKYWALIDVEFEKPEYAKAIINPGEVFENEIIITGTVADDGEFIADCDPGYKFDTNYPASKYFTKLTVEAIDGFFPIENPAPQLSNEQTLMEGNVWVNKSTGEIMFDPKKNRDDNRHHAVDAITIALTELVYVQKLSHYFGAIKDKERGIGDRPIFDPPWQGFDKDVKKAVDNILVSFARNNKVLSKISKTVVKSGKKYQSVGFAARGRLHREYYFGKHPSPVLHGKDPEKGGLLFEKDKDGNLVYFFHIRKPVTSIKKNKHVSKIVDQGIRDLIINRLKNDFNIDVSGSYTLPDNFFFDANKQPALFLPNKNGEPVPIKKVRLKEFIGNAVQLKDNMNQWVNPYNNHHVVIYEDFNGELKEQVVSFWDVIERQNQGDAIYKLPEDGSKLITTLQENDMFLLNLDNEQIESLSSQSISKEELSKHLYRVQKISSMYYTFRHHFASTVVNEKEELRIVSMAAWQRTKPIKVIINSTGHFSKL

SEQ ID NO: 23 - DNA, 4620 bp, unidentified
  source 1..4620: mol_type = unassigned DNA
atgagcaaaattttaggattagatttaggaactaacagcattgggtgggcagtggtggatgatacaaacaataaaatacttggtattggaagtaggatatttccagaaggagtaaaagctgatacaataggaaaaggagataaagaagaatcaaaaaatgcttcccgaagaactagtaggcaacaaagacgacaattttacagaaaacgacttcggaaaataaaattgcttagaacattaattgacttaaaaatgtgtccattaactcatttagagttagatgtttgggcgaaatgggagaaaacaaaaggaaaagcaggcagaaaagcaccagattcacccaattatgaagaatggcacagactaaatccttatgttttaagagaaaaggctttatatgaagatttgactctttttcaaatgggacgtgtgttatatcatttaatacagcgaagaggatttttgagtaataggaaaggaaaagacgatggagcaatatacaaaggaaaaccaggaatggttggcattaaagatacccaaaaacaaatttcagaaaaaaccctaggtagttttctacattcaatttttccgaaagaaggagaaccttttaagttaattaccgatgaaaatggcaatgaactccgagttagagcaagatacaccttgcgagatatgtatgtggaagaatttgaccaaatttggaacagacaagctaaagccttaggattagatgacaaaaacgcaacaaaaacaaaaatccaatttttaaatggaagtcttaaaaataacaggaacaaaaagaaaattgaaaaccttcaaaataagtatggtgttgaaaatgtaaaaattgaagagttaaaagacgggaaaagtaaaaaaacactttataaagtaactacaacaaccaatgttccactaaaaatatttttaggtggtaaaattgaacacgatgatgagggaaacgtgaagcataaaagccaagacagtgtgctattttggcagcgaccattgcgttcgcaaaaaggcttgttggcaaaatgtaggttcgagcctgattttaaggataaaaatggcaaatatttacaaagaggaaaaacgccttgccatttatcacatcctctgtatgaagaatttagagcgtatcaatacatcaacaacattgagtatggtaaaaaacagaaattagaagaaacacaacgacttcaagtattagagttaattaattcaaaagacagtaatttcaatttttcggaaattccgaaaaaattaaaaatggaatatgaaaaatggaattattcggacgaccaaaaagtagcagctaattctactactaaacatttaagtacattattttccaaagaaatttatgaagcaaacaaggaagaaatttggcattgttttcatttttttgaagacagcgacaatctaatagaaaaactaaaaaaagattttggtttagcagaaaaagacattgaaaaagttgataaaatcaatctaaaagaaggttattctaatgtttcgttaaaagcaattaaaaatattttaccctatttacaacaaggactgcaaatgagcgatgccaccattttaggtggcgttagaaatgtatttggcaaacgttgggattattttaaagatgctcacgagcaattggaaaacgatattttaaaaattaatcacgagaaaaataataaagaaggagaagcaattcaaaaaattaaaaattatttggctgacccagaaaataaatttggatttttaaaagacgacaaatcttttatcaagttgtatcatcacagccaagaaattgaagaaaaacaacttcaagatagattagatgcggttgaaaatttaagaaacccaattgtgcagcaaggtatcaacgaaactcgaagattagtaaaccaactaattggtgaatatgggaaatttgatagaattcaagttgaattaggaagagaagtaaaaaacagcaaaaaaggaaggcaagaacaaacgagaagaatttatgacaacactaccaaaaatgacgaagctcgaaaattgttaacagaatatggcttaaagcacgatagggaaaacatccaaaaagtgttgctctataaagaaatgcaggaaaaaggggtaactacagtttgtccttacaccaataaatccatcaatattagcgatgtgttgggcagagaaaataaaattcagattgagcatattattcctaaaagcgtttctttagatgatggttttgccaacaaaaccctttgcgatgccaagtttaatggtttgaaaggaaacttaactccctatcaattttatttaaaaaatagcgattcaaaattgtggggaggagcaacaagctgggaagatgtagagcaaagagcttacaaagtcttaccttacccaaaagccaaacgatttacatctaaaattaaagtagaagatgccgatgtaaaaagtagttttattgagcgacaattaaacgatatgcgttacatcagccgtaaaacaaaagaaattatgtcgcaagtttgcgaagatgtaagagttttgcctggtggtttaacagccgagttaaggcatttgtggggtttaaataatatacttcaacccgtaatgttggtcgatttgcccgattataaattagaagcaggaaaatcaataccacattatgtggtaatggatgaaaataataaacccgttttaaccttacctatttacaatgacaaaccaaaattaggagaaaacgaaacaaccattaccggcaaagtggagaaaggagtttttaaaacctccgaacaatttgtaaaacttgaagttaaaactcccgaattggtaaatggcgaatattggttgaatttaaaactttctgaaccaaaacaaatcatacgtatttttagagaaagaccagaaacctcagaaaatgaaatagttttgagaggaaaggttgaaaaggaaaaatttaaaaatgagggcattgggttggttactgccaaaggttttgataatggttcgtattgggcaaaatttaccattacaaacaaaaagtttgaagcacccgaaaaagataaacaacctaaaaaatcgggaaaacaagtattgttgtttggcgaagtaaaagaaggcttttttaaatcgtatatttattcgtgtgaaacaaatgaattagacggtaaatattggttgttgttggatattgatacagaaaatgcttcatttgaaagagccataaatgaacgtcctattgtttcggatgctaaaattattgtggaaggtagtgtcaatgatgaaggtatttttgtttctgaaattgatgctcaacatcaatttaaaaccaatgaaaaaccaggtaaatattggtttgtttttgatattcttaatgaattaaccgatttcaatgccatcgaacaacccaaaccacttttacaagaaaaacaaacattagttgaagggaatatttgggtagataaatacactggagaaattaaatttgacccaaagaaaaacagagacgaccaccgccaccacgctattgatgccttagtaatcgctttatcaaaacaaagttattttcagcaattgagtgcttataatgctgctcgtgatgctaaaaagaaattaaactcatttgaaaaagaacaattagattttcctgacccttggaatggttttcataccgatgccaaaaaagaagccgaaaaaatattgatttctcacaaacaaaaccgaaaagtactcaccgaagtacgaaagaaaattaccaaaaatggaaaaacatttacaagtgttggtaatgccgtacggggtcaattacacaaagaaaacgtgtatgggaaacgacaagctccctttgaaactacaaaaggttaccacattcgtaaaaaggtagtagatttaaaggacaatcaattggataaaattgtggacttagaaatcagaaaaattattataaatgcccgaaaagaagaagtaaccataaaaaaacacatagacgttttagaaaagcaaaaaaagaaagtgcatagcgatgaagaagaaactgctttaaacaaccaaatacaagatttgaaaaaacaaattgatttgctctacaccttaaaaaataaaaacggtgaacgagttcctattaaaaaagtgcgaattagagaagaaatgagcaatgcccaacaagtaaaggaaattaaccaatttgtaaaccctagaaataatcatcacgttttaatttataaaaaagaaaatggggatttagataaagatgtggttagtttttggacagcagttgagcggataaaacaaaataatgaaatagtacaattgcccagcgatggagttgaggttgtagaaacatttcaagaaaatgatatgtttcttctatttttagagaaagatagaaacatagaggatttaaaccaaaaggagatatttagccatttatttaaggttcaaaagattgctggagcagattattttatggaaatctgttttagaagacatacggatagcaaaatttcaaccgaagcggattatgtttatattaaaggatttagagagggcaaaacagggtggtatagttttaatccaataaaagttcaaataaccccaacaggcaaaatagaaaaaatataa

SEQ ID NO: 24 - DNA, 4590 bp, Bizionia saleffrena
  source 1..4590: mol_type = unassigned DNA
atggcgaagattttaggattagatttaggaaccaactcgattggttgggcagtagtatacgatgagaataatgagattgcaggaatgggaagtagaatattccctgagggcgttgtggcaaaaactattggtacaggcgaaaaggaagagtcaaaaaatgcagctagaagaaatagtagacaacaaagaaggcagttctatcggaaacgtttaagaaaaataaaattgctaagaactctaattgatttaaacatgtgtccgttgtcacacgccgaattagatatttgggcgaaatgggataaatcaaaaggtaagataggacgtatagctcctaattcaaaatcttatatagaatggcatatacaaaatccatattatttaagaaacaaagcattacacaaagacttaacgctttttgagatggggcgtgttttgtatcacattattcaaagaagggggtttttaagtaatagaaaaggaaatgatgaagggaaaatatacaaaggtaaagagggaatggttggtattgaccaaactcaaaaaagtatcgtaggcagtactctggggagttttttaaattcaatttatccggtagaaggcgaaccgtttaaattgattactgacgaaaatggtaatgagcttcgtgttagggcgagatacaccctacgtgatatgtatgttgaagaatttgataagatttggactattcaagcaaagcaattggggttattaggaaaggttggagaaaaaaataaactcacttttttgaaaggtaatttgagttcaaatagaaataaaaagaaaatagaaaagctgtataaaaaatacggggaagagaatgttaacattgaggaaattaaaaatgggaaatcagaaaaaagtacttttaaaatagtaacaaaatctattttacctttaaaagaattttttggcggagaaattgaatatgacgaagaaggaaaagtaagatataagagtgaagatagtctgctgttttggcaaagaccattgcgatctcaaaagggattgctgtcaaaatgtagatttgaacccactgttgaaaaaggaggcgtatatctacagaaaggaaaatcaccttgtcatctttctcatcccacctatgaagaatttagggcttaccagtatataaacaatattgaatatggtaaaaagcagaagttaaatgggatccaaagattaattgtgttggacttaataaattgtaaggatagtaattttgatttttctgaaattcccaaaaaactaaaaatgccttatgaaaaatggaattatagcgatgatcaaaaggttcctggtaattacacaacaaagcacttggctggattgttcccaaaagagatttgggaaaatcataagaataaaatatggcattgtttttatgattttaaggataatgatattttagtaaaaaagttgatttcagattttggtttagatgaaaagctatatgataaagtaaagaaaatcaatttaaaggaaggctattctaatgtttcattaaaagctgtaaaaaacatactgccatttttaagagaagggtatcgaatgagtgatgctactattttaggaggggtaatgaatgcttttggaaatctaattagctattttaaagattctgaagatgttttagtaaaagacattataaagattataagagacaaatcaaataaagaaggggaagccatcgagaaaattaaaatttatttggtggaaaataaattcggttttgttaatgacgacaaacgttttattaaattatatcatcatagtcaagatatcgaaagaaagtcgcttaaagaaaaacttgaccctgttgagaacctaagaaaccctatagtgcaacaaggtattaatgaaaccagaagattagtcaaccaactaattaaagagcatggtcagtttgatagaattcaagtggaattagggcgtaatatcaaaaacagtaaaaaaggaagacaagaacaatcaaacagaattaacgaaaatacagtaaaaaatgatgctgcaagagaattactgacagagtatggtttaaagcataatagaaacaatattcaaaaagttttgctttataaagagatgcaagacaggggagttgttactgtttgcccatacacaaatacttcaattaatataagtgatgtgctaggagcggaaaataagatacagatagaacacattatacctagaagtatttccttaaacgatagttttgccaataaaactttatgtgattcaaaatttaatacacttaaaggaaaactaacaccttatcaattttatcagaagaataacgaccctaaactttggggaggagcagagcgctgggaagatattgaacaaagagcatataaattattaccttattataaagcaaaatgcttcacttcaaaaatcaaaattgaagactcagatgttcaaagtagctttatagaaagacagttaaatgacatgcgctacattagtaaaaagactaaagaaataatgagtcaagtatgcgatgatgtgcgtgttttacctggaagtttaacagccgaattaagacatctttggggacttaacaatatacttcaaccagtaatggatttggatattactaacaataacataaaagaagatagagtctatccacattatgttgtaatggatgaagataataaggccgtttcaacaattgtgttatataataaaaagcctgcactgaaacctagagagactacgataactggaataataaataaggacgtttttaaaacaatagatgactatgttaacttaacctatgataccagggcacttaataatggaaagtattggcttaaactaaaactatcgcaaccaacaaagatggttcgaatctttaaagagcgtccagaaagtacagaaaaagaacttgttttaagaggtaaaatagatcgtgaaaagtttaagaataatagtgttggaatgattaacgcaaaaaatttcgataatggttcttattgggcgaagctagccataaaagacaaaaagtttgttttaccagaaatagagaaacaaccaaaaaaattgagaaatcaagttttattgtttggtgaagtgaaagataattattttacatcatttatttacgagtgtgaaaccaataaaatagatggtaaatattggcttttattggatgtagatttagataacgctaattttaaccgcgctattaacgaaatgccagcgtttgatttaaaccagataagaattgaaggtacactcaatagtgagggagtttttgtttcagaagttgatacagatcaccgttttaaatctaaggaaaaagcagggaaatattgggtggtgttcgacattgttgagaaaccaaatgagtttaatgctattgaaaatccaaagccaatcttagaagacaatcagactttgatagaaggtaatatttgggttgataaatatactggagaaataaaattcgaccctaaaaaaaacaggggtgatcatagacatcatgcaatagatgccttggttgttgctttatcaaagcaaagttattttcaacaattaagtgactataatgctcaacgcgaagcaaaatataaagggttgccatatgaaaaagaacaactaaattttccagaaccttggacggattttcatactacagcaaaaaataaagccgagaaaattttagtctcgcataaacaaaatagaaaagtgttgacacaagttagaaagcgcatcattaaaaacggacaaacatatacaagtgttggggatgcggttagaggacagttacataaagaaaatatttatggacaaagacaggcgccgtttgaaataagtaaaggttatcatattcggaagaaggtaagcgacctaaaagataaccagctaaaaaagatagttgatttagagattcgcaaaatcattgttaatgcaagagtagaagaagtgaaaatgcaaaagcaaataacgatattgctcacacagaaaaagaaatctcgtacagatgaagaagaattgattattgcaaatcaaatagaatcatttcaaaaagatattcaacaactttatactttaaaaaacaaaaatggagaacccgtaccaataaaaaaagtaagggtaagggaggaaatgtctaatgcgcagaaagttaaggaattaaatcagtatgtaaatccaaggaacaatcatcatattttaatttataaagatgaaaacggtgaattgaaagaatgtattaattcattttggaaagtagtagagcgacaaaaacaaaaacaaccaatatatcaactgcctaatacatacaataaaggcagaattataaacaccttagaggaaaatgatatgtttttattagggcttacagatgaggagtacaatgatagcaaaatgaactatgattttttaaccaagcatttgtttagagttcaaaaaatttctgaaggagattattcctttagacatcatttagcctccactttagaaaataaaaaggagcaagtcagaattggcagcttaaagaaatggaaagaaatgaatccaattaaagtgaaattaactgaaattggttctttaatttaa

SEQ ID NO: 25 - DNA, 4641 bp, unidentified
  source 1..4641: mol_type = unassigned DNA
atggcaaaaatactcggtttagatttaggaacaaattctatcggttgggcagttgttgatgatgaaaaaaaacaaatactcggaacaggaattagaatatttcccgaaggtgtagttgcgaaaacaatcggaacaggtgacagagaagtttcaaaaaatgctgcacgaagagaaagcagacaatctcgaagaggtttttacagacaccgtttaagaagaattaaactgttagaaacattgattgaatttaaaatgtgccctttgaccggtaaagagttaaaacaatggaaacagtacgataagaaaaaaggacaagccgggaagacttttccgagttcagaggaatttaacaaatggctgaaattaaatccttataaattaagagcaaaagcattaaacaacgatataagtctttttgaattaggcagaattttttatcatcttatacaaaggcgcggttttttaagtaaccgaaaagggaatgaagaaggtaaaatattttcaggtaaagataatatgaccggtattgatgaaaccaataaacagattaaagataaaacacttggcacatatctgaattctgttatcccgaaagaatatgaaccatataaaaatataattgacaaaaacggaaaagaattacgtgccagaggcagatatacgttaagagatatgtatgttgatgaatttgagaaaatttggcaaaggcaatcgaaacaactcgggcttaattctatacaaaaagaggtaaaaaaagaaagtattatatccggttcaatctataataaacgaaacaagcacagaattaaaaatttacaaaaaagcaaaggcaaagaaaatgttgaaataaaaggcaatatactgacaataaaagaacatattcacttaaaagaatatctcgccggtaaaatttattacgatgatgaggggaatttaaaacataaaagtcaggacagtgtcttattttatcaacgaccgctgcgttcacaaaaaggattacttgcaaaatgcagtttggaaggcagaaagttttatgacaaaaaagataaacgttggcgaacagcaggtccgacaccgtgtccgttgtcgcatcctgaatttgaagaattcagagctttacagtttataaacaatattgaatacggaatcaaaaagagacttgatgaaaatcaaagattgattttgttgaatttatttaacagaaaagatacaaattttgattttaagctaatcccgaaagaattaaaactaacttatgaaacatttaactatgcagatgatttaaaagcaccgggaaattatacaaatgccaaactcagtaaacttttcaatcccgatatttgggaaaagcacaaagagaatatctggcattgtttttatttttattctgataatgataaacttattaataaactaaaaacagattttggtttagaagaaaaaaacgaagaaaaagcagcaaaaattaagttaaaagacgattatgcaaatgtatcattaaaagctatcaggaatattacacctttcttgaaaaaaggatataaattaagtgatgcagttttgcttggaggtgttcgtaatgcttttaaaacaactgatacagaaaacaaaccctatgacaggtgggagaattttaacaacgaacatgaaaaaattgaaaaagatattatacgtataaataaagataagaaaaacaaagagggcgatgcagtaaaaaaaataaaggagtatttacataacaattgcggttttgaaaaagacgataaaaactttaaaaaactgtatcatcacagccaagaaattgaacaaaaaaagatacaagacaaacttgataaaattgaaaatttaagaaatccgattgttcagcaaggcttaaatgaacttcgcagacttgtaaataccttgattgatgaacacggtaaatttgacaaaataaaagttgaactcggaagaaacctgaaaatgggtaagaaacaacgtcaagaaactgaatttaaaataagagataataataagaaaaatgaagaggcaagagagaaactttccgaatacggtttagcccattcaagagaaaatattcaaaaatatttattgtggaaagaaattgaagatcgaagcggaacggcaaaatgtccttataccggaaaaactataaacatcagtgatttactcggtaaagaaaacaaatttcaaattgaacatatttttccgagaagcacatcacttgatgacagttttgcaaataaaacattttgcgatgcaaaatttaacggattgaaaggaaataaaacgccttataatttttataaaaaaaacaacgatgcaaaactctggggcggtgcaaagtcatgggatgaaataaaacaaagagcatttaaacttttaccgtacaaaaaagcaaagcgttttactgctgaaaaagttgatgaaaaagcaggaggttttattgaaagacaattgaatgatacacgctacatgagtaaaaaggcaaaagaaatactttcacaaatttgcccgaaagacaacataagagttatgcccggtagtgtaacatctgaattaagacatctctggggattgaataatattttacaacctgttgagatattagactttaaagaggcaaaaattaatattgataaactcgaaaagcattgggtagtttttgatgaaaaaggggaagttttaagtttttatcctatacataatgaaaaaccgaatttaaaaccgaatgaaattacaatttcaggttatttgaataataaaggtgaattttcttcaaaatatattgccttaaaatcagtatatgaagaattaaaaggtgataattttaaaggagaatattggaaaaaattaaaattatcaaataaaccgttacaaactgttaagatttttacggataaacctgctgtagatgatgatgaaattgttttaagaggaaaagttgcaaaaaaatattttgaaaacgatacaatcggaaagaaaattaaaacaaattttgaagacggctattattgggcaaaatttaaggtaaataatgttaaatttgaaaaacctataccgaaaaaacaacccaaaaagaagtcggggcaaattttattatacggaactgttgaagatgcaatttttaaatcatatatttttgaatgtcaaacaggagaggctgacggcaactattgggcaataattgatttagatttcgacactgttgatttttctaatacaatgaacccaaagcctgaaactgatgataataaaattctgatacaaggcagcataaacgaaaccggtttgttttcctctgatattgataataagcataattttgaaacaactttacctcaaggaaaatattggactatttttgaaattgaaggagaagatgcaaaactttatcctcaaataaatcttgaaccgaaattgcaggataaacaaaaagttgttgaaggaactgtttggatagataaatatacgggtgaaataaaatttgacccgaaaaagaacagagatgatcatcgacaccatgcaattgatgcaataactattgcacttacggaacaatcttatttgcaaaaattaagcacatataatgcacaattagatgaaaaaagaagaggcaaagcgaaaaaaccgacttttgaaatgccttgggacaatttctataacgatgcaaaaaaagcagcttccgaaatgctgatttcgcataaagcaaataataaggtcttaacaaaaattaataaagtaattcataaaaatggaagaacatatacaagtaaaggctatgctgccggagggcaattacataaagaaaacgtttacgggaaacgccaaactcccggtagtaaaaaaccgtattatcatatccgtaaatcggtagaaagcttaactaccgataaacaaattgataaaatagttgacggaaaaataagagcaattatcctacatggaaaagcacaagaaaaaagaataaaaaaagaaattgatgaattaaacaaacagtttaaaaaagcaaaaacagattttgaaaaagcagaaattcaaaagcaaataaaacaaaaacaacaagacataaaaatgttattttctttgccgaataaaaatggagaacgggttccgataaaaaaagtgagggtaaaagaaaatctcggtaatatgatgcaactgaaagataaaaaggtgtttgcagaaaagaaaggcaaagaagttgagctaaaccaatatgttaacccgcgaaataaccaccatgttgccatatataaaaatacggaaggggaattatttgaaaaagtcgttacattttgggaagccgtagagcgtaaaaaagaaggattatccgttattgataaaaacccgacagaaggcagtgtatttgttacatctttagaaattaacgatatgtttttattagggataaatgaagacaatattaattgggaaaatccgaattattcagatttaacaaaatacatttacagagtacaaaaagtatcatcaagttattatacgttcagacatcatcttgcgtcaaaacttgataattcaaagcaagaaattagtttgcaaagttttaaagcatggcaggaagtaaacccgattaaagtgaatattgatatactcggcagaatatcaaaagttgattga

SEQ ID NO: 26 - DNA, 4665 bp, Flavobacter branchio
  source 1..4665: mol_type = unassigned DNA
atggcaaaaatattaggattagatttaggcacaaacagcattggctgggcggtggtggatgacacaaaaaacaaaattattgacgctggagtaagaatttttccagaaggtgtgaaatcagataccatcggaaaaggagataaggaggaaagcaaaaacgctaagcgaagaacaagcagacaaaccagaagagggttttacagaaaaaggcttaggaaaattaaactattaagaacacttataaattttaatatgtgtcctttgaaacatgaagaactagatgtttggagtaaatgggataaaattaaaaagaaagaaggtagaatggcacctgaacttttcttacctgataatcatccttattgtttatggttaaagcaaaatccttatgaattaagaaataaagcactaattgaagatgtaacactatatgaatatggaagagtcttatatcatttaattcaaagacgtggttttttaagtaatagaaaaggaaaggatgacggtaaaatttacaaagggaaagaaggaattaaaggaatagaagatacacaaaaggagatacagaactctactttaggaaaatatttgtttgatattttgccaaaagagggagagccatttaaaattatacaagatgaaaagggtaatgaattaagggttcgttctcgatatacattacgagatatgtatattgctgaatttgaagcaatatggaatagacaagcaccacagttaggacttgataagattgattacttaaatgagaaaaagattttactaaaaggaggattaaaaaataaaaggaataataataaaattgaatatttcaaaaagagtaaagggatagataatgtagaaattattgaaataaaagatgaattgtctaatacggataaagaaaaatttattttaaaagtaatttcaaaaatacctttgaaagagtttttaggagggagaattgaaaaagatgaagatgaaaatatcaaatttaaaagcaatgatagtgttttgttttggcaacgcccattgcgatctcaaaaaaatttactttctaaatgtcgatttgagccagatttaaaagatgacaatggaaagtttattcaaaaaggaaagacaccatgtcatttatcacatcttttgtttgaagaattcagagcttatggatatattaataatattgaatatggaaaaaaacaaaaattagaagaaactcaacgggagcaagtcttagaattaattaattctaaagacagtaatttcaagttttctgaagttaaaaagaaattaaaattggagtatgaaacttttaattttgatgacgatgctccggttaatggaaattatacaacaaaacatctaaattcattatttccaaaagaaattcttgaaaaagaatatgttatcaaagttaataatattgatattgttgaatatggttatgaacgcatttggcatttatttcattattgcgatgataatgatgttttaatcgaaaaattgcaaaaggaatatggtttagaagaaaagaatattgaaaaagtttctaaaacaaatttaaaagaaggatattcaaatgtttctttaaaagcaattaaaaatattttgccttatctaaaggaaggaagaagattgagtgatgcggtaattcttggtggcgttagaaatgcttttggaaaaaagtgggaattatacaaagaagatcataaggaattagagaaagatttattgaaaataaatcaagataaatctaacaaagaaggcgaagccatagaaaagattaaacattatttaatcgaaaataaatatggttttgttgaaaatgatattagatttaaaaagttataccatcatagtcaagaagtgatcgaggttaaacaaaaagataaaatagaagatattgataatttaagaaatcctatagtccaacaaggagtaaatgaggtaaaacgattagtcaatttattaattgaagaacatggtaaatttgaccaaataagggttgagttaggaagagatttaaagaacaacaaaatgaaacgtcaagaattaacttataaaatctctcaaaacactgctaaaaatgaaaatgcaaaacagctattaaccgagtatgcattaagacatagtagagaaaatattcaaaaagtactattgtatcaagaactgcaagaaagaggaactattgctgtttgtccgtacacaaataaaacgataaatatttctgatgtactaggaagggcaaacaaaattcaaattgaacatataataccaaaaagtatttcattggatgatagttttgctaataaaaccttatgtgatgcaaaatttaatggactaaaaggtgagcgcactccttatgaattttatcaaataaataaagatgtaaatttatggggtggtgcaaaaacttgggaagaaattgaaagaagagtttttactgtgttgccttatcctaaagcaaaacgatttacttcaaaaacaaaatttgataaaaacgattttatagaaagacaacttaacgatactcgttatataagtaaaaaaactgcggaaattttatctcaaatttgtggtgatgttcgagtaatgccaggaagtttaacagctgaattaagaagactgtggggcttgaataatattttacaaccagcctttacaattgatattccaaatttgcatattgacgaaaataaacacattcctcattatgtagttttagataaagataaaaatccaattgcttctcaaaaaatttataatgaaaaacctaaattaaaaccaaatgaaacaaccttgtttggtaaagttgataaaggtatttttaaatcaaaagagcaatatataaaatttcaagttgacacgcctgaattaataaatggcgaatattgggcaaaactgttgttgtcaaatcctaaaagtattgttagggtttttaaagaaaaaccagaaaatacagaagatgaaatagtagttagaggtaaaatagagaaagaaaagtttaagcatgaagggttaaacacaatcaatgcacaagggaaagacaatggtgtttattgggcaaaactaactatttcaaataaagaatttaaaaaacctataaaagaagaccaacctaagaaaaatggaaaacaaatcttgttatttggagaagtaaaagaaggtgtattttcttcttacgtatatcaatgtgatgctgaaaaagtagaagatggtaaatattggttagtccttgatattgattttgaaaaagttgattttgaaaaagctttaaatgacaaatcaaccgtaaaagaaaatcaaattgtaatccagggaattacaaatgatgataatgtatttgtttctgatatggacaaccaacatcagtttacaatgaatgaaggcaaaggcaaatattatgtattgtttgatattatttctgaagtagatgatttttatcctgtaaaaacggaaatccctaaaatagaagaaggacaagcattagtggaaggtactgtgtgggtagataaatattcgggtgaaataaagtttgaccccaaaaagaatagagaagaccatcgtcatcacgctatagatgctttagtaattgcgttaacaaaagaaacttattttcaacagttaagtaaacacaatgcctcgtctgaaaacaaaaaaagaggcatagaatatgataacgaccaattagattttgcagaaccatggaatggttttcataccgatgctcaaaaaacaataaatagtattttaatttctcataaacagaatagaaatatactaactaaaatcaccaagaagattacaaaaaatggtaaaacctataaaagtgaaggaatggcggttagaggacaattgcacaaagaatttgtctttggttttagaactccaccaaatggagatgaaggttatcacataagaaaatctgtggaaagtttggttacagataaacagataaataaaattgtagacacaagcatcagagaaataattataaaagcaagaagagccgaaattccattgtataaagaaattgaaaacttgtcaaaacaattgaaaaatccaaggctatctgaaatcgaagaaaaaaacattaatgataaaataagtcaattaagaaatgatattaaacaattatatacgttacctaataaaaaaggtgatccaattccaataaaaaaagtaagagttgccgaagaaatggggaaagcacagaaattaaaatcttctataaatcaatttgttaatccaaggaataaccatcatattgttatttataaagatgaagaaggacagcttaaagacacagttactaatttttggaaagtagtagaaagagtaaaacaaaaagaagaaatttataggattccatctcccgtagttggtcaatctgttccaaaagaacttattttaacctttcaagaaaatgatatgttcatacttggactttctgaagatgaatataatgataacattaataactcatcatttttaagtaattatttgtatagagttcaaaaaatatctgatggagattattcattcagacatcatttagcatccacagtaacaaataaaaaagaagaaataaggattgcaagtatgaaaaaattccaagaaatgaacccaattaaaattgtagttaatatattgggtaaaaacaagtaa

SEQ ID NO: 27 - DNA, 4857 bp, Flavobacterium ajazii
  source 1..4857: mol_type = unassigned DNA
atggcaaaaattttaggattagatttaggtacgaactcgattgggtgggcggttgtagatgatatggataaaaaaataattgattcaggagtgcggattttccctgaaggagttgttgcaaaaactattggtactggagataaagaagaaagcaaaaacgcaagtaggcgaaatagccgtcaacagcgacgtggtttttatcgtaaaaggttaaggaaaataaaattactgagaactcttattaatcttgatatgtgcccccttaaaaacgaggaacttgatatttggagtaaatgggataagcagaaaaagaaagaaggcagaatggctccagaaattcatttacctgaaagtcattcttattgtttgtggttgaaacaaaatccttatttgttaagagataaatcattaaatgaagatttaactctacaagagtttggcaggattttatatcatttaatacaaagaagaggttttttaagtaatagaaaagggaaagatgatggaaagatttataaagggaaagatggtgtaaaaggtattgaggaaactcaaaaagaattacaaaattcaacattaggaaaatatttgttcaatattttgccaaaagatggagaaccttttaagttaattcaggatgaaaatggtaatgaattgcgagttcggtctagatatactttgcgcgatatgtatgtagcagaatttgaagcaatttggaatagacaggcttgcaatttaggtcttgaaaccattaagtgctcaaatgagaaaaaagttttgttaaaaggcggtttgaaaagcaaaagaaataatagtaaaatagaacatctaaaaaagataaaaggagaagagaatattgaagtaattgagttaaatgataaattgtcaaaaaatgataagcagaaatatttgcttaaaattaaaacaaaactttctttaaaagaatttttggcgggtagaatagacaaagatgaggagggagttgtgaaattcaagagtaatgaaagtgttttgttttggcaaagagctttacgttcacaaaaaggacttcttgctaaatgtagatttgaacctgatttgaaagatgaaaatggtaagtttatacaaaagggtaaaactccctgtcacatatcacatcctttatatgaagagtatcgtgcattacaatatatcaataacattgaatatggtaagaaacaacgattggaagaatcacagcgattgcaagtgttggaactaataaattctaaagacaagaaatttgattttatagagataaaaaagaaattgaaattggaatatgaaacttttaattatgaagacgactcactagttgttggtaattatacaacaaaatatctgaatgctttattcccaaaatcgatacttgaaaaagaatttttaataatagtaaataaggaagagaaattagaatatggctatgagcgtatttggcatttatttcatttttgtgaagacaatgataagttaatctgtagtttagaaaaactgattaaagagacaaaaatagattatattttaaataaagcaataattgaaaaaatagaaaaagtaaatttaaaagaagactattcaagtgtttctctgaaagctataaaaaacattttgccctatctaaaaaagggattgcgtttaagtgatgctgtcatattagcaggtgtcagaaatgcttttggtaaaaaatgggaattatataaaaatgaccatattgaattagagaaagatttattgaaaataaatcacgaaaaatccaataaagaaggagaagcaattgagaaaataaaagtatatctaattgaaaacaaatatggctttgtcgaagaggatgttcgatttaaaaaattgtatcaccatagccaagagattattcaagtaaaacaaaaagataaaattgataccgttcaaaatttaaggaatccaattgtgcaacaaggagtaaatgaaactaaacgtattgtaaatgaattgattgctcaaaaaggaaagtttgatcaaattaggattgaacttggacgtgatttaaagaatagtaaaacgaaaagagaagaattaggttataaaattagagaaaatgataagaaaaatgaaaaggcaaaggaattattgatagaatttggtttgcgacaaagcagagagaatatccaaaaagtgttattgtatcaagaaatgccgatttctatttgtccttatactaacaaaacgataaatattaatgatttacttggtaggggaaataaatttcagatagaacatatcattccaaaatcaatttctttagacgatagttttgcaaataaaacattgtgtgattcaaaatttaatggtttaaaaggaaatagaacccctcacgatttttatgaaattaacaaagatccaaatctttggggaggtgcgaaatcatgggaagaaattgaacgacgtgtttttaaggttttgccctatccaaaagcaaagcgatttgtgtcgaaaacaaaatttgaaaccaacgattttattcaacgacaattaaatgacactcgttatataagtaaaaaaacgagcgaaattttatctcaagtttgttccgatgttcgtgtgatgccaggaagtttaaccgccgaattgcgtcgactttgggggttgaataatattttacaacccattattacagtagatattccaaatttacatattgaagagaataaacatataccgcactatgttgttttagacaatgataaaaagcctgttttgtcacaacgaatttattatgacagaccaacaactaataaaaatgaaacacttttatctggaaatatcgataagggagtttttaaatcgaaggaacagtatgttaaatttcaggttgatgcaccagatttagaaaatggcgattattgggcaaagttagttttgtctaggcccaaaaatattgtacaagtatttaaagacaagccaagtactttagaaaatgaaattgttttaaggggtaagattgaaagagataaattcaaaaatgatggattgaatgctataaatgcgactggcaaggaaaatggaatttattgggcaacatttactattgaaaaaaaagaatttaaaaaacctgctaaagaagagcaacccaagaaaaatggaaagcaaatacttctatttggggaagtaaaagacggtcttttctcctcgtatatttatgaatgcgaaacagaaattgaagatggaaagtattggcttatcatagatgtggattttgaaaaggttgattttgaaaaagccttaaacgaaagacctttagttagagaaaatcaaatattgattcaaggaactacaaatgatgaaaatatatttgtttctgatatggataatcaacatcattttgaaatggaagaaggcaaagggaaatattatgttttgtttgacatcgtttctgaagtaactgaattttcacctgttaaaaccgctaaaccagttcttgaagaaggacaaacaatagttgaaggtacaacttggattgataaatatacgggcgaaataaaatttgacccaatcaaaaaccgagaagatcatcgccatcacgccattgatgctttagttattgccttaacgaaagaatcttttttccaacggttgagcaaacaaaacgcttcaactgaaaacaaaaaaagagggaatgaacacgaaaaagaacatttagattttccggaaccttggaatggttttcataacgatgccaaaaatgtcataaatggtatcctgatttcccacaagcaaaacaaaaacatcttaacaaaaatcaccaagaaaattactaaaaatggacaaacgtttaaaagtgagggtatggcagtaagagggcaattgcacgaaactactttttatggaaagaatttacattgtaaagagaaggaaatcatacagcgtttaccaatttcaaagttgaaatttgcgccagcaaaaggaggtggagcaagtatttatgatataattgatgataatattagaaaattaattatttcaagtgttacaaattctttcaaacaaattcttcaaaaatataatgatgtaagtacaacctttgaggaaattaaagagttaaagaaaaaatataataaaggtaaaattgatgaagccatagaaaaactaggtgtaattgaaaaaatggaaacagattcgaatagattacaaaaaaaatcagaaattgataaaaatcagaaagaaataaataggattaaatcaacaataagcgaaacaattgaaagttttctaaaagatgagacgttttatttgccaaatgaaggaaaaagattcaaacggctaaataaaattgacgttggcaaagaaagaaatcctgttccaataaaaaaagtacgaataaaaaaagtaatgaaaaatgcagaaacaataaatcaaaaaattggacagtatgtaaaccctggaaacaatcatcatatattaatctataaagacgaaaatgaggagttgaaagatgatattactaatttttggaaagttgtggaaagagtaaaacaaaaagatgaaatctataaactacctgaaatagaaattgataaaccagcccctaaagaacttatattaacttttcaggaaaacgatatgtttttgttagggatttctaatgatgagtacaatgacaataaactagaccatacttttttaagtaattatttgtatcgggttcagaaaatttcagatggagattattcgtttaggcaccatattgcatcaacagttacaaataaaatggaggagattagaataacaagtatgaaaaagtatcacgagatgaatccaataaaagtagaaatttctgtcttaggaaaaatttcaaaaatgtaa

SEQ ID NO: 28 - DNA, 4677 bp, Flavobacterium alvei
  source 1..4677: mol_type = unassigned DNA
atggcaaaaatattaggattggatttagggacaaactcgattgggtgggcgattgtggatgatgttgataaaaaaataattgattctggagtgcgaatttttcctgaaggagttgttgcaaaaactattggaacaggagataaagaagaaagtaaaaatgcgagtaggcgtaatagtcgccaacaacgacgtggtttttatcgtaaaaggttaagaaagataaaattattaagaactcttattaatctcaatatgtgtcctcttaaaaatgaggaactagatatttggagtaaatgggataaacaaaaaaagaaggaagtacgaatggctcctgaaattcatttgcctgaaaatcattcttattgtttgtggttgaagcaaaatccctatttattaagagataaagcattaaatgaagatttaactctacaagaatttggtagagtgttgtatcatttaattcaaagaagaggttttttgagcaatcgtaaaggaaaagatgatgggaagatttataaaggaaaagacggtgttaaaggtattgaggatactcaaaaagaattacaaaattctacattaggaaaatatttgttcgatattttgccaaaggatggagaaccttttaaagtaattcaagatgaaaatggtaatgaattgcgagttcgttcaagatatacgttgcgtgatatgtatgtggctgaattcgaaacaatttggaataggcaagctgcgaatttaggtcttgaaaccattgaatacttaaatgagaaaaaagttttgctaaaaggcggtttaaaaaacaaaagaaataatagtaaaatcgaacatctcaaaaagataaaaggagaaaacaatgttgaagtagttgagttgaatgatgaattgtcaaaaaatgataagcagaaatatttacttaaaattaaaaccaaacttcctttaaaagaattcttggcgggtagaatagataaagatgaggagggagtcttgaaatttaaaagtaatgatagtgttttgttttggcaaagatctttacgttctcaaaaaggacttttagctaaatgcagatttgagccagatttgaaagatgaaaatgggaagtttatacaaaagggtaaaacaccttgccacatatcacatcctttatatgaagaatatcgagctttacaatatatcaataacattgaatacggtaagaaacaacgattggaggaatcacagcgtttgcaagtgttggaattaataaactcaagagacaagaaatttgattttatagagataaaaaagaaattgaaattagaatatgaaacgtttaattatgaagacgatgctccagttataggtaattatacaacaaaacatttgaacgctttatttccaaaatcgatacttgaaaaagaattttctataaccgtaaacgaggaagaaaaaacagaatatggatatgagcgtatttggcatttatttcatttttgtgaagacaatgataagttaattagaggtatagaaaaacttattaaagagtctaaaatagattatgttttaaatgaagcaataattgaaaaaatcgaaaaagtaaatataaaagaagattattcaagtgtttctctgaaagccataaaaaatatcttgccctatctgaaaatgggattgcgtttaagcgatgctgttatattagctggagttagaaatgcttttggcaaaaaatgggaattatataaaaatgaccatattgaattagagaaagatttgctcaaaataaatcaccaaaagtcaaacaaagaaggagaagcaattgagaaaataaaagtctatttgattgaaaataaatatggatttattgaagatgatgttcgatttaaaaaactgtatcaccatagtcaagaggtagttcaagtaaaactaaaagataaaattgatactgttcaaaatttgagaaatccaattgtgcaacaaggagtaaatgaaactaaacgccttgtaaatgaattgattgctcagcaaggaaagtttgatcaaattagagttgaacttgggcgggatttaaagaatagtaaaacgaaacgagaagaattagggtataagattagagaaaatgacaagaaaaacgaaaaggcaaaagaattattgattgaatttggtttgcgacaaagtagagagaacattcaaagagtacttttgtatcaagaaatgccaatttctatttgtccttatacaaacaaaacaataaacatatcggatttattaggaggtaaaaataaatttcaaatcgagcatatcatcccaaaatcaatctctttagatgatagttttgcaaataaaacattgtgtgattcagaatttaatggtttaaaaggaaatagaactcctcacgatttttatgaaattaataaagatccaaatctttggggaggtgcaaaatcgtgggaagaaattgaacgacgtgtttttaaagttttgccctacccaaaagcaaagcgatttgtgtcgaaaacaaaatttgaaaccaacgattttatccaacgacaattaaatgacactcgttatataagtaaaaaaacgagcgaaatcttatctcaagtttgttctgatgttcgtgtaatgccaggaagtttaacagccgaattgcgtcgactttgggggttgaataatattttacagcctattatgacggtggatattccaaatttacatattgaagaaaataaacatatacagcactatgtagttttagacaatgataaaaagcctgttttgtcacaacgaatttattatgacagaccaacaacaaatgaaaatgaaacacttttgtctggaagtattgaaaagggagtttttaaatcgaaagagcagtatgttaaatttcaggtagatgcaccagatttagcaaatggcgattattgggcaaaactagttttgtctaatccaaaaaatattcttcaagtatttagagacaaaccaagcactttggaaaatgaaatagttttaagaggtaagattgaaaaggataaattcaaaaatgatggattgaatgccataaatactactggcaaggaaaacggaacttattgggctacattttcaataactaataaggattttaaaaaaccagttaaagaagagcaacccaagaaaaacggaaagcaaattcttctatttggagaagtaaaagaaggtcttttttcttcctatatttatgaatgtgaagcagaattagaagatggaaagtattggcttatcatagatgtagattttgaaaaggtttcgtttgaaaaagcattaaacgaaagaccgcttgtaaaagaaaatcaaattttgattcaaggcactacaaatgatgaaaatacatttgtttcggatatggacaatcaacatcattttgaaatggaagaaggcaaagggaaatattatgttttgtttgacatcgtttctgaagtaactgaattttcgccagttaaaactaccaagccagttctcgaagaaggacaaacaatagttgaagggacaacttgggttgataaatatactggtgaaataaaatttgacccaatcaaaaaccgtgaagaccatcgccatcacgccattgatgctttagttattgctttaacaaaagaatcctttttccagcggttgagtaaacaaaatgcctcaactgaaaacaagaaaagagggaatgaacacgaaaaagagcatttagattttccagaaccttggaatggatttcataccgatgccaaaaatgtcatcaatagtattctaatctcacacaaacaaaacaaaaacatcctaacaaaaatcaccaagaagattaccaaaaacggacaaacttttaaaagtgtaggtatggctgtgagagggcagctgcacaaagaaaatctgtatgggaacaggacaccacctaatggagatagtggatttcatatcagaaaatctattgatagtttaaaaacggataaacaaatagaaaagattgttgatattaaggttaaagaaataattataaaggcaagaaaagcagaaattcctttgtataaagaaattgaacaactttcaaaacaattaaaatcaccaaaaaacacagaaatagaggaaaaggagcttaaagaaaatattgaagcattgcatactaaaattaaattgctttatactttacctaataaaaatggtgaacccgttccaataaaaaaagttagagttagtgaggaaatgagtaatgcacagcaattaaaatcaaaagtaaaacaatatgtaaacccaagaaacaatcatcatatagtaatctataaagacgaaaatgaggagttgaaagatgatattactaatttttggaaagtagtagaaagagttaaacaaaaagatgaaatctataaactacctgaaatagaaattggtaaaccagctcctaaagaacttgtgttaacttttcaggaaaacgatatgtttttgttaggactttctaatgatgagttcaatgataataaacaagactttacttttttaagtaattatttgtatcgagttcagaaaatttcagatggagattattcatttagacaccatctcgcgtcaactgtcacaaataaaatggaggaagttagaatagcgagtatgaaaaagtatcaagaaatgaatccaataaaagttagaatttcatttactggtaaaattgaaaagttgtaa

SEQ ID NO: 29 - DNA, 4656 bp, Flavobacterium orientale
  source 1..4656: mol_type = unassigned DNA
atggggaaaatattaggtttggatttggggacaaatagtattgggtgggcattgattgatgatatagaaaataaaattattgactcaggagtgcgtatttttcccgaaggagtagttgcaaaaactattggacaaggtgagaaagaagaaagtaaaaatgcaagtcgacgaaatagtcgtcagcaaagacgtggtttttatagaaaaagattacgtaaaattaaattattgagaacacttattaatttaaaaatgtgccctttgagtcattctgaactagatatatggagtaaatgggataagaaaaaaaagaaagatggcagaatggcacctgaaatccatttaccagaaaatcatccttattgtatatggttaaaacaaaatccatatttgttgagagatagagctttaaatgaagatatatctctacaagaatttggtagagtgttatatcatttaattcagagaagaggttttttgagcaatcgcaaaggaaaggaagatggaaaaatatataaaggaaaagatggagtaaaaggtattgaggacactcaaaaagaattacaaaattcaactttgggaaaatatctgtttgatattttgcctaaagatagcgaaccttttaaagtaattcaagatgaaaatggtaatgaattgcgtgttcgctcaagatatactttgagagatatgtatatagccgaatttgaagcaatatggaatagacaagcctataatttggggcttgatattattgaatatactaacgagaaaaagatattgttgaaagggggtctaaaaaataaaagaaacaacagtaaaattgagcatttaaaaaagattaaaggagaaaataatgttgaagttattgaattgattgatgaattatcgattaatgataatcaaaaatatttattgaaaataaaaactaaacttcctttaaaagattttttagcaggaaaaattgaaaaaaacgaagaaggagaattaaagtttaaaagcaatgaaagtgttttgttttggcaaagacctttgcgttctcaaaaaggacttcttgctaaatgtagatttgagcctgatttgaaagatgaaaatggtaaattcatacaaaagggtaaaacaccttgcccattatcacatcctttatatgaagaatatcgtacattgcaatatatcaataatattgaatatggaaaaaagcaaagattggaagagtcacagcgtttgcaagtattggaattaattaactctaaagaaaagaaatttgattttatagaaataaaaaggaagttaaaattagagtatgaaacatttaattatgcagacgactcaccagttattggcaattatacaacaaaacaactgaatgtattatttccaaaatcaatacttgaaaaagaatactcaataacagttaatgatcaagaaactatagagtatggttatgagcgtatttggcacttgtttcattattgtgatgataatgatatattgttggaaaaacttcaaactatttatggtttaactgatactgataaagaaaaaatttctaaaataaatttaaaagaagattattccagtgtttcattaaaagctataaaaaacattttgccttatctaaaaaaaggattgcgtttaagtgatgctgtaattttgggtggtgttagaaacgcttttggtaaaaaatgggagttatataaaaatgaccatttcgaactagaaaaagatttattaaaaataaatcacgaaaaatctaataaagaaggagaagctattgaaaaaattaaagattatttaattgaaaataaatacggatttgcagagaatgatattcgatttaaaaaattgtatcatcatagtcaagaggttattaaagtagagcaaaaagataaaatagatactgttgaaaatttgagaaatcccattgttcaacaaggcgttaatgaaacaaaacgattagtaaatgaattgattacacttcatggaaaatttgaccaaattaaagttgaacttggtagagatttaaagaacagtaaaactaaacgtgaagaattaggttataaaataagagaaaacgataaaaaaaacgaaaaggcaaaagaattattaattgaatttggtttgcgacaaagtagagataatattcaaaaagtcttgttatatcaagaaatgccaatttctgtttgtccttataccaataaaacaataaatatttcagatgtactcggtagggaaaataagtttcaaatagagcatattataccaaaatcaatttccttagatgatagttttgcaaataaaacattatgtgattcaaaatttaatggtttaaaaggaaatagaacaccttacgatttttatgaaatcaataaagaccctatactgtggggaggtgctaaatcttgggaagagattgaaatacgagcttttaaagttttgccttaccccaaagcaaaacgatttgtatctaaaacaaaatttgaaaccaatgattttatacaaagacaattaaatgatactcgttatataagtaagaaaacaagtgaaatcctttcgcaagtatgttcagatgttcgtgtgatgccaggtagtttaactgcggaattgaggagactttggggcttgaataatattctgcaacccataatgacggtagatattcctaatctacacattgaagaaaacaaacatatacctcactatgtagttttagacaatgaaaaaaaaccaattttatcacaacgaatttattttgataaaccaattacgaaagaaaatgaaacacttttgtctggaagtattgaaaagggagtttttaaatcgaaagaacagtatgttaaatttcaagtaaatgcaccagaattagcaaatggcgattattgggcaaaattagtattgtctaatccaaaaaatattgtccaagtatttaaggataagccaaatgctttagaaaatgaaattgttttaagaggtaaaattgaaaaggaaaaattcaagaatgatggtttgaatgccataaatgctattggtaaggataacggaacgtattgggcaacattttcaataattaatagggattttaaaaaacctgttaaagaagagcaacccaaaaaaaatggaaagcaaatacttctatttggggaagtaaaggaaggtcttttttcttcgtatatttatgactgtgaggcagaaattgaagatggaaagtattggctcattatagatgttaattttgaaaatgtttcttttgaaagagcattaaacgaaaaaccgttggtaaaagagaatcaaattttgattcaaggaactacgaatgatgaaaatatatttgtttcggatatggacaatcagcatcattttgaaattgaagaaggcaaagggaaatattatgtattgtttgatattgtttctgaagtaactgaattttctcctgttaaaaccactattccagtcctcgaagaagggcagtcgttagttgaaggaacaacttgggttgataaatatacgggagaaataaaatttgacccaattaaaaaccgggaagatcaccgccaccacgccattgatgctcttgttattgcattaacaaaagaatcttttttccaacggttaagtaaacaaaatgcttcaaatgaaaataagaaaagagggattgaacacgaaaaagaacatttagactttccagaaccttggaatggttttcataatgaagcaaaaaatgttattaatagtattctcatttctcacaagcaaaacaaaaaaatccttacgaaaattaccaaaaagattaccaaaaatgggcaaacttttaaaagtaatggaatggctgttagaggacagttgcataaggaaaatttatatggatatagaacaccaccaattggagaagctgggtatcatattagaaaatctgttgaaagtttaaaaaccgataaacaattggaaaagattgttgatgtaaaaattaaagaaataataattaaagcaagaaaggcagaaattcctttgtataaagaagtagaacaactttctaaacaactgaaatctccaaaaaatacagaaatcgaagaaaaagaaattaaagaaaaaattgtagcattacatacgaaaattaaatcactgtatactttgccaaataaaaatggagagccagttccaataaaaaaagtgagagtttcggaagaaatgggaaaagctcaacaattaaaaccttctataaaacaatatgtgaatccaagaaataatcatcatattgtaatttttaaagatgaaaatgaggagttaaaagatgatattacaaatttttggaaagttgtagaaagagttaagcaaaaagatgaaatctataaacttcctgatttagaaattggaaaaccagccccaaaagaactagtattaacttttcaagaaaatgatatgtttttgttgggactttctaatgaggagtataatgataataaacaagaccttgtttttctaagtaattatttatatcgagttcagaaaatatcagatggggattattcatttagataccatcttgcatccactgtcacaaataaaaaggaggaagtaagaataacgagtatgaaaaagtatcaagaaatgaatccaataaaagtgaatatttcggttttaggaaggattgaaaaactgtaa

SEQ ID NO: 30 - DNA, 4599 bp, unidentified
  source 1..4599: mol_type = unassigned DNA
atggcaaaagtattaggtttggatttggggacgaactcgattgggtgggcagtggttgatgatgataaacaaaagatattcggtatggggagccggatttttccggaaggtgttgttgctaaaacaataggaaaaggagaaaaggaagaatcgaaaaacgcagcgcgaaggaacagtaggcaacaaagacgtcagtattacagaaagcgtttaaggaaaataaaactgttgagaacacttattgatttagatatgtgtccattaactcataaagaacttgatttatgggcaaaatgggataaatatagaggaaaggctgggcgaattgctccaagttcaaaagtttatattgaatggcatcgacaaaatccttatcacttaagagataaggccctaaacgaagacttaagcttacatgaaatggggcgtgttttgtaccatttaattcaaagacgaggttttttgagcaatagaaaaggaaaggatgatggtaaaatctacaaaggtaaagaaggaatggttggtatcgaccaaactaaaaatagtatcgataacggtacgttaggcagtttcttaaattcaatttatccgccagaaggagaaccattccaattaataactgacgaaaacggcaatgaacttcgtgtgagagcaagatacacgttgcgggatatgtacgttgaagaatttgataaaatatgggagcgacaggcaaagccattgggattgttggaaaaaacaggagaaaaaaagaagctgacttttctaaaaggaaatttggatgccaatagaaacaagaagaaaatagaaaaattatatcaaaagtatggggaaccaaacgttaccattacagaaatagaaaataaagatactagaaaaagtatttttaaggttgtggttaaatcgattgtgcccttaaaggaattttttggaggagaaatcgaatatgataaagaaggaacaattaaacacaaaagtcaagatagtgtcttgttctggcaaagacccttgcgatctcaaaaagggctattggcaaaatgccgatttgaaccaactgtaactaaaaatggtaaatatctgcaaagaggaaaatctccttgccatttgtcacatccagtttatgaagaattcagagcctaccagtttataaacaatattgaatacggtcgaaaacaaaaacttaatgaataccaaagattgcaagttttggctttgatgaatggcaaagacagtaattttaatttttcggaaattcctaaaaagctcaaaatggaatatgaaaaatggaactacgccgatgaccagaaagtgccaggaaattataccacaaaacatttgtcgagtttattttctaaggaagtatgggaaaaatacaaacatgagatttggcactgctttatggattttgaagacaatgacaatttaattgaaaaattgatttcagattttgatttaaatgaaaaactaatcgacaaagtagaaagaattaatcttaaagagggttattccaatgtgtcgttaaaagccataaagaatattctgccttttctaagagaaggatttcaaatgagtgatgccactattttcggaggtgtaatgaatgcttttggtgatcaaataaactatttcaaagattcaaaagaacaattaaaaaaagatgttctgaatattgtaagagacaaaaccaataaagagggagatgccattgagaaaatcaaatcctatttggttgaaaacgattttggatttacaccagatgataaacggtttataaaattgtatcaccatagtcaggatatagaaacgaaagagttgaaagacaaactggatgccattgaaaatttaagaaacccgattgttcaacaaggactcaatgaaaccagacggcttgtcaaccatttgattgatgaatatgggaaatttgatagaattcaagttgaattgggtcgtgatgtcaaaaacagtaaacaaggaaggcaaaatcagtcgaataggatcaaagaaaatacaacaaaaaatgatgctgccagaaaattacttaccgaatacgggttaaggcacactagagttaacctccaaaaagtcttgctctataaagaaatgcaagatcgtggcgttgtcactgtttgtccttacacaaacatttccatcaatataagtgacgtcttgggaagggaaaacaagatacagatagagcacatcatgcccaaaagcacctcattaaatgatagttttggaaataaaactttgtgtgattctaaattcaatggactaaaaggtaatcttacaccctatcaattttacttaaaaaacaatgatccgaaaatatggggtggcgcagaaacctgggaagacatcgagcaaaggatttataggttgttgccatatcctaaagcaaaacgatttacttcaaaaataaagatcgaagatgctgatgtacaaaatagctttatcgaaagacagttgaacgacacccggtatatcagcaaaaagaccaaagaaatcatgagtcaggtttgcgaagatgtgagagtgctgcccggagggcttacttcggaattaaggcacttatggggtttgaataatatcttacagccggttatgaacttggacatcaacctcaagaacatggaagaggatcgagtctatccacattatgttgtaatggacgaaaacaataatctcatttcgtcattaccaatttataatgaaaagccaacattggaagctaatcaaaccactatcacagggaaagtaaataaaggtgtattcaaaacgttaaacaactatgtcaatataactgttagtgtgccagaattgaccaatggaaattattggttaaaattgaacatttctgaacccaaaaaagtggtgcgtatctttaaggagcgaccggaaagtacagaaaacacgattgtattaagagggaatgtagaacgggaaaaatttaaaaataatagtatcggactagtttctgccaaagctattgacaatgggtcatattgggcgaaattgccagttatagataaaaaattcattgttccagaaaaagaaaagcaacccaaaaaaacaggaaagcagattctgttgtttggcgaagtaaaagataattatttcaaatcgtacatctatgaatgcgaaactaatacgacggatggaaaatattggctgctattggatcttgatacagagcatccagtttatgagagagccattaacgaaaaaccgagctttgcttcaaaccaaatttgtattgaaggaacggtaaatgaccaaggtgtttttgtttcagagatagatgcagagcatcaatttgctaccaacgaaaaggaaggaaagtattgggtggtgtatgacatcgtaaatgagccaaatgaattcaatgccattgagaaccaaaaacccccgatagaaaaaaatcaatcgttgatagagggaaacatttgggtggataaatacacaggggagataaaatttgaccccaagaaaaatagggatgatcataggcaccatgccatagatgccttggttattgccttgtcaaaacaaagttattttcaacaattaagcacatacaatgcacagcgggtggcaaaatacaaaggattgccttttgaaaaagaccaattaaatttccctgtaccatggttgaatttccatacgtcggcaaaaaaggaagctgaaaagatcttggtgtcacacaagcaaaatcgaaaagtgttgacacaagtaagaaaacgcattactaaaaatggcaaaacttttacaagtgtaggagacgccgtaaggggtcaattgcataaggaaaatatttatgggcaaagacaggcacctgcggaagttagtaaaggatatcacatgcggaagaaagtgagcgatttaaaagacaaccagctaaagaaaatcgttgacccagaaatacgaagaattattacgaaagcgagaatagaagaagttaaaatcaataaagaaatcgctttattggaaaaacaaaaaaagcaaaaaggagttagtgatgaagaggaattggttatcgtagatcaaatcaactcgttaaaaaatgacatccaacaactctatactttaaaaaataaaaacggagaacgcgttcctattaaaaaggtaaggttaagggaagaaatgtccaatgcacaaaaattaaaagaggcaaatcaatatgtgaatccgaggaataatcatcatataattatctatgaagataaaatgggagaacttaaagaaactatagtttctttttgggaggttgtagaaaggcaaaaacaaaatcaagaaatttatcaacttcctgaatttgataagcacggtaaaattataaataccttacaagaaaacgatatgtttgtgctgggcttggatgatgaagagtttaatgaaaataaattagactctaagtttttaatacaatatttatatcgcgttcaaaaaatatcagcgggagattattcgtttagacatcatttggcatccaccctgagaaacaaggaagaggaagttagagttacaagtttaaagagatggaatgaaataaatcctataaaagtaaatctgacagaaacaggttcaataattgaagcctaa

SEQ ID NO: 31 - DNA, 4677 bp, Kaistella flava
  source 1..4677: mol_type = unassigned DNA
atggcaaaaattttaggattagatttaggaacaaactcgattggttgggcgatagtggatgatttggagaatgaaattctagatgtaggagtaaggatatttccagagggagttatttcgaaaactattggctatggagataaagaagaaagtaagaatgcgaaaagaagaaatagcagacaacttaggaagcaatattataggaagagaataagaaaaataaaattattaagaactcttattaatttcgaaatgtgtcctttggagcataatgagcttgatgtttggagtaaatgggataagcaaaaaaagaaagaaggcagaatggctcctgaattatttcttcctaaaagtcattcttataatctatggttaaagcaagatccgtataagctacgtaatagggctttacgtgaggatttaacattgttggaatttggtagagttttatatcatttgattcagcgaaggggatttttaagtaataggaaaggtaaagatgatggtgcaatttataaaggaaaagagaacgtaaaaggaattgatgctactcttaaagagttgcaggattctactttaggcaattatttgttccatattttaccaaaagatggagaaccttataaaattattcaagatgaagatggtaatgagctaagagttagatctagatatacattgagagatatgtatgttgcagagtttgaagcaatttggaatcggcaatcaaaacacctaggattggaaacagttgaatgtctgagcgagaagacaagatttcttaatggtagtatcgaaaataaaaggaatgctaaaaagatcgaacatcttcaattaacaaaaggaactgaaaatgtagaaattaaatatataagtgatgaactttcaaaagatgataagaaaaaaatattattaaaggtgacaactaaattaccattaaaagaatttttggctggtaaaatagaaaaagatgaagaagaaaatctaaaatttaagagtaatgagagtgttttgttttggcaaagacctttgaaatctcaaaaaaaattattagctaaatgtagattcgagccagatttgaaagatgagtttggaaaatttattcaaaaaggtaaaacaccttgccacttatcgcatccattatttgaacaatttagagcatataagtttataaataatatagaatatggtaaaaaacaacgtcttgaagaatctcaaaggttagaagttttagagttgattaattcaaaagatactaatttcaagttttctgaaattcaaaaaaaattgaaattagaatacgaaacatttaactatgacaaagattcctcagtaagtggaagctacacaaccaaaaatttaaattcagtatttcctaaatcaatacttgaaaaagaatttgtagtaaaggtcaatggagaagatgttatagaatatggtatagatcgtatttggcatttatttcattattgtgaagacaatgatattttaattaaaatattgcaaaacgactacaatttagaagaggataaaatagataaaatagccaaaattaatttaaaagagggatattcaagtttttcattgaaagcgattaagaatattttaccatatttaaaagaaggtaaaagagaaagtgatgctgttattcttggaggtgtacgaaatgcttttgggaaaagatggaaatattttgctaatgagtatgaagaattaaaaaataatattttaaaaattaattacgataaaacgaataaagaaggagactcaattgaaaagattaaaaggtatttgattgataaaaaatatggatttgtagaaagcgatatttcatttaaaaaattgtaccaccacagtcaagaagttgaaataaagaaacagttcaacaaaattggagatattgaaaatttaagaaatccaattgtgcagcaaggcgtgaatgaagtgaaaagaattgttaatgaattaattgttcagcatggtaaatttgatcaaataagagttgagttaggtagagatttaaaaaatggtaaaacaaaacgacaagaattaacgaataggatttctcaaaatacgacaaagaatgaaaatgcaaaacaattactaacggaatacggtctaagccacggaagagaaaatattcaaaaagttctattgtatcaggaattgcaagaaaaaggaattctcccagtttgtccttatacaaataaaacaatcaatatttcagatgttttaggtagaggaaataaaattcaaatagaacatattattcccagaagtgtctcattggatgatagttttgcaaacaaaacgttgtgtgattcaaaatttaatggtttaaaaggagagagaacaccttatgaattttatcaactcaacaaagatgtcaatttatggggcggagcaaaaagttgggaagaaattgaaagaagagcgtttaaagttttgccttatcaaaaagcaaaaagatttgtttctcagacaaaatttgataaaaacacttttatcgaaagacaattaaatgacactcgttatataagtaaaaaaacagcaggaatattaagtcaagtttgtgatgatgttcgagttatgccagggagcttaacggctgaattgcgaagattgtggggtttaaacaatatacttcaacctgcttttacaattgatattccaaatttatatattgatgcagaaagacatattccacattatgttgtttttgataaagataaaaatccaattagttcacaaagaatttttaatgcaaaaccagatttattacaaaatgaaacaactctttttggtaatgtagataaaggactttttaagtctaaagaacaatacgtgaaatttcagattgaaaccccagatttactacatggagagtattggacaaaattgaaattatcaaatcctaaaaatattgttcgagtgtttaaggaaaaacccgaaacatcagaaaaggaaatagtcgttagagggaaaatagaaaaagaaaaatttaagactgaagggcttaatatgataaatgcagttggtaatgaaaatggaacctattgggctaaactcactatacttaataaaacatttaagaaaccaacaaaagaagaccaacctaaaaaaaatggaaaacaaatactcttatatggggaagtaaaagaaaaagttttttcttcctatgtatatcaatgtgacacaaatcaagaagatggaaaatattggttaataattgatattgatgttgaaaatgtgctttttgacaaagccatgaatgaaaaaccattagcaaatgactatcaaattgttattcaaggaacaacaaacaatgaaaatgtttttgtttcagatatggacagtcaacaccattttaatatggatgaaggtcaagggaagtattatgttttgtttgatattatttcgaaagttgaaggcttttatccagttaaaacacaagagcagaaattagaagatggacaaacgcttgttgaaggtacggtttgggttgataaatacacaggagaaattaaatttgatccaaagaaaaaccgtgaagatcatcgacatcacgctattgacgctatagtaattgcattgaccaaagaaaaatattttcaagaactaagtaaacacaatgcatcatctgaaaataaaaaaagaggaaatgagtatgaaaaagaacatttaaactttatagagccttggactggttttcataccgacgctcaaaaagtagtaaacggtattctaatttcatttaagcaaaacaagaatattttaacagaagttagaaaaaagatcgtaaaaaatggaaagacctatttaagtgtgggagattccgtccgcggtcaattgcatatggagaatgtttatggtaaaagaacaccaccggagggtaaagaggggtttcatagaagagagtctgttgaaagtttagtttctgaaaaacagataaataagattgtggatactacaatcaggaaaataataattaatgcaagaaaagaagaaatcccgatatataaggaaattgaaagtttagctaaacaattgaaatcatctaaattaaatgattttgaagaggtttctttaaacgaaaaaattatagagttgcgtttgcaaattaaacaactttacacgttgccaaataaaaaaggagaacctgttcctataaaaaaagttagagtttctcaagaaattggaaatgcccaacagctaaaacctactattaagcaatatgtaaatccaagaaaaaatcatcatattgttatttataaagatgaaaatggcgaattgaaagatagtattacaaatttttggaatgttgttgaaagagttaaacataaagacaaaatatataaactacctgaagataaatatgtagaaccagaactgaaagagttagttttaacattacaagaaaatgatatgtttttgttaggactttcaaacgaagagtataatgataattttgagaatagttctttcttgagtagttatttatatagagttcaaaaaatatctgacggggatttttcttttagacatcacttagcatctacagtaacaaataaaaatgaagaaattagggttgctagtatgaaaaagtatcaggaaattaatccaataaaagttaaaattgacttgcaaggtaaaatccaaataactggtaaggattaa

SEQ ID NO: 32 - DNA, 4560 bp, Pontiella sulfatireligans
  source 1..4560: mol_type = unassigned DNA
atgagcaagcgcattcttgggttggatttgggaacaaacagcattggttgggcggttattgacgaggtgcaggaaaagattgttgatgccggggtgcggatctttcccgaaggcgtggtgagcaagaccattggatctggcgataaggaggaaacgaaaaatgcgacgcgccggaaaaaccgtcaaacacgtcggcaattttatcggaaacgcatgcgcaaggcgaagctgctggaggttttgattgaacagggcatgtgcccgctgtcacttaaggagcttgatttgtggcggcgttgggacaaggagaagaaatcgcaaggccgaaaatttcctgccagtgatgagttcatgaagtggatacgcttgagtccttatgaattgcgtgcgcgggcggttgcgggaactgtatctgcacacgagctagggcgcgtgttctaccatatgatccagcgccggggttttctaagcagtcggaaaggcgaagcaaaggattcagaaaccttgtttgaaaagggaaaacccggtgttttgccgatcaatacgacagtgtccgaaatggcaaaaacaggaagtcagtcatttggacagtatttgaatgcggtggttccgaaaaccggcaagccatacgcgaaaagctgtgattccgagggaaatgagattcgagcccgcgggcgctatgctacccggcagatgtatatagaggagtttgagcaaatctggaaggcgcaggctgacaagctgggattggatgggcgcacggttgtttacaggaaggttcggaagcttcagggaggagaggactcggcacgcaatcaaaagaaggcggaaaagctgaggaagaaattcggggcggatcgggtctgctttgagcggaaaggaaagcatacctttttggtttcaactcaagacatgccgttcaaggaattgttggcgggcaaaatctgggaggaagatggccgactaaagttcaagagcaacgaaagcgtattgttttggcaacgtgacctgcgctcgcaaaaaggaacattgggcaattgtacgtttgaagacggactgcccgtgatccagttggatggcacggttcggcgcggcaaagacggcaagcctgtaacctacagcaagaaaccatgcccggtttcccatccggcgttcgagctgtttcgtgcacatcagttcatcaacaatatcaagtatggaaacgggcaacggctgacgagagagcaacgcgcgactgtgttggaactgatgaacagcaaggatcgggactttgatttcaaggaagtggtgaaaaagctggagctgttttcggagcagtttaattacgaaaaggattttaaggttcccgcgaacagcacgatttgcaagctaagcaagcttgataagggcaaggagtggcagagtgtgctggaggaacacttgccggaaatctggcattgtttctacacctatacagattccgatatgctctgtgagaagattgcccggacatacggcgttcggcttgacctaggcaaggtagataaggttcggtttaaggatgactattcccgggtctcattaaaagccatcgggaacattcttcctttcctcaaaaaaggactattttacagcacgtcggtcattttgggtggcgtgaaaaatgcctttggacggcgttgggatgactacctaacggatgaaacaaaggaaaaaattcaggctgacgtattggaggtcttaaaggaaaagaatggagaaggggatgttattgagaagatcaaggcatacctttccgatccggcaaaccgttatgctttttctgagaatgcccccgcttttggaaagctctaccaccatagtcaggatgttgagaggcgaaattcaaatgcggccaaactgtctcctgttaaagatgttcggaatcctatcgtgcagcaggcgttgcatgaaatgcggatggtggtgaatgcgctgatggccaagtatcgggaagacgatccaaacttcagattcgaattcatccacgttgaaatggggcgcgacctgaagaacggcaagaaacggcggagggaactgagcagggttattgacgaaaacaaaaagaagaacgatgaagctcgcgaatggttggcggagtatggtttgcggccttcccgcgaaaaccttcaaaaaatgcttttatttatggagcttgaaaagcgggcaggacgtgcgcaaagcccatacacagggaaaacgatcccgctatccaatttgttgggtgcgaataataccatacaaattgaacatatcatgccctacagcaagtcgttgaatgattcattcggcaataaaacgctctgcgagtcagaaatcaacggaatgaaaggtgacgagactccttatgagttctacctacgcaactccgatcctggtttgtggggcgcatccagttgggatgatgtggcaaggcgtgcatttgcattgcttccatatgagaaggcaaggcgtttcgcttcgaaggaacctgtcgatgctgagaagtttgtggtacgtcaactgaacgacacccgttacatcgcaaagaaagcggtggaggtgctgtcggaaatttgcagagatgtccatgtaatgcccgggcagctcacttccgagttgcgccacctttggggcctcaacaatgttctggaaagttctatcggcgcagatcggttgcttggtgatctggcagcatccgatgtgggggactattaccttgtgcttgatcaggacgggcaggcgatctcgttgtcgaagcggaagaacaatcagccggataccgatgcgaatgaaatcttgggggccggaggtgtggaaaaaagaaaattcgatgccaagcatctcgtattaaaactcgatgcacctgaagaagcggatggtaagtattgggcgcgattggttttaaaacccaatctaaagctgacgccaaagtatctagaacggccggatgagggagaggactacatcgttttcaagggcatgattcgaaaaggtaagttcaatcatgacagtatcccggtagttctggatgcggaacaagatgacggctcctgctgggtcaaactttttctgaaaagcaagcgtcccgtggatgctaaatcaaatccaaggacgacgaagaaggagataaagctgtttgggcaggtaaaggacggcgtgtttgaatgctatatctacaaatgcaagaccgatgcacccgacggaaagtattggttgattctggaacccgatttcgatgctccccagtttgtcagggcagtgaatccgcctccagagcatgccgatagtgaaatcataatgcagggaacagtggatgaatccggcatatttgtttcggatatcgaccgtgattttgttgcaaaaacagacaaggtgccaggtaagtattacgctgtcgccgaactcttcagccatacgccccgcttccatcttatggaaaacccggtgcctgacattgccaaggggcagagggtggttgagggaacggtgtggatcgacaaatattcagcagaaataaagtttgatcccaagaagaaccgcgaagaccaacggcaccacgccgttgatgccataactatcgcgctcacaaagcgtagctatttgaatgcattgagccgctttcgctcaaaggagcaccctgaagaatacaaacctgtcttcagtgcaccgtgggagggttttgaaaacgacgtgaagtccgccgtcgaaaaaattctcgtttcttatcgcaaggatgagcgggtggtcagcaaaggaaaaaaaggcacggctgtgcgcggtcagttgcataaggacaacgtatttggtgaatatgttaatcttactgatggatcgaaatcatatcatcgacgcaaagcgctgactgctttggaaacgcataaacaggcagaaaagattgtagatgagacgattaaaaaattggttttagaccatttgcgggataactgcgggattgatatcaaatcgaaaaagggggtgaaaatacctgctgatgcatttttcaaagatggcaagccacgattgtttttacctccgaatccgaagaaaaacggcagcccagttccgattaagaaagtccgtctggaagaaaccattaacaatgcccgtcagtggaaaaaaggaaaaaaccaatatgtgaatccccgaaataatcatcatgtgttggtttacaaggattcagaaggtgagttgaaggaacaaattgtctccttctgggatgcggtacagcgcactatgaaaaaacaggatgtttatgaattgcccgacgatgcggatgagcaggtgcttgtcttggagaagaacgatatggttgttctgggagctggtgacatcgagtttggagaaataagttgcgatccggcgttgttgagtcagaagctgtatagagttcagaaactctattctggagacgatgggtttgagtatgtttttcggtatcacttggcttcgaccctaaacaataaaaacgaagaggtgatgattaaaagcttcaaaaaattcaggggcgttaacccgatcaagataggaatatctgagctgggggagatagctcgatgttaa

SEQ ID NO: 33 - DNA, 4530 bp, unidentified
  source 1..4530: mol_type = unassigned DNA
atgataactttaggtctggatatcggtgtcagtagtctaggtacagctatcgtcgacactcagaagaatcaaatactccacacaggagtctatgtattccccgcaggtaaagaggctttcggcaccaataaggagaagccaaaaaacgagacgcgcagggtcgcaaggcaaggtcgcagacaaaactataggcgtcgtttgcgtcgctctcagttgctcaagtttctctatcagctcagtgagctcaatagccccgaaggggatcgctatgtgccacttacttttgaggagatagatgcttggctctcctgggataaggaggggaagacggcggcacgtcagttcccagatagtgatgccttcagagcttggatggcgatgaacccttatgagattcgctcaagagcggtagaggggagcatcacgagggccgagtttggtcgtctattgtaccacatcatccagcgtcgtggcttccaatcttcacgcaagatgaaggcagatgagggagcgatggcgaaaggaaaagatgacatcatcggctacacccaaaccaagtcggagataggagagcagacccttgggcaatatctgtatcagataagtgctaaggagggagagccatttacccaacgcgaggagaaacccagagggaggtatactctccgcagtatgtatgtggaggagatggagcgtatttgggatcagcaggctaagcgacttggtctagaggaactagagatagagggtactcccgtcaaggagctcttgatcggtcagattagccacgatctagagagtggagagctactgtacaagtccaatgatagcgtcctcttttggcaacgcccacttcgttcgcagaagcacttaatcaataagtgtagccttgagagtcgtaagatgtacgacccagtgaagcaacgccactatatccaaggtccacgtgtcgcagcagtttctcaccctaccagtgaatttcatagggcacttcaggtgctgagcaatgtgagggtagaggggcgtacactctatgatatgggattattccaaacctccctcgacttcctctgctcaaaaaacagcaagactatcaaggtggctgacctgaaaaggcacctcagcctagtatccaagctcaattatgatgatgaccaatccctcccagggtgccctaccatcgccagcatcaccgacctttttgaaaagggtgaattgagctaccactatggcgagaccgataccatatatatagatatctggcagaagctactcttttatgacgatgagagactgcttgagaagaacatcgccgactatgccaagggcagggggttaaagctcaaagccgactttgctaagcgactagatggtatcaatctagttgatgattacggatctatctccattcatgcaatggacaatatcattccattcctactccaaggacgaagccacacagaggcgatattcctcggtggtgtgaaaaatgcatttggtctgaggtactactacttcgagaaagaggagcagatgtggcaagatctcctccagatcattaatgagccagcctcaaagggtgagaagatcgaccgactgatagagttcctaagcaatcccgacgagcagaccggtttggacgaaaagcgactgcggatgaagctctaccatcccacccagtcggtgcaaaagatagagatacagaatcggctaggggaggtggataatctccgtaatcctattgtggagcaagccctttgggcggtgcggaggcaggtcaatgctcttattgatcagatgattgaacagaccggcaaccccaacttcagatttgatcgcatcaccatagagctctcccgtgacctcaagtcgtctcttgaccgacgcctcaagatacgccaggagcaagaggccaatcgggcagaaaatgaggaggcggtgatagagctaagggcacgaggtgtgcgggtcactagagataacatccttaaataccgactgcataaggagctccaacagcaagatggcattgcccgctgcccttataccggcaagaatatcaacccctcttacctctacgatggcaataacttcttccagatagagcacattattcctcgaagcatttccctagatgatagtttcggaaataagaccctctgcgagtccaacttcaatagagagaagggcaatatgaccccttacgagttttactccgtcaactctagcaaaaccctctggggtgctgaggattgggacgagatcacacaccgcgtaaggcgagtgctaccagcccgtaaagcaagtcgctttctatccaaacggagtgcccaagagcagctagaggaactaccgagtaagatgctcaatgataccgcttatatgtcggtaaaggcgcgtgaatatctcaccaccatttgcggtgatgtccgttccgttacaggacatatcactgccgacctcaggcggatgtggcaacttaataagatcctaggcgagacctatgaatgtgccccaggtgccgtgaacgaggagctgatagcaagaggcgataccgatgtctatgccctcctagatagcgagtcacaggtggtcaatcgtgtggcgcctaagctcaatctcatccccaatcccacagcagatgagatccgtatccccgtggtagtcaaggggactaagctataccgcgacggcaaggagtaccccaagccagcccatcttgatctcagtggagtgacgggcgaggccctcatcgcttaccacgcagtaggcggttccccacactgccagcccctctttaaccccaagccctctgccaaggatatgctagtggttccagcgaccattaatagcaagaaccaattgactagcgactatattgaggcactccgaaaagtcaagaggagtagttatcctaccgagccaggaaagtactgggtcactcttcagcttaaaggcgaacccatttgggagctacctagtaataaaaagagagtggcagctaatgcggtaagagtatcgggccgactgaatggcaacgccttctggagccctctctttgagaccgaagtagatagtagtgtggtcaatatggaggagcaccgcaatcgcaccgcctatctcaatctagactacgacaccgcagtgttcaccaagatgcttcgccctgcaccagaggtaggtgagggagagttactactcacaggagttgccaaccacggttcgctccttctggatcaatgcgatggcaagacaatcgatgtacagcatttgaatctccaagagcagaccaagtattatgtctccttgcggtacggtgagaccctttctcctgaagagcgggtcaccatctatccactttacgccaaggctcccacaccaagaggtggagaagtcctgctagagggaaagctcttcctacttgataagtctcaaggcctttacgctttggagccgattaagaatagagatgatcataggcaccacgctgtagatgccttggtgattgccttggccaagcaaaactacgtcacggagctttcaacgtacaaccggcaagacgactcctaccgacaaggagaggctctaaagccccgcttcgacctaccgtgggagggctttagagaagaggcagagcaatctatcaatgatatcttggtggtacataaaaagaatcgagtggatcccctctccaaggtgacccgtaagatcaccaagaatggcaaggtctatacctccgaaggttacgccgtaagaggtgccctacataaggctactatctatggccagagaaaagcacctgggcagacagagatgggctaccactataggaagagcatcaccgaagtgaccaagatgagccaactgaataaagtggtagatgcggctattaggaaagagatgctgcggatactcaaggaggagttggggctagacataagtaatccaaagcttgttttccctaagaatgcctttatcgatgaggatggcaaaaccaaagtctatcttcctaatcgtaatggaggagaacctgtcccagtcaagaaggtgagattatgtgataagtttaataatgtggtaagattgaaagaggccaatcaatatgttgacccacaaaataatcaccacgtattgatttatgaagcactagatggcagtcttcaagagagtgtcgtaactatgtgggatgtggttaatagacggaatagaggtgaggagatattccagcttccacccgatggcaagagaatccttttcaagcttatgcgtgataggatgtacttgatcggaatcaccaaagaagaggtacttctccacctttctgacaccgcctatctcagcaagtacctctatagagtgcagaagttatcatcaaaggattatttctttagaaaggctactgctagtaccactaattatgatagagagatgatcagagtgtcttcactgagcttctttgaacccgagaaggataatgtacactcggtggagatcagtctcacgggtaaagtaacattgaaggatgattaa

SEQ ID NO: 34 - DNA, 4536 bp, unidentified
  source 1..4536: mol_type = unassigned DNA
atgatcacactaggtctagatatcggtgtcagtagtctggggacagctatcgtcgacactcagaagaaccaaatactccacacaggagtctatgtattccccgcaggtaaagaggctttcggcaccaataaggagaagccaaaaaacgagacgcgcagggtcgcaaggcaaggtcgcagacaaaactataggcgtcgtttgcgtcgctctcagttgctcaagtttctctatcaactcagtgagctcaatagtccggaaggggatcgctatgtgccacttacttttgaggagatagatgcttggctctcctgggataaggaggggaagacggcggcacgccagtttcccgatagcgaagcttttagagcttggatggcgatgaacccttatgagattcgctcaagagcggtagaggggagcatcacgagggccgagtttggtcgtctattgtaccacatcatccagcgacgtggcttccaatcttcacgcaagatgaaggcagatgagggagcgatggcgaaaggaaaagatgacatcatcggctacacccaaaccaagtcggagataggagagcagacccttgggcaatatctgtatcagataagtgctaaggagggagagccatttacccaacgcgaggagaaacccagagggaggtatactctccgcagtatgtatgtggaggagatggagcgcatttggggtcaacagtctaagcgactcggtctagagggacttgagatagagggcactcccgtcaaagagctcttgatcggacagattagccacgatccagagagtggagagctgctctataagtccaatgatagcgtcctcttttggcaacgtccacttcgttcgcaaaagcatctaatcaataagtgtagccttgagagccgcaagatgtacgacccagtgaagcaacgccactatatccaagggccacgtgtagctgctgtatctcatcctactagcgaatttcatagggctcttcaggtgctgagcaatgtgagggtagagggacgtacgctctatgatatggggttattccaaacctccctcgacttcctctgctccaaaaacagcaagaccatcaaagtggctgacctgaaaaggcacctcagcttagtctccaagctcaattacgacgatgaccaaaccctcccagggtgccctactatcgccagtatcaccgacctttttgaaaagggtgcattgagctaccactatggcgagaccgataccatatatatagatatctggcagaagctactcttttacgacgatgagagactacttgagaagaacatcgccgactatgcccagagcaaggggttaaagctcaaagccgactttgccaagcgactagagggtatcaatctcgttgatgattacggctccatctccattcatgcaatggataatatcattccatttctgctccaagggcgaagccacacagaggcgatattcctgggaggcgttaagaatgcatttggtccgaggtactactactttgagaaggaggagcagatgtggcaagagctactccagattatcaatgagccagcctctaagggcgagaagatcgaccgtctgatacagttcctaagcaatcccgacgagcagaccggtttggacgaaaagcgactacggatgaagctctaccaccctacccagtcggtgcaaaagatagagatacagaacagactaggggaagtagataatctccgcaaccccatcgtggagcaagcactttgggcggtacggaggcaggtcaatgctcttattgatcaaatgattgagcagactggcaaccccaactttaggttcgatcgcatcaccatagagctctctcgtgacctcaagtcgtcaaaggagcggcgatccaaaatacaacaagagcaagaagccaatcgggcagaaaatgaggaggcagtaatgtggctaaaagcattggacttgccatatacaggcgataatatcttgaaataccgactgcataaggagctccgactgaaaggaggtggcgttgcccgctgcccctatactgggaaagatatacgcctggctcaactgtacgatggcaacaacctcttccaaatagagcatattatccctagaagtatctccctagataatagttttggaaataagaccctctgcgagtccaacttcaatagagagaaaggtaatatgaccccttacgagttttactccgtcaactctagcaaaaccctctggggtgctgaggattgggacgagatcatacaccgcgtaaggcaagtgctaccagcacgtaaggcgagtcgctttctatccaagcggagtgcccaagagcagcgggaggagctaccgagtaagatgctcaatgataccgcttatatgtcggtaaaagctcgtgaatacctcacctctatctgtagtgatgtacgtactgtatcggggagtatcactgctaaccttagaagattatggggtctcaataagatcctaggcgagacatacgagtgtctgccagggactgtaagcaaggagttagcagctggggaagaaaaggaggtctatgcaattctggatactaaatcaaatgtgattacaagagtagttcccaagttcaatccaattccctatcgaacagcagatgagatccgtatccccgtagtggtcaaggggactaagctattctgcgatggcaaagagtaccccaagccagcgcatcttgatctcaatggagtatcaggtgaggccctccttgcctatcatgcagtaggtggtatcccacactgtcagcccctctttaaccccaagccctctaataaaggtgtactagtagttacagctactcgtagtggtaagagcaacttaattagcaacgatattgacgtacttgataagattaagaagagtcgttatcctaccgagccaggaaagtactgggtcactcttcagcttaaaggcgaacccatttgggagtttcctagcaataaaaagagagtggcagccaatgcggtaagagtaccaggtcgattaaaaggcaacactttcgtgagttctttctttgagaccgaagaggagaacagtgttgtcaatatggaggaacaccgcaatcgcacggcctacctcaatctagactacgacaccgcagagtttacgaagatgcttcgccctgaaccagcggtaggagagggagagctactactcacaggagtcgtcaatcacggttcgctccttctggatcagtgcgacggcaagacaattgatgtacagcatttgaacctaaaagaacagactaggcattatgtactattacagtatggtaaaaacctttcatccaaagagcaagtaacaatttatcctatttacaataaagctccaaaatcaagaaaagatgaggtattacttgagggaaagttcttcgcacttgacaaggataagagcctctacgctttggagccggttaagaatagagatgaccataggcaccacgctgtagacgccctggtgattgcactagccaaacaaaactacgtcacggagctttcaacttacaacaggcaagatgactcctaccgacaaggagggaggctaaagccacgcttcgacctaccgtgggatggctttagagaagaggcagagcaatccatcaatgatatcttggtggtacataaaaagaaccgagtggatcccctctccaaggtgacccgtaagatcaccaagaatggcaaggtctatacctctgaaggttacgccgtaagaggtgccttgcacatggataccatctatggtcagaggaaagcaccaggacaggcagaggtgggctaccactctaggaagagcatcaccgaagtgaccaatatgagccaactgaataaagtggtggatgccgctattaggaaagagatgctgcggatactcaaggaggagttggggcttgatacaagcaattcatccttaactttcccaaagaatgcctttatcgatgaggatggtaagaccaaagtctatctccccaatcgcaatggtggagagcctgtgccggtcaagaaggtgaggctaagggagaattttagttcgcctcgatgtctaaaaaaccaaaaccaatttgtgaagacagagaataatcaccacatgcttgtttacaagacaaagaaaggtaagtttaatgagtctattgtaacgttttgggatgtcgttgaaaggttgaacagaggtgaagcaatattccaattgccagaggatggggaaaagattatatttaagcttatggctaatgattattttatcataggtctaaagaagcaggagataattagcaacctatccaatccttcactgttaagtaagtatttgtttcgggttacctctttgtcatcaagagactatcgctttagactcgtcgcagctagtacagataaaaacaagcatgaaaatagaagaattagaactcctaatttctttgatcccactaaagagaatatccatgctgttgaagttgatctcaatggtaacattagattaaaagaggatgattaa

SEQ ID NO: 35 - DNA, 4695 bp, unidentified
  source 1..4695: mol_type = unassigned DNA
atgcgtaaagaacaatatatcctcgggcttgacttagggacaagcagtcttggctgggctgttatcgtacgtgagggagctagcgcaacctctatcctaaatgccggatccactgtgtttccgtccggaaaagcgatggtcaatggagtcgagaaaaccaacaacgaagagaggcgtaatagccgtcaggcgaggaggcaactggctcgccgtcgtctgcgcaagcagcatctcttgagactgcttatagagaaagacatgtgtcccctgacacagacagagctggacggctgggtgaagtgggataagagcagaccctccgaggagaggcgacgcttcccggagtcaccccgcttcagggagtggctggcccttgacccttaccgactcagggcagaggctgtagatacgccccttgaggatgacgccgacggaggctttacggctcgtgaaaagctggggcgtatcctttactccatcatccagcacagaggcttcctctcatcgcgaagtgggaaggaggacggatctatctataccagcacgatggagaaggatgggaaggtcggcatcgatgagacgagagccgctctcggagatcgcacactcggtcagtatctctacagcattgccccaaaggagggtgacaccttaagtgatcaggctgagagagtacgctcccgctacacgctgagggagatgtatgtggaggagtttgaccggatatggacgaggcaggcaccactactcggtctcgatagggagcaggtggagacggtacactaccggtacttcaccggtaagagtgactcgtatgaggcaagacgtcgactagagaggtatcggaccaaggatccgtcagctgtgatagagaccttgtccggtgacgagcaccgcttcgtctacaagactaccacttctcttaaggagcatctggcgggtaagatttggtacgacaaggatgggaccatcaggtttgtctccaaggagagtaccctcttctggcagagacctctgagatcacagaagagtatgctggacaagtgtagccttgagtcgcacaaggttaaggacagaggcacaaatcgattcatcacccagggaccaacggttgtctctgcctctcatccctactatgagctgtacagagtcctgcagaccctacttaatatgaggatacgggaccggaagctcgatctaatgatgctccagtcaatgcttgacaagcttctcacttatggaaagaatactaaggtctctaagcttaagaaagacctccgactgactcttgaccttaactttccggatgactacgagctgcccggatgccccacgattagtgggataactaagctccttgacagtaagaagatgccctcccgtaagggagatgcgagtcccgttgatcgactggtcatcccctttgacctcgatctatatacccggatctgggagaagctcttcttctacgacgacaatcatctgctggcgcaaggtctgagggggattgagggtgccgcctttgtggaggacctcgaggacaagctcgccaagcttcatctcagcgaggactatggcaacctctccctccacgctctgcgtaacatcctaccctatctcctacgtggggaggagctctacagtgctgtgctgctgggcggtatacgtaatgctgtgcgggatgatgctctgttggacgatgatgcgctgaggttgtggatccacacccagggacctctggcgcgtggtgagatgatagataggatcgtggccttgctgcaggatcccgatgggccatttcaactcagtactccggagttgaagctgcgtcagcgactctatcatccctctcaaggcacagctaagcaccggctgatggatcagctcccgccggtggagaacctccgcaaccctatggtcgagcaggcgctgtgggcgatgaggagactggttaattatctcctcaataactatcgctccacgatccgaccggactttacctttgacgagatacacgtggagctggcccgggacctcaagctatccactcagaagagggcagagatgagaaagcgtcagaagaaagccgaggaggacaacgatatggcaagggactatctgcgcagccaaggtctgagcacctctcgagaaaatctccagaagtacaagctctatatggaacttgccggtcgtagtatgggtgtcgtcacttgtccgtataccggcaaggtcatacgtggactgagcgctgcacttgatggcaacagcaagttccagatcgagcatatcgtcccccgtacaatgacctacgatgacagctttgccaataagacgctctgtgatgcggacttcaataggaagaagggcaacttgaccccgtggcagttttatcagcaggatccaagccccaagctctggggtgccgactcgtgggaggaggtggagcacagggcattccgtctcctgccgtaccgcaaggcgacccgcttcgtgagtaagaccatagaggatcagctctccggggaccagctggtcgataccgcctacattagccgtaaggctaaggagtacctctcctacatctgtccccaggaccatatccgactctatcccggtcgggtgaccgcaaggctgaggcaactttggggactcaattcaattctctcgcaaggacccactatcaccaagggtcagctcccggcggggctttcgctctcaggtgatcaaattcctgccatcgtcattggcaaaaagaagagcggtgtactcagtcgcattctgcctaagtacacatctcgcccggagcgagaggagcgtgacgtggtcctgctggtaagggtgcagaagggaaaggtatatagtgccaggtacagtgagctgccactatcctgggacaaggctacttctcttgctgacgggacatattacattgtcctccgtagtacagaagtgctccgcgtggagcgaatgtttgctgacgaacctcgactgagcgaggatcagctgagagcaagggttaccatcgagctgaatagagatggacagcccaagctctcctccagggagatggatgtgcgcattctaaaggatacgcctttgcccactgccccgggtaattactggctgacgatacccctcatctctgtcccggaggttactcctgagcatccgagagcaaaggttgcccccgacacaatcaaggtcacgggatcagtggagcagggtgtatttatctccaaggtgtaccggcagggtgctgtggctatggcggatggcgtggcgcagggcatccccgcccctatgctatcagaggcgtccttcactaagatcaagctggatgctcctgtgatcacctccggagaggtggtactcacgggtgccatcgacaggagtgacctcttcgtctgtgaccaggacgatgagtttagggtacatatcgactcatttgatgagccggggaagtactatgctgtcttctcctttgacccgatggcgtcctacatcctgtcgtatgaccaaacacctaacctgagcagtgacgaggtggtcctcgagggcgagctgatctataatgataaggatgaccggatccacttcagtccggcaaagaatagggatgaccaccgacaccatgctctcgatgcaattgtcatcgcatgcaccgggcagaaggtgctgacagcgatgtctacctataacgctcacgctgatgactacgagagaggtgcaggcagcaggcctacatttgcctctccctgggagggcttccgtgacgacgtgagagaagcgatcgaggccgttctcgtcaagcacaagcagagtaccaagatcctcaataaggtgacgaagcagatcaccaaggatggtcgcactcatcgctccaagggccttgccgtacgtggcaagctccacaaggagaccttttatgggcagcgacagtcacctgaggagcctgctccaacaatgcactatcgagtgcccattacttcaatcaccaaatcaaagcaactaaatcaagtggcagacccgacaatccgggcgttgatgatccggcgattgcaagatatgggagtggataccacgcaggaagactacgcaattccaggcaatgcgttcgtggacaaggcgggtaactacaccatcctcgtgccgaataagcgtggagtccccgttcccgtgaagaaggttaggcgtagtagggttgttaataatacggtccggctcgatggttacaataaagaggtggagactagtaacaatgattatgtgcttatctatgaggaccgggaaggcaattattgtgaggaagtggtgaccttctgggactctgcagatcgattgctgcatggacaggatagagtacagctcccaatggatgtaaagagagtcgttatgacccttcgcatcaatgatatgttcgttatcggtatgagcgaagacgagctacggagtgctgtcgggtctagtgattatggagtcatctctgatcacctatatagagtgcagaagctctcttcgatgcattatgtattcagactgcatagagccagtactctcaatcatgacgaggagctctatacaattcgcagctttagtgcactcaaagagtctactcccattaaggtgataattgacacagacggcacaatctctatagcggcacgagatgatcaagcgaacaatcaccatcagtag

SEQ ID NO: 36 - DNA, 4542 bp, unidentified
  source 1..4542: mol_type = unassigned DNA
atgggaaaagttttaggactcgatctcggaacaaattcgatcggctgggcgattgtcgaagaggagagggatgagataataggaagcggagtaagaatattcccttccgctgcggataatatcggaacttcaaaagaatcgttaaaaaatgccgtaagaagaaacgctcgtcaaataagaagacaattttttagaagaaaattaagaaaaataaaattactggaagttttaataaaatataatatgtgtcctttatctacagaattgttaaatggatggaaaaaaagagttaagggagaaatacaaaaatttcctcaatctgaggagtttaacagatggttaaaactcaaccctctatttttaagagataaggcgactaaagaacaattaagtttacaagaatttggccgcgttttatataatctgattcaaaaaagaggttttataagcaatcgtaaaaccttaaacgataaaggcgccatgtataaagggaaacctgaagaaagaatacttggaatagcgcaaactgaaaaagaattagaaaaaaaagcgactttaggaagtttcctgtataacatttttaaaaaagacggcgagaaatataaattttctgttgaaagagtaagaggaagatatacgttaagacaatggtatattgatgaatttaatgtattatgggataaacaaactcccaatttgggcttggacaaaaaaaatgaaatttataaaaagagatcatacataggaaacattaattcgtcaagagcaagaaaaaaaattgagatacttaaaaaacaaggtaaaaccttcaatatcgaaaatggttacattatagtagaagaacatattgcgttaagagacttgatcggcgatcctaactttggaattttattttttcaaagacctcttcgttcgcaaaaacatttgctttcgttatgctctttcgagagccgtaaaatactaataaataataacggagaaaaaattgagaaagcggttggcaaaagaatatgttcagcctctcatccggactttgagaaatacagagcatacaaatttataaattctatcaaatatggggcagatttgtcactaaacgaaagcgaaagaaaattattagccgaatttatgctttcaaaagataaagcgtttgactttaaaaaaatttcagagtttttagatatgagatctgaaaaatttaattatgccgatgattttaaagtttttggttctccttatacggcaacggtaaaaaaatcggtatcggaagatctatggaacaataaaaaaaaatatgaagaactatggaacgttttttacttttataaagatggagacaaattaaaaaataaattaaaaaaagattttaacataaacgacgaaaaaatattaaataaattagaaggtatgaaattgtctgaagattattcaaacttatctaaaaaagcgattgtgaatattttagaatttcttgtaaaaggttatcaagagcatattgcaattcttcttggcggagttaaaaacgctattggtaaagataagtggtcgtcgtataataaagaaaaaataaaaaaaattgaggatgaagtttatcggatggcaaaagaaaaaatgaaaagcggagatttaatcaaaaatattgtaaatttagtttatgaatatgaaaaaatcccaataaataagatttatgaaaaaatatatcatcattccgaagaagtaatccaaaataaaacgatatcgcataaattaggagaaattgagaatttaagaaatccgatagttcaacaatctcttaatgaaatgaagaaattagttaataaactaatcgataaatatttagacgaagatgaaaacttcaaatctataaatatcgaattagctagagagttaaaacttccgaaatctaagagacaactaatatataaagaaaattccgacagagagaaaataaatagatcggcatgcgacatacttgacgaatatggcttggctcattcaagggaaaatatacacaaatatctgctttataaagagcttgaaactgaaaacgggatagcaatttgtccttatacgggaaaaactatcggaataagcgatattttaggcgatagtaacgaatttcaagtagaacatatcataccttattcgatatctttagacgatagtttaaagaacaaaactctttgcgagtctacagaaaataataaaaaagggaatcgtactccttatcaattttataaaaacaatcctgttcattgggaaaaggttagagaaagagcttataaattattgccatatgaaaaagcaaaaaaatttacgtcgcaacaaaatttccaacaagatgattttatagagaggcaattaaacgatactcgatatataacaaaaaaagcgctagaatatttaaaaacaatttgcgtcgacgtattctctttacccggaagtttaaccgccgatttgagaaataaatggggactaaataatatagtaaggaatccgtctttaattgatacaaactcaattaaaatttccggatacggaaagtattgggccgaatatggggatagcgaaaatcctaacaaaattacgaaattatatcctaaatacaacaaaaaacctattccggatagcaacgaaattatcattaccggagaaattgccaataatgaatttactccggatagcaattttgaattaaaactatctaaaataaaattatcccaaaaatatgaagacggcaaacattgggcaagcgttaagtttgatcctaatttacaatctattcaacctgttaaaaagccgtatccaataaaagataaagataatttgattttgattgggaatatgattaaaaatcaatttattaataagcaatttaaatttaccgttatacctagcgcttttcaagatgataaagaaagaccgtcatattgtaaattaacaattgatataaataatatctcatttaaacaaatcaagccaaaaacgactgaaaatactattattttacctggaaaagttagttcgaatcatttttttgtgtttcctgacattttttctaaaaaagttgaacttccggatggaaggtattatgcggaaataccatacttccctgaaaaaacagagatatcccctttattaaatattaaaccgtcaattgaaaacaaacaaatactactgtacggaatggtaagaaaaaacgcgtttgttccgaatagtaatcctaatttaaaaattaatttaaaagatattatttcagacggtcaatattgggcggtttttaatctcacgggagaaataggaaaatttgtcagaaaagaaaatatacccccaaaagttgaaaatcagacaaaattagaagaaggcgaaataagatacaatagcgaatctgaagagtattatttttacagcggaaaagacagaaccgatcatagacatcacgcgatagacgctattacaattgctcttgctaaaagaggtcatcttcaaaaattaagcgcatttaacgcaaagaaaatagagagaaagagaaatataaataaaataaacgaaaaattaagatttgaaacgccatgggataattttacaaacgatgcaaagaaatcaatcgaaaatattttgatatcccataaatatcataatccgactgtaaaaaaagttattaaaaattttaagaaaaacggaaaaagttttagaggcgtaggtttatctgtgagaggagctttgcataaagatactgtttacggtttgcataaacgtccggtaatttcttacgatggaaaatttacttttaataatggaaaaatcgttactgagaatgaaaattattatcatgtaagaaaaaaattaagcgatctgaaagatagaaagcaaatcgaaaagatcgttgattatagaattaaagaaattatatacagattaatagaacaatcgggcggattcattaaggataatattccaagcggagctctctttgaaattgatgaagatgcaaaatcattaaaaagtaaagtatttttaccaaacaaaaaaggcgaacccattccaatctataaagtaagaatacgtgaaaatatttcaaacgctagatgcgtaaaaccttcttataaagttgacgacattgaatttaatcaatatgttaatccaaggaataatcatcatattatgatttacaaaaacaaaaacggagatttgtcggaagacgtcgttcagttttgggacgtaatacagaggaaaatgaataatcaacctatttatcaacttcccgaagatggagtcgaaatcgtaactactatggaaatcaacgatatgtttatactcggaattagagggacaaaagaggacgcgttaaaacttagcaaagaaactctctctctacatctttatagagttcagaaattatcttcaaactattatactttcaggtctcatcttgattcaacaacagagcaaattacagaaatgagaattcagagtttttcaaaatggattgaagaaaaccctatcaaggttaaaatatctgagattggcgagatttcctga

SEQ ID NO: 37 - DNA, 4479 bp, unidentified
  source 1..4479: mol_type = unassigned DNA
atggaaacaactttaggcattgatttaggtaccaatagtataggaatagccctcacagaccagaaacaagcccggattctgtacagcggcatacgcatttttccggaaggcatcaccaaagacaccatcggacaaggagaaaaagaagaatcccggaatgccacacgccgcatgaaaagacaaatgcggcggcaatatttccggaaaagactgcggaaaatcaaacttttagaaatactcgttgcctgtaatatgtgccctttacaaccggaagacatccggcgttggaaaaactgggacaaacacctaaaatctaccgcccggcaattccctgactcaccggcattccgagaatggctgaaactgaatccttatgaattacggaaaaaaggcgttacggaaaatatcagccggcaagaattaggacgcatcttgtatcattttatacaaagaagaggattcctgagtagccggaaaggtaaagaagacggaaaaatatttacaggcaaagaccaaatgaccggtattgatgaaacccggaaaaatttacaaaaccggacactgggaacatatctctatgaaattgcccctaaaaaaggagaaaaatatcgtttccgcacggaaagggtaagagcacggtataccttacgggatatgtacgtccgcgaatttgagacgctttggaaccgtcaggccgcccacttagaacttgacaacaaacaaatgccgcaaaaacgccatatatacttaaaaggtacttccacaaacaaccgcaatcgtaaaataatcactcacctacaacaaaaatatggtacggaaaatataagcattgaaaattcccgcatcaccgtatcctatttacttccgctgaaagaattactgggaggtaaaatcgaacaacaggacgggaacttgaaatttaaatgcaatgaaagtgtgctgttctggcagcgctctctccgcacacaaaaatccttactttcaaaatgtgtttttgaaggacggcgtttttatgattccattcaccggaaatggattaccgccggacctacgccagcacccctgtcccatcccgaatttgaggaattccgggcttatcagtttatcaataatatcacttacggaaaaggagaacacctgaatgccacccaaagagaagacctttttaaactcatgtgttccgaaagcaaggaattcaactttgacaaaatacccaagcatttaaaactatacgaaaaattcaatttcgacaaacagaccaaagtaccggcctgcaccacaatcagccaattgcaaaaactgtttccgccagaaacatggcaacaacaccgggaggaaatctggcactgtttctatttttatgatgacaatgaactgctttttcaaaaactgaaaaaagattacaacctgcaactcaatgaaagcgaaaaattaaagaaaatacggcttgctgaaggttacggacacgtatctctcaaagcaatacggcggattaatccctatctgcagaaagggtatgctttcagtactgcagtacttttaggaggtatccggaatgctttcggaaagcgtttcgaatatttcaaagaatctgaaaccgaaatagaacaggctgtctgccggattttgaaagaaaggaacacagaaggaggaacaatcctaaaaatcaaagactatctgacaaaaaatcaatacggctttactgaacatgaccgggcgtttcaaaaactgtatcaccacagtcaacctgtcaatgccaaaacattacaagaaaaattaccagcaacagaaaacctacgaaatcccatcgtacaacaagcattgaatgagttacgacggactgtcaataaattattgtccgactgccgccaaaaatacggacctgaatttcaattcgaccacattcacgtggaaatgggcagagaccttcgaagttccaaaactgagcgggaaaagcaaacccgcttcatccgggaaaatgaaaagaaaaatgaggctgcccgccagaaactacgggaattcggtcttcaacccaatcgggaaaacatacaaaaatatttgttatacaaggaaatagaagacaaggcaggcatcgtcagttgtccgtacaccgggaaaaccatcaaaatttctgatgcattagggagtgacggatgtgtacaaattgaacacattataccctacagcatatccctggatgacagtttcgccaataaaacgttatgtgaggctactttcaacagggaaaaaggagaacggacaccttatgaattttatgaaaaaaatccctcttcctctctatggggagacgtccattgctgggaagacgtcgaagaacgggcgtttcgtctgcttccttaccccaaagcccaacgtttcatccgacggaaaaatcccgatacaaatgaattcatccgccgtcagttgaatgacacccgttatatgggaaaaaaagccgtcgaatacttaagtagcatttgcaaagacgtgaaagcatttcccggacaattaacagctgaactccggcatttatggggtctgaacaatatattgcaatccgttcccgacattacatttgcaacccccgtacctgtcggaatgcgaaaaaattattacttaatcacaaacgaacagtaccaggttactcagatccttccccaagaagcagaccggcctcagacagaaaataatgaagttttcattaccggtacagtcaaacacaacagttttaaatatcggggaatgcaagactatccgacagaaagaaatgacgggaaatacggggcccggctgaaattatccgcccctgtcgcctggcatcctctgtttgcccaaaaaccaaattcctcggaagaacagcttgtattaaaaggaaaaatcgacaaaggcatatttgtctgtgactacctgaaacaaaaaataaaaacaaatttacccgacggcagctattgggtaagtcttccggtccggaaccgttctttccaggacagtgaatctgttaaccgccccaaattaactccacagcaagtacagctctacggcaccatccatgagggaagattccattgtgacacctaccaatgccaaactacaggaggtgacggtaaattctggtgtatactggatgtcgataccgaacagcctgaatttaccagaatcaaaaacgaattccccgcactggaagaaagacagattattcttaccggcagcatagacgaaaaaggcatcttttgtgcagatgatgacttaaattataaagtatcaaccactctttccaaagggaaatattacggaatctttccctgggaatcccaacagccggagttaagcgaaatagaatttacagccccaaaaatcaacaaagacgaaaagctgattgaaggacaaatctgggtggatgagcacaccggagaaatcaagtttgatccaaagaaaaacagagaggaccagcgtcaccatgccattgatgccattgttatcgccttatcctctcaaagcctgttccaacggttaagcactttcaatgcccaacgggaaaacaaaaaacggggaatcaactccacagaacactttccgctaccctggtccgggttcatcggagatgtaagtcaaagtgtgaaaaacatcctggtatcctataaacaaaacctccgtacgcggtgtaaaatcagtaaaaccctcatcaaagacggacggcaaatccatagtgtgggcaatgcagtccggggacaactccacaaagacagcatatacggacagcgacttgctcccggaaatacggaaaagacctaccatatccgaaaagatatccgggacctcaaaaccaaaaaacacatcgaaaaagtagttgatcccaccatccggcaaatgctcctgacacatttacaagaaaattgtcacatcaatacaaaccaggagttcgatattccttctgatgctttctttaaagacgacaaataccggattttccttcccaacaaatacggagaacctgtacccgttaaaaaagtccggataaaagaaaaaatggaaaatgcagagcaactgaaaaataacgtcaatcaatttgtcaacccacgaaacaatcaccatgtcatgttgtacatcgacagagagggcaatctgaaagaagaaatcgtatctttctggacagtcatcgaaagacaaaaccaacaccagcctgttttccaacttccggccgacggcaaagcaccaatatgcaccttgcaaatcaacgacacttttattatcggactaaaagacgaagaactggaagtataccggaatgaccgttccaccctaagcaaatacctctaccgggtgcaaaaattatccagtatgtattatactttccggcatcatctggcttctacaatcaaaaatgagaaagaagaagttcacattagaagccttgaggcctggaaacaagcaaatccggttaaagtatacattgatgaaatcggtaacctcactttttaa

SEQ ID NO: 38 - DNA, 4554 bp, unidentified
  source 1..4554: mol_type = unassigned DNA
atgacaaaagtattgggtcttgatctgggaacgaacagtattggatgggctgttgtggataaggaaggggagaaaatattggatgctggagtacgtatctttccggaaggagtagagccaacaacaattggaaaaggagataatgaacaatcaaagaatgctacaagaagagagaaaaggcaattgagaaggcatttttatagaaaaaggctacgcaaaataaagctacttgaagtattgataggtcaaaaaatgtgtcctttgagaatagaggatttacgtaagtggaaaaactggaattcaaaagaaaaagccgaaggaagaagatttccttcgacaattgaatttgacagatggatacaaatgaacccatatgaattgagagataaagctttaagagaacaagttactttgtttgagttcggaagaattatgtatcatttaatacaacggagaggttttttaagtagcagaaaaggagaacaagatcccaaaacactttttgaaaaagggaagccggatgaaaatattttacctgttaacgagactaaggaaaaaattggcaatactagtcttggctcatatttaaatagtattgcatataaagaaaatactccgtacaaaacaagaactgacgtaaaagggaatgagattagagttcgaggtcgatatacggttagaaagatgtatgtcgaagagtttatgaaaatttgggacaaacaaaatgaatattatgatctagatgtaattaaacttaaaacgagaaagattagagagttaagaggttcacttaatggtgcaaggaatagaaataaattagaatatctggtaagtaaatatggtgaagaaaatgttataattgaacaaaaatcaaataatattacacaagttattacaattagtgaaattcttttaaagaagtatctgggaggagaaatatattatgttgaaaatgaagaaggagaaaaaaagctaaaattcagaagtaatgagagtgtgttgttttggcaaagacctttaaaatcacaaaaaagtttattgacagattgcaggtttgaaagtaatctgcctgttattatgtcaaatggtgaaataagaaaagataaagaaggtaagtttgttttaagaagtaaaaagccttgcccgatatctcaccctgaatttgaattgtttagagcttatcaatttgttaataatattcgttttggtagaaatacaaatctaacaaatgagcaaaaggatacagtgttagatcttattaattcaaaagatgcaagtttttacttttcagatattagaaaaaaattagaattaacttacgagaagtttaattatgatgataagtttaaagttgttggaaatcccacaataaagaaattatcatctctttttgataagaaaatctgggaagaaaactatcaggatatttggcattgcttttatttttacgatgataatcagaagttatttgaaaaacttaagaaagattatgattattcaaaaggtatagacctgttggtaaaagttaaattaaaagatggttatagtaatgtgtcattaaaagctattagaaatatattaccatttttgaagaagggatatcaatatgataaagcagtaatattgggcggagttaaaaatgcttttggaaaaagatgggaatactttaaagattcacattacaaaattgagaatgaaataattagtattctttctgaagataatgcagaaggggacgcaatctgtaaaatcaaagaacacctgagttcacctgtattcaactatggttttagcaaggatgataattggtttgcgcatttgtaccatcatagccaggaaattgagaaggcatcgaaattttctgataaattgcctattccggaaaacttgcgtaacccaattgtgcagcaagggattaacgagatgcgtagattggttaattctttaatggagaaatatcaaaaccaatatggagatgattttcgatttgatgcaattaaagttgaaatggggagagatttgcgaaatagtaaatctgccagacaagaattaacattcagaatcagagacaacgagtcaaaaaacgaagaagcacgtgcaaggcttgctgagtatggtctgcagcaatcgagaagtaatattcataagtatttgatgtggaaagaaattgaagagaaagcaggagcagcaaaatgtccatacactggtaaaactatttccattagtgatttattaggaacagataattctgtgcaaattgagcatattattccatacagtgtctctttagatgatggatttggtaataaaacaatctgtgaatctcattttaataatttaaaaggagaattaactccttatcaattttatcaaaaaagatctgataaagatttgtggggcgtgaattcctgggatgcagtagttgaaagagcttttcagattttgccatataataaggcaaaaagatttacttcaaaaaaagattttgagaaatcttcctttattgaaagacaattaaatgatagcagatatattgcaaaaaaatctattgaattactttcaaatatctgtgagaatgttcgtgttatgccgggacaactaacagcagagctaagacatctatggggaataaataatgtattgcaaaagcctgttgttggaataaatgaccatgtgttaaaattggctaaagaagaaactgtaaaatgttatttagtggttgacgaagagaacaatgtaatagactttgtaaaaaagcagaatgatagacccaacactaaggagggagaaatcttaattccagtttcagtaaataagaataagttaataagcaatttgttttcatttgatatagacgtcgatgatttacaagatggtaaatattggattaaagttaacttaagtgatgattttacagtaatcccaaagcatattgataaagctaatgtggatgatgacagaattgtattcagaggtaaaattgagaaacaagtattcaaaaatgacacttcgggaaatgttaataaaaccggtaaagaagatggtgtttattgggcaaactttggtattaaagacaaaaagtttgtagatttaaaaactaataaggaacgtccgaaaactaaatcaaagaaacaaattgtgctttttggtaatgtagccaatgatgaatttaaatgctacatttataattgtcagacaaatctacctgatggtaaatactggttaatattagatattgattataatgatgtgtcttttgagaaagctattaatgagaagcctgaattacgtaatgatcaggtttgtttaagtgctacagttgatcaaaattcagaattaatactggatgttgataaaacttataaaaagcaggtaggattagctccgggtaaatattattctgtgtatcaaatagttgattatacaaaagaactttgtccaattgagaatcaaaagccaaaagttgcaaatgaacaaaaaattattgagggtagtatatgggttgacaaatatacaggagaagcaaaatttgatcctaaaaagaacagagatgaccatagacaccacgcaatagatgcaattgtgattgctctgacagaacaatcttatttacaacatttaagtacgtataatgcgaaacaaaaggcacggcaaagaggcaagattgatagtacagaaaagttttctctgccttggacaggctttataaatgacgtgcataaaaaagctgatgaaatacttatttcatataaaaagcaaaacaaagttctcactaaaaacaaaaaaggttttagtgttaggggacaattgcataaagaaaatgtttttggaaaaagacaagccccaaatcaagaacaagggtatcatagaagaacgaaagttacagatttagaaaacaataagcatatagcaaaagttgttgatgatagtgttcgtaaaataattttagaccatttaaggtataactgtgctgtggatataagtaatcctaaagggtttaaagtgccaaaagatgcatttgttaaagatggagaatggcaattgtttttaccaaacaaaagaggagataaggtgccaattaaaaaggttagaattaaggaatctattggaaatgcagcacaattaaaatcaaatattaatcagtttgtgaatcctaggaataatcatcatgttgttatttatgaagactttgacggtaatttacaagaaaatgtcgttcaattttggacagttgttgaaagacaaatgaatggtgaggagatatacaaattgccacatgatggtaaaaatattattactacactcgaaataaatgatatgtttattttaggattaagtgatgaagaatttgaagataataaaagcaattatgcatttttaagcaaatatttgtatagggtacaaaaagtgtcgtcaatgtactatacttttagatatcatttagcatcgacggttactaataatgaacaggaattttctattcgaagtatgaaatcatttgtcgaagctaatccaattaagttaaagataaatgaacacggaaaattagtttaa

SEQ ID NO: 39 - DNA, 4617 bp, unidentified
  source 1..4617: mol_type = unassigned DNA
atggcaaaagtattaggactggatttagggacaaactcgattgggtgggcggttgttgaagatgagcaaaacatgatacttggtatggggagtaggatatttcctgagggtgtgaaggcagacacaataggaaaaggtgataaagaagaatcaaaaaatgctgctcgacgaaatagcagacaacaaaggcgacagtattatagaaaacgactcagaaaaataaaattattacgtaccctcattgatttagatatgtgtccgcttactcataaagagttagatggttgggctaaatgggataaaaagaaaggtaaggcaggtcgtaaagcaccaagttctaaacaatacgaagcttggcacagacaaaatccttattatttaagaaacaaggcattgcatcaagacttaacattacaagagtttggaagggtactctatcacttaatacaacgccgtggttttttaagtaatcgtaagggaaaagatgatggagctatatataaaggtaagccaggtatgactggaattacagaaactcaaaagcagatttcagaagaaacattaggtagttatttatattccatttacccaaaggaaggcgagccatttaaaatgataactgacgaaaacggaaatgaacttcgggttagagctcgctatactttgcgtgatatgtatgttgaagagtttgataaaatatggaagaggcaagctagtgttttgggtttagacaataaaaccgcatcaaaaactaaaactcagtttttaagaggaagtttagaaagtaatcgaaataaaaagaaagttgaaaaattaaaagagaagtatggaaatgaaaatgttgttattgaaaaaattcaagataaagaaagcaataaaacccttcacaaaataacaactacgactactgtttcattaaaagaatttttgggtggagaaatacagtatgatgaagatgggacattgaaatacaaaagtcaagacagcgtgcttttctggcaaagaccattgcgctctcaaaaaggacttctggcaaaatgtaggtttgaacctgatgtaaaaaaagatggtaaatatttgcaaaaaggtaaaaccccttgtcatttgtcacaccctttatatgaagagtttagagccttgcagttcatcaataatattggatatggtagaaaacaaaagctggatgaaacacaaagggttcaagtcttagaaatcataaactcaaaggatagtaattttaatttctcagagatacctaaaaagttaaagatgacttatgagaaatggaactatgcggatgatcaaaaagttcccggaaactacacaacaaagcatttaagtttactttttgagagtgatgtatgggaatcccataaagaagaaatatggcatagttttcatttttttgaagataacgacttattgatagagaaattatcaaatgattttggtttagcaaacaaggattttgaaaaggttgaaaatattaatttaaaagaaggttattctaatgtttctttaaaagctattaaaaacataactctttatttaagaaaaggatatcaaatgagtgatgccactatattgggaggtgttagaaatgcttttggaaatagatgggattattttactgactcacatcaacaaatagaaaaggatgttttagaaataaacagaacaaaaggaaatcaagaaggagaagccattgaaaaaataaagaagtatttggcaaacccaaacaataattttggttttactgaaaacgacaagtcatttataagattatatcatcacagtcaagagattgaagaaaagccattacaggataaattggatgaagtggaaaacttgcgaaatcccatcgtacaacaagggcttaatgaaacaagaagaattgtaaataccctcattgataaatatggaaagtttgataggattcaggtagaattaggacgggatgtaaaaaacagcaaaaccggcagacaacaactagccaatcgtatcaaggaaaacacttctaaaaatgatgcagcaagacagttgctaacagaatttggattaaaacatagtagagaaaatgtgcaaaaggtgcttctttataaagaaatgcaagaaagaggtgtggttactgtttgtccttataccaataaatccatcaatattaaagacgttttaggccgtgaaaacaagatacagatagaacacattatacctaaaagtgtttctcttgacgatggatttgcaaataaaacgctgtgcgactccgcatttaatggattaaaaggaaatcttacaccttatcagttttatttaaaaaacagtgatgcgtcactatggggtggcgcaaccagttgggaagaaatcgaacagcgagcctatcggcttctgccttacccaaaagctaaacgattcacttctaaaattaaagtagaagatgccgacgtaaaaagcgactttatagaacgacaattaaatgatacacgatacattagtaaaaaaacaaaagaaatactatcgcaagtttgtaaagacgtgagggtgcttcctggtgcgctaacatcagaattaaggcacctttggggtctcaataatattctgcaaccagtaatgaatttaaacattcaggttaacgagctcgataaagataaagtgtacccacattatgtagttttaaatgggaatcaagaagctgtaaatgcggttccaatttacaatcaaaaaccaaaactaggtactaatgaaaccactattgccggaaagatagataaaggagtttttaaaactactgaaaagtatatgaaacttaaagtagatgctccggaattaccaaacggcgactattggttaaaactgaagctctcagagcctaaaaacaccgtgcgtatttttaaggaaagacctgttgtggatgaaaaccaaatagtgttaaggggtaaaatagaacgcagtaagtttaaaaacgatggtattggcacagtcaacgccaatcgatttgacaacggaagttattgggcgacatttaatgtgcttaaaaagcattttgaaaaaccacaaaaagacaaacaacccaagaaaacaagcaaacaattattgttgtttggagaagttaaaggcaataggtttaaatcgtttatttatgaatgtgaaaccaatgaaagtgacggtaaatactggctattgcttgatgtagatgttgagaatgtaaattttgataaagccattagtgagaaacctgaagtaaatcaaaatagtatattgattgaaggcagtataaacgatgaaggactttttgtttcagaaattgaccctgaacaccagtttgaaacacaagaaaaagaaggaaagtattgggtggtttttgatattgaacaaactcctaatgaatttagtgccattgaaaacttaaaacccaccttagaagaaaaccaaaccttaattgaaggtgctatttgggtagataaatttaccggtgaaataaagtttgaccccaagaaaaatagagaagaccatcgacatcacgccatagatgcgttggttgtagctttgtcaaaacaaagctatttccaacagttaagcaggtataatgctgctcgtgttgctaagaagaaattaaacccatttgacaaagaacaattagattttccggaaccttgggatggatttcatactgcagccaaaaaaaaagctgaaaagatattggtgtcacacaagcaaaaccgaaaagtgttgaccgaagtaaggaagagaattactaaaaacggtaagacttacaccagtgttggtgacgccgtgagagggcaattgcataaagagaatgtatatggacaacgccaagcgccttttgaaactacaaaaggctatcatattcgaaaaaatgtagcggatttgaaagacaatcaattgaaaaaaatagtggatttagaaatccgtaaaatcatcattaacgccagaaaagaggaagctattttgaaaaagcaaattgatgctctggaaaaacaaaaaaagaaagtacgtactgatgaagaagaaaacactttgagtgaacaaattaatactctaaaaaatgatattattaatctctatactttgaaaaacaaaaatggcgcacccgtacccattaaaaaagtgcgtataagagaaaaaatgacaaatgctgaaaaagttaaggatgaaaatcaatgggttaatccgcaaaacaaccaccacattttaatttatagaaatcataaaggggagttagataaagacgtagttaatttttggacagctgttgaaaggatgaaacaaaacattgatttagttcagttgccgagtgatggtattgaagttatagaaacatttcaagaaaatgatatgtttttgttgtttttggaagataatgtagaaatatcaacactcgatgataatcaaatctttaatcatttatataaggtacagaaaatagctggagctgattactttatggagatttgttttagacagcatactgacagtaaaccgtcaacagaagctgattatgtttacattaaagggtttggtaacggtaaaacaggttggaacagattcaatccaattaaggtaaaactttctgaaactggaaaatttgaaaaaatataa

SEQ ID NO: 40 - DNA, 4698 bp, unidentified
  source 1..4698: mol_type = unassigned DNA
atgtgtaaaaaacaatataccctcgggcttgacttagggacaagcagtcttggctgggctgtcactgtacgtgagggagttagcgtaggctctatcctcaatgccggagtcactgtgtttccgtccgggaaagcgatggtcaatggagtcgagaaaacaaacaacgaagagcgacgtaatagccgtcaggcgaggaggcaactggctcgccgccgcctgcgcaagcagcatctactgagactgcttatagagaatgacatgtgtcccctgacgcagacggagctagacggatggatgaagtgggataagagcaagccgaccgaggagagacggcgcttcccggactcgccccgtttcagggagtggctggcccttgacccataccgactgagagctgaggctgtagatacgccccttgaggatgaccccgacggaggctttacagctcgtgaaaagctggggcgtatcctttactccatcatccagcacagaggtttcctctcttcgaggagtggaaaggaagacggatctatttatacaagtacgatggagaagaacgggaaagtcggcatcggtgagacgagagccgctctcggagatcgcacgctcggtcagtacctctacagcattgccccaaaggaaggggatcccttcagtgatcagacggatagagtacgatctcgctacacactgagggagatgtatgtggaggagtttgaccggatctggacgagacaggcacccctgcttggtctcgatagggagcaggtggagacgatccactaccggtacttcaccggtaagagtgactcgtatgaggcaagacgtcgactggagaggtatcgggccaaggatccgtcagctgtgatagagaccttgtccggtgacgagcaccgcttcgtttacaagactaccacttctcttaaggagcatctggcgggtaagatttggtacgacaaggatgggaccatcaggtttgtctccaaggagagtaccctcttctggcagagacctctgagatcgcagaagggtatgctggacaagtgtagccttgagtcgcataaggttaaggacagaggcacaaatcgattcatcacccagggaccaacggttgtctctgcctctcatccctactatgagctgtacagagtgctgcagaccttacttaatatgagagtacggggccagaagcttgatctgatgatgctccagtctatgctcgacaagctccttacttatggaaagaatacaaaggtctctaagctcaagaatgacctccgactgactgctgaccttaacttccctgatgactttgagctgcccggatgtcccacgattagtggtataactaagctccttgacagtaagaagatgccctcccgtaagggagatgcgagtcccagtgatcgactggtcatcccctttgacctcaatctctatacccggatctgggagaaacttttcttctacgatgacaatcatctgctggcgcaaggtctgaagaagatagagggtgcggtctttgtggaggatctcgaggacaagcttgctaagctgcatctcagcgaggattatggcaacctctcgctccacgccctgcgtaacatcctgccctatctcctacgtggtgaggagctctacgacgcggtgttgctcggcggcatacgtaatgcggtacgggatgttgcgctggtggatgatcacgatgatgcgctgagatcgtggatccactctcagggacctctggagcgcggggagatgatagataggatcgttgccttgctgcaggacccggacggaccatttcatctcgatactccggagctgaagctgcgtcagcgactctatcatccctctcagggcacagctaagcaccagctgatggatcagctcccgccggtggagaaccttcgcaaccccatggtcgagcaggcgctatggtcgatgaggagactggttaattatctcctcaataactatcgctccacgatccgaccggactttacctttgacgagatccatgtggagcttgcccgggacctcaagctatccactcagaagagggcagagatgagacagaggcagaagaaagcggaggaggacaacgatatggcaagggactacctgcgcagccaggggctgagcacctctcgagagaatctccagaagtacaagctctatatggagattgcaggtcgtagtatgggggtcgtcacttgtccgtataccggcaaggtcatacgtgggctgagtgctgcacttgatggcaacagcaacttccagatagagcatatcgtcccccgcacagtgacttacgacgacagctttgccaataagacactctgcgatgcagacttcaataggaagaagggcaacctgaccccgtggcagttttatcagcaggatccgagccctaagctctggggtgctgactcgtgggaggaggtagagcgcagggcattccgtctcctgccgtatcgcaaggcgcttcgcttcgtaagtaagaccgtagaggatcagctctccggagatcagctggtcgacaccgcctatattagtcgaaaggctaaggagtacctctcctacatctgtccgcaggaccatatccgactctatcccggtcgagtgaccgcaagacttcggcagctctggggactcaactcaatcctctctcaaggtcccactgtcaccaaagaccagctcccgacggagtgcgcactctctactgatcagatccctgctgtcgtcatcggcaataagaagagcggcgagctcagttgcatactacctaagtacacatctcgcccggagcgggaggagggtgatgtggtcctgctggtaagggtgcagaaggggaagatatacagtgccaggtatagtgagctgccactatcgtgggacaaggctacttctcttgcggacgggacatattacattgtcctccgtagtacagaagtgctccgcgtggagcgaatgtttgctgacgaacctcgattgagcgaggatcagctgagagcaagggttaccatcgagctgaatagagatggacagcccaagctctcctccagggagatggatgtccgcattctgaaggatatgcctttgcccgctgccccgggtaattactggctgacgatacccctcgtctctgtcccggaggttactcctgagcatccgagagcaaaggttgcccccgacacaatcaaggtcacgggatcagtggagcagggtgaatttatctccaaggtgtaccggcagggtggtgtggctatggcagatggcgtggcgcaagtcatcctcacccctatgctatcagaggcgtccttcactaagatcaagctagatgctcctgtgatcacctccggagaggtggtgctcacgggtgccgtcgacacgagtgacctcttcgtctgtgaccaggacgatgagcttcgagtgcagattgactcatttgatgagccggggaagtactatgctgtcttctcctttgacccgatggcgtcgtacatcctttcgtatgaacaagcaccaaagctgagtagtgacgaggtggtacttgatggcgaactgatctataatgataaggacgaccggatccacttcagtcctgcaaagaatagggatgaccaccgacaccatgctctcgatgcaattgtcatcgcctgcaccggacaaaaggtgctgacagcgatgtctacctataacgcacatgcagatgattacgagagaggtacaggcaacagacccacattttcctctccctggcagggcttccgtgacgatgtgagagtggcgatcgagtccgtactcgtcaagcacaagcagagtaccaagatcctcaataaggtgacgaaaaagatcaccaaggacggtcgcactcatcgctcccagggtcttgccgtgcgtggcaagctccataatcaaacattctttggcctccgccagtccccccaagagagtttcttaagtctacacaagagagagagtattcaaaatatcgaatcttattcttcactatcaagagttgctgatgtcaccatcagagatctgatgattagccgcttgagggatattggcatagatactacacgaaaagagtattcaataccaaaggaagcccatgcgtttgtagatgaggagggaaactataccattcacctccctaacaagcgaaaaggaggtgctccaattcccgtgaaaaaggtgagactgtgtgagcgttcaggctctccaatacaaattgcgggttacaataagtgggttaaaccagagaacaatgaatatctaatggtttatacaaactatgacaataagatccagtatctgtgcgtgccgttttttgaagaagtgaacaataagataagaggtgttgatgggtggaaaaaactgaaagacctaaaggaggttttgctggttttacggattaatgatatggttctctgggggctgaatacaaatgaagcacgtagtattctccaaagtggggattatagagaggcttcaaaatatttgtatagggtgaatgctatctctgcaggagaatttgagttgcgtcatcattctgatgccagaactgacaagagcagtcgcacaagaatcaccccaggtactttgaggaaacgtgggttgcctattaaagtagaggtgagtacgatgggtgaattgtcagttgtaaggatagcggagagcgatgattaa

SEQ ID NO: 41 - DNA, 4698 bp, unidentified
  source 1..4698: mol_type = unassigned DNA
atgtgtaaaaaacaatatatcctcgggctggacttggggacaagcagtcttggctgggctgtcatcgtacgtgagggagatagtgcaagatccatccttaataccggatccagtgtgtttccgtccggaaaaacgatggtcggtagcaaagagaggtctaacaatgaagagcgacgtaacttccgccaagcgaggaggcaattggctcgccgtcgtctgcgcaagcagcatctcctgagattgcttatagagaaagatatgtgtcccctgacgcagaaagagctggacgggtgggtgaagtgggataagagcaagccctccgaggagagacgacgcttcccgcagtcaccccgcttcagggagtggctggcccttgacccttaccgactgagagctgaggctgttgatacgccccttgaggacgaccccgatggaggctttaccgcacgtgaaaagctggggcgtatcctttactccatcattcagcacagaggcttcctctcatcgcgaagtgggaaggaggacggatctatctataccagcacgatggagaaggatgggaaggtcggcatcgatgagacgagagccgccctcggagatcgcacactcggtcagtatctctacagcattgctccaaaggagggtgacccctttagtgatcagactgagagagtacggtcccgctacacgctgagggagatgtatgtggaggagtttgaccggatatggacgaggcaggcaccagtactcggtctcgatagggagcaggtggagacggtacactaccggtacttcaccggtaagagtgactcgtatgaggcaagacgtaggcttgagaggtatcggaccaaggatccgtcagctgtgatagagaccttgtccggtgacgagcaccgcttcgtctacaagactaccacttctcttaaggagcatctagcgggtaagatttggtacgacaaggatgggaccatcaggtttgtctccaaggagagtaccctcttctggcagagacctctgagatcacagaagggtatgctggacaagtgtagccttgagtcgcacaaggtcaaggacagaggcacaaatcgattcatcacccagggaccaacggttgtctctgcctctcatccctactatgagctgtacagagtcctgcagaccctacttaatatgaggatacgggaccggaagctcgatctaatgaggctccagtcaatgcttgataagcttctcacttatggaaagaatactaaggtctctaagcttaagaaagacctccgactgactcttgaccttaactttccggatgactacgagctgcccggatgccccacgattagtgggataactaagctccttgacagtaagaagatgccctcccgtaagggagatgcgagtcccgttgatcgactggtcatcccctttgacctcgatctatatacccggatctgggagaagctcttcttctacgacgacaatcatctgctggcgcaaggtctgagggggatagagggtgccgcctttgtggaggacctcgaggacaagctcgccaagctgcatctcagcgaggactatggcaacctctccctccacgctctgcgtaacatcctgccctatctcctccgtggggaggagctctacagtgctgtgctgctgggcggtatacgtaatgctgtgcgggatgatgcgctggtggacgatcacgatgatgcgctgaggtcgtggatccacacccagggacctctggcgcgtggtgagatgatagataggatcgtggccttgctgcaggatcccgacgggccatttcacctcagtactccggagttgaagctgcgtcagcgactctatcatccctctcagggcacagctaagcaccggctgatggatcagctcccgccggtggagaacctccgcaaccccatggtcgagcaggcgctatgggcgatgaggagactggttaattatctcctcaataactatcgctccacgatccgaccggactttacctttgacgagatacacgtggagctggcccgggacctcaagctatcaactcagaagcgggcagaggtgaggacgcgtcagaagaaagccgaggaggacaacgatatggcaagggactacctgcgcagccaaggtctgagtacctctcgagaaaatctccagaagtacaagctctatatggagcttgccggtcgtagtatgggggtcgtcacttgtccgtataccggcaaggtcatacgtggactgagtgctgcattggatggcaacagtatgttccagatcgagcatatcgtcccccgtacaatgacctacgacgacagctttgccaataagacactatgcgatgcggacttcaataggaagaagggcaacctgaccccgtggcagttttatcagcaggatccaagcccaaagctctggggtgccgactcgtgggaggaggtggagcatagggcattccgtctcctaccatatcgcaaggcaacccgcttcgtgagtaagaccatagaggatcagctctccggggatcagctggtagataccgcctacattagccgtaaggcaaaggaatacctctcctacatctgtccccaggaccatatccgactctatcccggtcgggtgaccgcaaggctgaggcaactctggggactcaattcaatcctctcgcaaggacccactatcaccaagggtcagctcccagcggggcttgcgctctcaggtgatcaaattcctgccatcgtcattggcaaaaagaagagtggcgtactcagtcgcattctgcctaagtacacctctcgcccggagcgagaggagcgtgacgtggtcctgctggtaagggtgcagaagggaaagatatatagtgccaggtacagcgagctgccactattctgggacaaggctgcctccctggcggacgggacatattactttgtcctccgtagttcagaagtggtccgtgtggagcgaatgtttacagaccaacctcgactgagggaggatcagctgagagccagggttaccattgagctgaataaagacggacagcccaagctctcctccagagagatggatgtccgcatcctgaaggatgtccctttgcccactgccccgggtaattactggctgacgataccccttgtctcagtccctgaggttactccggagcatccgagagcaaaggttgccaccgacacgatcaaggtctcgggatcagtggagcagggtgtgtttgtctccaaggtgtataggcagggtggtgtggctatggaggatggcttggcgcaggtcatcctcactccggacctctcagaggtgtccttcaccaagatcaagctggatgctcctgcgatcacctcgggggaggtggttctcacgggtgccattgacaggagtgatctcttcgtctgtgaccaggatgatgaacttcgagtgcagatagactcatttgatgagccggggaagtactatgctgtcttctcctttgacccgatggcgtcctacatcctgtcgtatgaccaaacacctaacctgagcagtgacgaggtggtactcgagggtgatctgatctataatgataaggacgataggatccacttcagtccgtcgaagaatagggatgatcaccgacaccacgcacttgatgccatagtcattgcctgcacaggacagggagtgctgaccgcaatgtctacctataacgctcacgctgatgattatgagagagggacaggcactaagcctacatttgcctctccctgggagggcttccgtgacgatgtgagggaagcgatcgaggccgttctcgtcaagcacaagcagagtaccaagatcctcaataaggtgacgaagcagatcaccaaggatggtcgcactcatcgctccaagggccttgccgtacgtggcaagctccacaaggagaccttctatgggcagcgacagtcacctgaggagcctgctccgacaatgcactatcgagtgcccattacttcaatcaccaaatcaaagcaactaaatcaagtggcagacccgacaatccgggcgttgatgatccggcgattgcaagatatgggagtggataccacgcaggaagactacgcaattccaggcaatgcgttcgtggacaaggcgggtaactacaccatcctcctaccgaataagcgtggagtccccgtcccggtgaagaaggttaggcgtagaagggttgttaataatacggttcagcttgatggttacaataaggaggtggagactagtaacaatgattttgcgcttatctatgaggatcaagagggtaattattgtgaggaggtggtgaccttttgggactctgcagatcgattgttgcatggacaggatagagtacagctcccaatggatgcaaagagagtcgtcatgaccctgcgcatcaatgatatgttcgttatcggcatgagcgaagacgagctgcggagtgctatcgggtctagtgattatgggggtatctctgatcacctatatagagtgcagaagctctcttcgatgcattatgtattcagactgcatagagccagtactctcaatcatgacgaggaactctatacaattcgcagctttagtgcactcaaagagtctactcccattaaggtgataattgacacagacggcacaatctctatagcgacacgagatgatcaagcgaacaatcaccatcag

SEQ ID NO: 42 - DNA, 4476 bp, unidentified
  source 1..4476: mol_type = unassigned DNA
atggaaacaactttaggtattgatttaggtacaaatagtatcggatttgctataatcgatcaggatcagacaaaaatagtaaattgtggagtacgtatttttccggaaggagtgaataaagataccatcggacaaggacataaagaagaatcccggaatgctacacgccgaatgaaaagacaaatccgtcggcaatatttccggaaaaagttacgaaaaattaaacttttggaagtattggtccaatacgatatgtgccccctcaaatctgaagaagtgcggcaatggaaaaattgggacaagcatcaaaagtcagccacccgtgaatttccggatacacctgaattgagggaatggctgaaactgaacccctatgaactccggcaaaaagctattacagatgacattacccgccaggaattgggccgaatcttctaccacctgattcaaagacggggctttctaagtagccggaaaggaaaggaagacggaaaaatattcacgggtaaagaccagatgacaggtatcgatgaaacccggaaaaatttacaacatcgtacattgggcgcctatctttatgaaatttcccaaaaaaaaggagaaaaatacaaattccgttccgaaagagttagagccagatacactctgcgcgatatgtatatccgggagtttgaattgatctggaaccgtcaggcgcctcatcttggattaagcaatgaacaaactacacaaaaacggaatatttttttaaaaggctcatgttgcaatatccggaaccgtaaaattatcgatcatttaaagttgaaatacggtaaagaaaacgtcagcataaaaaattcacgtgtaacggttttatcacaggttcctctgaaagaattactgggaggaaaaattgaatacaatggagagaatttaaaatttaaatgcaatgaaagtgtgctcttctggcaacgcccactccgttcacaaaaatcgttactttcaaaatgtgtttttgaaggaaagcatttttacgattaccgccataagaaatggattaccatcggcccaacaccagctcctctgtcccatccggaatttgaagagttccgtgcattccagtttatcaacaatatcacatacggcaaaagcgaacatctgaatgctgcacaaagagaaaaggtcttccaactaatgtgtaaggaaagtaaggatttcagcttcggcaaacttcaaaaacacttaaatctgttcgaaaaattcaattttgatgagggaaccaaagtacctgtttgtaccaccatcagccaattacaaaaattattccccgaagagatttggcagcaacaacgggaagaaatctggcattgtttttacttttatgatgacaatgaactgttattcctaaaactaaaacaggattataacttacaaattgatgatgttgagaaaataaaaaaaatcaggcttgcagaaggttatggccgtgtatccctgaaagccatcagacggattaatccttatctacagaaaggatacacctatagcaccgcagtgattttgggtggtatccggaatgccttcggtagccgttttgaatatttcagggactcggaaccagaaatagaacaggacgtctgtcagatattaaaagaaaaaaacacagagggggaagcaatgcaaaaaatcaagaaatatctcactcatcattcctatggatttactgaaaacgatagggctttccaaagattatatcatcacagtcaacccttagagatgaaggaattggaagaaaaactgcctgcaacagagaatttacgcaatccgatcgttcaacaagccctgaatgaactgagacggacagtcaataaattattgtcagactgccgccggaaatacggagaatctttccaattcgaccacattcatatagaattaggcagggaacttcggaattccaagagtgaacgggaaaaacaaacccgctccatccgggataatgaaaagaaaaatgaagcagcaaagcttaagcttcaggaatttggtttacaaccgagccgggataatatacaaaaataccttctatatagggaaatagaagaaaaaaacggcatcgtctgctgtccttataccggaaaaaccatcaatatcagcgacgtattgggcagcgaaggatgtatccagatcgaacacatcattccttacagtatatccctagacgatagtttcgccaataaaaccctctgtgacgcaacctttaaccggaataaaggtgaactcagcccctatgatttttatcacaaaaatccctcacccgaagcctggggccttcattgctgggaagatatagaagaacgggcatttcgcttattaccctactccaaagccaaacgttttatccgccgtaaaaatccagacagaaacgaattcatcagccggcaattgaacgatacccgttatatgggcaaaaaggctgtcgaatacttgagtacaatctgtaaagatgtaaaagcgtttccaggtcagctgacagccgagctccggcatttatggggattgaacaatatgctgcaatctgttcctgaaatatcacttacgcttactgatcctatacccggaaaaagaaaaaattattatgtagtgacaaacgatcaaaatcaggtcgtacgtctctctccgaaagaagcagacaagccacaaacagaaaaaaatgaactgctcttaacaggaagcgtcgaaggcaagatattcaagtataaaggtataagagaattccctaccgaactttcagacggaagatatcgtctccggttgaaactctctgccccgtcctcttggcaccttctgtttgccgagaaaccagtatcgttagaaaatcaacttgtcataaagggaaaaattgaaaaaggaatctttgtctgtgaccgtctgaagcagaaaatcaaaaccgacttacccaatggcagttactggatcagtcttcccgtcaccaaacgtttcttcgtggaaggtgaatctgtaattaacagtaaactgacatcccggcaaatacagcttttcggaactaccagagaggggatttttcaatgttccacttaccgttgccggacgacaggtgcagacgggaaattctggtgtactctggaagtagataccgatcatcctgaattcacaaaaatcaaaaacgaacctccgtatatcgaggacgaccaaattgttctaagaggaagtgtagacgagcaaggaacattttatgctgacgatgacccacattatcaattaactacttcacttcccaaagggaaatattatggtatttttgtaattgaacaccgttatccggaattacttgaactggaatataccgctcctaaaattggtaaaaatgaaaacctcattgaggggcatatctgggtggacgagcacacgggagaaatccggttcgatccgaagaaaaaccgggaagatcaacgccatcatgccatcgacgccattgtcatcgccctatcttccctgcgtctttttcagtgtttaagcacatataatgccaaacgggaaaataaaaagcggggtatcaattccaccagctcttttcccttaccttggccgggatttattcaggatgtaagccaaagcgtagaagaaatattgatctcctataaacaaaacctccggacactttgcaaaatcagcaaaacaatagtaaaagagggacgcaaaattcaaagcgtaggaaatgccgttcgcggacagcttcataaggatactgtttacggacagcgtcaggcaccgggaagtctagagaaagcattccacatccgcaaagatatccgcgaacttaaaacaaacaaacacatagaaaaagttgtagatttaaccatccggcagttgattgtgaaacatttacaggaaaattatcacatcgacaccacgaaggaatacactgttccagccgatgccttttttaaagatggtttttaccggattttcctacccaataaacatggtgaaccggttccggtaaaaaaaatacggatgaaggaaaaaatgggaaatgccgaacaattaaaaaatagtttgaatcaatatgttaatccccaaaataatcatcatgttatgctgtatacggatataaatggcaatctgaaagaagaagtagtgactttctggacagtcattgagaggcaaaatcagggacaacctatctttcaactccctcttgacggacaggaaacgatctgtaccctacagatcaatgatacttttgtcctaggtttaaagaatgaagaactggaggtgtatagaaatgaccgtgccgtattgagcaaatatctttatcgggtacagaagttatccggtatggattatactttccggcatcatctagcttcaacactaaagaatgaaaaggatgaaatccggatagcaagcctagatgcatggaaacgagctaatccggttaaagtccatatcgatgaagttggtaaccttactttt

SEQ ID NO: 43 - DNA, 4560 bp, unidentified
  source 1..4560: mol_type = unassigned DNA
atggcaaaaattttaggactcgacttaggaacaaacagcattggatgggctgtggtgaatgcagaaattgaaaatggaaaagtgaaagaatataatggaatagatgattccggtgtaagaatttttcctgaaggtgttgttgaaaaaggaacatctaaagaaatatctaaaaatgcaacgagaacagaaaaaagacaaacacgtcggcagttttaccgaaaacgaagacgaaaaataaaactgttgcaagttcttatagatcagaatatgtgtccgcttacgcatgaagaattagatgtgtggaaaaaatgggattctgtaaaaaaagttgaaggaagaaagtttccaagtaccgagaaatttaaagcatggttgtcattaaacccctatgagcttagagcaaaaggcgttaaagaagaactttcattagaagagtttggcagagttctctatcatttagttcaacgtagaggttttttgagtggtagaaaggttaaagaagacggaactattttttctaaaggaaaacctgatgagaatattttgccaatatatgctactcaagaaaaaataaaagattctactcttggagcatatttatattctattcttccacaagaaaacaaaccgtatactacaataaaagatgaaaatggtgtagaaatccgtgttagaggtcgctatactgtaagagatatgtatgtgtgggagtttgagaaaatatggcaatgtcaaaataagtatagaaactttaatgaagttttggttaattactcaaaatctcgaaaattgagagggaatatttcttcaagtaagaaccaaaagaaattagaaaaacttaaacaaaagtacggagagcaaaatgttcatataaaaaaagacactttagtatcttatgaaaaaattccattaaaagatttattagttgggaatatctggtttgaagataatgaaaatggcgagaaaacattaaaatttaagtcaagtgaaagttctcttttttggcaaagaccattacgttctcaaaaacaatatttatctgattgcagatttgagaataatatgccggttattaaaaaaaatggtcagtttttaatgaatccaaaaggggaagttcaaacaaatggaaaaaaaccatgtccgatttctcatcctgattttgaattgtttcgtgcatatcaatttataaataatatatcgtatggcaagaataatcgcttaacccaagagcaaaaagaattggtgttagatttaataaatgcaaatgaaaataactttgatttttctaaaataccgcaaaagttaaaattaacatacgaaaagtttaattatgatgataagcataaggttccgggaaactatactcataaaaaagtaaaaccattattttctaaagaaatatgggaaaaacattataatgaaatttggcactgcttccacttttacgatgatagcgatatgctccttgaaaagttgaaaaaaaactatgatttttccggcagtattgaagatgtgaaaaagatagtacttaaagatgattatagtaatgtttcattaaaagcaatacgaaatattttaccctatttagaaaaagggtatatgtatgatagagctgtgattttaggaggtgtgaaaaatgcgtttgcttctcgttgggatagatttttaacaattgaaaattttgagcaaaacttggaacgtgatattattgctattctcaaagaaaaaaataaagaaggtgaagcaattaataaaataaaaaaatatgtaatagaaaatcagtatggttttgttgaaaatgatccgtttttttctcgtttatatcaccattcgcatgaaatagaagctaaagaaattcaagaaaaagtacctttattagaaaatcttcggaatcctattgtacagcaagctcttcatgaaacaagacgtctcgtaaatactttaattaaaaagtattctgaacaaaatggcgtaacctttaggtttgataaaattcatattgaaatggggcgagacttaaggcaaaataaaagtcagcggcttgatgcaacatttaggaataatagaaataatgcaaaaaataatgaagctcgtgaacgtttagctgaatatggtttgcgtccgtcgcgagaaaatattcaaaagtatttactgtataaagaaatagaagaacgtgcgggtactgttcagtgcccctataccggaaaaacaatttctgtttcttctcttttagggagtaataatgcaattcaaattgaacatattataccgtatagtgtttccttagatgacggttttgcaaataaaacattatgcgaaactcattttaacaaattaaaaagtgaaaaaacaccatatcaattttatcaagaaaattctgactctaaactttggggggcatcttcttgggatgaggtaaaagatagagcgtttaaaattttaccgtatgccaaagcaaagcgttttgtgtctgaaaaagattttgaatcaaatgattttattgaacggcaacttaatgatagcagatatatagctaagaaagctgtagaattactctctgctgtttgtgaagatgtgagagcatttccgggacaagttacttctgagttacgacatttatggggagtaaataatgtgttgtctcctattaagaatttgtctaaagcacattttgatgttgatgaagaaagacgagtaccatattatgttattgtaaataatgaaacaaatgaaattctttctatacagagaaaactaaatgaaaaaccaaaaacaaaacctcaagaaatacttttgccggtatatataaataaaggaaaaattgagtctcaaaaatactcgtttagtaatgaagttgctcatttagaagatggtaaatactgggctcatgtagttgttgaaaatacggtatcttttatacctaaatatatttcaaaacctatagtagatgaaaatgcaataattttaaagggattagtggaaaaaggaagttttaaaaatgatacacttaaaagtagttttaaaacagtagaaaaggacggatattattggggaacatttgcagttacgaataaatcttttgagttgcctcaaaaagacaagcaacctgctccaaaaacaaatcaaatcgtattatttgggaatgttattaatggtgtttttacttgttatatttactcgtgtaaaacagattttcccgatgggaaatgttggataatacttgatattgaccctaattcaatagtgtatacacgagctaataatgatgaaccacaaatacatgaaaatcaactccttttaacagccgttgttgatgatacagcgaaagctatagttgatgtagataaagatgctgtgtttgaaacagataaacaacccgggaaatattatgcagtttttacagtgaaaagtgtagatgatttttcgaaaattgaaaatgctttgcctacatgtaataaaggagagagagttatagagggtgatgtgtggcttgataaatacaccggagaaattcagtttgatcccaaaaagaatagagaagaccacagacatcatgctattgatgctattgtaattgcgttaacacaacaaaattttttacaaagattaagtactgagcatgctaaaatgaaagataaagagcgtggtattatgaacactacagaaaagtttcctttaccatggaattcttttgtttcagatgtgaaacatatcgcagaaggcatgcttgtttcgtataagcaaaacacgaaaattctaactaaaaacaaaaagggagtgagtgtgagaggagcattacataaagaaacagtatatggaaaacgaaaagctccacaatcacaacagcggtttcatattcgcaaaaaaataacaggtctgtctaatgaaaaagatattgcgaaaattgtagattcatctattgtaaaattaattgaaaatcatttaagagatacgtgtggtgttgatatttctaagtcatatacaataccgaaagatgcattttataaagatggagaatggcggttatttttacctaacaaaaacggagaacctgtgcctattaaaaaaattagattaaaagaagttattggtaatgcagtacaattaaaatcagaattaaatcaatatgtaaatccgcggaataatcatcatgttattatatttaaagatgaagaaggtaatttacacgagcaagtagtacagttttgggaggttgtagagcgtttaagaaataatgaaaaagtatatcaattaccggtatctgaatatgggaaatctacaccaaaagaaatagttacaacattgcaagaaaatgatatgtttattcttggtctttcagatgaagagttttctgataatagacataatcatgcatttttgagcaggtatttgtatagagttcagaaaatatctagtggagattattcttttcgatttcatttagcatcttctataaataataaacaggaagaggttaggattgctagtattaaagcttgggaaaagaataatccaataaaagctgttattaccccattaggcaaaatagaaccaatagaa

SEQ ID NO: 44 - DNA, 4605 bp, unidentified
  source 1..4605: mol_type = unassigned DNA
atgacaaaaattttaggacttgacctgggaaccaacagcataggatgggctgtggtagataaagaagcaaataaaattatcgatgcgggcgtacgaatctttacagaaggcatcgtaaaggaaacaattggcaaaggcgacaaggaggaatcaaaaaatgctgttaggagacaattcaggcaggccaggagacagcattatagaaaaagacttcgtaaaattaaattattacaagttttgattgcccatgatatgtgtcctttaaaagaagaaaacttaaataaatggaagaactgggataaaaagacaaaaactgcaggcaaaaagtttccggatacagttgaatttattgattggcttaagttaaacccttatatactgagaaataaagctattaatggagaaattacacgacacgaatttggaagaatattgtaccattttatacaacgtaggggatttttgagttccagaaagggcagtgaagatggcaccatctacaaaggaaatgataaaatggatgggattagtgaaacaaagagtttattaaaagataatagcaccttaggtgaaaacttatacaaggtttatccaacagaaaataagccatttaaacgtacccttattgacaataaagaaattagaatacgagcaaggtatactttacgtgaaatgtacgtccatgaattcgaacagatatggaaaaggcaggctgagccacttcaattaaataacctgaaagtttcagtaaataaggacatcaacttggaaacttcagcacaatcgcctaaaagcaaacaaatgattgaaaagcttcagaataagttcggcgaatcaaatattgagataaaagataaacgtgttacaattagaaagcaagttccattcaaggagtttcttgccggaaaaattgaacaaacagatgagggtatcaattttaaatctaatgaaagtgttttattttggcaacgaccccttcgctctcaaaaaggtctgcttggtaaatgtacattcgagaatcacaagttttttgataaagcgaaaaataaatggattacagttggttcaaatccttgtccgatatcccatcccgattttgaattgttcagggcttaccaatttatcaataatattcgttttggcagatcaacaaaattaaatgatgaacagaagcaaaaggttctgaaccttataaataaatctgataccagcattgagttcggcaaaattcccaaagagcttaaactaacctatgaaaactttaattatgatgaaaaacttaaaataccaggtaatcccacgcataaacgaattaagggcttttttaccgataagcagtgggaaaaccattatgaagatatatggcattgtttttatttttatgaggatgctaacttactaaagcagaaattgataaatagctatggcctcagtgagggagaagccgaaaaagctagcaaaattcgactggcagatggatatggtagtgtatcactaaaagccatccgaaatatcctttattttctgagattaggttatcgttattctactgctgttgtccttggaggtgtaaaaaatgcgtttggaaaagaaaggtggaacaggtttgaaacattccattccgaaataatccttaaagtagttcaacttgtagaaaatgaattacataaggaatatgaggtggttgaaaaacttaaactcatgttggctgaccctgaaaatcagtttggttttgtcgaaaatgatcgatcatttcaaaaactgtatcaccattctcaggcaattgagaaaaaaactgtggtaaaacggttaagtgaaattgaaaatatgagaaatcctattgtacaaaaagggcttaatgaaatgcggaggcttgtgaacgaattgctcgacaattattcaaacaacccggagtatgggccaagtttcagttttgataaaattcatgttgaaatgggtcgcgacataaaaaacagcaagaaaatgaggcaggaaatggtctttaaaataagagagggtgaaaatgaaaatgaagatgcacgtctccggctagcagaatttggattaaaaccaagcagggaaaacattacaaaatatcgactattcaaggaaattcaatcaaaatttggcagtgtggtttgcccctacacaagaaaaacagtgaaaataactgatttattgggaagggataacctttatcaaatagaacatatcatcccattttctgtatcactggatgatagttttgggaacaaaaccatttgtgaatcaaattttaatagagacaaaggcgaacttacaccttatgaattttatcaaaaaaacagtgaccctgacctttggggtgctgaatcatgggaagaggttgaacaacgtgcttttgccttattaccctatcgaaaagcaaagaaatttgttacaaaaaagcgtgatgaagacagcggttttattgctcgtcagctcaatgatatgcgctacatcagtaaaaaagctgcggaaatattaagtgagatatgccaagatgtacgtgtattgcctggtcaacttacagctgaacttcgcagattgtggggtttgaacaatatcattcagcctgttttacctttggacttgccaggctataatgttgatgctgatttaaagttaccacattatcttgtgcttgacaaatatggcaagccaactgaaattaaacctatacttgcctcaagaccagaagcatcagcaaaccaaattctcataccaggttttattacagaaaagaaacaattcattgcagatagaaattatcgacacctgaagtttgaactacaaacagatggcttaaaggctggcaaatattgggcaaaacttaatgtaggaaagccgatcgtgttgacaagaatatttacggaaagacctccaattaatccagaaactgttgtttacagaggtaaaattgaaaaaggctatttcacgcatgacagcctgggaagaaaactaaaagcaaacgaacctgacggaatttattggctcaaattacaagttaagggaaaggaatttattgagcctgaaaaagataaacaacccaaagcaggtaaaaaccagattttgctttttggaactgttcaaaatggcttgttcaccagttatatttatcaatgtgaaacagaaatgttgccgggtaaatattgggctttgatagatgttgaatttgaaaagcccgaatacgctaaggcaattattaatcctggtgaagtttttgaaaatgaaattatcattactggaaccgttgcagacgatggtgagtttattgcggattgcgatcccgggtataaatttgatactaattatcctgcaagtaaatatttcacaaagcttactgttgaagcaatagatggcttttttcctatcgaaaatccagcaccccaattaagcaacgagcaaacactaatggaaggcaatgtatgggtgaataaatcaactggtgagattatgttcgaccccaagaaaaaccgtgacgataaccgacaccatgctgttgatgccatcaccattgctttaacggaactggtttatgttcaaaaactaagccattactttggcgccataaaggacaaagagcgcggaataggtgacaggccaatatttgaccctccctggcagggttttgacaaagacgtaaagaaagctgttgataacatcttggtgtcttttgcacgtaacaacaaggtgttgagtaagataagcaaaactgttgttaaatcaggaaagaagtaccaaagtgttggcttcgcggcacggggtaggttacacagggagtattatttcggaaaacatccaagccctgttttgcatggcaaggacccggaaaaaggaggacttctctttgaaaaagataaagacggaaatcttgtttacttttttcacataagaaagccagtaacaagcattaagaaaaataaacatgtaagcaaaattgtggatcaaggtatacgagacttaattattaatcgattgaaaaatgattttaatatagatgtttctggaagttatacattaccggacaatttcttttttgacgctaataaacagcctgcgcttttcttaccaaacaaaaatggcgaacctgtgcctattaaaaaggttaggctcaaagagtttattggcaatgcagttcagttaaaagataatatgaaccaatgggtaaatccttataacaatcaccatgtggtcatttacgaagatttcaatggagagttgaaggagcaagttgttagtttttgggatgtaattgaaagacaaaatcagggtgacgctatttacaaattgccagaagatggctcaaaactgattacgaccctccaggaaaacgatatgtttttgttaaatcttgataatgagcaaatcgaatcgcttagtagtcaaagcatttcaaaagaagagcttagcaagcatttatatcgggtgcaaaaaatatcaagtatgtattatacttttaggcatcactttgcctcaacagttgtaaatgaaaaagaagaacttagaattgttagtatggcggcttggcagcgtactaagccaataaaagtgataattaactccacaggtcatttcagtaaatta

SEQ ID NO: 45 - protein, 1509 aa, Chlamydia trachomatis
MITLGLDIGVSSLGTAIVDTQNNQILHTGVYVFPAGKEAFGTNKEKPKNETRRVARQGRRQNYRRRLRRSQLLKFLYQLSELNSPEGDRYVPLTFEEIDAWLSWDKEGKTAARQFPDSDAFRAWMAMNPYEIRSRAVEGSITRAEFGRLLYHIIQRRGFQSSRKMKTDEGAMAKGSGAIIGYTQTKSELGEQTLGQYLYQISAKEGEPFSQREEKPRGRYTLRSMYVEEMERIWGQQSKRLGLEGLEIEGTPVKELLLGQISHDPESGELLYKSNDSVLFYQRPLRSQKHLINKCSLESRKMYDPLKQRHYIQGPRAAAVSHPTSEFHRALQVLSNVRVEGRTLYDMGLFQTSLDFLCSKNSKTIKVADLKRHLSLVSKLNYDDDQTLPGCPTIASITDLFEKDELSYHYGETDTIYIDIWQKLLFYDDERLLEKNIADYAKGKGLKLKADFAKRLEGINLVDDYGSISIHAMDNIIPFLLQGLSHTEAIFLGGVKNAFGPRYYYFEKEEQMWQELLQIINEPASKGEKIDRLIEYLSNPDEQTGLDEKRLRMKLYHPTQSVQKIEIQNRLGMVDNLRNPIVEQALWAVRRQVNALIDQMIEQTGNPNFRFDRITIELSRDLKSSLDRRLKIRQEQEANRAENEEAVIELRARGVRVTRDNILKYRLHKELQQQDGIARCPYTGKNINPSHLYDGNNFFQIEHIIPRSISLDDSFGNKTLCESNFNREKGNKTPYEFYSVNSSKALWGAEDWDEITHRVRRVLPARKASRFLSKRSAQEQLEELPSKMLNDTAYMSVKAREYLTSICGDVRSVTGHITADLRRMWQLNKILGETYECAPGAVSEELIARGDTDVYALLDSESQVVNRVAPKLHLIPNPTADEIRIPVVVKGTKLYRDGKEYPKPAHLDLNGVTGEALIAYHAVGGTPYCQPLFNPKPSTKDMLVVPATINSKNQLTSDYIEALRKVKKSQYPTDPGKYWVTLQLKGEPIWELPSNKKRATDNAVRVSGRLNGNAFWSPLFETEVDSGVVNMEEHRNRTAYLNLDYDTAEFTKMLRPAPEVGVGELLLTGVANHGLLLLDQCDGKTIDVQHLNLQEQTKYYVSLRYGETLSPEERVTIYPLYAKTPTPRGGEVLLEGKLFLLDKSQGLYALEPVKNRDDHRHHAVDALVIALAKQNYVTELSTYNRQDDSYRQGEALKPRFDLPWKGFREEAEQSINDILVVHKKNRVAPLSKVTRKITKKGKVYTSEGYAVRGALHKATIYGQRKAPGETEVGYHSRKSIAEVTKMSQLNKVVDAAIRKEMLRILKEEFGLDISNPKLVFPKNAFIDEEGKTKVYLPNRNGGESVPVKKVRLCDRFNNAVRLKETNQYVDPQNNHHVLIYQDQDGNLQESVVTMWDVVDRRNRGEEIFQLPPDGKRILFKLMRDRMYLIGITREEILLHLSDTAYLSKYLYRVQKLSSMYYVFRKATASTTNYDREMIRVQSLPFFEPDKDNIHSVEITLTGQVKLKDD

SEQ ID NO: 46 - protein, 1564 aa, Chryseobacterium humi
MAKILGLDLGTNSIGWAIVDDKNSKILDTGVRIFPEGVVAKTIGSGDKEESKNAKRRESRQTRRGFYRKRLRKIKLLRTLINFKMCPLEHFELDVWSKWDKIKKKEGRMAPELFLPENSAYNLWLKQNPYILRDKALSKDLTLTEFGRVLYHLIQRRGFLSNRKGKDDGKIYGGKEGIVGIDKTNEQIKNSTLGKFLFKILPKEGEPFKIIEDELGNELRVRSRYTLRDMYIAEFEAIWNRQAKHLGLDNIYYSNVKNVLLKGSLKNKKNERKINHLKKIKGNGNIEIITLNNNLSKNEKPKYLLKIATKLPLKEFLAGRIEKDEDGKIKFKSNESVLFWQRPLRSQKKLLAKCRFEPDLKDDNGKFIQKGKTPCHLSHPLYEEYRAYQYVNNIEYGKKQRLEESQRKQVIDLINSKDGNFKFSEIKKKLKLSYETFNYSDDSSVNGNYMIKNLNSIFPKEILEKEFVVNVNKEDIIEYGYERIWHLFHFCEDNDVLIKKLQDNFDLSENDIEKISKINLKEGYSNVSLKAIKNILPYLKEGKLLSDAVILGGVRNVFGKKWHEFIDFHEKIEKDILYINHQKSNKEGEAIEKIKKYLIDYKFGFTENDNRFKKLYHHSQDVVEVKLQKKIGPVEDLRNPIVQQGVNEVRRLVNELLDVHGKFDQVRVELGRDLKNNKSKREEIGYKIRENTAKNEKAKELLIEYGLRQSRDNVQKVLLFQEMLDRGTVAVCPYTNKTINISDVLGNENKMQIEHIIPKSISLDDSFANKTLCDSKFNGLKGERTPYEFYQINNDANLWGGSKSWEEIERRTFRILPYSKAKRFTSKTKFETNDFIQRQLNDTRYISKKTKEILSQICEDVRVMPGSLTAELRKLWGLNNILHPAFVLDLPALHIDANKHIPHYVVLDEDNVPILSQRIYNDKPILKANQTSITGFVDNEIFKSKEKYVKFEVKAPNLSNGEYWAKLLLSNPKSIVRVFKEKPKSTENEIVVRGKIEKEKFKHEGLSGINAEGKENGTYWAKLSIVDKKFKKPKKDDQPKKNGKQILLFGAVKEGVFSSYMYECDADLEDGKYWLILDIDFENVEFEKALNERPEVLENQILIQGITNDENVFVSDMDCQHQFLMNEGKGKYYVLFDIISEVNEFYPIKTKSPELENGQSLTEGTIWVDKYTGEIKFDPKKNREDHRHHAIDALVIALTKETYFQQLSRRNASSENKKRGNEYEKEQLDFAEPWKEFHVDAQKSINSILISHKQNKNILTKITKKITKNGKTYKSEGMSVRGQLHDKTYYGHNKNCKADEYVYRVDINNLSFSQLTNIIDLNLKAVIVEGVIKKFEESEKLLFKKMNSKVQNLDKSKVKKIKAKVDKILKETSFFMPNEGKRYIRLNIIEPANFERMPVPIKKVRIRKVLRNAEKLNNEIKQFVDPQNNHHVLIYKDENGNLKEEVVTFWTVVERKRTGEPVYKLPSDGKKIVTTLQINDMFLLGLNEDEINWENPNYNHLKEHLYRVQKFTSGDFYFRIAKTSSIQNGEEKQQINSFGLGKNGWLTHNPIKVKISVSGKILKV

SEQ ID NO: 47 - protein, 1458 aa, Salinispira pacifica
MPYTLGLDLGSSSLGWALVDEEQQNIIKTGVRVFPGAATDIGTGKESYKNQDRRLSRQARRQTLRRKQRRIVLLRTLVDFKMCPLSKDELESYAKYRKGKKQEFPSSSEFNSWLKLNPYELRDRALHEDISRFEFGRILYHIIHHRGFKTGRKSNEDGALYKGNEDTVGVDETKNLLQQYATLGEYLNSLRIKSDGNERLRGRYTLRNWYVDEFNKIWERQAGKLGLENETVHSRNKRYYGPAGSSEHRRKLQAYKKAGISFEIEGDFICREQTVSLKDYLGNPDKGILFYQKPLKSQKHLLSKCRYEKRKSVCQISHPEFEYYRAMQFANQIEYGAGEKLNETDRKDVIILLLSKDKAVKMSDLKKKIQKTGVQFNYADDHKVPGSPTTAFISKVTDTSNVENIDWELFHSIWNDLVFYDKSEKLKEKFQRSYNIDVSIEQLNKYKINTDYSSLSLKAVRNINPFLQMGMLYNDAVIFAGIRRVIGEKSFDANADEIISHVSKLKEKNNRDVNQSFTDALREYVMQTFNISMNQTYKLYHHSQEMSVQNNRYYLSELPNLRNPIVQQALQELRTLVNTLVEQYLPDGEQFSKIKVELARELKLPRDVRRRMHWENNQRAERNNDAKIELQKFGLSPSRENIQKYILYKEIQNSVQGQSVQCPYTGQSLAITDVFGNDNKVQVEHIIPYSISLDDSLSNKTLCIAEENGKKGEKTPYQFYKSDPDHWEVVKARAFTLLPYEKAKRFVSTRDYEPEAFLQRQLNDTRYISKAARDYLSTICDQVYVFPGSLTAKVRRMWGLNSILSPPIKTNFEGNAGMYWAVMNDDGVIIELSEQELPPPAIRASDILISGEMKNGIFKVDRKFTGQSQTREVSVEHRTYDSPGWMVYSLLSVEKYLPLFSKLPSQDTKSIWLKGRAQVKDRESPSLMLNSLPVKLMLPPGEYDDNAPYWVNIPVNGELRWEKRGEKPGKNELLIKRTKHRDGKVEAFIDKRKCLFDIPPQDNKSALIIPLDLQEILQVVRMFTPTPAIAEHQFLIKGKIDQSYRFTAESNFRIALDAPEGTPSGPAFAIANCEPRDMRFYPKKNIKPKNGKGKNVLSGFVFQEDGSLVFNPGKNRMDHRHHAIDALTIALLNTGQLRELSGYFLKIKKRQRYSAPKPQFPLPWDNFRADALNSINSILVVHRQNREIIKRIKKNTRLNGRTYVSQGMSVRGQLHKDTVYGKHYIPGSDEPGYHVRKQITDLTSLSKIADAGTHEILRTFVEENGYSIDEKEKVKGEWFTKGNDYQVIMPSSGKPGNPIKKVRIRENLGNAVQWHTGINQYVNPRNNYAFIIYRDSDGNYKEHPVTFWDAVQARLAGKSVYQLPEPGKIITTLHINDTFLLGLGDEQLQGDLPQELVSRHLYRVQKLSSMYYCFRLHTESTLDRDFEPYYLGIQSFKAWKEQNPLRVKVATTGEVNLE

SEQ ID NO: 48 - protein, 1498 aa, unidentified
METTLGIDLGTNSIGLALVDQEEHQILYSGVRIFPEGINKDTIGLGEKEESRNATRRAKRQMRRQYFRKKLRKAKLLELLIAYDMCPLKPEDVRRWKNWDKQQKSTVRQFPDTPAFREWLKQNPYELRKQAVTEDVTRPELGRILYQMIQRRGFLSSRKGKEEGKIFTGKDRMVGIDETRKNLQKQTLGAYLYDIAPKNGEKYRFRTERVRARYTLRDMYIREFEIIWQRQAGHLGLAHEQATRKKNIFLEGSATNVRNSKLITHLQAKYGRGHVLIEDTRITVTFQLPLKEVLGGKIEIEEEQLKFKSNESVLFWQRPLRSQKSLLSKCVFEGRNFYDPVHQKWIIAGPTPAPLSHPEFEEFRAYQFINNIIYGKNEHLTAIQREAVFELMCTESKDFNFEKIPKHLKLFEKFNFDDTTKVPACTTISQLRKLFPHPVWEEKREEIWHCFYFYDDNTLLFEKLQKDYALQTNDLEKIKKIRLSESYGNVSLKAIRRINPYLKKGYAYSTAVLLGGIRNSFGKRFEYFKEYEPEIEKAVCRILKEKNAEGEVIRKIKDYLVHNRFGFAKNDRAFQKLYHHSQAITTQAQKERLPETGNLRNPIVQQGLNELRRTVNKLLATCREKYGPSFKFDHIHVEMGRELRSSKTEREKQSRQIRENEKKNEAAKVKLAEYGLKAYRDNIQKYLLYKEIEEKGGTVCCPYTGKTLNISHTLGSDNSVQIEHIIPYSISLDDSLANKTLCDATFNREKGELTPYDFYQKDPSPEKWGASSWEEIEDRAFRLLPYAKAQRFIRRKPQESNEFISRQLNDTRYISKKAVEYLSAICSDVKAFPGQLTAELRHLWGLNNILQSAPDITFPLPVSATENHREYYVITNEQNEVIRLFPKQGETPRTEKGELLLTGEVERKVFRCKGMQEFQTDVSDGKYWRRIKLSSSVTWSPLFAPKPISADGQIVLKGRIEKGVFVCNQLKQKLKTGLPDGSYWISLPVISQTFKEGESVNNSKLTSQQVQLFGRVREGIFRCHNYQCPASGADGNFWCTLDTDTAQPAFTPIKNAPPGVGGGQIILTGDVDDKGIFHADDDLHYELPASLPKGKYYGIFTVESCDPTLIPIELSAPKTSKGENLIEGNIWVDEHTGEVRFDPKKNREDQRHHAIDAIVIALSSQSLFQRLSTYNARRENKKRGLDSTEHFPSPWPGFAQDVRQSVVPLLVSYKQNPKTLCKISKTLYKDGKKIHSCGNAVRGQLHKETVYGQRTAPGATEKSYHIRKDIRELKTSKHIGKVVDITIRQMLLKHLQENYHIDITQEFNIPSNAFFKEGVYRIFLPNKHGEPVPIKKIRMKEELGNAERLKDNINQYVNPRNNHHVMIYQDADGNLKEEIVSFWSVIERQNQGQPIYQLPREGRNIVSILQINDTFLIGLKEEEPEVYRNDLSTLSKHLYRVQKLSGMYYTFRHHLASTLNNEREEFRIQSLEAWKRANPVKVQIDEIGRITFLNGPLC

SEQ ID NO: 49 - RNA, 99 nt, synthetic construct
  source 1..99: mol_type = other RNA
gttgtggtttgattaaagagaaatctttaatcttatcacaataaggctatatgccgtagatgaaaatctttagtcccgcttcggtgggacttttttttt

SEQ ID NO: 50 - RNA, 19 nt, synthetic construct
  source 1..19: mol_type = other RNA
gttgtggtttgattaaaga

SEQ ID NO: 51 - RNA, 15 nt, synthetic construct
  source 1..15: mol_type = other RNA
gttgtggtttgatta

SEQ ID NO: 52 - RNA, 12 nt, synthetic construct
  source 1..12: mol_type = other RNA
gttgtggtttga

SEQ ID NO: 53 - RNA, 10 nt, synthetic construct
  source 1..10: mol_type = other RNA
gttgtggttt

SEQ ID NO: 54 - RNA, 20 nt, synthetic construct
  source 1..20: mol_type = other RNA
tctttaatcttatcacaata

SEQ ID NO: 55 - RNA, 16 nt, synthetic construct
  source 1..16: mol_type = other RNA
taatcttatcacaata

SEQ ID NO: 56 - RNA, 13 nt, synthetic construct
  source 1..13: mol_type = other RNA
tcttatcacaata

SEQ ID NO: 57 - RNA, 11 nt, synthetic construct
  source 1..11: mol_type = other RNA
ttatcacaata

SEQ ID NO: 58 - RNA, 14 nt, synthetic construct
  source 1..14: mol_type = other RNA
aggctatatgccgt

SEQ ID NO: 59 - RNA, 12 nt, synthetic construct
  source 1..12: mol_type = other RNA
aggctatatgcc

SEQ ID NO: 60 - RNA, 14 nt, synthetic construct
  source 1..14: mol_type = other RNA
agatgaaaatcttt

SEQ ID NO: 61 - RNA, 12 nt, synthetic construct
  source 1..12: mol_type = other RNA
agatgaaaatct

SEQ ID NO: 62 - RNA, 28 nt, synthetic construct
  source 1..28: mol_type = other RNA
agtcccgcttcggtgggacttttttttt

SEQ ID NO: 63 - RNA, 20 nt, synthetic construct
  source 1..20: mol_type = other RNA
agtcccgcttcggtgggact

SEQ ID NO: 64 - RNA, 19 nt, synthetic construct
  source 1..19: mol_type = other RNA
agtcccgcttcggtgggac

SEQ ID NO: 65 - RNA, 98 nt, synthetic construct
  source 1..98: mol_type = other RNA
gttgtggtttgattaaagagaaatctttaatcttatcacaataaggctatatgccgtagatgaaaatctttagtcctgcttcggtaggactttttttt

SEQ ID NO: 66 - RNA, 19 nt, synthetic construct
  source 1..19: mol_type = other RNA
gttgtggtttgattaaaga

SEQ ID NO: 67 - RNA, 15 nt, synthetic construct
  source 1..15: mol_type = other RNA
gttgtggtttgatta

SEQ ID NO: 68 - RNA, 12 nt, synthetic construct
  source 1..12: mol_type = other RNA
gttgtggtttga

SEQ ID NO: 69 - RNA, 10 nt, synthetic construct
  source 1..10: mol_type = other RNA
gttgtggttt

SEQ ID NO: 70 - RNA, 20 nt, synthetic construct
  source 1..20: mol_type = other RNA
tctttaatcttatcacaata

SEQ ID NO: 71 - RNA, 16 nt, synthetic construct
  source 1..16: mol_type = other RNA
taatcttatcacaata

SEQ ID NO: 72 - RNA, 13 nt, synthetic construct
  source 1..13: mol_type = other RNA
tcttatcacaata

SEQ ID NO: 73 - RNA, 11 nt, synthetic construct
  source 1..11: mol_type = other RNA
ttatcacaata

SEQ ID NO: 74 - RNA, 14 nt, synthetic construct
  source 1..14: mol_type = other RNA
aggctatatgccgt

SEQ ID NO: 75 - RNA, 12 nt, synthetic construct
  source 1..12: mol_type = other RNA
aggctatatgcc

SEQ ID NO: 76 - RNA, 14 nt, synthetic construct
  source 1..14: mol_type = other RNA
agatgaaaatcttt

SEQ ID NO: 77 - RNA, 12 nt, synthetic construct
  source 1..12: mol_type = other RNA
agatgaaaatct

SEQ ID NO: 78 - RNA, 27 nt, synthetic construct
  source 1..27: mol_type = other RNA
agtcctgcttcggtaggactttttttt

SEQ ID NO: 79 - RNA, 20 nt, synthetic construct
  source 1..20: mol_type = other RNA
agtcctgcttcggtaggact

SEQ ID NO: 80 - RNA, 19 nt, synthetic construct
  source 1..19: mol_type = other RNA
agtcctgcttcggtaggac

SEQ ID NO: 81 - RNA, 98 nt, synthetic construct
  source 1..98: mol_type = other RNA
gttgtggtttgataacaaagaaatttgtatcttatcacaataaggctatatgccgaagttcaaaagaactatgcctctgcttcggcagaggctttttt

SEQ ID NO: 82 - RNA, 19 nt, synthetic construct
  source 1..19: mol_type = other RNA
gttgtggtttgataacaaa

SEQ ID NO: 83 - RNA, 15 nt, synthetic construct
  source 1..15: mol_type = other RNA
gttgtggtttgataa

SEQ ID NO: 84 - RNA, 12 nt, synthetic construct
  source 1..12: mol_type = other RNA
gttgtggtttga

SEQ ID NO: 85 - RNA, 10 nt, synthetic construct
  source 1..10: mol_type = other RNA
gttgtggttt

SEQ ID NO: 86 - RNA, 19 nt, synthetic construct
  source 1..19: mol_type = other RNA
tttgtatcttatcacaata

SEQ ID NO: 87 - RNA, 15 nt, synthetic construct
  source 1..15: mol_type = other RNA
tatcttatcacaata

SEQ ID NO: 88 - RNA, 13 nt, synthetic construct
  source 1..13: mol_type = other RNA
tcttatcacaata

SEQ ID NO: 89 - RNA, 11 nt, synthetic construct
  source 1..11: mol_type = other RNA
ttatcacaata

SEQ ID NO: 90 - RNA, 14 nt, synthetic construct
  source 1..14: mol_type = other RNA
aggctatatgccga

SEQ ID NO: 91 - RNA, 12 nt, synthetic construct
  source 1..12: mol_type = other RNA
aggctatatgcc

SEQ ID NO: 92 - RNA, 16 nt, synthetic construct
  source 1..16: mol_type = other RNA
agttcaaaagaactat

SEQ ID NO: 93 - RNA, 14 nt, synthetic construct
  source 1..14: mol_type = other RNA
agttcaaaagaact

SEQ ID NO: 94 - RNA, 26 nt, synthetic construct
  source 1..26: mol_type = other RNA
gcctctgcttcggcagaggctttttt

SEQ ID NO: 95 - RNA, 20 nt, synthetic construct
  source 1..20: mol_type = other RNA
gcctctgcttcggcagaggc

SEQ ID NO: 96 - RNA, 20 nt, synthetic construct
  source 1..20: mol_type = other RNA
gcctctgcttcggcagaggc

SEQ ID NO: 97 - RNA, 99 nt, synthetic construct
  source 1..99: mol_type = other RNA
gttgtggtttgattaaagagaaatctttaatcttatcacaataaggctatatgccgtagatgtaaaatctttagtcctgcttcggtgggactttttttt

SEQ ID NO: 98 - RNA, 19 nt, synthetic construct
  source 1..19: mol_type = other RNA
gttgtggtttgattaaaga

SEQ ID NO: 99 - RNA, 15 nt, synthetic construct
  source 1..15: mol_type = other RNA
gttgtggtttgatta

SEQ ID NO: 100 - RNA, 12 nt, synthetic construct
  source 1..12: mol_type = other RNA
gttgtggtttga

SEQ ID NO: 101 - RNA, 10 nt, synthetic construct
  source 1..10: mol_type = other RNA
gttgtggttt

SEQ ID NO: 102 - RNA, 20 nt, synthetic construct
  source 1..20: mol_type = other RNA
tctttaatcttatcacaata

SEQ ID NO: 103 - RNA, 16 nt, synthetic construct
  source 1..16: mol_type = other RNA
taatcttatcacaata

SEQ ID NO: 104 - RNA, 13 nt, synthetic construct
  source 1..13: mol_type = other RNA
tcttatcacaata

SEQ ID NO: 105 - RNA, 11 nt, synthetic construct
  source 1..11: mol_type = other RNA
ttatcacaata

SEQ ID NO: 106 - RNA, 14 nt, synthetic construct
  source 1..14: mol_type = other RNA
aggctatatgccgt

SEQ ID NO: 107 - RNA, 12 nt, synthetic construct
  source 1..12: mol_type = other RNA
aggctatatgcc

SEQ ID NO: 108 - RNA, 15 nt, synthetic construct
  source 1..15: mol_type = other RNA
agatgtaaaatcttt

SEQ ID NO: 109 - RNA, 13 nt, synthetic construct
  source 1..13: mol_type = other RNA
agatgtaaaatct

SEQ ID NO: 110 - RNA, 27 nt, synthetic construct
  source 1..27: mol_type = other RNA
agtcctgcttcggtgggactttttttt

SEQ ID NO: 111 - RNA, 20 nt, synthetic construct
  source 1..20: mol_type = other RNA
agtcctgcttcggtgggact

SEQ ID NO: 112 - RNA, 19 nt, synthetic construct
  source 1..19: mol_type = other RNA
agtcctgcttcggtgggac

SEQ ID NO: 113 - RNA, 97 nt, synthetic construct
  source 1..97: mol_type = other RNA
gttgtggtttgattaaagagaaatctttaatcttatcacaataaggctatatgccgtagatgaaaatctttagtcctgcttcggtgggacttttttt

SEQ ID NO: 114 - RNA, 19 nt, synthetic construct
  source 1..19: mol_type = other RNA
gttgtggtttgattaaaga

SEQ ID NO: 115 - RNA, 15 nt, synthetic construct
  source 1..15: mol_type = other RNA
gttgtggtttgatta

SEQ ID NO: 116 - RNA, 12 nt, synthetic construct
  source 1..12: mol_type = other RNA
gttgtggtttga

SEQ ID NO: 117 - RNA, 10 nt, synthetic construct
  source 1..10: mol_type = other RNA
gttgtggttt

SEQ ID NO: 118 - RNA, 20 nt, synthetic construct
  source 1..20: mol_type = other RNA
tctttaatcttatcacaata

SEQ ID NO: 119 - RNA, 16 nt, synthetic construct
  source 1..16: mol_type = other RNA
taatcttatcacaata

SEQ ID NO: 120 - RNA, 13 nt, synthetic construct
  source 1..13: mol_type = other RNA
tcttatcacaata

SEQ ID NO: 121 - RNA, 11 nt, synthetic construct
  source 1..11: mol_type = other RNA
ttatcacaata

SEQ ID NO: 122 - RNA, 14 nt, synthetic construct
  source 1..14: mol_type = other RNA
aggctatatgccgt

SEQ ID NO: 123 - RNA, 12 nt, synthetic construct
  source 1..12: mol_type = other RNA
aggctatatgcc

SEQ ID NO: 124 - RNA, 14 nt, synthetic construct
  source 1..14: mol_type = other RNA
agatgaaaatcttt

SEQ ID NO: 125 - RNA, 12 nt, synthetic construct
  source 1..12: mol_type = other RNA
agatgaaaatct

SEQ ID NO: 126 - RNA, 26 nt, synthetic construct
  source 1..26: mol_type = other RNA
agtcctgcttcggtgggacttttttt

SEQ ID NO: 127 - RNA, 20 nt, synthetic construct
  source 1..20: mol_type = other RNA
agtcctgcttcggtgggact

SEQ ID NO: 128 - RNA, 19 nt, synthetic construct
  source 1..19: mol_type = other RNA
agtcctgcttcggtgggac

SEQ ID NO: 129 - RNA, 97 nt, synthetic construct
  source 1..97: mol_type = other RNA
gttgtggtttgattaaagagaaatctttaatcttatcacaataaggctatatgccgtagatgtaaaatctttagtcctgcttcggtgggactttttt

SEQ ID NO: 130 - RNA, 19 nt, synthetic construct
  source 1..19: mol_type = other RNA
gttgtggtttgattaaaga

SEQ ID NO: 131 - RNA, 15 nt, synthetic construct
  source 1..15: mol_type = other RNA
gttgtggtttgatta

SEQ ID NO: 132 - RNA, 12 nt, synthetic construct
  source 1..12: mol_type = other RNA
gttgtggtttga

SEQ ID NO: 133 - RNA, 10 nt, synthetic construct
  source 1..10: mol_type = other RNA
gttgtggttt

SEQ ID NO: 134 - RNA, 20 nt, synthetic construct
  source 1..20: mol_type = other RNA
tctttaatcttatcacaata

SEQ ID NO: 135 - RNA, 16 nt, synthetic construct
  source 1..16: mol_type = other RNA
taatcttatcacaata

SEQ ID NO: 136 - RNA, 13 nt, synthetic construct
  source 1..13: mol_type = other RNA
tcttatcacaata

SEQ ID NO: 137 - RNA, 11 nt, synthetic construct
  source 1..11: mol_type = other RNA
ttatcacaata

SEQ ID NO: 138 - RNA, 14 nt, synthetic construct
  source 1..14: mol_type = other RNA
aggctatatgccgt

SEQ ID NO: 139 - RNA, 12 nt, synthetic construct
  source 1..12: mol_type = other RNA
aggctatatgcc

SEQ ID NO: 140 - RNA, 15 nt, synthetic construct
  source 1..15: mol_type = other RNA
agatgtaaaatcttt

SEQ ID NO: 141 - RNA, 13 nt, synthetic construct
  source 1..13: mol_type = other RNA
agatgtaaaatct

SEQ ID NO: 142 - RNA, 25 nt, synthetic construct
  source 1..25: mol_type = other RNA
agtcctgcttcggtgggactttttt

SEQ ID NO: 143 - RNA, 20 nt, synthetic construct
  source 1..20: mol_type = other RNA
agtcctgcttcggtgggact

SEQ ID NO: 144 - RNA, 19 nt, synthetic construct
  source 1..19: mol_type = other RNA
agtcctgcttcggtgggac

SEQ ID NO: 145 - RNA, 105 nt, synthetic construct
  source 1..105: mol_type = other RNA
gttgtggtttgattaaagagaaatctttaatcttatcacaataaggctatatgccgtagatgaaaatctttagtcctgcttcggtgggactttttcttttttttt

SEQ ID NO: 146 - RNA, 19 nt, synthetic construct
  source 1..19: mol_type = other RNA
gttgtggtttgattaaaga

SEQ ID NO: 147 - RNA, 15 nt, synthetic construct
  source 1..15: mol_type = other RNA
gttgtggtttgatta

SEQ ID NO: 148 - RNA, 12 nt, synthetic construct
  source 1..12: mol_type = other RNA
gttgtggtttga

SEQ ID NO: 149 - RNA, 10 nt, synthetic construct
  source 1..10: mol_type = other RNA
gttgtggttt

SEQ ID NO: 150 - RNA, 20 nt, synthetic construct
  source 1..20: mol_type = other RNA
tctttaatcttatcacaata

SEQ ID NO: 151 - RNA, 16 nt, synthetic construct
  source 1..16: mol_type = other RNA
taatcttatcacaata

SEQ ID NO: 152 - RNA, 13 nt, synthetic construct
  source 1..13: mol_type = other RNA
tcttatcacaata

SEQ ID NO: 153 - RNA, 11 nt, synthetic construct
  source 1..11: mol_type = other RNA
ttatcacaata

SEQ ID NO: 154 - RNA, 14 nt, synthetic construct
  source 1..14: mol_type = other RNA
aggctatatgccgt

SEQ ID NO: 155 - RNA, 12 nt, synthetic construct
  source 1..12: mol_type = other RNA
aggctatatgcc

SEQ ID NO: 156 - RNA, 14 nt, synthetic construct
  source 1..14: mol_type = other RNA
agatgaaaatcttt

SEQ ID NO: 157 - RNA, 12 nt, synthetic construct
  source 1..12: mol_type = other RNA
agatgaaaatct

SEQ ID NO: 158 - RNA, 34 nt, synthetic construct
  source 1..34: mol_type = other RNA
agtcctgcttcggtgggactttttcttttttttt

SEQ ID NO: 159 - RNA, 20 nt, synthetic construct
  source 1..20: mol_type = other RNA
agtcctgcttcggtgggact

SEQ ID NO: 160 - RNA, 25 nt, synthetic construct
  source 1..25: mol_type = other RNA
agtcctgcttcggtgggactttttc

SEQ ID NO: 161 - RNA, 105 nt, synthetic construct
  source 1..105: mol_type = other RNA
gttgtggtttgattaaagagaaatctttaatcttatcacaataaggctatatgccgtagatgaaaatctttagtcctgcttcggtgggacttcttcttttttttt

SEQ ID NO: 162 - RNA, 34 nt, synthetic construct
  source 1..34: mol_type = other RNA
agtcctgcttcggtgggacttcttcttttttttt

SEQ ID NO: 163 - RNA, 25 nt, synthetic construct
  source 1..25: mol_type = other RNA
agtcctgcttcggtgggacttcttc

SEQ ID NO: 164 - RNA, 97 nt, synthetic construct
  source 1..97: mol_type = other RNA
gttgtggtttgattaaagagaaatctttaatcttatcacaataaggctatatgccgtagatgaaaatctttagtcctgcttcggtaggacttttttt

SEQ ID NO: 165 - RNA, 19 nt, synthetic construct
  source 1..19: mol_type = other RNA
gttgtggtttgattaaaga

SEQ ID NO: 166 - RNA, 15 nt, synthetic construct
  source 1..15: mol_type = other RNA
gttgtggtttgatta

SEQ ID NO: 167 - RNA, 12 nt, synthetic construct
  source 1..12: mol_type = other RNA
gttgtggtttga

SEQ ID NO: 168 - RNA, 10 nt, synthetic construct
  source 1..10: mol_type = other RNA
gttgtggttt

SEQ ID NO: 169 - RNA, 20 nt, synthetic construct
  source 1..20: mol_type = other RNA
tctttaatcttatcacaata

SEQ ID NO: 170 - RNA, 16 nt, synthetic construct
  source 1..16: mol_type = other RNA
taatcttatcacaata

SEQ ID NO: 171 - RNA, 13 nt, synthetic construct
  source 1..13: mol_type = other RNA
tcttatcacaata

SEQ ID NO: 172 - RNA, 11 nt, synthetic construct
  source 1..11: mol_type = other RNA
ttatcacaata

SEQ ID NO: 173 - RNA, 14 nt, synthetic construct
  source 1..14: mol_type = other RNA
aggctatatgccgt

SEQ ID NO: 174 - RNA, 12 nt, synthetic construct
  source 1..12: mol_type = other RNA
aggctatatgcc

SEQ ID NO: 175 - RNA, 14 nt, synthetic construct
  source 1..14: mol_type = other RNA
agatgaaaatcttt

SEQ ID NO: 176 - RNA, 12 nt, synthetic construct
  source 1..12: mol_type = other RNA
agatgaaaatct

SEQ ID NO: 177 - RNA, 26 nt, synthetic construct
  source 1..26: mol_type = other RNA
agtcctgcttcggtaggacttttttt

SEQ ID NO: 178 - RNA, 20 nt, synthetic construct
  source 1..20: mol_type = other RNA
agtcctgcttcggtaggact

SEQ ID NO: 179 - RNA, 19 nt, synthetic construct
  source 1..19: mol_type = other RNA
agtcctgcttcggtaggac

SEQ ID NO: 180 - RNA, 97 nt, synthetic construct; identical to SEQ ID NO: 113
  source 1..97: mol_type = other RNA

SEQ ID NO: 181 - RNA, 19 nt, synthetic construct
  source 1..19: mol_type = other RNA
gttgtggtttgattaaaga

SEQ ID NO: 182 - RNA, 15 nt, synthetic construct
  source 1..15: mol_type = other RNA
gttgtggtttgatta

SEQ ID NO: 183 - RNA, 12 nt, synthetic construct
  source 1..12: mol_type = other RNA
gttgtggtttga

SEQ ID NO: 184 - RNA, 10 nt, synthetic construct
  source 1..10: mol_type = other RNA
gttgtggttt

SEQ ID NO: 185 - RNA, 20 nt, synthetic construct
  source 1..20: mol_type = other RNA
tctttaatcttatcacaata

SEQ ID NO: 186 - RNA, 16 nt, synthetic construct
  source 1..16: mol_type = other RNA
taatcttatcacaata

SEQ ID NO: 187 - RNA, 13 nt, synthetic construct
  source 1..13: mol_type = other RNA
tcttatcacaata

SEQ ID NO: 188 - RNA, 11 nt, synthetic construct
  source 1..11: mol_type = other RNA
ttatcacaata

SEQ ID NO: 189 - RNA, 14 nt, synthetic construct
  source 1..14: mol_type = other RNA
aggctatatgccgt

SEQ ID NO: 190 - RNA, 12 nt, synthetic construct
  source 1..12: mol_type = other RNA
aggctatatgcc

SEQ ID NO: 191 - RNA, 14 nt, synthetic construct
  source 1..14: mol_type = other RNA
agatgaaaatcttt

SEQ ID NO: 192 - RNA, 12 nt, synthetic construct
  source 1..12: mol_type = other RNA
agatgaaaatct

SEQ ID NO: 193 - RNA, 26 nt, synthetic construct
  source 1..26: mol_type = other RNA
agtcctgcttcggtgggacttttttt

SEQ ID NO: 194 - RNA, 20 nt, synthetic construct
  source 1..20: mol_type = other RNA
agtcctgcttcggtgggact

SEQ ID NO: 195 - RNA, 19 nt, synthetic construct
  source 1..19: mol_type = other RNA
agtcctgcttcggtgggac

SEQ ID NO: 196 - RNA, 99 nt, synthetic construct
  source 1..99: mol_type = other RNA
gttgtggattgacagttcgaaagaactgtcatttcacaataaggctatatgccgaaggtctctgacctattaccccgcttcggcggggtttcttttttt

SEQ ID NO: 197 - RNA, 18 nt, synthetic construct
  source 1..18: mol_type = other RNA
gttgtggattgacagttc

SEQ ID NO: 198 - RNA, 15 nt, synthetic construct
  source 1..15: mol_type = other RNA
gttgtggattgacag

SEQ ID NO: 199 - RNA, 12 nt, synthetic construct
  source 1..12: mol_type = other RNA
gttgtggattga

SEQ ID NO: 200 - RNA, 10 nt, synthetic construct
  source 1..10: mol_type = other RNA
gttgtggatt

SEQ ID NO: 201 - RNA, 19 nt, synthetic construct
  source 1..19: mol_type = other RNA
gaactgtcatttcacaata

SEQ ID NO: 202 - RNA, 16 nt, synthetic construct
  source 1..16: mol_type = other RNA
ctgtcatttcacaata

SEQ ID NO: 203 - RNA, 13 nt, synthetic construct
  source 1..13: mol_type = other RNA
tcatttcacaata

SEQ ID NO: 204 - RNA, 11 nt, synthetic construct
  source 1..11: mol_type = other RNA
atttcacaata

SEQ ID NO: 205 - RNA, 14 nt, synthetic construct
  source 1..14: mol_type = other RNA
aggctatatgccga

SEQ ID NO: 206 - RNA, 12 nt, synthetic construct
  source 1..12: mol_type = other RNA
aggctatatgcc

SEQ ID NO: 207 - RNA, 16 nt, synthetic construct
  source 1..16: mol_type = other RNA
aggtctctgacctatt

SEQ ID NO: 208 - RNA, 13 nt, synthetic construct
  source 1..13: mol_type = other RNA
aggtctctgacct

SEQ ID NO: 209 - RNA, 28 nt, synthetic construct
  source 1..28: mol_type = other RNA
accccgcttcggcggggtttcttttttt

SEQ ID NO: 210 - RNA, 18 nt, synthetic construct
  source 1..18: mol_type = other RNA
accccgcttcggcggggt

SEQ ID NO: 211 - RNA, 21 nt, synthetic construct
  source 1..21: mol_type = other RNA
accccgcttcggcggggtttc

SEQ ID NO: 212 - RNA, 92 nt, synthetic construct
  source 1..92: mol_type = other RNA
gttgtggattgattactttgaaaaaagtaatcttttcataacaaggctatatgccgaagttattacggctccacttcggtggagcctttttt

SEQ ID NO: 213 - RNA, 19 nt, synthetic construct
  source 1..19: mol_type = other RNA
gttgtggattgattacttt

SEQ ID NO: 214 - RNA, 15 nt, synthetic construct
  source 1..15: mol_type = other RNA
gttgtggattgatta

SEQ ID NO: 215 - RNA, 12 nt, synthetic construct
  source 1..12: mol_type = other RNA
gttgtggattga

SEQ ID NO: 216 - RNA, 10 nt, synthetic construct
  source 1..10: mol_type = other RNA
gttgtggatt

SEQ ID NO: 217 - RNA, 20 nt, synthetic construct
  source 1..20: mol_type = other RNA
aaagtaatcttttcataaca

SEQ ID NO: 218 - RNA, 16 nt, synthetic construct
  source 1..16: mol_type = other RNA
taatcttttcataaca

SEQ ID NO: 219 - RNA, 13 nt, synthetic construct
  source 1..13: mol_type = other RNA
tcttttcataaca

SEQ ID NO: 220 - RNA, 11 nt, synthetic construct
  source 1..11: mol_type = other RNA
ttttcataaca

SEQ ID NO: 221 - RNA, 23 nt, synthetic construct
  source 1..23: mol_type = other RNA
aggctatatgccgaagttattac

SEQ ID NO: 222 - RNA, 12 nt, synthetic construct
  source 1..12: mol_type = other RNA
aggctatatgcc

SEQ ID NO: 223 - RNA, 26 nt, synthetic construct
  source 1..26: mol_type = other RNA
ggctccacttcggtggagcctttttt

SEQ ID NO: 224 - RNA, 20 nt, synthetic construct
  source 1..20: mol_type = other RNA
ggctccacttcggtggagcc

SEQ ID NO: 225 - RNA, 20 nt, synthetic construct
  source 1..20: mol_type = other RNA
ggctccacttcggtggagcc

SEQ ID NO: 226 - RNA, 92 nt, synthetic construct
  source 1..92: mol_type = other RNA
gttgtggattgattactttgaaaaaagtaatctcttcataacaaggctatatgccgaagttattacggctccacttcggtggagcctttttt

SEQ ID NO: 227 - RNA, 20 nt, synthetic construct
  source 1..20: mol_type = other RNA
aaagtaatctcttcataaca

SEQ ID NO: 228 - RNA, 16 nt, synthetic construct
  source 1..16: mol_type = other RNA
taatctcttcataaca

SEQ ID NO: 229 - RNA, 13 nt, synthetic construct
  source 1..13: mol_type = other RNA
tctcttcataaca

SEQ ID NO: 230 - RNA, 11 nt, synthetic construct
  source 1..11: mol_type = other RNA
tcttcataaca

SEQ ID NO: 231 - RNA, 92 nt, synthetic construct; identical to SEQ ID NO: 212
  source 1..92: mol_type = other RNA

SEQ ID NO: 232 - RNA, 19 nt, synthetic construct
  source 1..19: mol_type = other RNA
gttgtggattgattacttt

SEQ ID NO: 233 - RNA, 15 nt, synthetic construct
  source 1..15: mol_type = other RNA
gttgtggattgatta

SEQ ID NO: 234 - RNA, 12 nt, synthetic construct
  source 1..12: mol_type = other RNA
gttgtggattga

SEQ ID NO: 235 - RNA, 10 nt, synthetic construct
  source 1..10: mol_type = other RNA
gttgtggatt

SEQ ID NO: 236 - RNA, 20 nt, synthetic construct
  source 1..20: mol_type = other RNA
aaagtaatcttttcataaca

SEQ ID NO: 237 - RNA, 16 nt, synthetic construct
  source 1..16: mol_type = other RNA
taatcttttcataaca

SEQ ID NO: 238 - RNA, 13 nt, synthetic construct
  source 1..13: mol_type = other RNA
tcttttcataaca

SEQ ID NO: 239 - RNA, 11 nt, synthetic construct
  source 1..11: mol_type = other RNA
ttttcataaca

SEQ ID NO: 240 - RNA, 23 nt, synthetic construct
  source 1..23: mol_type = other RNA
aggctatatgccgaagttattac

SEQ ID NO: 241 - RNA, 12 nt, synthetic construct
  source 1..12: mol_type = other RNA
aggctatatgcc

SEQ ID NO: 242 - RNA, 26 nt, synthetic construct
  source 1..26: mol_type = other RNA
ggctccacttcggtggagcctttttt

SEQ ID NO: 243 - RNA, 20 nt, synthetic construct
  source 1..20: mol_type = other RNA
ggctccacttcggtggagcc

SEQ ID NO: 244 - RNA, 20 nt, synthetic construct
  source 1..20: mol_type = other RNA
ggctccacttcggtggagcc

SEQ ID NO: 245 - RNA, 92 nt, synthetic construct; identical to SEQ ID NO: 226
  source 1..92: mol_type = other RNA

SEQ ID NO: 246 - RNA, 20 nt, synthetic construct
  source 1..20: mol_type = other RNA
aaagtaatctcttcataaca

SEQ ID NO: 247 - RNA, 16 nt, synthetic construct
  source 1..16: mol_type = other RNA
taatctcttcataaca

SEQ ID NO: 248 - RNA, 13 nt, synthetic construct
  source 1..13: mol_type = other RNA
tctcttcataaca

SEQ ID NO: 249 - RNA, 11 nt, synthetic construct
  source 1..11: mol_type = other RNA
tcttcataaca

SEQ ID NO: 250 - RNA, 96 nt, synthetic construct
  source 1..96: mol_type = other RNA
gttgtggattgatcagtttgaaaaaacgatcttttcacaacaaggctatatgccgtagttgattctacgctcccgcttcggcgggagctttttttt

SEQ ID NO: 251 - RNA, 19 nt, synthetic construct
  source 1..19: mol_type = other RNA
gttgtggattgatcagttt

SEQ ID NO: 252 - RNA, 15 nt, synthetic construct
  source 1..15: mol_type = other RNA
gttgtggattgatca

SEQ ID NO: 253 - RNA, 12 nt, synthetic construct
  source 1..12: mol_type = other RNA
gttgtggattga

SEQ ID NO: 254 - RNA, 10 nt, synthetic construct
  source 1..10: mol_type = other RNA
gttgtggatt

SEQ ID NO: 255 - RNA, 19 nt, synthetic construct
  source 1..19: mol_type = other RNA
aaacgatcttttcacaaca

SEQ ID NO: 256 - RNA, 15 nt, synthetic construct
  source 1..15: mol_type = other RNA
gatcttttcacaaca

SEQ ID NO: 257 - RNA, 13 nt, synthetic construct
  source 1..13: mol_type = other RNA
tcttttcacaaca

SEQ ID NO: 258 - RNA, 11 nt, synthetic construct
  source 1..11: mol_type = other RNA
ttttcacaaca

SEQ ID NO: 259 - RNA, 12 nt, synthetic construct
  source 1..12: mol_type = other RNA
aggctatatgcc

SEQ ID NO: 260 - RNA, 12 nt, synthetic construct
  source 1..12: mol_type = other RNA
aggctatatgcc

SEQ ID NO: 261 - RNA, 14 nt, synthetic construct
  source 1..14: mol_type = other RNA
gtagttgattctac

SEQ ID NO: 262 - RNA, 14 nt, synthetic construct
  source 1..14: mol_type = other RNA
gtagttgattctac

SEQ ID NO: 263 - RNA, 28 nt, synthetic construct
  source 1..28: mol_type = other RNA
gctcccgcttcggcgggagctttttttt

SEQ ID NO: 264 - RNA, 20 nt, synthetic construct
  source 1..20: mol_type = other RNA
gctcccgcttcggcgggagc

SEQ ID NO: 265 - RNA, 20 nt, synthetic construct
  source 1..20: mol_type = other RNA
gctcccgcttcggcgggagc

SEQ ID NO: 266 - RNA, 96 nt, synthetic construct
  source 1..96: mol_type = other RNA
gttgtggattgatcagtttgaaaaaacgatctcttcacaacaaggctatatgccgtagttgattctacgctcccgcttcggcgggagctttttttt

SEQ ID NO: 267 - RNA, 19 nt, synthetic construct
  source 1..19: mol_type = other RNA
aaacgatctcttcacaaca

SEQ ID NO: 268 - RNA, 15 nt, synthetic construct
  source 1..15: mol_type = other RNA
gatctcttcacaaca

SEQ ID NO: 269 - RNA, 13 nt, synthetic construct
  source 1..13: mol_type = other RNA
tctcttcacaaca

SEQ ID NO: 270 - RNA, 11 nt, synthetic construct
  source 1..11: mol_type = other RNA
tcttcacaaca

SEQ ID NO: 271 - RNA, 100 nt, synthetic construct
  source 1..100: mol_type = other RNA
gttgcggtttgatctttgagaaatcaaagatcttatcgcaataaggctatatgccgaaggtcgtttcgacctatatctccgcttaggcggagtttttttt

SEQ ID NO: 272 - RNA, 19 nt, synthetic construct
  source 1..19: mol_type = other RNA
gttgcggtttgatctttga

SEQ ID NO: 273 - RNA, 15 nt, synthetic construct
  source 1..15: mol_type = other RNA
gttgcggtttgatct

SEQ ID NO: 274 - RNA, 12 nt, synthetic construct
  source 1..12: mol_type = other RNA
gttgcggtttga

SEQ ID NO: 275 - RNA, 10 nt, synthetic construct
  source 1..10: mol_type = other RNA
gttgcggttt

SEQ ID NO: 276 - RNA, 20 nt, synthetic construct
  source 1..20: mol_type = other RNA
tcaaagatcttatcgcaata

SEQ ID NO: 277 - RNA, 16 nt, synthetic construct
  source 1..16: mol_type = other RNA
agatcttatcgcaata

SEQ ID NO: 278 - RNA, 13 nt, synthetic construct
  source 1..13: mol_type = other RNA
tcttatcgcaata

SEQ ID NO: 279 - RNA, 11 nt, synthetic construct
  source 1..11: mol_type = other RNA
ttatcgcaata

SEQ ID NO: 280 - RNA, 14 nt, synthetic construct
  source 1..14: mol_type = other RNA
aggctatatgccga

SEQ ID NO: 281 - RNA, 12 nt, synthetic construct
  source 1..12: mol_type = other RNA
aggctatatgcc

SEQ ID NO: 282 - RNA, 19 nt, synthetic construct
  source 1..19: mol_type = other RNA
aggtcgtttcgacctatat

SEQ ID NO: 283 - RNA, 15 nt, synthetic construct
  source 1..15: mol_type = other RNA
aggtcgtttcgacct

SEQ ID NO: 284 - RNA, 24 nt, synthetic construct
  source 1..24: mol_type = other RNA
ctccgcttaggcggagtttttttt

SEQ ID NO: 285 - RNA, 16 nt, synthetic construct
  source 1..16: mol_type = other RNA
ctccgcttaggcggag

SEQ ID NO: 286 - RNA, 16 nt, synthetic construct
  source 1..16: mol_type = other RNA
ctccgcttaggcggag

SEQ ID NO: 287 - RNA, 93 nt, synthetic construct
  source 1..93: mol_type = other RNA
gctgtgatttgatgtaagaaattacatcttatcacagcaaggctatatgccgaaggatgtaatcctatactcccgcttcggtgggagtttttt

SEQ ID NO: 288 - RNA, 17 nt, synthetic construct
  source 1..17: mol_type = other RNA
gctgtgatttgatgtaa

SEQ ID NO: 289 - RNA, 15 nt, synthetic construct
  source 1..15: mol_type = other RNA
gctgtgatttgatgt

SEQ ID NO: 290 - RNA, 12 nt, synthetic construct
  source 1..12: mol_type = other RNA
gctgtgatttga

SEQ ID NO: 291 - RNA, 10 nt, synthetic construct
  source 1..10: mol_type = other RNA
gctgtgattt

SEQ ID NO: 292 - RNA, 18 nt, synthetic construct
  source 1..18: mol_type = other RNA
ttacatcttatcacagca

SEQ ID NO: 293 - RNA, 16 nt, synthetic construct
  source 1..16: mol_type = other RNA
acatcttatcacagca

SEQ ID NO: 294 - RNA, 13 nt, synthetic construct
  source 1..13: mol_type = other RNA
tcttatcacagca

SEQ ID NO: 295 - RNA, 11 nt, synthetic construct
  source 1..11: mol_type = other RNA
ttatcacagca

SEQ ID NO: 296 - RNA, 14 nt, synthetic construct
  source 1..14: mol_type = other RNA
aggctatatgccga

SEQ ID NO: 297 - RNA, 12 nt, synthetic construct
  source 1..12: mol_type = other RNA
aggctatatgcc

SEQ ID NO: 298 - RNA, 15 nt, synthetic construct
  source 1..15: mol_type = other RNA
aggatgtaatcctat

SEQ ID NO: 299 - RNA, 13 nt, synthetic construct
  source 1..13: mol_type = other RNA
aggatgtaatcct

SEQ ID NO: 300 - RNA, 25 nt, synthetic construct
  source 1..25: mol_type = other RNA
actcccgcttcggtgggagtttttt

SEQ ID NO: 301 - RNA, 20 nt, synthetic construct
  source 1..20: mol_type = other RNA
actcccgcttcggtgggagt

SEQ ID NO: 302 - RNA, 19 nt, synthetic construct
  source 1..19: mol_type = other RNA
actcccgcttcggtgggag

SEQ ID NO: 303 - RNA, 101 nt, synthetic construct
  source 1..101: mol_type = other RNA
gttgtggtttgatgtaattgaaaaaattacatcttatcacaataaggctatatgccgaaggataaaatgtcctatactcctgcttcggtaggagttttttt

SEQ ID NO: 304 - RNA, 19 nt, synthetic construct
  source 1..19: mol_type = other RNA
gttgtggtttgatgtaatt

SEQ ID NO: 305 - RNA, 15 nt, synthetic construct
  source 1..15: mol_type = other RNA
gttgtggtttgatgt

SEQ ID NO: 306 - RNA, 12 nt, synthetic construct
  source 1..12: mol_type = other RNA
gttgtggtttga

SEQ ID NO: 307 - RNA, 10 nt, synthetic construct
  source 1..10: mol_type = other RNA
gttgtggttt

SEQ ID NO: 308 - RNA, 21 nt, synthetic construct
  source 1..21: mol_type = other RNA
aaattacatcttatcacaata

SEQ ID NO: 309 - RNA, 16 nt, synthetic construct
  source 1..16: mol_type = other RNA
acatcttatcacaata

SEQ ID NO: 310 - RNA, 13 nt, synthetic construct
  source 1..13: mol_type = other RNA
tcttatcacaata

SEQ ID NO: 311 - RNA, 11 nt, synthetic construct
  source 1..11: mol_type = other RNA
ttatcacaata

SEQ ID NO: 312 - RNA, 14 nt, synthetic construct
  source 1..14: mol_type = other RNA
aggctatatgccga

SEQ ID NO: 313 - RNA, 12 nt, synthetic construct
  source 1..12: mol_type = other RNA
aggctatatgcc

SEQ ID NO: 314 - RNA, 17 nt, synthetic construct
  source 1..17: mol_type = other RNA
aggataaaatgtcctat

SEQ ID NO: 315 - RNA, 15 nt, synthetic construct
  source 1..15: mol_type = other RNA
aggataaaatgtcct

SEQ ID NO: 316 - RNA, 26 nt, synthetic construct
  source 1..26: mol_type = other RNA
actcctgcttcggtaggagttttttt

SEQ ID NO: 317 - RNA, 20 nt, synthetic construct
  source 1..20: mol_type = other RNA
actcctgcttcggtaggagt

SEQ ID NO: 318 - RNA, 19 nt, synthetic construct
  source 1..19: mol_type = other RNA
actcctgcttcggtaggag

SEQ ID NO: 319 - RNA, 102 nt, synthetic construct
  source 1..102: mol_type = other RNA
gttgtggtttgattaaagagaaatctttaatcttatcacaataaggctatatgccgtaggtaaaaaaccttgagtcccgcttcggtgggacttgtttttttt

SEQ ID NO: 320 - RNA, 19 nt, synthetic construct
  source 1..19: mol_type = other RNA
gttgtggtttgattaaaga

SEQ ID NO: 321 - RNA, 15 nt, synthetic construct
  source 1..15: mol_type = other RNA
gttgtggtttgatta

SEQ ID NO: 322 - RNA, 12 nt, synthetic construct
  source 1..12: mol_type = other RNA
gttgtggtttga

SEQ ID NO: 323 - RNA, 10 nt, synthetic construct
  source 1..10: mol_type = other RNA
gttgtggttt

SEQ ID NO: 324 - RNA, 20 nt, synthetic construct
  source 1..20: mol_type = other RNA
tctttaatcttatcacaata

SEQ ID NO: 325 - RNA, 16 nt, synthetic construct
  source 1..16: mol_type = other RNA
taatcttatcacaata

SEQ ID NO: 326 - RNA, 13 nt, synthetic construct
  source 1..13: mol_type = other RNA
tcttatcacaata

SEQ ID NO: 327 - RNA, 11 nt, synthetic construct
  source 1..11: mol_type = other RNA
ttatcacaata

SEQ ID NO: 328 - RNA, 18 nt, synthetic construct
  source 1..18: mol_type = other RNA
aggctatatgccgtaggt

SEQ ID NO: 329 - RNA, 12 nt, synthetic construct
  source 1..12: mol_type = other RNA
aggctatatgcc

SEQ ID NO: 330 - RNA, 41 nt, synthetic construct
  source 1..41: mol_type = other RNA
aaaaaaccttgagtcccgcttcggtgggacttgtttttttt

SEQ ID NO: 331 - RNA, 39 nt, synthetic construct
  source 1..39: mol_type = other RNA
aaaaaaccttgagtcccgcttcggtgggacttgtttttt

SEQ ID NO: 332 - RNA, 33 nt, synthetic construct
  source 1..33: mol_type = other RNA
aaaaaaccttgagtcccgcttcggtgggacttg

SEQ ID NO: 333 - RNA, 95 nt, synthetic construct
  source 1..95: mol_type = other RNA
gttgtggattgatcagtttgaaaaaacgatcttttcacaacaaggctatatgccgtagttgattctacgctcccgcttcggcgggagcttttttt

SEQ ID NO: 334 - RNA, 19 nt, synthetic construct
  source 1..19: mol_type = other RNA
gttgtggattgatcagttt

SEQ ID NO: 335 - RNA, 15 nt, synthetic construct
  source 1..15: mol_type = other RNA
gttgtggattgatca

SEQ ID NO: 336 - RNA, 12 nt, synthetic construct
  source 1..12: mol_type = other RNA
gttgtggattga

SEQ ID NO: 337 - RNA, 10 nt, synthetic construct
  source 1..10: mol_type = other RNA
gttgtggatt

SEQ ID NO: 338 - RNA, 19 nt, synthetic construct
  source 1..19: mol_type = other RNA
aaacgatcttttcacaaca

SEQ ID NO: 339 - RNA, 15 nt, synthetic construct
  source 1..15: mol_type = other RNA
gatcttttcacaaca

SEQ ID NO: 340 - RNA, 13 nt, synthetic construct
  source 1..13: mol_type = other RNA
tcttttcacaaca

SEQ ID NO: 341 - RNA, 11 nt, synthetic construct
  source 1..11: mol_type = other RNA
ttttcacaaca

SEQ ID NO: 342 - RNA, 12 nt, synthetic construct
  source 1..12: mol_type = other RNA
aggctatatgcc

SEQ ID NO: 343 - RNA, 12 nt, synthetic construct
  source 1..12: mol_type = other RNA
aggctatatgcc

SEQ ID NO: 344 - RNA, 14 nt, synthetic construct
  source 1..14: mol_type = other RNA
gtagttgattctac

SEQ ID NO: 345 - RNA, 14 nt, synthetic construct
  source 1..14: mol_type = other RNA
gtagttgattctac

SEQ ID NO: 346 - RNA, 27 nt, synthetic construct
  source 1..27: mol_type = other RNA
gctcccgcttcggcgggagcttttttt

SEQ ID NO: 347 - RNA, 20 nt, synthetic construct
  source 1..20: mol_type = other RNA
gctcccgcttcggcgggagc

SEQ ID NO: 348 - RNA, 20 nt, synthetic construct
  source 1..20: mol_type = other RNA
gctcccgcttcggcgggagc

SEQ ID NO: 349 - RNA, 95 nt, synthetic construct
  source 1..95: mol_type = other RNA
gttgtggattgatcagtttgaaaaaacgatctcttcacaacaaggctatatgccgtagttgattctacgctcccgcttcggcgggagcttttttt

SEQ ID NO: 350 - RNA, 19 nt, synthetic construct
  source 1..19: mol_type = other RNA
aaacgatctcttcacaaca

SEQ ID NO: 351 - RNA, 15 nt, synthetic construct
  source 1..15: mol_type = other RNA
gatctcttcacaaca

SEQ ID NO: 352 - RNA, 13 nt, synthetic construct
  source 1..13: mol_type = other RNA
tctcttcacaaca

SEQ ID NO: 353 - RNA, 11 nt, synthetic construct
  source 1..11: mol_type = other RNA
tcttcacaaca

SEQ ID NO: 354 - RNA, 96 nt, synthetic construct; identical to SEQ ID NO: 250
  source 1..96: mol_type = other RNA

SEQ ID NO: 355 - RNA, 19 nt, synthetic construct
  source 1..19: mol_type = other RNA
gttgtggattgatcagttt

SEQ ID NO: 356 - RNA, 15 nt, synthetic construct
  source 1..15: mol_type = other RNA
gttgtggattgatca

SEQ ID NO: 357 - RNA, 12 nt, synthetic construct
  source 1..12: mol_type = other RNA
gttgtggattga

SEQ ID NO: 358 - RNA, 10 nt, synthetic construct
  source 1..10: mol_type = other RNA
gttgtggatt

SEQ ID NO: 359 - RNA, 19 nt, synthetic construct
  source 1..19: mol_type = other RNA
aaacgatcttttcacaaca

SEQ ID NO: 360 - RNA, 15 nt, synthetic construct
  source 1..15: mol_type = other RNA
gatcttttcacaaca

SEQ ID NO: 361 - RNA, 13 nt, synthetic construct
  source 1..13: mol_type = other RNA
tcttttcacaaca

SEQ ID NO: 362 - RNA, 11 nt, synthetic construct
  source 1..11: mol_type = other RNA
ttttcacaaca

SEQ ID NO: 363 - RNA, 12 nt, synthetic construct
  source 1..12: mol_type = other RNA
aggctatatgcc

SEQ ID NO: 364 - RNA, 12 nt, synthetic construct
  source 1..12: mol_type = other RNA
aggctatatgcc

SEQ ID NO: 365 - RNA, 14 nt, synthetic construct
  source 1..14: mol_type = other RNA
gtagttgattctac

SEQ ID NO: 366 - RNA, 14 nt, synthetic construct
  source 1..14: mol_type = other RNA
gtagttgattctac

SEQ ID NO: 367 - RNA, 28 nt, synthetic construct
  source 1..28: mol_type = other RNA
gctcccgcttcggcgggagctttttttt

SEQ ID NO: 368 - RNA, 20 nt, synthetic construct
  source 1..20: mol_type = other RNA
gctcccgcttcggcgggagc

SEQ ID NO: 369 - RNA, 20 nt, synthetic construct
  source 1..20: mol_type = other RNA
gctcccgcttcggcgggagc

SEQ ID NO: 370 - DNA, 6862 bp, synthetic construct
  source 1..6862: mol_type = other DNA
taatacgactcactatagggagaccacaacggtttccctctagagagacaataaccctgataatgcttcaataatattgaaaaaggaagagtatgcctaagaagaagagaaaggtgggtaccatcacactgggcctcgatatcggagtgtctagcctgggaaccgccatcgtggacacccagaagaatcagatcctgcacaccggcgtgtacgtgttccctgccggaaaagaggccttcggcaccaacaaagagaagcccaagaacgagacaagaagagtggcccggcaaggcagacggcagaactatagaagaaggctgcggcggagccagctgctgaagtttctgtatcagctgagcgagctgaacagccccgagggcgatagatacgtgcccctgacctttgaggaaatcgacgcctggctgagctgggacaaagagggaaaaaccgccgccagacagttccctgatagcgaggcctttagagcctggatggccatgaatccctacgagatcagatccagagccgtggaaggcagcatcaccagagccgaatttggccggctgctgtaccacatcatccagagaagaggcttccagagcagccggaagatgaaggccgatgaaggcgccatggccaagggcaaagacgacatcatcggctacacccagaccaagtctgagatcggcgaacagaccctgggccagtacctgtaccagatctctgccaaagagggcgagcccttcacacagagagaggaaaagcccagaggccggtacaccctgcggtctatgtacgtggaagagatggaacggatctggggccagcagagcaagagactgggactcgaaggactggaaatcgagggcacccctgtgaaagagctgctgatcggacagatctctcacgaccctgagagcggcgagctgctgtataagagcaacgacagcgtgctgttctggcagaggccactgagaagccagaagcacctgatcaacaagtgcagcctggaatctcggaagatgtacgaccccgtgaagcagcggcactacatccagggacctagagtggctgccgtgtctcaccctacctctgagtttcacagagccctgcaggtcctgagcaacgtccgagttgagggcagaaccctgtacgacatgggcctgtttcagaccagcctggacttcctgtgcagcaagaacagcaagaccatcaaggtggccgacctgaagagacacctgagcctggtgtccaagctgaactacgacgacgatcagaccctgcctggctgtcctacaatcgcctccatcaccgatctgttcgagaaaggcgccctgagctaccactacggcgagacagacaccatctacatcgacatctggcagaagctgctgttctacgacgatgagcggctgctggaaaagaatatcgccgactacgcccagagcaagggcctgaagctgaaagccgatttcgccaagcggctggaaggcatcaacctggtggatgactacggcagcatctccatccacgctatggacaatattatcccattcctgctgcaaggccgcagccacacagaggccatttttctcggcggcgtgaagaacgccttcggacccagatattactactttgagaaagaggaacagatgtggcaagaactgctgcagatcatcaacgagcctgccagcaagggcgagaagatcgacagactgatccagtttctgagcaaccccgatgagcagacaggcctggacgagaagagactgcggatgaagctgtatcaccccacacagtccgtgcagaagattgagatccagaaccggctgggcgaagtggacaacctgagaaaccctatcgtggaacaggccctgtgggctgtgcggagacaagtgaatgccctgatcgaccagatgatcgagcagaccggcaatcccaacttcagattcgaccggatcaccatcgagctgagcagggacctgaagtccagcaaagagcggagaagcaagatccaacaagagcaagaagccaaccgggccgagaacgaagaggccgttatgtggctgaaggccctggatctgccctacaccggcgacaacatcctgaagtaccggctgcacaaagagctgagactgaaaggcggcggagtggccaggtgtccttacaccggaaaggacatcaggctggcccagctgtacgacggcaacaatctgttccagatcgagcacatcattccccggtccatcagcctggataacagctttggcaacaagaccctgtgcgagagcaacttcaaccgcgagaagggcaacatgaccccttacgagttctacagcgtgaacagctccaagacactgtggggagccgaggactgggacgagatcatccacagagttagacaggtgctgcccgccagaaaggcctccagatttctgtccaagagaagcgcccaagagcagcgcgaggaactgcccagcaagatgctgaacgataccgcctacatgagcgtgaaggccagagagtacctgacctccatctgctctgatgtgcggaccgtgtctggctctatcaccgccaatctgagaaggctgtggggcctgaacaagatcctgggcgaaacctatgagtgtctgcccggcaccgtgtctaaagaactggccgctggcgaagagaaagaagtgtacgccatcctggataccaagtccaacgtgatcacaagagtggtgcccaagttcaaccccattccttaccggaccgccgatgagatcagaatccctgtggtggtcaagggcaccaagctgttctgtgacggcaaagagtaccccaagcctgctcacctggacctgaatggtgtttctggcgaagccctgctggcctatcatgccgttggaggaatccctcactgccagcctctgttcaaccctaagcctagcaacaagggcgtgctggtggtcacagccacaagaagcggcaagagcaacctgatctccaacgacatcgacgtgctggataagatcaagaagtccagatatcccaccgagcctggcaagtactgggtcacactgcagctgaagggcgaacccatctgggagttccccagcaacaaaaagagagtggccgccaacgccgtcagagtgcctggtagactgaagggcaataccttcgtgtccagcttcttcgagacagaggaagagaacagcgtcgtcaacatggaagaacaccggaacagaaccgcctacctgaacctggactacgacaccgccgagttcaccaaaatgctgaggcctgaaccagccgtcggagaaggcgaactgcttctgacaggcgtggtcaatcacggctctctgctgctggaccagtgcgacggaaagaccatcgacgtccagcatctgaacctgaaagagcagaccagacactacgtgctgctgcagtacggcaagaacctgtcctccaaagagcaagtcacaatctaccccatctacaacaaggcccctaagagccggaaggacgaagtgctgctcgagggcaagttcttcgccctggacaaggacaagagcctgtacgccctggaacctgtgaagaacagggacgaccatagacaccatgccgtggatgccctggttattgccctggccaagcagaattacgtgaccgagctgtccacctacaaccggcaggacgatagctacagacaaggcggcagactgaagcccagattcgatctgccttgggacggcttcagagaggaagccgagcagagcatcaatgacatcctggtggtgcacaagaagaacagagtcgaccctctgagcaaagtgacccggaagatcaccaagaatggcaaggtgtacacaagcgagggctacgccgttagaggcgctctgcacatggataccatctacggccagagaaaagccccaggacaggccgaagtgggctaccacagcagaaagtccattaccgaagtgaccaacatgagccagctcaacaaggtggtggacgccgccatcagaaaagagatgctgagaatcctgaaagaagaactcggcctggacacctccaacagcagcctgacattccccaagaatgcctttatcgacgaggacggcaagacaaaggtgtacctgcctaacagaaacggcggcgagcccgtgcctgttaagaaagtgcggctgagagagaatttcagcagccccagatgcctgaagaaccagaaccagttcgtgaaaaccgagaacaaccaccacatgctggtgtacaagacgaagaagggaaagttcaacgagagcatcgtgaccttttgggacgtcgtggaacggctgaatagaggcgaggccatctttcagctgcccgaggatggggagaagatcatcttcaagctgatggccaacgactacttcatcatcgggctgaagaaacaagagatcatctccaacctgtcaaaccccagtctgctgagcaagtacctgttcagagtgacaagcctgagcagcagggactacagattcagactggtggccgcctccaccgacaagaacaagcacgagaacagacggatcaggacccctaacttcttcgaccccaccaaagagaacatccacgccgtcgaagtggatctgaacggcaacattaggctgaaagaggacgacggatcctacccatacgatgttccagattacgcggccgctccaaaaaagaaaagaaaagttgcggctagccatcatcaccatcaccatcatcattaaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcgatcccgcgaaat

SEQ ID NO: 371 - DNA, 2768 bp, synthetic construct
  source 1..2768: mol_type = other DNA
ctgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaacacatttccccgaaaagtgccacctgacgtcctcgagtcccgcataatcgaaatgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgctaatacgactcactataggaagagcagagccttggtctcgttgtggattgattactttgaaaaaagtaatcttttcataacaaggctatatgccgaagttattacggctccacttcggtggagccttttttgaattctctagcataaccccttggggcctctaaacgggtcttgaggggttttttgacctaggctaggggatatattccgggtaccccgcttcctcgctca

SEQ ID NO: 372 - DNA, 5009 bp, synthetic construct
  source 1..5009: mol_type = other DNA
tcgagtctttacactttatgcttccggctcgtatgttgtgtggaattgtgagcggataacaatttcacacatgattacggattcaacgtcgtgactggtaaaacccgggcgttacccaacttaatcgccttgcagcacatccccctttcgccagcaggcgtaataaggaaaggattcatgtactatttgaaaaacacaaacttttggatgttcggtttattctttttcttttacttttttatcatgggagcctacttcccgtttttcccgatttggctacatgatatcaaccatatcagcaaaagtgatacgggtattatttttgccgctatttctctgttctcgctattattccaaccgctgtttggtctgctttctgacaaactcggtctacgcaaatacctgctgtggattattaccggcatgttagtgatgtttgcgccgttctttatttttatcttcgggccactgctgcagtacaacattttagtagggtcgattgttggtggtatttatctaggctttagttttaacgccggtgcgccagcagtagaggcatttattgagaaagtcagccggcgcagtaatttcgaatttggtcgcgcgcggatgtttggcagtgttggctgggcgctggttgcctcgattgtcgggatcatgttcaccattaataatcagtttgttttctggctgggctctggcagttgtctcatcctcgccgttttactctttttcgccaaaacggacgcgccctcaagtgccacggttgccaatgcggtaggtgccaaccattcggcatttagccttaagctggcactggaactgttcagacagccaaaactgtggtttttgtcactgtatgttattggcgtttcctccacctacgatgtttttgaccaacagtttgctaatttctttacttcgttctttgctaccggtgaacagggtacccgcgtatttggctacgtaacgacaatgggcgaattacttaacgcctcgattatgttctttgcgccactgatcattaatcgcatcggtgggaagaatgccctgctgctggctggcactattatgtctgtacgtattattggctcatcgttcgccacctcagcgctggaagtggttattctgaaaacgctgcatatgtttgaagtaccgttcctgctggtgggctcctttaaatatattactagtcagtttgaagtgcgtttttcagcgacgatttatctggtcagtttcagcttctttaagcaactggcgatgatttttatgtctgtactggcgggcaatatgtatgaaagcataggtttccaaggcgcttatctggtgctgggtctggtggcgctgggcttcaccttaatttccgtgttcacgcttagcggcccgggcccgctttccctgctgcgtcgtcaggtgaatgaagtcgcttaaaggcctcgatgcagctagcatgctaatctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggggtagcgattgaaaacgatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttttggggaatataatcttctagacatacaatggaagagcagagccttggtctcnnnnnnnnaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacc

SEQ ID NO: 373 - DNA, 10775 bp, synthetic construct
  source 1..10775: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgcctaagaagaagagaaaggtgggtaccatcacactgggcctcgatatcggagtgtctagcctgggaaccgccatcgtggacacccagaagaatcagatcctgcacaccggcgtgtacgtgttccctgccggaaaagaggccttcggcaccaacaaagagaagcccaagaacgagacaagaagagtggcccggcaaggcagacggcagaactatagaagaaggctgcggcggagccagctgctgaagtttctgtatcagctgagcgagctgaacagccccgagggcgatagatacgtgcccctgacctttgaggaaatcgacgcctggctgagctgggacaaagagggaaaaaccgccgccagacagttccctgatagcgaggcctttagagcctggatggccatgaatccctacgagatcagatccagagccgtggaaggcagcatcaccagagccgaatttggccggctgctgtaccacatcatccagagaagaggcttccagagcagccggaagatgaaggccgatgaaggcgccatggccaagggcaaagacgacatcatcggctacacccagaccaagtctgagatcggcgaacagaccctgggccagtacctgtaccagatctctgccaaagagggcgagcccttcacacagagagaggaaaagcccagaggccggtacaccctgcggtctatgtacgtggaagagatggaacggatctggggccagcagagcaagagactgggactcgaaggactggaaatcgagggcacccctgtgaaagagctgctgatcggacagatctctcacgaccctgagagcggcgagctgctgtataagagcaacgacagcgtgctgttctggcagaggccactgagaagccagaagcacctgatcaacaagtgcagcctggaatctcggaagatgtacgaccccgtgaagcagcggcactacatccagggacctagagtggctgccgtgtctcaccctacctctgagtttcacagagccctgcaggtcctgagcaacgtccgagttgagggcagaaccctgtacgacatgggcctgtttcagaccagcctggacttcctgtgcagcaagaacagcaagaccatcaaggtggccgacctgaagagacacctgagcctggtgtccaagctgaactacgacgacgatcagaccctgcctggctgtcctacaatcgcctccatcaccgatctgttcgagaaaggcgccctgagctaccactacggcgagacagacaccatctacatcgacatctggcagaagctgctgttctacgacgatgagcggctgctggaaaagaatatcgccgactacgcccagagcaagggcctgaagctgaaagccgatttcgccaagcggctggaaggcatcaacctggtggatgactacggcagcatctccatccacgctatggacaatattatcccattcctgctgcaaggccgcagccacacagaggccatttttctcggcggcgtgaagaacgccttcggacccagatattactactttgagaaagaggaacagatgtggcaagaactgctgcagatcatcaacgagcctgccagcaagggcgagaagatcgacagactgatccagtttctgagcaaccccgatgagcagacaggcctggacgagaagagactgcggatgaagctgtatcaccccacacagtccgtgcagaagattgagatccagaaccggctgggcgaagtggacaacctgagaaaccctatcgtggaacaggccctgtgggctgtgcggagacaagtgaatgccctgatcgaccagatgatcgagcagaccggcaatcccaacttcagattcgaccggatcaccatcgagctgagcagggacctgaagtccagcaaagagcggagaagcaagatccaacaagagcaagaagccaaccgggccgagaacgaagaggccgttatgtggctgaaggccctggatctgccctacaccggcgacaacatcctgaagtaccggctgcacaaagagctgagactgaaaggcggcggagtggccaggtgtccttacaccggaaaggacatcaggctggcccagctgtacgacggcaacaatctgttccagatcgagcacatcattccccggtccatcagcctggataacagctttggcaacaagaccctgtgcgagagcaacttcaaccgcgagaagggcaacatgaccccttacgagttctacagcgtgaacagctccaagacactgtggggagccgaggactgggacgagatcatccacagagttagacaggtgctgcccgccagaaaggcctccagatttctgtccaagagaagcgcccaagagcagcgcgaggaactgcccagcaagatgctgaacgataccgcctacatgagcgtgaaggccagagagtacctgacctccatctgctctgatgtgcggaccgtgtctggctctatcaccgccaatctgagaaggctgtggggcctgaacaagatcctgggcgaaacctatgagtgtctgcccggcaccgtgtctaaagaactggccgctggcgaagagaaagaagtgtacgccatcctggataccaagtccaacgtgatcacaagagtggtgcccaagttcaaccccattccttaccggaccgccgatgagatcagaatccctgtggtggtcaagggcaccaagctgttctgtgacggcaaagagtaccccaagcctgctcacctggacctgaatggtgtttctggcgaagccctgctggcctatcatgccgttggaggaatccctcactgccagcctctgttcaaccctaagcctagcaacaagggcgtgctggtggtcacagccacaagaagcggcaagagcaacctgatctccaacgacatcgacgtgctggataagatcaagaagtccagatatcccaccgagcctggcaagtactgggtcacactgcagctgaagggcgaacccatctgggagttccccagcaacaaaaagagagtggccgccaacgccgtcagagtgcctggtagactgaagggcaataccttcgtgtccagcttcttcgagacagaggaagagaacagcgtcgtcaacatggaagaacaccggaacagaaccgcctacctgaacctggactacgacaccgccgagttcaccaaaatgctgaggcctgaaccagccgtcggagaaggcgaactgcttctgacaggcgtggtcaatcacggctctctgctgctggaccagtgcgacggaaagaccatcgacgtccagcatctgaacctgaaagagcagaccagacactacgtgctgctgcagtacggcaagaacctgtcctccaaagagcaagtcacaatctaccccatctacaacaaggcccctaagagccggaaggacgaagtgctgctcgagggcaagttcttcgccctggacaaggacaagagcctgtacgccctggaacctgtgaagaacagggacgaccatagacaccatgccgtggatgccctggttattgccctggccaagcagaattacgtgaccgagctgtccacctacaaccggcaggacgatagctacagacaaggcggcagactgaagcccagattcgatctgccttgggacggcttcagagaggaagccgagcagagcatcaatgacatcctggtggtgcacaagaagaacagagtcgaccctctgagcaaagtgacccggaagatcaccaagaatggcaaggtgtacacaagcgagggctacgccgttagaggcgctctgcacatggataccatctacggccagagaaaagccccaggacaggccgaagtgggctaccacagcagaaagtccattaccgaagtgaccaacatgagccagctcaacaaggtggtggacgccgccatcagaaaagagatgctgagaatcctgaaagaagaactcggcctggacacctccaacagcagcctgacattccccaagaatgcctttatcgacgaggacggcaagacaaaggtgtacctgcctaacagaaacggcggcgagcccgtgcctgttaagaaagtgcggctgagagagaatttcagcagccccagatgcctgaagaaccagaaccagttcgtgaaaaccgagaacaaccaccacatgctggtgtacaagacgaagaagggaaagttcaacgagagcatcgtgaccttttgggacgtcgtggaacggctgaatagaggcgaggccatctttcagctgcccgaggatggggagaagatcatcttcaagctgatggccaacgactacttcatcatcgggctgaagaaacaagagatcatctccaacctgtcaaaccccagtctgctgagcaagtacctgttcagagtgacaagcctgagcagcagggactacagattcagactggtggccgcctccaccgacaagaacaagcacgagaacagacggatcaggacccctaacttcttcgaccccaccaaagagaacatccacgccgtcgaagtggatctgaacggcaacattaggctgaaagaggacgacggatcctacccatacgatgttccagattacgcggccgctccaaaaaagaaaagaaaagttgaattcggcggcagcggcgccaccaacttcagcctgctgaagcaggccggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 374 - protein, 9 aa, synthetic construct
YPYDVPDYA

SEQ ID NO: 375 - protein, 7 aa, synthetic construct
PKKKRKV

SEQ ID NO: 376 - protein, 19 aa, synthetic construct
ATNFSLLKQAGDVEENPGP

SEQ ID NO: 377 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 378 - DNA, 27 bp, synthetic construct
  source 1..27: mol_type = other DNA
tacccatacgatgttccagattacgct

SEQ ID NO: 379 - DNA, 21 bp, synthetic construct
  source 1..21: mol_type = other DNA
ccaaaaaagaaaagaaaagtt

SEQ ID NO: 380 - DNA, 57 bp, synthetic construct
  source 1..57: mol_type = other DNA
gccaccaacttcagcctgctgaagcaggccggcgacgtggaggagaaccccggcccc

SEQ ID NO: 381 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 382 - RNA, 22 nt, synthetic construct
  source 1..22: mol_type = other RNA
gccttgcataggaagttcccaa

SEQ ID NO: 383 - RNA, 22 nt, synthetic construct
  source 1..22: mol_type = other RNA
gtctggttgctggggctcatgc

SEQ ID NO: 384 - RNA, 22 nt, synthetic construct
  source 1..22: mol_type = other RNA
cgtgagtaaacctgaatctttg

SEQ ID NO: 385 - RNA, 22 nt, synthetic construct
  source 1..22: mol_type = other RNA
tgccttgcataggaagttccca

SEQ ID NO: 386 - RNA, 22 nt, synthetic construct
  source 1..22: mol_type = other RNA
atcctcttgtcccacagatatc

SEQ ID NO: 387 - RNA, 22 nt, synthetic construct
  source 1..22: mol_type = other RNA
agagacccacacaccggaggag

SEQ ID NO: 388 - RNA, 22 nt, synthetic construct
  source 1..22: mol_type = other RNA
tccagaagacaccttcttcccc

SEQ ID NO: 389 - RNA, 22 nt, synthetic construct
  source 1..22: mol_type = other RNA
tggcaagagacccacacaccgg

SEQ ID NO: 390 - RNA, 22 nt, synthetic construct
  source 1..22: mol_type = other RNA
gcctgttgctgcagtccgggct

SEQ ID NO: 391 - RNA, 22 nt, synthetic construct
  source 1..22: mol_type = other RNA
ggaagagcagagccttggtctc

SEQ ID NO: 392 - DNA, 4530 bp, synthetic construct
  source 1..4530: mol_type = other DNA
atcacactgggcctcgatatcggagtgtctagcctgggaaccgccatcgtggacacccagaagaatcagatcctgcacaccggcgtgtacgtgttccctgccggaaaagaggccttcggcaccaacaaagagaagcccaagaacgagacaagaagagtggcccggcaaggcagacggcagaactatagaagaaggctgcggcggagccagctgctgaagtttctgtatcagctgagcgagctgaacagccccgagggcgatagatacgtgcccctgacctttgaggaaatcgacgcctggctgagctgggacaaagagggaaaaaccgccgccagacagttccctgatagcgaggcctttagagcctggatggccatgaatccctacgagatcagatccagagccgtggaaggcagcatcaccagagccgaatttggccggctgctgtaccacatcatccagagaagaggcttccagagcagccggaagatgaaggccgatgaaggcgccatggccaagggcaaagacgacatcatcggctacacccagaccaagtctgagatcggcgaacagaccctgggccagtacctgtaccagatctctgccaaagagggcgagcccttcacacagagagaggaaaagcccagaggccggtacaccctgcggtctatgtacgtggaagagatggaacggatctggggccagcagagcaagagactgggactcgaaggactggaaatcgagggcacccctgtgaaagagctgctgatcggacagatctctcacgaccctgagagcggcgagctgctgtataagagcaacgacagcgtgctgttctggcagaggccactgagaagccagaagcacctgatcaacaagtgcagcctggaatctcggaagatgtacgaccccgtgaagcagcggcactacatccagggacctagagtggctgccgtgtctcaccctacctctgagtttcacagagccctgcaggtcctgagcaacgtccgagttgagggcagaaccctgtacgacatgggcctgtttcagaccagcctggacttcctgtgcagcaagaacagcaagaccatcaaggtggccgacctgaagagacacctgagcctggtgtccaagctgaactacgacgacgatcagaccctgcctggctgtcctacaatcgcctccatcaccgatctgttcgagaaaggcgccctgagctaccactacggcgagacagacaccatctacatcgacatctggcagaagctgctgttctacgacgatgagcggctgctggaaaagaatatcgccgactacgcccagagcaagggcctgaagctgaaagccgatttcgccaagcggctggaaggcatcaacctggtggatgactacggcagcatctccatccacgctatggacaatattatcccattcctgctgcaaggccgcagccacacagaggccatttttctcggcggcgtgaagaacgccttcggacccagatattactactttgagaaagaggaacagatgtggcaagaactgctgcagatcatcaacgagcctgccagcaagggcgagaagatcgacagactgatccagtttctgagcaaccccgatgagcagacaggcctggacgagaagagactgcggatgaagctgtatcaccccacacagtccgtgcagaagattgagatccagaaccggctgggcgaagtggacaacctgagaaaccctatcgtggaacaggccctgtgggctgtgcggagacaagtgaatgccctgatcgaccagatgatcgagcagaccggcaatcccaacttcagattcgaccggatcaccatcgagctgagcagggacctgaagtccagcaaagagcggagaagcaagatccaacaagagcaagaagccaaccgggccgagaacgaagaggccgttatgtggctgaaggccctggatctgccctacaccggcgacaacatcctgaagtaccggctgcacaaagagctgagactgaaaggcggcggagtggccaggtgtccttacaccggaaaggacatcaggctggcccagctgtacgacggcaacaatctgttccagatcgagcacatcattccccggtccatcagcctggataacagctttggcaacaagaccctgtgcgagagcaacttcaaccgcgagaagggcaacatgaccccttacgagttctacagcgtgaacagctccaagacactgtggggagccgaggactgggacgagatcatccacagagttagacaggtgctgcccgccagaaaggcctccagatttctgtccaagagaagcgcccaagagcagcgcgaggaactgcccagcaagatgctgaacgataccgcctacatgagcgtgaaggccagagagtacctgacctccatctgctctgatgtgcggaccgtgtctggctctatcaccgccaatctgagaaggctgtggggcctgaacaagatcctgggcgaaacctatgagtgtctgcccggcaccgtgtctaaagaactggccgctggcgaagagaaagaagtgtacgccatcctggataccaagtccaacgtgatcacaagagtggtgcccaagttcaaccccattccttaccggaccgccgatgagatcagaatccctgtggtggtcaagggcaccaagctgttctgtgacggcaaagagtaccccaagcctgctcacctggacctgaatggtgtttctggcgaagccctgctggcctatcatgccgttggaggaatccctcactgccagcctctgttcaaccctaagcctagcaacaagggcgtgctggtggtcacagccacaagaagcggcaagagcaacctgatctccaacgacatcgacgtgctggataagatcaagaagtccagatatcccaccgagcctggcaagtactgggtcacactgcagctgaagggcgaacccatctgggagttccccagcaacaaaaagagagtggccgccaacgccgtcagagtgcctggtagactgaagggcaataccttcgtgtccagcttcttcgagacagaggaagagaacagcgtcgtcaacatggaagaacaccggaacagaaccgcctacctgaacctggactacgacaccgccgagttcaccaaaatgctgaggcctgaaccagccgtcggagaaggcgaactgcttctgacaggcgtggtcaatcacggctctctgctgctggaccagtgcgacggaaagaccatcgacgtccagcatctgaacctgaaagagcagaccagacactacgtgctgctgcagtacggcaagaacctgtcctccaaagagcaagtcacaatctaccccatctacaacaaggcccctaagagccggaaggacgaagtgctgctcgagggcaagttcttcgccctggacaaggacaagagcctgtacgccctggaacctgtgaagaacagggacgaccatagacaccatgccgtggatgccctggttattgccctggccaagcagaattacgtgaccgagctgtccacctacaaccggcaggacgatagctacagacaaggcggcagactgaagcccagattcgatctgccttgggacggcttcagagaggaagccgagcagagcatcaatgacatcctggtggtgcacaagaagaacagagtcgaccctctgagcaaagtgacccggaagatcaccaagaatggcaaggtgtacacaagcgagggctacgccgttagaggcgctctgcacatggataccatctacggccagagaaaagccccaggacaggccgaagtgggctaccacagcagaaagtccattaccgaagtgaccaacatgagccagctcaacaaggtggtggacgccgccatcagaaaagagatgctgagaatcctgaaagaagaactcggcctggacacctccaacagcagcctgacattccccaagaatgcctttatcgacgaggacggcaagacaaaggtgtacctgcctaacagaaacggcggcgagcccgtgcctgttaagaaagtgcggctgagagagaatttcagcagccccagatgcctgaagaaccagaaccagttcgtgaaaaccgagaacaaccaccacatgctggtgtacaagacgaagaagggaaagttcaacgagagcatcgtgaccttttgggacgtcgtggaacggctgaatagaggcgaggccatctttcagctgcccgaggatggggagaagatcatcttcaagctgatggccaacgactacttcatcatcgggctgaagaaacaagagatcatctccaacctgtcaaaccccagtctgctgagcaagtacctgttcagagtgacaagcctgagcagcagggactacagattcagactggtggccgcctccaccgacaagaacaagcacgagaacagacggatcaggacccctaacttcttcgaccccaccaaagagaacatccacgccgtcgaagtggatctgaacggcaacattaggctgaaagaggacgac

SEQ ID NO: 393 - DNA, 4548 bp, synthetic construct
  source 1..4548: mol_type = other DNA
acaaaggtgctgggcctcgatctgggcacaaattctatcggctgggccgtcgtggacaaagagggcgagaaaattctggatgccggcgtgcggatcttccccgaaggtgttgagcctacaaccatcggcaagggcgacaacgagcagagcaagaacgccaccagaagagagaagcggcagctgcggagacacttctaccggaagcggctgagaaagatcaagctgctggaagtgctgatcggccagaagatgtgccctctgagaatcgaggacctgcggaagtggaagaactggaacagcaaagagaaggccgagggcagaagattccccagcaccatcgagttcgaccggtggattcagatgaacccctacgagctgagagacaaggccctgagagaacaagtgaccctgttcgagttcggcagaatcatgtaccacctgatccagcggagaggcttcctgtctagcagaaagggcgagcaggaccccaagacactgttcgagaagggcaagcccgacgagaacatcctgcctgtgaacgagacaaaagagaagatcggcaacaccagcctgggcagctacctgaactctatcgcctacaaagagaacaccccttacaagacccggaccgacgtgaagggcaacgagattagagtgcggggcagatacaccgtgcggaagatgtacgtggaagagttcatgaagatctgggacaagcagaacgagtactacgacctggacgtgatcaaactgaaaacccggaagatcagagagctgaggggcagcctgaacggcgccagaaacagaaacaagctggaatacctggtgtctaagtacggcgaggaaaacgtgatcatcgagcagaagtccaacaacatcacccaagtgatcaccatcagcgagatcctgctgaagaagtacctcggcggcgagatctactacgtggaaaacgaggaaggcgagaagaagctgaagttccggtccaacgagagcgtgctgttttggcagaggcccctgaagtcccagaagtccctgctgaccgattgcagattcgagagcaatctgcccgtgatcatgtccaatggcgagatccggaaggacaaagaaggcaagttcgtcctgcggagcaagaagccctgtcctatctctcaccccgagttcgagctgttcagagcctaccagttcgtgaacaatatcagattcggccggaacaccaacctgaccaatgagcagaaagacaccgtgctggatctgatcaacagcaaggacgccagcttctacttctccgacatccgcaagaagctggaactgacctacgagaagttcaactacgacgacaagttcaaggtcgtgggcaaccccaccatcaagaaactgagcagcctgttcgacaagaagatttgggaagagaactaccaggacatctggcactgcttttacttctacgacgataaccagaagctgtttgagaagctcaagaaggactacgactacagcaagggcatcgacctgctggtcaaagtgaagctgaaagacggctacagcaacgtgtccctgaaggccatccggaatatcctgcctttcctgaagaagggctaccagtacgacaaggccgtgatcctcggcggagtgaagaatgcctttggcaagagatgggagtacttcaaggacagccactacaagatcgagaacgagatcatcagcatcctgagcgaggacaatgccgaaggcgacgccatctgcaagatcaaagagcatctgtctagccccgtgttcaattacggcttctccaaggacgacaattggttcgcccacctgtaccaccacagccaagagattgagaaggccagcaagttcagcgacaagctgcccattcctgagaacctgagaaaccccatcgtgcagcagggcatcaacgagatgcggagactggtcaacagcctgatggaaaagtaccagaaccagtacggggacgacttcagattcgatgccatcaaggtggaaatgggccgcgacctgaggaactccaagtctgccagacaagagctgaccttcagaatccgggacaacgagtccaagaacgaagaggccagagccagactggccgagtatggactgcagcagagcagaagcaacatccacaagtacctgatgtggaaagagatcgaggaaaaggctggcgccgctaagtgtccttacaccggcaagaccatctccatctccgatctgctgggcaccgacaactctgtgcagatcgagcacatcatcccttactccgtgtctctggacgacggcttcggcaacaagacaatctgcgagagccacttcaacaacctgaagggcgaactgaccccttaccagttttaccagaagcggagcgacaaggacctgtggggcgttaactcttgggacgccgtggtggaacgggcctttcagatcctgccatacaacaaggccaagcggttcaccagcaagaaggatttcgagaagtccagcttcatcgagagacagctgaacgacagccggtatatcgccaagaagtccatcgagctgctgagcaacatttgcgagaacgtgcgcgtgatgcctggacagctgacagccgaactgagacatctgtggggaatcaacaacgtgctgcagaaacccgtcgtggggatcaacgatcacgtgctgaagctggccaaagaagagacagtgaagtgctacctggtggtggacgaagaaaacaacgtgatcgacttcgtgaagaagcaaaacgacaggcccaacacgaaagaaggcgaaattctgatccccgtgtccgtgaacaagaacaagctgatctccaacctgttcagcttcgacatcgacgtggacgacctgcaggacggcaagtactggatcaaagtcaacctgagcgacgatttcacagtgatccccaagcacatcgataaggccaatgtggacgacgaccggatcgtgttccggggcaagatcgaaaaacaggtgttcaagaacgacacctccggcaacgtgaacaaaaccggcaaagaggatggcgtctactgggccaactttggcatcaaggacaagaagttcgtcgacctcaagaccaacaaagagcggcccaagaccaagagcaaaaagcagatcgtgctgttcggcaatgtggccaacgatgagttcaagtgctacatctacaactgccagaccaacctgcctgatggcaagtattggctgatcctggacatcgactacaacgacgtgtccttcgagaaagccatcaatgagaagcccgagctgcggaacgatcaagtgtgcctgtctgccaccgtggaccagaacagcgaactgattctggacgtggacaagacctacaagaagcaagttggactggcccctggaaagtactacagcgtgtaccagatcgtggactacaccaaagagctgtgccccatcgagaaccagaaacctaaggtggccaatgaacagaagatcattgagggcagcatctgggtcgacaagtacaccggcgaggccaagttcgaccctaagaagaacagggacgaccaccggcaccacgccattgatgccattgtgattgccctgaccgagcagtcctacctgcagcacctgagcacctacaatgccaagcagaaagcccggcagagaggaaagatcgacagcaccgagaagtttagcctgccttggaccggcttcatcaacgatgtgcacaagaaagccgatgagatcctgatcagctacaaaaagcaaaacaaggtgctgacgaagaacaagaaaggcttcagcgtgcggggccagctgcacaaagaaaacgtgttcggaaagcggcaggcccctaatcaagagcagggctatcacagacggaccaaagtgaccgacctggaaaacaacaagcacattgccaaggtggtggatgacagcgtccgcaagatcatcctggaccacctgagatacaactgcgccgtggacatcagcaaccccaagggcttcaaagtgcccaaggatgcctttgtgaaggacggcgagtggcagctgttcctgcctaacaaaaggggcgataaggtgccaatcaagaaagtgcgcatcaaagagtctatcggcaacgccgctcagctcaagagcaacatcaaccagtttgtcaaccctcggaacaaccaccacgtcgtgatctacgaggacttcgacggcaacctgcaagagaacgtggtgcagttctggacagtggtggaaagacagatgaacggcgaagagatctataagctgccccacgatggcaagaacatcatcaccacactggaaatcaacgacatgttcatcctgggactgtccgacgaagagttcgaggacaacaagagcaattacgccttcctgagcaagtatctgtaccgggtgcagaaagtgtccagcatgtactacaccttcagataccacctggccagcaccgtgaccaacaacgaacaagagttcagcatccggtccatgaagtccttcgtggaagccaatcctatcaagctgaagattaacgagcacggcaagctcgtg

SEQ ID NO: 394 - DNA, 4692 bp, synthetic construct
  source 1..4692: mol_type = other DNA
tgcaagaagcagtacaccctgggcctcgatctgggcacatcttctcttggatgggccgtgacagtcagagaaggcgtgtccgtgggatctatcctgaatgccggcgtgacagtgtttcccagcggcaaggctatggtcaacggcgtggaaaagaccaacaacgaggaacggcggaacagcagacaggctagaaggcagctggccagaagaaggctgagaaagcagcatctgctgcggctgctgatcgagaacgacatgtgtcctctgacacagaccgagctggacggctggatgaagtgggataagagcaagcccaccgaggaaagaaggcggttccctgacagccccagattcagagaatggctggctctggacccctacagactgagagccgaagccgtggatacccctctggaagatgatcccgatggcggctttaccgccagagagaagctgggcagaatcctgtacagcatcatccagcaccggggctttctgagcagcagatctggcaaagaggacggcagcatctacaccagcaccatggaaaagaacggcaaagtcggcatcggcgagacaagagccgctctgggagatagaacactgggccagtacctgtactctatcgcccctaaagagggcgaccccttcagcgatcagacagacagagtgcggagccgctacaccctgcgagagatgtacgtggaagagttcgaccggatctggaccagacaggcacctctgctgggactcgacagagaacaggtggaaaccatccactaccggtacttcaccggcaagagcgacagctacgaagccagaaggcggctggaacggtacagagccaaagatcctagcgccgtgatcgagacactgagcggcgacgagcacagattcgtgtacaagaccaccaccagcctgaaagagcacctggccggcaagatttggtacgacaaggatggcaccatccgcttcgtgtccaaagagagcaccctgttctggcagaggcccctgagatcacagaaaggcatgctggacaagtgcagcctggaaagccacaaagtgaaggacagaggcaccaaccggttcatcacacagggccctacagtggtgtctgccagccatccttactacgagctgtacagagtgctgcagaccctgctgaatatgagagtgcggggccagaaactggacctgatgatgctccagtccatgctggataagctgctgacctacggcaagaacaccaaggtgtccaagctgaagaacgacctgagactgaccgccgacctgaacttccccgatgattttgagctgcctggctgccctaccatcagcggcattacaaagctgctggacagcaagaagatgcccagcagaaaaggcgacgcctctcctagcgatagactggtcatccccttcgacctgaatctgtacaccagaatctgggagaagctgttcttctacgacgacaaccatctgctggcccagggcctgaagaaaatcgagggcgccgtgttcgtcgaggacctggaagataagctggccaagctgcacctgagcgaggactacggaaatctgtctctgcacgccctgcggaacatcctgccttatctgctgagaggcgaggaactgtacgatgccgtgctgctcggaggcattagaaacgccgttagagatgtggccctggtggacgatcacgacgatgccctgaggtcctggattcactctcagggaccactggaacggggcgagatgatcgatagaatcgtggctctgctgcaggacccagacggcccttttcacctggatacacccgagctgaagctgcggcaaagactgtatcaccctagccagggaacagccaagcaccagctgatggatcagctccctcctgtggaaaacctgagaaaccccatggtggaacaggccctgtggtccatgagaaggctggtcaactacctgctgaacaactacagaagcaccatcagacccgacttcaccttcgacgagattcacgtggaactggcccgggatctgaagctgagcacacagaaacgggccgaaatgcggcagcggcagaaaaaggccgaagaggacaacgacatggccagagactatctgagaagccagggactgagcaccagcagagagaacctgcaaaagtacaagctgtacatggaaatcgccggcagatccatgggcgtcgtgacatgtccttacaccggcaaagtgatcagaggcctgagcgctgccctggacggcaacagcaattttcagatcgagcacatcgtgccccggaccgtgacctacgatgatagctttgccaacaagaccctgtgcgacgccgacttcaaccggaagaagggcaatctgaccccttggcagttctaccagcaagacccatctcctaagctgtggggagccgacagctgggaagaagtggaaagacgggccttccggctgctgccctatagaaaggccctgagatttgtgtccaagacggtcgaggatcagctgtccggcgatcaactggtggataccgcctacatctccagaaaggccaaagagtacctgagctacatctgcccacaggaccacatcagactgtaccctggcagagtgacagccagactgaggcagctttggggcctgaacagcattctgtcccagggacctaccgtgacaaaggaccagctgccaacagagtgtgccctgagcactgatcagatccctgccgtggtcatcggcaacaagaagtctggcgagctgtcctgcattctgcctaagtacaccagccggcctgagagagaggaaggcgacgttgtcctgctcgttcgggtgcagaagggaaagatctacagcgccagatacagcgagctgcccctgtcttgggacaaagctacaagcctggccgacggcacctactacatcgtgctgagaagcaccgaggtgctgagagtggaaaggatgttcgccgacgagcccagactgtccgaagatcagctgagagccagagtgaccatcgagctgaatagagatggccagccaaagctcagcagcagagaaatggacgtgcgcatcctgaaggacatgcctttgcctgccgctcctggcaactactggctgacaattcctctggtgtccgtgcctgaagtgacccctgaacatcctagagccaaggtggcccctgacaccatcaaagtgacaggctctgttgagcagggcgagttcatcagcaaggtgtacaggcaaggcggcgtggcaatggctgatggcgtggcacaagtgatcctgacacctatgctgtccgaggccagcttcaccaagatcaagctggatgccccagtgatcacaagcggcgaagttgtgctgaccggcgctgtggataccagcgatctgttcgtgtgcgaccaggacgacgagctgagggtgcagatcgattccttcgatgagcccggcaagtactacgccgtgtttagcttcgaccccatggccagctacatcctgagctatgagcaggcccctaaactgtcctccgatgaggtggtgctcgacggcgagctgatctacaacgataaggacgacagaatccacttcagccccgccaagaaccgggacgatcatagacatcacgccctggatgccatcgtgatcgcctgtactggacagaaagtgctgacagccatgagcacctacaacgcccacgccgatgattacgagagaggcaccggcaacagacccaccttttctagcccttggcaaggcttcagagatgacgtgcgcgtggccatcgagtctgtgctggttaagcacaagcagtctaccaagatcctgaacaaagtgaccaagaagatcaccaaggacggccggacacacagatctcaaggactggccgtcagaggaaagctgcacaaccagacctttttcggcctgaggcagagccctcaagagagcttcctgagcctgcacaagagagagagcatccagaacatcgagagctacagcagcctgagcagagtggccgacgtgaccatcagagatctgatgatcagcaggctgagagacatcggcatcgacaccaccagaaaagagtatagcatccccaaagaggcccacgccttcgtcgatgaggaaggcaactacaccatccatctgcctaacaagcggaaaggcggcgctcccattcctgtgaagaaagtgcggctgtgcgagagaagcggctctcctatccagatcgccggctacaacaaatgggtcaagcccgagaacaacgagtacctcatggtgtacacaaactacgacaacaagatccagtacctctgcgtgccattcttcgaggaagtgaacaacaaaatcagaggcgtggacggatggaagaagctgaaggatctgaaagaggtgctgctggttctgcggatcaacgatatggtgctgtggggcctcaacaccaacgaggccagatctattctgcagtccggcgactacagagaggccagcaagtatctgtatcgcgtgaacgccatctcagccggcgagtttgagctgagacaccacagcgacgccagaaccgataagtccagcaggaccagaatcacccctggcacactgagaaagagaggcctgcctatcaaggtggaagtgtctaccatgggagagctgtctgttgtgcggatcgccgagtccgatgat

SEQ ID NO: 395 - DNA, 4695 bp, synthetic construct
  source 1..4695: mol_type = other DNA
tgcaagaagcagtacatcctgggcctcgatctgggcacatcttctctcggatgggccgtgatcgtcagagaaggcgatagcgccagatccatcctgaataccggcagctctgtgttccccagcggcaagacaatggtcggaagcaaagagcggagcaacaacgaggaacggcggaactttagacaggccagaaggcagctggccagaagaaggctgagaaagcagcatctgctgcggctgctgatcgagaaggacatgtgccctctgacacagaaagagctggacggctgggtcaagtgggacaagagcaagcctagcgaggaaagacggcggttccctcagagccctagattcagagaatggctggctctggacccctacagactgagagccgaagccgtggatacccctctggaagatgatcccgatggcggctttaccgccagagagaagctgggcagaatcctgtacagcatcatccagcaccggggctttctgagcagcagatctggcaaagaggacggcagcatctacaccagcaccatggaaaaggacggcaaagtgggcatcgacgagacaagagccgctctgggagatagaacactgggccagtacctgtactctatcgcccctaaagagggcgaccccttcagcgatcagaccgagagagtgcggagccggtacaccctgagagagatgtacgtcgaagagttcgaccggatctggaccagacaggctcctgtgctgggactcgacagagaacaggtggaaaccgtgcactaccggtacttcaccggcaagagcgatagctatgaagccagacggcggctggaacggtacagaacaaaagaccccagcgccgtgattgagacactgtctggcgacgagcacagattcgtgtacaagaccaccaccagcctgaaagagcacctggccggcaagatttggtacgacaaggatggcaccatccgcttcgtgtccaaagagagcaccctgttctggcagaggcccctgagatcacagaaaggcatgctggacaagtgcagcctggaaagccacaaagtgaaggacagaggcaccaaccggttcatcacacagggccctacagtggtgtctgccagccatccttactacgagctgtacagagtgctgcagaccctgctgaacatgcggatcagagacagaaagctggacctgatgcggctgcagtccatgctggataagctgctgacctacggcaagaacaccaaggtgtccaagctgaagaaggacctgagactgaccctggatctgaacttccccgacgattacgagctgcctggctgtcctacaatcagcggcatcaccaagctgctggacagcaagaagatgcccagcagaaaaggcgacgcctctcctgtggatagactggtcatccctttcgacctggacctgtacaccagaatctgggagaagctgttcttctacgacgacaaccatctgctggcccagggcctgagaggaattgaaggcgccgctttcgtcgaggacctggaagataagctggccaagctgcacctgagcgaggactatggaaatctgagcctgcacgccctgcggaacatcctgccttatctgctgagaggcgaggaactgtactctgccgtgctgctcggaggcattagaaacgccgttagagatgacgccctggtggacgaccatgacgatgccctgaggtcctggattcatacccagggacctctggccaggggcgagatgattgatagaattgtggccctgctgcaggacccagacggcccttttcatctgagcacacccgagctgaagctgcggcaaagactgtatcaccctagccagggcacagccaagcacagactgatggatcagctgcctccagtggaaaacctgagaaaccccatggtggaacaggccctgtgggccatgagaaggctggtcaactacctgctcaacaactacagaagcaccatcagacccgacttcaccttcgacgagattcacgtggaactggcccgcgatctgaagctgtctacacagaaaagggctgaagtgcggacccggcagaagaaagccgaagaggacaacgacatggcccgggactatctgagaagccagggactgagcaccagcagagagaacctgcaaaagtacaagctgtacatggaactcgccggcagatctatgggcgtcgtgacatgtccctacacaggcaaagtgatcagaggcctgtctgctgccctggacggcaacagcatgtttcagatcgagcacatcgtgccccggaccatgacctacgatgacagctttgccaacaagaccctgtgcgacgccgacttcaaccggaagaagggcaatctgaccccttggcagttctaccagcaagacccatctcctaagctgtggggagccgacagctgggaagaagtggaacatagagccttccggctgctgccttaccggaaggccaccagatttgtgtctaagaccatcgaggaccagctgtccggcgatcagctggtggatacagcctacatctccagaaaggccaaagagtacctgagctacatctgcccacaggaccacatcagactgtacccaggcagagtgacagctcggctgagacagttgtggggcctgaacagtatcctgagtcagggccccaccatcacaaaaggacaactgcctgctggactggccctgagcggagatcagatccctgccatcgtgatcggcaagaaaaagtccggcgtgctgtctagaatcctgcctaagtacaccagccggcctgagcgggaagaaagagatgtggttctgctcgtgcgggtgcagaagggaaagatctactccgccagatacagcgagctgcccctgttttgggacaaagccgcttctctggccgacggcacctactactttgtgctgagaagctccgaggttgtgcgcgtggaacggatgttcaccgaccagcctagactgcgggaagatcagctgagagccagagtgaccatcgaactgaacaaggacggccagcctaaactgtcctccagagaaatggacgtgcgcatcctgaaggacgtgccacttccaaccgctccaggcaactactggctgacaatccctctggtgtccgtgcctgaagtgacccctgaacaccctagagccaaggtggccacagacaccatcaaagtgtctggaagcgtcgaacagggcgtgttcgtgtctaaggtgtacagacaaggcggcgtggccatggaagatggcctggctcaagtgatcctgacacctgatctgtccgaggtgtcctttaccaagatcaagctggacgctcctgccatcacatctggcgaagttgtgctgaccggcgccatcgatagaagcgacctgttcgtgtgcgaccaggacgacgagctgagagtgcagatcgactccttcgatgagcccggcaagtactacgccgtgttcagctttgaccccatggccagctacatcctgtcctacgaccagacacctaacctgagcagcgacgaagtggtgctggaaggcgacctgatctacaacgataaggacgacagaatccactttagccccagcaagaaccgggacgaccacagacatcatgctctggacgccattgtgatcgcctgtactggacagggcgtcctgacagccatgagcacatacaatgcccacgccgatgactacgagagaggcaccggcacaaagcctaccttcgcttccccatgggagggcttcagagatgatgtgcgggaagccattgaggccgtgctggtcaagcacaagcagagcaccaagatcctgaacaaagtgaccaagcagatcacaaaggacggacggacccacagatctaaaggcctggccgtcagaggaaagctgcacaaagagacattctacggccagcggcagagccctgaagaacctgctcctaccatgcactacagggtgcccatcaccagcatcacaaagtccaagcagctgaaccaggtggccgatcctacaattcgggccctgatgatccgcagactgcaggacatgggcgttgacaccacacaagaggactacgccattcctggcaacgcctttgtggacaaggccggcaactacaccatcctgctgcctaacaagagaggcgtgcccgttcctgtgaagaaagtgcggagaaggcgggtcgtgaacaacacagtgcagctggatggctacaacaaagaggtcgagacatccaacaacgacttcgccctgatctatgaggaccaagagggcaattactgcgaggaagtggtcaccttctgggacagcgccgatagactgctgcacggacaggatagagtgcagctccccatggatgccaagagggtcgtgatgaccctgaggatcaacgatatgttcgtgattggcatgtccgaggacgagctgcggagcgccattggaagttctgattacggcggcatcagcgaccacctgtaccgcgttcagaaactgtctagcatgcactatgtgttcagactgcaccgggccagcacactgaaccacgacgaagaactgtacacaatcagaagcttcagcgccctgaaagaaagcacccctatcaaagtgattatcgacaccgacgggaccatcagcattgccacaagagatgaccaggccaacaatcaccaccag

(19) United States
(12) Patent Application Publication
(10) Pub. No.: US 2026/0218174 A1
(43) Pub. Date: Jul. 30, 2026

(54) Omni Xl 1-22 CRISPR Nucleases

(71) Applicant: EmendoBio Inc., Wilmington, DE (US)

(72) Inventor: Nurit MERON, Ramat Gan (IL)

(21) Appl. No.: 19/145,695

(22) PCT Filed: Jan. 3, 2024
(86) PCT No.: PCT/US2024/010143

§ 371 Date: Jul. 3, 2025

Related U.S. Application Data
(60) Provisional application No. 63/478,292, filed on Jan. 3, 2023.

(51) Int. Cl. C12N 15/11; C12N 9/22

(52) U.S. Cl. CPC C12N 15/111; C12N 9/222; C07K 2319/09; C12N 2310/20

(57) ABSTRACT

The present invention provides a non-naturally occurring composition comprising a CRISPR nuclease comprising a sequence having at least 90% identity to the amino acid sequence selected from the group consisting of SEQ ID NOs: 1-22 or a nucleic acid molecule comprising a sequence encoding the CRISPR nuclease.

This application claims the benefit of U.S. Provisional Application No. 63/478,292, filed Jan. 3, 2023, the contents of which are hereby incorporated by reference. This application also refers to various publications, the disclosures of which are hereby incorporated by reference to, inter alia, provide additional description of the art and features in the art that can be employed with this invention.

### REFERENCE TO SEQUENCE LISTING

This application incorporates-by-reference nucleotide sequences in the file named “230103_91 820-PRO_Sequence_Listing_AWG.xml”, which is 522 kilobytes in size, and which was created on Dec. 15, 2022 in the IBM-PC machine format, having an operating system compatibility with MS-Windows, which is contained in the XML file filed Jan. 3, 2024 as part of this application

### FIELD OF THE INVENTION

The present invention is directed to, inter alia, composition and methods for genome editing.

### BACKGROUND OF THE INVENTION

The Clustered Regularly Interspaced Short Palindromic Repeat (CRISPR) systems of bacterial and archaeal adaptive immunity show extreme diversity of protein composition and genomic loci architecture. The CRISPR systems have become important tools for research and genome engineering. Nevertheless, many details of CRISPR systems have not been determined and the applicability of CRISPR nucleases may be limited by sequence specificity requirements, expression, or delivery challenges. Different CRISPR nucleases have diverse characteristics such as: size, PAM site, on target activity, specificity, cleavage pattern (e.g. blunt, staggered ends), and prominent pattern of indel formation following cleavage. Different sets of characteristics may be useful for different applications. For example, some CRISPR nucleases may be able to target particular genomic loci that other CRISPR nucleases cannot due to limitations of the PAM site. In addition, some CRISPR nucleases currently in use exhibit pre-immunity, which may limit in vivo applicability. See Charlesworth et al., Nature Medicine (2019) and Wagner et al., Nature Medicine (2019). Accordingly, discovery, engineering, and improvement of novel CRISPR nucleases is of importance.

### SUMMARY OF THE INVENTION

Disclosed herein are compositions and methods that may be utilized for genomic engineering, epigenomic engineering, genome targeting, genome editing of cells, and/or in vitro diagnostics.

The disclosed compositions may be utilized for modifying genomic DNA sequences. As used herein, genomic DNA refers to linear and/or chromosomal DNA and/or plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of a DNA sequence at the target site(s) in a genome.

Accordingly, in some embodiments, the compositions comprise a Clustered Regularly Interspaced Short Palindromic Repeat (CRISPR) nucleases. In some embodiments, the CRISPR nuclcase is a CRISPR-associated protein.

### OMNI CRISPR Nucleases

Embodiments of the present invention provide for CRISPR nucleases designated as an “OMNI” nuclease as provided in Table 1.

This invention provides a method of modifying a nucleotide sequence at a target site in the genome of a mammalian cell comprising introducing into the cell (i) a composition comprising a CRISPR nuclease having at least 95% identity to an amino acid sequence selected from the group consisting of SEQ ID NOs: 1-22 or a nucleic acid molecule comprising a sequence encoding a CRISPR nuclease which sequence has at least 95% identity to a nucleic acid sequence selected from the group consisting of SEQ ID NOs: 23-44 and (ii) a DNA-targeting RNA molecule, or a DNA polynucleotide encoding a DNA-targeting RNA molecule, comprising a nucleotide sequence that is complementary to a sequence in the target DNA.

This invention also provides a non-naturally occurring composition comprising a CRISPR associated system comprising:
one or more RNA molecules comprising a guide sequence portion linked to a direct repeat sequence, wherein the guide sequence is capable of hybridizing with a target sequence, or one or more nucleotide sequences encoding the one or more RNA molecules; and
an CRISPR nuclease comprising an amino acid sequence having at least 95% identity to the amino acid sequence selected from the group consisting of SEQ ID NOs: 1-22 or a nucleic acid molecule comprising a sequence encoding the CRISPR nuclease; and
   wherein the one or more RNA molecules hybridize to the target sequence, wherein the target sequence is adjacent to the 3′ end of a complimentary sequence of a Protospacer Adjacent Motif (PAM), and the one or more RNA molecules form a complex with the RNA-guided nuclease.

This invention also provides a non-naturally occurring composition comprising:
a CRISPR nuclease comprising a sequence having at least 95% identity to the amino acid sequence selected from the group consisting of SEQ ID NOs: 1-22 or a nucleic acid molecule comprising a sequence encoding the CRISPR nuclease; and
one or more RNA molecules, or one or more DNA polynucleotide encoding the one or more RNA molecules, comprising at least one of:
a nuclease-binding RNA nucleotide sequence capable of interacting with/binding to the CRISPR nuclcasc; and
a DNA-targeting RNA nucleotide sequence comprising a sequence complementary to a sequence in a target DNA sequence,
wherein the CRISPR nuclease is capable of complexing with the one or more RNA molecules to form a complex capable of hybridizing with the target DNA sequence.

### DETAILED DESCRIPTION

According to some aspects of the invention, the disclosed compositions comprise a Clustered Regularly Interspaced Short Palindromic Repeats (CRISPR) nuclease and/or a nucleic acid molecule comprising a sequence encoding the same.

Table 1 lists novel CRISPR nucleases, as well as substitutions at one or more positions within each nuclease which convert the nuclease to a nickase or catalytically dead nuclease. For example, the catalytic site of any one of the CRISPR nucleases provided herein may be modified such that the nuclease has nickase activity, such that it is capable of performing single-strand DNA cuts. Alternatively, the catalytic site of any one of CRISPR nucleases provided herein may be modified such that the nuclease has no nuclease activity, i.e. a dead nuclease. Throughout the text CRISPR nucleases are referred to, however, any of these nucleases may be modified to have nickase activity (i.e. nucleases which create a single-strand DNA break as opposed to a double-strand break) or to have no nuclease activity (i.e. a catalytically dead nuclease).

Table 2 provides crRNA, tracrRNA, and single-guide RNA (sgRNA) sequences, and portions of crRNA, tracrRNA, and sgRNA sequences, that are compatible with each listed CRISPR nuclease. Accordingly, a crRNA molecule capable of binding and targeting an OMNI nuclease listed in Table 2 as part of a crRNA: tracrRNA complex may comprise any crRNA sequence listed in Table 2. Similarly, a tracrRNA molecule capable of binding and targeting an OMNI nuclease listed in Table 2 as part of a crRNA: tracrRNA complex may comprise any tracrRNA sequence listed in Table 2. Also, a single-guide RNA molecule capable of binding and targeting an OMNI nuclease listed in Table 2 may comprise any sequence listed in Table 2.

For example, a crRNA molecule of OMNI-XL-12 nuclease (SEQ ID NO: 12) may comprise a sequence of any one of SEQ ID NOs: 232-235; a tracrRNA molecule of OMNI-XL-12 nuclease may comprise a sequence of any one of SEQ ID NOs: 236-244 and 246-249; and a sgRNA molecule of OMNI-XL-12 nuclease may comprise a sequence of any one of SEQ ID NOs: 231-249. Other crRNA molecules, tracrRNA molecules, or sgRNA molecules for each OMNI nuclease may be derived from the sequences listed in Table 2 in the same manner.

Any one of these nucleases can target a desired DNA target sequence via a guide RNA molecule. The nuclease-guide complex will also carry any molecule attached to the complex to the target site. Thus, this disclosure also contemplates fusion proteins comprising CRISPR nucleases and a DNA modifying domain (e.g., a deaminase, a nuclease, a nickase, a recombinase, a methyltransferase, a methylasc, an acctylasc, an acetyltransferase, a transcriptional activator, or a transcriptional repressor domain), as well as the use of such fusion proteins in correcting mutations in a genome (e.g., the genome of a human subject) that are associated with disease, or generating mutations in a genome (e.g., the human genome) to decrease or prevent expression of a gene.

In some embodiments, any of the CRISPR nucleases provided herein may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

Thus, any one of the CRISPR nucleases, nickases, or dead-nucleases may be fused (e.g. directly fused or fused via a linker) to another DNA modulating or DNA modifying enzyme, including, but not limited to, base editors such as a deaminase, a reverse transcriptase (e.g. for use in prime editing, see Anzalone et al. (2019)), an enzyme that modifies the methylation state of DNA (e.g. a methyltransferase), or a modifier of histones (e.g. a histone acetyl transferase). Indeed, the OMNI-50 nucleases, nickases, inactive nucleases described herein may be fused to a DNA modifying enzyme or an effector domain thereof. Examples of DNA modifiers include but are not limited to: a deaminase, a nuclease, a nickase, a recombinase, a methyltransferase, a methylase, an acetylase, an acetyltransferase, a reverse transcriptase, an helicase, an integrase, a ligase, a transposase, a demethylase, a phosphatase, a transcriptional activator, or a transcriptional repressor. In some embodiments, any of the CRISPR nucleases provided herein are fused to a protein that has an enzymatic activity. In some embodiments, the enzymatic activity modifies a target DNA molecule, the CRISPR nucleases described herein or fusion proteins thereof, may be used to correct or generate one or more mutations in a gene associated with disease, or to increase, correct, decrease or prevent expression of a gene.

The invention provides a non-naturally occurring composition comprising a CRISPR nuclease comprising a sequence having at least 90% identity to the amino acid sequence selected from the group consisting of SEQ ID NOs: 12, 16, 18, 19, 1-11, 13-15, 17, and 20-22, or a nucleic acid molecule comprising a sequence encoding the CRISPR nuclease.

In some embodiments, the composition further comprises one or more RNA molecules, or a DNA polynucleotide encoding any one of the one or more RNA molecules, wherein the one or more RNA molecules and the CRISPR nuclease do not naturally occur together and the one or more RNA molecules are configured to form a complex with the CRISPR nuclease and/or target the complex to a target site.

In some embodiments, the CRISPR nuclease comprises a sequence having at least 90% identity to the amino acid sequence set forth in SEQ ID NO: 1, and at least one RNA molecule comprises a sequence selected from the group consisting of SEQ ID NOs: 49-64.

In some embodiments, the CRISPR nuclease comprises a sequence having at least 90% identity to the amino acid sequence set forth in SEQ ID NO: 1 and at least one RNA molecule is a CRISPR RNA (crRNA) molecule comprising a guide sequence portion and a sequence selected from the group consisting of SEQ ID NOs: 50-53.

In some embodiments, the composition further comprises a transactivating CRISPR RNA (tracrRNA) molecule comprising a sequence set forth in the group consisting of SEQ ID NOs: 54-64.

In some embodiments, the CRISPR nuclease comprises a sequence having at least 90% identity to the amino acid sequence set forth in SEQ ID NO: 1 and at least one RNA molecule is a single-guide RNA (sgRNA) molecule comprising a guide sequence portion and a sequence selected from the group consisting of SEQ ID NOs: 49-64.

In some embodiments, the CRISPR nuclease comprises a sequence having at least 90% identity to the amino acid sequence set forth in SEQ ID NO: 2, and at least one RNA molecule comprises a sequence selected from the group consisting of SEQ ID NOs: 65-80.

In some embodiments, the CRISPR nuclease comprises a sequence having at least 90% identity to the amino acid sequence set forth in SEQ ID NO: 2 and at least one RNA molecule is a CRISPR RNA (crRNA) molecule comprising a guide sequence portion and a sequence selected from the group consisting of SEQ ID NOs: 66-69.

In some embodiments, the composition further comprises a transactivating CRISPR RNA (tracrRNA) molecule comprising a sequence set forth in the group consisting of SEQ ID NOs: 70-80.

In some embodiments, the CRISPR nuclease comprises a sequence having at least 90% identity to the amino acid sequence set forth in SEQ ID NO: 2 and at least one RNA molecule is a single-guide RNA (sgRNA) molecule comprising a guide sequence portion and a sequence selected from the group consisting of SEQ ID NOs: 65-80.

In some embodiments, the CRISPR nuclease comprises a sequence having at least 90% identity to the amino acid sequence set forth in SEQ ID NO: 3, and at least one RNA molecule comprises a sequence selected from the group consisting of SEQ ID NOs: 81-96.

In some embodiments, the CRISPR nuclease comprises a sequence having at least 90% identity to the amino acid sequence set forth in SEQ ID NO: 3 and at least one RNA molecule is a CRISPR RNA (crRNA) molecule comprising a guide sequence portion and a sequence selected from the group consisting of SEQ ID NOs: 82-85.

In some embodiments, the composition further comprises a transactivating CRISPR RNA (tracrRNA) molecule comprising a sequence set forth in the group consisting of SEQ ID NOs: 86-96.

In some embodiments, the CRISPR nuclease comprises a sequence having at least 90% identity to the amino acid sequence set forth in SEQ ID NO: 3 and at least one RNA molecule is a single-guide RNA (sgRNA) molecule comprising a guide sequence portion and a sequence selected from the group consisting of SEQ ID NOs: 81-96.

In some embodiments, the CRISPR nuclease comprises a sequence having at least 90% identity to the amino acid sequence set forth in SEQ ID NO: 4, and at least one RNA molecule comprises a sequence selected from the group consisting of SEQ ID NOs: 97-112.

In some embodiments, the CRISPR nuclease comprises a sequence having at least 90% identity to the amino acid sequence set forth in SEQ ID NO: 4 and at least one RNA molecule is a CRISPR RNA (crRNA) molecule comprising a guide sequence portion and a sequence selected from the group consisting of SEQ ID NOs: 98-101.

In some embodiments, the composition further comprises a transactivating CRISPR RNA (tracrRNA) molecule comprising a sequence set forth in the group consisting of SEQ ID NOs: 102-112.

In some embodiments, the CRISPR nuclease comprises a sequence having at least 90% identity to the amino acid sequence set forth in SEQ ID NO: 4 and at least one RNA molecule is a single-guide RNA (sgRNA) molecule comprising a guide sequence portion and a sequence selected from the group consisting of SEQ ID NOs: 97-112.

In some embodiments, the CRISPR nuclease comprises a sequence having at least 90% identity to the amino acid sequence set forth in SEQ ID NO: 5, and at least one RNA molecule comprises a sequence selected from the group consisting of SEQ ID NOs: 113-128.

In some embodiments, the CRISPR nuclease comprises a sequence having at least 90% identity to the amino acid sequence set forth in SEQ ID NO: 5 and at least one RNA molecule is a CRISPR RNA (crRNA) molecule comprising a guide sequence portion and a sequence selected from the group consisting of SEQ ID NOs: 114-117.

In some embodiments, the composition further comprises a transactivating CRISPR RNA (tracrRNA) molecule comprising a sequence set forth in the group consisting of SEQ ID NOs: 118-128.

In some embodiments, the CRISPR nuclease comprises a sequence having at least 90% identity to the amino acid sequence set forth in SEQ ID NO: 5 and at least one RNA molecule is a single-guide RNA (sgRNA) molecule comprising a guide sequence portion and a sequence selected from the group consisting of SEQ ID NOs: 113-128.

In some embodiments, the CRISPR nuclease comprises a sequence having at least 90% identity to the amino acid sequence set forth in SEQ ID NO: 6, and at least one RNA molecule comprises a sequence selected from the group consisting of SEQ ID NOs: 129-144.

In some embodiments, the CRISPR nuclease comprises a sequence having at least 90% identity to the amino acid sequence set forth in SEQ ID NO: 6 and at least one RNA molecule is a CRISPR RNA (crRNA) molecule comprising a guide sequence portion and a sequence selected from the group consisting of SEQ ID NOs: 130-133.

In some embodiments, the composition further comprises a transactivating CRISPR RNA (tracrRNA) molecule comprising a sequence set forth in the group consisting of SEQ ID NOs: 134-144.

In some embodiments, the CRISPR nuclease comprises a sequence having at least 90% identity to the amino acid sequence set forth in SEQ ID NO: 6 and at least one RNA molecule is a single-guide RNA (sgRNA) molecule comprising a guide sequence portion and a sequence selected from the group consisting of SEQ ID NOs: 129-144.

In some embodiments, the CRISPR nuclease comprises a sequence having at least 90% identity to the amino acid sequence set forth in SEQ ID NO: 7, and at least one RNA molecule comprises a sequence selected from the group consisting of SEQ ID NOs: 145-163.

In some embodiments, the CRISPR nuclease comprises a sequence having at least 90% identity to the amino acid sequence set forth in SEQ ID NO: 7 and at least one RNA molecule is a CRISPR RNA (crRNA) molecule comprising a guide sequence portion and a sequence selected from the group consisting of SEQ ID NOs: 146-149.

In some embodiments, the composition further comprises a transactivating CRISPR RNA (tracrRNA) molecule comprising a sequence set forth in the group consisting of SEQ ID NOs: 150-160, 162, and 163.

In some embodiments, the CRISPR nuclease comprises a sequence having at least 90% identity to the amino acid sequence set forth in SEQ ID NO: 7 and at least one RNA molecule is a single-guide RNA (sgRNA) molecule comprising a guide sequence portion and a sequence selected from the group consisting of SEQ ID NOs: 145-163.

In some embodiments, the CRISPR nuclease comprises a sequence having at least 90% identity to the amino acid sequence set forth in SEQ ID NO: 8, and at least one RNA molecule comprises a sequence selected from the group consisting of SEQ ID NOs: 164-179.

In some embodiments, the CRISPR nuclease comprises a sequence having at least 90% identity to the amino acid sequence set forth in SEQ ID NO: 8 and at least one RNA molecule is a CRISPR RNA (crRNA) molecule comprising a guide sequence portion and a sequence selected from the group consisting of SEQ ID NOs: 165-168.

In some embodiments, the composition further comprises a transactivating CRISPR RNA (tracrRNA) molecule comprising a sequence set forth in the group consisting of SEQ ID NOs: 169-179.

In some embodiments, the CRISPR nuclease comprises a sequence having at least 90% identity to the amino acid sequence set forth in SEQ ID NO: 8 and at least one RNA molecule is a single-guide RNA (sgRNA) molecule comprising a guide sequence portion and a sequence selected from the group consisting of SEQ ID NOs: 164-179.

In some embodiments, the CRISPR nuclease comprises a sequence having at least 90% identity to the amino acid sequence set forth in SEQ ID NO: 9, and at least one RNA molecule comprises a sequence selected from the group consisting of SEQ ID NOs: 180-195.

In some embodiments, the CRISPR nuclease comprises a sequence having at least 90% identity to the amino acid sequence set forth in SEQ ID NO: 9 and at least one RNA molecule is a CRISPR RNA (crRNA) molecule comprising a guide sequence portion and a sequence selected from the group consisting of SEQ ID NOs: 181-184.

In some embodiments, the composition further comprises a transactivating CRISPR RNA (tracrRNA) molecule comprising a sequence set forth in the group consisting of SEQ ID NOs: 185-195.

In some embodiments, the CRISPR nuclease comprises a sequence having at least 90% identity to the amino acid sequence set forth in SEQ ID NO: 9 and at least one RNA molecule is a single-guide RNA (sgRNA) molecule comprising a guide sequence portion and a sequence selected from the group consisting of SEQ ID NOs: 180-195.

In some embodiments, the CRISPR nuclease comprises a sequence having at least 90% identity to the amino acid sequence set forth in SEQ ID NO: 10, and at least one RNA molecule comprises a sequence selected from the group consisting of SEQ ID NOs: 196-211.

In some embodiments, the CRISPR nuclease comprises a sequence having at least 90% identity to the amino acid sequence set forth in SEQ ID NO: 10 and at least one RNA molecule is a CRISPR RNA (crRNA) molecule comprising a guide sequence portion and a sequence selected from the group consisting of SEQ ID NOs: 197-200.

In some embodiments, the composition further comprises a transactivating CRISPR RNA (tracrRNA) molecule comprising a sequence set forth in the group consisting of SEQ ID NOs: 201-211.

In some embodiments, the CRISPR nuclease comprises a sequence having at least 90% identity to the amino acid sequence set forth in SEQ ID NO: 10 and at least one RNA molecule is a single-guide RNA (sgRNA) molecule comprising a guide sequence portion and a sequence selected from the group consisting of SEQ ID NOs: 196-211.

In some embodiments, the CRISPR nuclease comprises a sequence having at least 90% identity to the amino acid sequence set forth in SEQ ID NO: 11, and at least one RNA molecule comprises a sequence selected from the group consisting of SEQ ID NOs: 212-230.

In some embodiments, the CRISPR nuclease comprises a sequence having at least 90% identity to the amino acid sequence set forth in SEQ ID NO: 11 and at least one RNA molecule is a CRISPR RNA (crRNA) molecule comprising a guide sequence portion and a sequence selected from the group consisting of SEQ ID NOs: 213-216.

In some embodiments, the composition further comprises a transactivating CRISPR RNA (tracrRNA) molecule comprising a sequence set forth in the group consisting of SEQ ID NOs: 217-225 and 227-230.

In some embodiments, the CRISPR nuclease comprises a sequence having at least 90% identity to the amino acid sequence set forth in SEQ ID NO: 11 and at least one RNA molecule is a single-guide RNA (sgRNA) molecule comprising a guide sequence portion and a sequence selected from the group consisting of SEQ ID NOs: 212-230.

In some embodiments, the CRISPR nuclease comprises a sequence having at least 90% identity to the amino acid sequence set forth in SEQ ID NO: 12, and at least one RNA molecule comprises a sequence selected from the group consisting of SEQ ID NOs: 231-249.

In some embodiments, the CRISPR nuclease comprises a sequence having at least 90% identity to the amino acid sequence set forth in SEQ ID NO: 12 and at least one RNA molecule is a CRISPR RNA (crRNA) molecule comprising a guide sequence portion and a sequence selected from the group consisting of SEQ ID NOs: 232-235.

In some embodiments, the composition further comprises a transactivating CRISPR RNA (tracrRNA) molecule comprising a sequence set forth in the group consisting of SEQ ID NOs: 236-244 and 246-249.

In some embodiments, the CRISPR nuclease comprises a sequence having at least 90% identity to the amino acid sequence set forth in SEQ ID NO: 12 and at least one RNA molecule is a single-guide RNA (sgRNA) molecule comprising a guide sequence portion and a sequence selected from the group consisting of SEQ ID NOs: 231-249.

In some embodiments, the CRISPR nuclease comprises a sequence having at least 90% identity to the amino acid sequence set forth in SEQ ID NO: 13, and at least one RNA molecule comprises a sequence selected from the group consisting of SEQ ID NOs: 250-270.

In some embodiments, the CRISPR nuclease comprises a sequence having at least 90% identity to the amino acid sequence set forth in SEQ ID NO: 13 and at least one RNA molecule is a CRISPR RNA (crRNA) molecule comprising a guide sequence portion and a sequence selected from the group consisting of SEQ ID NOs: 251-254.

In some embodiments, the composition further comprises a transactivating CRISPR RNA (tracrRNA) molecule comprising a sequence set forth in the group consisting of SEQ ID NOs: 255-265 and 267-270.

In some embodiments, the CRISPR nuclease comprises a sequence having at least 90% identity to the amino acid sequence set forth in SEQ ID NO: 13 and at least one RNA molecule is a single-guide RNA (sgRNA) molecule comprising a guide sequence portion and a sequence selected from the group consisting of SEQ ID NOs: 250-270.

In some embodiments, the CRISPR nuclease comprises a sequence having at least 90% identity to the amino acid sequence set forth in SEQ ID NO: 14, and at least one RNA molecule comprises a sequence selected from the group consisting of SEQ ID NOs: 271-286.

In some embodiments, the CRISPR nuclease comprises a sequence having at least 90% identity to the amino acid sequence set forth in SEQ ID NO: 14 and at least one RNA molecule is a CRISPR RNA (crRNA) molecule comprising a guide sequence portion and a sequence selected from the group consisting of SEQ ID NOs: 272-275.

In some embodiments, the composition further comprises a transactivating CRISPR RNA (tracrRNA) molecule comprising a sequence set forth in the group consisting of SEQ ID NOs: 276-286.

In some embodiments, the CRISPR nuclease comprises a sequence having at least 90% identity to the amino acid sequence set forth in SEQ ID NO: 14 and at least one RNA molecule is a single-guide RNA (sgRNA) molecule comprising a guide sequence portion and a sequence selected from the group consisting of SEQ ID NOs: 271-286.

In some embodiments, the CRISPR nuclease comprises a sequence having at least 90% identity to the amino acid sequence set forth in SEQ ID NO: 15, and at least one RNA molecule comprises a sequence selected from the group consisting of SEQ ID NOs: 287-302.

In some embodiments, the CRISPR nuclease comprises a sequence having at least 90% identity to the amino acid sequence set forth in SEQ ID NO: 15 and at least one RNA molecule is a CRISPR RNA (crRNA) molecule comprising a guide sequence portion and a sequence selected from the group consisting of SEQ ID NOs: 288-291.

In some embodiments, the composition further comprises a transactivating CRISPR RNA (tracrRNA) molecule comprising a sequence set forth in the group consisting of SEQ ID NOs: 292-302.

In some embodiments, the CRISPR nuclease comprises a sequence having at least 90% identity to the amino acid sequence set forth in SEQ ID NO: 15 and at least one RNA molecule is a single-guide RNA (sgRNA) molecule comprising a guide sequence portion and a sequence selected from the group consisting of SEQ ID NOs: 287-302.

In some embodiments, the CRISPR nuclease comprises a sequence having at least 90% identity to the amino acid sequence set forth in SEQ ID NO: 16, and at least one RNA molecule comprises a sequence selected from the group consisting of SEQ ID NOs: 303-318.

In some embodiments, the CRISPR nuclease comprises a sequence having at least 90% identity to the amino acid sequence set forth in SEQ ID NO: 16 and at least one RNA molecule is a CRISPR RNA (crRNA) molecule comprising a guide sequence portion and a sequence selected from the group consisting of SEQ ID NOs: 304-307.

In some embodiments, the composition further comprises a transactivating CRISPR RNA (tracrRNA) molecule comprising a sequence set forth in the group consisting of SEQ ID NOs: 308-318.

In some embodiments, the CRISPR nuclease comprises a sequence having at least 90% identity to the amino acid sequence set forth in SEQ ID NO: 16 and at least one RNA molecule is a single-guide RNA (sgRNA) molecule comprising a guide sequence portion and a sequence selected from the group consisting of SEQ ID NOs: 303-318.

In some embodiments, the CRISPR nuclease comprises a sequence having at least 90% identity to the amino acid sequence set forth in SEQ ID NO: 17, and at least one RNA molecule comprises a sequence selected from the group consisting of SEQ ID NOs: 319-332.

In some embodiments, the CRISPR nuclease comprises a sequence having at least 90% identity to the amino acid sequence set forth in SEQ ID NO: 17 and at least one RNA molecule is a CRISPR RNA (crRNA) molecule comprising a guide sequence portion and a sequence selected from the group consisting of SEQ ID NOs: 320-323.

In some embodiments, the composition further comprises a transactivating CRISPR RNA (tracrRNA) molecule comprising a sequence set forth in the group consisting of SEQ ID NOs: 324-332.

In some embodiments, the CRISPR nuclease comprises a sequence having at least 90% identity to the amino acid sequence set forth in SEQ ID NO: 17 and at least one RNA molecule is a single-guide RNA (sgRNA) molecule comprising a guide sequence portion and a sequence selected from the group consisting of SEQ ID NOs: 319-332.

In some embodiments, the CRISPR nuclease comprises a sequence having at least 90% identity to the amino acid sequence set forth in SEQ ID NO: 18, and at least one RNA molecule comprises a sequence selected from the group consisting of SEQ ID NOs: 333-353.

In some embodiments, the CRISPR nuclease comprises a sequence having at least 90% identity to the amino acid sequence set forth in SEQ ID NO: 18 and at least one RNA molecule is a CRISPR RNA (crRNA) molecule comprising a guide sequence portion and a sequence selected from the group consisting of SEQ ID NOs: 334-337.

In some embodiments, the composition further comprises a transactivating CRISPR RNA (tracrRNA) molecule comprising a sequence set forth in the group consisting of SEQ ID NOs: 338-348 and 350-353.

In some embodiments, the CRISPR nuclease comprises a sequence having at least 90% identity to the amino acid sequence set forth in SEQ ID NO: 18 and at least one RNA molecule is a single-guide RNA (sgRNA) molecule comprising a guide sequence portion and a sequence selected from the group consisting of SEQ ID NOs: 333-353.

In some embodiments, the CRISPR nuclease comprises a sequence having at least 90% identity to the amino acid sequence set forth in SEQ ID NO: 19, and at least one RNA molecule comprises a sequence selected from the group consisting of SEQ ID NOs: 354-369.

In some embodiments, the CRISPR nuclease comprises a sequence having at least 90% identity to the amino acid sequence set forth in SEQ ID NO: 19 and at least one RNA molecule is a CRISPR

RNA (crRNA) molecule comprising a guide sequence portion and a sequence selected from the group consisting of SEQ ID NOs: 355-358.

In some embodiments, the composition further comprises a transactivating CRISPR RNA (tracrRNA) molecule comprising a sequence set forth in the group consisting of SEQ ID NOs: 359-369.

In some embodiments, the CRISPR nuclease comprises a sequence having at least 90% identity to the amino acid sequence set forth in SEQ ID NO: 19 and at least one RNA molecule is a single-guide RNA (sgRNA) molecule comprising a guide sequence portion and a sequence selected from the group consisting of SEQ ID NOs: 354-369.

In some embodiments, the amino acid sequence of the CRISPR nuclease comprises a conserved insert sequence consisting of 343 consensus positions, wherein each position may be either an amino acid or a gap such that the full amino acid sequence of the conserved insert region is provided by linking together amino acids and removing gaps,
wherein consensus position 1 of the conserved insert region is an asparagine (N) residue;
wherein consensus position 4 of the conserved insert region is a leucine (L) residue;
wherein consensus position 45 of the conserved insert region is a lysine (K) residue;
wherein consensus position 50 of the conserved insert region is a proline (P) residue;
wherein consensus position 61 of the conserved insert region is a valine (V) residue;
wherein consensus position 63 of the conserved insert region is a valine (V) residue;
wherein consensus position 87 of the conserved insert region is a leucine (L) residue;
wherein consensus position 94 of the conserved insert region is a glycine (G) residue;
wherein consensus position 162 of the conserved insert region is a glycine (G) residue;
wherein consensus position 164 of the conserved insert region is a tyrosine (Y) residue;
wherein consensus position 165 of the conserved insert region is a tryptophan (W) residue;
wherein consensus position 184 of the conserved insert region is a arginine (R) residue;
wherein consensus position 197 of the conserved insert region is a glycine (G) residue;
wherein consensus position 204 of the conserved insert region is a phenylalanine (F) residue;
wherein consensus position 232 of the conserved insert region is a leucine (L) residue;
wherein consensus position 241 of the conserved insert region is a phenylalanine (F) residue;
wherein consensus position 243 of the conserved insert region is a lysine (K) residue;
wherein consensus position 249 of the conserved insert region is a proline (P) residue;
wherein consensus position 258 of the conserved insert region is a leucine (L) residue;
wherein consensus position 273 of the conserved insert region is an aspartic acid (D) residue;
wherein consensus position 280 of the conserved insert region is a glutamine (Q) residue;
wherein consensus position 291 of the conserved insert region is a tyrosine (Y) residue;
wherein consensus position 316 of the conserved insert region is a proline (P) residue;
wherein consensus position 327 of the conserved insert region is a glycine (G) residue; and/or
wherein consensus position 343 of the conserved insert region is a proline (P) residue.

In some embodiments, the amino acid sequence of the CRISPR nuclease comprises a conserved insert consisting of 343 consensus positions, wherein each consensus position may be either an amino acid or a gap such that the full conserved insert sequence is provided by linking together amino acids and removing gaps,
wherein consensus position 1 of the conserved insert region is an asparagine (N) residue;
wherein consensus position 2 of the conserved insert region is a lysine (K), asparagine (N), or serine(S) residue;
wherein consensus position 3 of the conserved insert region is an isoleucine (I), methionine (M), or valine (V) residue;
wherein consensus position 4 of the conserved insert region is an isoleucine (I), leucine (L), or valine (V) residue;
wherein consensus position 8 of the conserved insert region is a proline (P), serine(S), or tyrosine (Y) residue;
wherein consensus position 19 of the conserved insert region is a cysteine (C), isoleucine (I), or leucine (L) residue, or a gap;
wherein consensus position 29 of the conserved insert region is an isoleucine (I), valine (V), tryptophan (W), or tyrosine (Y) residue;
wherein consensus position 50 of the conserved insert region is a proline (P) residue;
wherein consensus position 56 of the conserved insert region is an aspartic acid (D), glutamic acid (E), or glutamine (Q) residue;
wherein consensus position 65 of the conserved insert region is a glutamic acid (E), glycine (G), or asparagine (N) residue, or a gap;
wherein consensus position 66 of the conserved insert region is a glycine (G), histidine (H), lysine (K), asparagine (N), or threonine (T) residue;
wherein consensus position 76 of the conserved insert region is a glutamic acid (E) or arginine (R) residue, or a gap;
wherein consensus position 77 of the conserved insert region is a histidine (H) or leucine (L) residue, or a gap;
wherein consensus position 87 of the conserved insert region is a glutamic acid (E), leucine (L), or arginine (R) residue;
wherein consensus position 89 of the conserved insert region is a glycine (G), leucine (L), or glutaminc (Q) residue, or a gap;
wherein consensus position 90 of the conserved insert region is an aspartic acid (D), lysine (K), asparagine (N), or serine(S) residue, or a gap;
wherein consensus position 91 of the conserved insert region is a glycine (G), asparagine (N), or tyrosine (Y) residue, or a gap;
wherein consensus position 92 of the conserved insert region is a glutamic acid (E), phenylalanine (F), or valine (V) residue, or a gap;
wherein consensus position 94 of the conserved insert region is a glycine (G) residue;
wherein consensus position 96 of the conserved insert region is an alanine (A), histidine (H), or tyrosine (Y) residue;
wherein consensus position 100 of the conserved insert region is a leucine (L), valine (V), or tyrosinc (Y) residue;
wherein consensus position 105 of the conserved insert region is a glycine (G), lysine (K), or asparagine (N) residue, or a gap;
wherein consensus position 113 of the conserved insert region is a phenylalanine (F), histidine (H), lysine (K), or tyrosine (Y) residue;
wherein consensus position 117 of the conserved insert region is an alanine (A) or proline (P) residue;
wherein consensus position 125 of the conserved insert region is an isoleucine (I), arginine (R), or valine (V) residue;
wherein consensus position 128 of the conserved insert region is an alanine (A), glycine (G), threonine (T), or valine (V) residue;
wherein consensus position 132 of the conserved insert region is a leucine (L) residue, or a gap;
wherein consensus position 133 of the conserved insert region is an asparagine (N) residue, or a gap;
wherein consensus position 134 of the conserved insert region is a lysine (K) or arginine (R) residue, or a gap;
wherein consensus position 135 of the conserved insert region is an aspartic acid (D) residue, or a gap;
wherein consensus position 136 of the conserved insert region is a glycine (G) or serine(S) residue, or a gap;
wherein consensus position 140 of the conserved insert region is a phenylalanine (F) or leucine (L) residue;
wherein consensus position 147 of the conserved insert region is a phenylalanine (F) residue or a gap;
wherein consensus position 156 of the conserved insert region is a aspartic acid (D), arginine (R), or serine(S) residue, or a gap;
wherein consensus position 162 of the conserved insert region is a glycine (G) or serine(S) residue;
wherein consensus position 164 of the conserved insert region is a cysteine (C) or tyrosine (Y) residue;
wherein consensus position 165 of the conserved insert region is a lysine (K) or tryptophan (W) residue;
wherein consensus position 176 of the conserved insert region is a valine (V) or tryptophan (W) residue, or a gap;
wherein consensus position 186 of the conserved insert region is an alanine (A), lysine (K), or arginine (R) residue, or a gap;
wherein consensus position 187 of the conserved insert region is a threonine (T) residue or a gap;
wherein consensus position 195 of the conserved insert region is a leucine (L) or valine (V) residue;
wherein consensus position 197 of the conserved insert region is a glycine (G) residue;
wherein consensus position 199 of the conserved insert region is an isoleucine (I), leucine (L), threonine (T), or valine (V) residue;
wherein consensus position 204 of the conserved insert region is a phenylalanine (F) residue;
wherein consensus position 208 of the conserved insert region is a proline (P) residue or a gap;
wherein consensus position 209 of the conserved insert region is an aspartic acid (D) residue or a gap;
wherein consensus position 211 of the conserved insert region is a phenylalanine (F) or tyrosine (Y) residue;
wherein consensus position 215 of the conserved insert region is a glutamic acid (E) or valine (V) residue, or a gap;
wherein consensus position 216 of the conserved insert region is an aspartic acid (D) or glutamic acid (E) residue, or a gap;
wherein consensus position 217 of the conserved insert region is an asparagine (N) or serine(S) residue, or a gap;
wherein consensus position 218 of the conserved insert region is a serine(S) residue or a gap;
wherein consensus position 219 of the conserved insert region is a valine (V) residue or a gap;
wherein consensus position 225 of the conserved insert region is an aspartic acid (D), histidine (H), or proline (P) residue;
wherein consensus position 226 of the conserved insert region is a glycine (G) or arginine (R) residue;
wherein consensus position 235 of the conserved insert region is an aspartic acid (D), glutamic acid (E), phenylalanine (F), methionine (M), or asparagine (N) residue;
wherein consensus position 241 of the conserved insert region is a phenylalanine (F), isoleucine (I), or tyrosine (Y) residue;
wherein consensus position 249 of the conserved insert region is a glycine (G), proline (P), or serine(S) residue;
wherein consensus position 260 of the conserved insert region is an alanine (A) or glycine (G) residue;
wherein consensus position 268 of the conserved insert region is an alanine (A), phenylalanine (F), or leucine (L) residue;
wherein consensus position 273 of the conserved insert region is an aspartic acid (D) or asparagine (N) residue;
wherein consensus position 274 of the conserved insert region is a glycine (G) residue, or a gap;
wherein consensus position 288 of the conserved insert region is a glycine (G), serine(S), or threonine (T) residue;
wherein consensus position 289 of the conserved insert region is a lysine (K), glutamine (Q), or arginine (R) residue;
wherein consensus position 290 of the conserved insert region is a histidine (H) or tyrosine (Y) residue;
wherein consensus position 303 of the conserved insert region is a glutamic acid (E) or lysine (K) residue, or a gap;
wherein consensus position 304 of the conserved insert region is a glutamic acid (E) residue, or a gap;
wherein consensus position 305 of the conserved insert region is a glutamine (Q) or arginine (R) residue, or a gap;
wherein consensus position 306 of the conserved insert region is a valine (V) residue, or a gap;
wherein consensus position 307 of the conserved insert region is a threonine (T) residue, or a gap;
wherein consensus position 316 of the conserved insert region is a proline (P) or glutamine (Q) residue;
wherein consensus position 326 of the conserved insert region is an aspartic acid (D) or glutamic acid (E) residue;
wherein consensus position 327 of the conserved insert region is a glycine (G) residue;
wherein consensus position 331 of the conserved insert region is an alanine (A) or leucine (L) residue, or a gap;
wherein consensus position 333 of the conserved insert region is an aspartic acid (D) or asparagine (N) residue;
wherein consensus position 338 of the conserved insert region is a glutamic acid (E), leucine (L), or arginine (R) residue;
wherein consensus position 339 of the conserved insert region is an alanine (A), isoleucine (I), or tyrosine (Y) residue;
wherein consensus position 341 of the conserved insert region is a phenylalanine (F) or leucine (L) residue; and/or
wherein consensus position 343 of the conserved insert region is a proline (P) or serine(S) residue.

In some embodiments, the CRISPR nuclease amino acid sequence is other than SEQ ID NOs: 45-48.

In some embodiments, the CRISPR nuclease is a nickase having an inactivated RuvC domain created by an amino acid substitution at a position provided for the CRISPR nuclease in column 5 of Table 1.

In some embodiments, the CRISPR nuclease is a nickase having an inactivated HNH domain created by an amino acid substitution at a position provided for the CRISPR nuclease in column 6 of Table 1.

In some embodiments, the CRISPR nuclease is a catalytically dead nuclease having an inactivated RuvC domain and an inactivated HNH domain created by substitutions at the positions provided for the CRISPR nuclease in column 7 of Table 1.

For example, a nickase may be generated for the OMNI-XL-12 nuclease by inactivating its RuvC domain by substituting an aspartic acid residue (D) in position 7 of the amino acid sequence of OMNI-XL-12 (SEQ ID NO: 12) for another amino acid e.g. alanine (A). Substitution to any other amino acid is permissible for each of the amino acid positions indicated in columns 5-7, unless otherwise indicated in Table 1. Other nickases or catalytically dead nucleases can be generated using the same notation in Table 1.

In some embodiments, the CRISPR nuclease utilizes a protospacer adjacent motif (PAM) sequence provided for the CRISPR nuclease in columns 2-3 of Table 3.

The invention also provides a method for modifying a nucleotide sequence at a DNA target site in a cell-free system or the genome of a cell comprising introducing into the cell any one of the compositions described above. In some embodiments, the composition comprises a CRISPR nuclease and a crRNA: tracrRNA complex or a sgRNA molecule.

In some embodiments, the CRISPR nuclease effects a DNA break in a DNA strand adjacent to a protospacer adjacent motif (PAM) sequence provided for the CRISPR nuclease in columns 2-3 of Table 3, and effects a DNA break in a DNA strand adjacent to a sequence that is complementary to the PAM sequence. For example, the OMNI-XL-12 nuclease with the appropriate targeting sgRNA or crRNA: tracrRNA complex is capable of forming a DNA break in strand adjacent to a NRNNCCNN or NRTNCCRN sequence and in a DNA strand adjacent to a sequence that is complementary to a NRNNCCNN or NRTNCCRN sequence. In some embodiments, the DNA strand is within a nucleus of a cell.

In some embodiments, the CRISPR nuclease is a nickase having an inactivated RuvC domain created by an amino acid substitution at a position provided for the CRISPR nuclease in column 5 of Table 1, and effects a DNA break in a DNA strand adjacent to a sequence that is complementary to the PAM sequence.

In some embodiments, the CRISPR nuclease is a nickase having an inactivated HNH domain created by an amino acid substitution at a position provided for the CRISPR nuclease in column 6 of Table 1, and effects a DNA break in a DNA strand adjacent to the PAM sequence.

In some embodiments, the CRISPR nuclease is a catalytically dead nuclease having an inactivated RuvC domain and an inactivated HNH domain created by substitutions at the positions provided for the CRISPR nuclease in column 7 of Table 1, and effects a DNA break in a DNA strand adjacent to the PAM sequence.

In some embodiments, the cell is a eukaryotic cell or a prokaryotic cell.

In some embodiments, the cell is a mammalian cell.

In some embodiments, the cell is a human cell.

In some embodiments, the CRISPR nuclease comprises an amino acid sequence having at least 100%, 99%, 98%, 97%, 96%, 95%, 94%, 93%, 92%, 91%, 90%, 89%, 88%, 87%, 86%, 85%, 84%, 83%, or 82% amino acid sequence identity to a CRISPR nuclease as set forth in any of SEQ ID NOs: 1-22. In an embodiment the sequence encoding the CRISPR nuclease has at least 95% identity to a nucleic acid sequence selected from the group consisting of SEQ ID NOs: 23-44.

According to some aspects of the invention, the disclosed compositions comprise DNA constructs or a vector system comprising nucleotide sequences that encode the CRISPR nuclease or variant CRISPR nuclease. In some embodiments, the nucleotide sequence that encode the CRISPR nuclease or variant CRISPR nuclease is operably linked to a promoter that is operable in the cells of interest. In some embodiments, the cell of interest is a eukaryotic cell. In some embodiments the cell of interest is a mammalian cell. In some embodiments, the nucleic acid sequence encoding the engineered CRISPR nuclease is codon optimized for use in cells from a particular organism. In some embodiments, the nucleic acid sequence encoding the nuclease is codon optimized for E. coli. In some embodiments, the nucleic acid sequence encoding the nuclease is codon optimized for eukaryotic cells. In some embodiments, the nucleic acid sequence encoding the nuclease is codon optimized for mammalian cells.

In some embodiments, the composition comprises a recombinant nucleic acid, comprising a heterologous promoter operably linked to a polynucleotide encoding a CRISPR enzyme having at least 100%, 99%, 98%, 97%, 96%, 95%, 94%, 93%, 92%, 91%, 90% identity to any of SEQ ID NOs: 1-22. Each possibility represents a separate embodiment.

In an embodiment of the composition, the CRISPR nuclease has at least 75%, 80%, 85, 90%, 95%, or 97% identity to the amino acid sequence as set forth in SEQ ID NO: 1 or the sequence encoding the CRISPR nuclease has at least a 75%, 80%, 85, 90%, 95%, or 97% sequence identity to the nucleotide sequence as set forth in SEQ ID NO: 23.

In an embodiment of the composition, the CRISPR nuclease has at least 75%, 80%, 85, 90%, 95%, or 97% identity to the amino acid sequence as set forth in SEQ ID NO: 2 or the sequence encoding the CRISPR nuclease has at least a 75%, 80%, 85, 90%, 95%, or 97% sequence identity to the nucleotide sequence as set forth in SEQ ID NO: 24.

In an embodiment of the composition, the CRISPR nuclease has at least 75%, 80%, 85, 90%, 95%, or 97% identity to the amino acid sequence as set forth in SEQ ID NO: 3 or the sequence encoding the CRISPR nuclease has at least a 75%, 80%, 85, 90%, 95%, or 97% sequence identity to the nucleotide sequence as set forth in SEQ ID NO: 25.

In an embodiment of the composition, the CRISPR nuclease has at least 75%, 80%, 85, 90%, 95%, or 97% identity to the amino acid sequence as set forth in SEQ ID NO: 4 or the sequence encoding the CRISPR nuclease has at least a 75%, 80%, 85, 90%, 95%, or 97% sequence identity to the nucleotide sequence as set forth in SEQ ID NO: 26.

In an embodiment of the composition, the CRISPR nuclease has at least 75%, 80%, 85, 90%, 95%, or 97% identity to the amino acid sequence as set forth in SEQ ID NO: 5 or the sequence encoding the CRISPR nuclease has at least a 75%, 80%, 85, 90%, 95%, or 97% sequence identity to the nucleotide sequence as set forth in SEQ ID NO: 27.

In an embodiment of the composition, the CRISPR nuclease has at least 75%, 80%, 85, 90%, 95%, or 97% identity to the amino acid sequence as set forth in SEQ ID NO: 6 or the sequence encoding the CRISPR nuclease has at least a 75%, 80%, 85, 90%, 95%, or 97% sequence identity to the nucleotide sequence as set forth in SEQ ID NO: 28.

In an embodiment of the composition, the CRISPR nuclease has at least 75%, 80%, 85, 90%, 95%, or 97% identity to the amino acid sequence as set forth in SEQ ID NO: 7 or the sequence encoding the CRISPR nuclease has at least a 75%, 80%, 85, 90%, 95%, or 97% sequence identity to the nucleotide sequence as set forth in SEQ ID NO: 29.

In an embodiment of the composition, the CRISPR nuclease has at least 75%, 80%, 85, 90%, 95%, or 97% identity to the amino acid sequence as set forth in SEQ ID NO: 8 or the sequence encoding the CRISPR nuclease has at least a 75%, 80%, 85, 90%, 95%, or 97% sequence identity to the nucleotide sequence as set forth in SEQ ID NO: 30.

In an embodiment of the composition, the CRISPR nuclease has at least 75%, 80%, 85, 90%, 95%, or 97% identity to the amino acid sequence as set forth in SEQ ID NO: 9 or the sequence encoding the CRISPR nuclease has at least a 75%, 80%, 85, 90%, 95%, or 97% sequence identity to the nucleotide sequence as set forth in SEQ ID NO: 31.

In an embodiment of the composition, the CRISPR nuclease has at least 75%, 80%, 85, 90%, 95%, or 97% identity to the amino acid sequence as set forth in SEQ ID NO: 10 or the sequence encoding the CRISPR nuclease has at least a 75%, 80%, 85, 90%, 95%, or 97% sequence identity to the nucleotide sequence as set forth in SEQ ID NO: 32.

In an embodiment of the composition, the CRISPR nuclease has at least 75%, 80%, 85, 90%, 95%, or 97% identity to the amino acid sequence as set forth in SEQ ID NO: 11 or the sequence encoding the CRISPR nuclease has at least a 75%, 80%, 85, 90%, 95%, or 97% sequence identity to the nucleotide sequence as set forth in SEQ ID NO: 33.

In an embodiment of the composition, the CRISPR nuclease has at least 75%, 80%, 85, 90%, 95%, or 97% identity to the amino acid sequence as set forth in SEQ ID NO: 12 or the sequence encoding the CRISPR nuclease has at least a 75%, 80%, 85, 90%, 95%, or 97% sequence identity to a nucleotide sequence selected from the group consisting of SEQ ID NOs: 34 and 392. In an embodiment of the composition, the CRISPR nuclease has at least 75%, 80%, 85, 90%, 95%, or 97% identity to the amino acid sequence as set forth in SEQ ID NO: 13 or the sequence encoding the CRISPR nuclease has at least a 75%, 80%, 85, 90%, 95%, or 97% sequence identity to the nucleotide sequence as set forth in SEQ ID NO: 35.

In an embodiment of the composition, the CRISPR nuclease has at least 75%, 80%, 85, 90%, 95%, or 97% identity to the amino acid sequence as set forth in SEQ ID NO: 14 or the sequence encoding the CRISPR nuclease has at least a 75%, 80%, 85, 90%, 95%, or 97% sequence identity to the nucleotide sequence as set forth in SEQ ID NO: 36.

In an embodiment of the composition, the CRISPR nuclease has at least 75%, 80%, 85, 90%, 95%, or 97% identity to the amino acid sequence as set forth in SEQ ID NO: 15 or the sequence encoding the CRISPR nuclease has at least a 75%, 80%, 85, 90%, 95%, or 97% sequence identity to the nucleotide sequence as set forth in SEQ ID NO: 37.

In an embodiment of the composition, the CRISPR nuclease has at least 75%, 80%, 85, 90%, 95%, or 97% identity to the amino acid sequence as set forth in SEQ ID NO: 16 or the sequence encoding the CRISPR nuclease has at least a 75%, 80%, 85, 90%, 95%, or 97% sequence identity to a nucleotide sequence selected from the group consisting of SEQ ID NOs: 38 and 393.

In an embodiment of the composition, the CRISPR nuclease has at least 75%, 80%, 85, 90%, 95%, or 97% identity to the amino acid sequence as set forth in SEQ ID NO: 17 or the sequence encoding the CRISPR nuclease has at least a 75%, 80%, 85, 90%, 95%, or 97% sequence identity to the nucleotide sequence as set forth in SEQ ID NO: 39.

In an embodiment of the composition, the CRISPR nuclease has at least 75%, 80%, 85, 90%, 95%, or 97% identity to the amino acid sequence as set forth in SEQ ID NO: 18 or the sequence encoding the CRISPR nuclease has at least a 75%, 80%, 85, 90%, 95%, or 97% sequence identity to a nucleotide sequence selected from the group consisting of SEQ ID NOs: 40 and 394.

In an embodiment of the composition, the CRISPR nuclease has at least 75%, 80%, 85, 90%, 95%, or 97% identity to the amino acid sequence as set forth in SEQ ID NO: 19 or the sequence encoding the CRISPR nuclease has at least a 75%, 80%, 85, 90%, 95%, or 97% sequence identity to a nucleotide sequence selected from the group consisting of SEQ ID NOs: 41 and 395.

In an embodiment of the composition, the CRISPR nuclease has at least 75%, 80%, 85, 90%, 95%, or 97% identity to the amino acid sequence as set forth in SEQ ID NO: 20 or the sequence encoding the CRISPR nuclease has at least a 75%, 80%, 85, 90%, 95%, or 97% sequence identity to the nucleotide sequence as set forth in SEQ ID NO: 42.

In an embodiment of the composition, the CRISPR nuclease has at least 75%, 80%, 85, 90%, 95%, or 97% identity to the amino acid sequence as set forth in SEQ ID NO: 21 or the sequence encoding the CRISPR nuclease has at least a 75%, 80%, 85, 90%, 95%, or 97% sequence identity to the nucleotide sequence as set forth in SEQ ID NO: 43.

In an embodiment of the composition, the CRISPR nuclease has at least 75%, 80%, 85, 90%, 95%, or 97% identity to the amino acid sequence as set forth in SEQ ID NO: 22 or the sequence encoding the CRISPR nuclease has at least a 75%, 80%, 85, 90%, 95%, or 97% sequence identity to the nucleotide sequence as set forth in SEQ ID NO: 44.

According to some embodiments, there is provided an engineered or non-naturally occurring composition comprising a CRISPR nuclease comprising a sequence having at least 100%, 99%, 98%, 97%, 96%, 95%, 94%, 93%, 92%, 91%, 90%, 85%, 80% identity to the amino acid sequence selected from the group consisting of SEQ ID NOs: 1-22 or a nucleic acid molecule comprising a sequence encoding the CRISPR nuclease. Each possibility represents a separate embodiment.

In an embodiment, the CRISPR nuclease is engineered or non-naturally occurring. The CRISPR nuclease may also be recombinant. Such CRISPR nucleases are produced using laboratory methods (molecular cloning) to bring together genetic material from multiple sources, creating sequences that would not otherwise be found in biological organisms.

In an embodiment, the CRISPR nuclease further comprises an RNA-binding portion capable of interacting with a DNA-targeting RNA molecule (gRNA) and an activity portion that exhibits site-directed enzymatic activity.

In an embodiment, the composition further comprises a DNA-targeting RNA molecule or a DNA polynucleotide encoding a DNA-targeting RNA molecule, wherein the DNA-targeting RNA molecule comprises a guide sequence portion, i.e. a nucleotide sequence that is complementary to a sequence in a target region, wherein the DNA-targeting RNA molecule and the CRISPR nuclease do not naturally occur together.

In an embodiment, the DNA-targeting RNA molecule further comprises a nucleotide sequence that can form a complex with a CRISPR nuclease.

This invention also provides a non-naturally occurring composition comprising a CRISPR associated system comprising:
one or more RNA molecules comprising a guide sequence portion linked to a direct repeat sequence, wherein the guide sequence is capable of hybridizing with a target sequence, or one or more nucleotide sequences encoding the one or more RNA molecules; and
a CRISPR nuclease comprising an amino acid sequence having at least 95% identity to the amino acid sequence selected from the group consisting of SEQ ID NOs: 1-22 or a nucleic acid molecule comprising a sequence encoding the CRISPR nuclease;
wherein the one or more RNA molecules hybridize to the target sequence, wherein the target sequence is 3′ of a Protospacer Adjacent Motif (PAM), and the one or more RNA molecules form a complex with the RNA-guided nuclease.

In an embodiment, the composition further comprises an RNA molecule comprising a nucleotide sequence that can form a complex with a CRISPR nuclease (e.g. a tracrRNA molecule) or a DNA polynucleotide comprising a sequence encoding an RNA molecule that can form a complex with the CRISPR nuclease.

In an embodiment, the composition further comprises a donor template for homology directed repair (HDR).

In an embodiment, the composition is capable of editing the target region in the genome of a cell.

According to some embodiments, there is provided a non-naturally occurring composition comprising:
(a) a CRISPR nuclease, or a polynucleotide encoding the CRISPR nuclease, comprising:
   an RNA-binding portion; and
   an activity portion that exhibits site-directed enzymatic activity, wherein the CRISPR nuclease has at least 100%, 99%, 98%, 97%, 96%, 95%, 94%, 93%, 92%, 91%, 90%, 85%, 80% identity to any of SEQ ID NOs: 1-22; and
(b) one or more RNA molecules or a DNA polynucleotide encoding the one or more RNA molecules comprising:
   i) a DNA-targeting RNA sequence, comprising a nucleotide sequence that is complementary to a sequence in a target DNA sequence; and
   ii) a protein-binding RNA sequence, capable of interacting with the RNA-binding portion of the CRISPR nuclease,
wherein the DNA targeting RNA sequence and the CRISPR nuclease do not naturally occur together. Each possibility represents a separate embodiment.

In some embodiments, there is provided a single RNA molecule comprising the DNA-targeting RNA sequence and the protein-binding RNA sequence, wherein the RNA molecule can form a complex with the CRISPR nuclease and serve as the DNA targeting module. In some embodiments, the RNA molecule has a length of up to 1000 bases, 900 bases, 800 bases, 700 bases, 600 bases, 500 bases, 400 bases, 300 bases, 200 bases, 100 bases, 50 bases. Each possibility represents a separate embodiment. In some embodiments, a first RNA molecule comprising the DNA-targeting RNA sequence and a second RNA molecule comprising the protein-binding RNA sequence interact by base pairing or alternatively fused together to form one or more RNA molecules that complex with the CRISPR nuclease and serve as the DNA targeting module.

This invention also provides a non-naturally occurring composition comprising:
a CRISPR nuclease comprising a sequence having at least 95% identity to the amino acid sequence selected from the group consisting of SEQ ID NOs: 1-22 or a nucleic acid molecule comprising a sequence encoding the CRISPR nuclease; and
one or more RNA molecules, or one or more DNA polynucleotide encoding the one or more RNA molecules, comprising at least one of:
a nuclease-binding RNA nucleotide sequence capable of interacting with/binding to the CRISPR nucleasc; and
a DNA-targeting RNA nucleotide sequence comprising a sequence complementary to a sequence in a target DNA sequence,
   wherein the CRISPR nuclease is capable of complexing with the one or more RNA molecules to form a complex capable of hybridizing with the target DNA sequence.

In an embodiment, the CRISPR nuclease and the one or more RNA molecules form a CRISPR complex that is capable of binding to the target DNA sequence to effect cleavage of the target DNA sequence.

In an embodiment, the CRISPR nuclease and at least one of the one or more RNA molecules do not naturally occur together.

In an embodiment:
the CRISPR nuclease comprises an RNA-binding portion and an activity portion that exhibits site-directed enzymatic activity;
the DNA-targeting RNA nucleotide sequence comprises a nucleotide sequence that is complementary to a sequence in a target DNA sequence; and
the nuclease-binding RNA nucleotide sequence comprises a sequence that interacts with the RNA-binding portion of the CRISPR nuclcasc.

In an embodiment, the nuclease-binding RNA nucleotide sequence and the DNA-targeting RNA nucleotide sequence are on a single guide RNA molecule (sgRNA), wherein the sgRNA molecule can form a complex with the CRISPR nuclease and serve as the DNA targeting module.

In an embodiment, the nuclease-binding RNA nucleotide sequence is on a first RNA molecule and the DNA-targeting RNA nucleotide sequence is on a second RNA molecule, and wherein the first and second RNA molecules interact by base-pairing or are fused together to form a RNA complex or sgRNA that forms a complex with the CRISPR nuclease and serves as a DNA targeting module.

In an embodiment, the sgRNA has a length of up to 1000 bases, 900 bases, 800 bases, 700 bases, 600 bases, 500 bases, 400 bases, 300 bases, 200 bases, 100 bases, 50 bases.

In an embodiment, the composition further comprises a donor template for homology directed repair (HDR).

In an embodiment, the CRISPR nuclease is non-naturally occurring.

In an embodiment, the CRISPR nuclease is engineered and comprises unnatural or synthetic amino acids.

In an embodiment, the CRISPR nuclease is engineered and comprises one or more of a nuclear localization sequences (NLS), cell penetrating peptide sequences, and/or affinity tags.

In an embodiment, the CRISPR nuclease comprises one or more nuclear localization sequences of sufficient strength to drive accumulation of a CRISPR complex comprising the CRISPR nuclease in a detectable amount in the nucleus of a eukaryotic cell.

This invention also provides a method of modifying a nucleotide sequence at a target site in a cell-free system or the genome of a cell comprising introducing into the cell any of the compositions of the invention.

In an embodiment, the cell is a eukaryotic cell.

In another embodiment, the cell is a prokaryotic cell.

In some embodiments, the one or more RNA molecules further comprises an RNA sequence comprising a nucleotide molecule that can form a complex with the RNA nuclease (tracrRNA) or a DNA polynucleotide encoding an RNA molecule comprising a nucleotide sequence that can form a complex with the CRISPR nuclease.

In an embodiment, the CRISPR nuclease comprises 1, 2, 3, 4, 5, 6, 7, 8, 9, 10, or more NLSs at or near the amino-terminus, 1, 2, 3, 4, 5, 6, 7, 8, 9, 10, or more NLSs at or near carboxy-terminus, or a combination of 1, 2, 3, 4, 5, 6, 7, 8, 9, 10, or more NLSs at or near the amino-terminus and 1, 2, 3, 4, 5, 6, 7, 8, 9, 10, or more NLSs at or near carboxy-terminus. In an embodiment, 1-4 NLSs are fused with the CRISPR nuclease. In an embodiment, an NLS is located within the open-reading frame (ORF) of the CRISPR nuclease.

Methods of fusing an NLS at or near the amino-terminus, at or near carboxy-terminus, or within the ORF of an expressed protein are well known in the art. As an example, to fuse an NLS to the amino-terminus of a CRISPR nuclease, the nucleic acid sequence of the NLS is placed immediately after the start codon of the CRISPR nuclease on the nucleic acid encoding the NLS-fused CRISPR nuclease. Conversely, to fuse an NLS to the carboxy-terminus of a CRISPR nuclease the nucleic acid sequence of the NLS is placed after the codon encoding the last amino acid of the CRISPR nuclease and before the stop codon.

Any combination of NLSs, cell penetrating peptide sequences, and/or affinity tags at any position along the ORF of the CRISPR nuclease is contemplated in this invention.

The amino acid sequences and nucleic acid sequences of the CRISPR nucleases provided herein may include NLS and/or TAGs inserted so as to interrupt the contiguous amino acid or nucleic acid sequences of the CRISPR nucleases.

In an embodiment, the one or more NLSs are in tandem repeats.

In an embodiment, the one or more NLSs are considered in proximity to the N- or C-terminus when the nearest amino acid of the NLS is within about 1, 2, 3, 4, 5, 10, 15, 20, 25, 30, 40, 50, or more amino acids along the polypeptide chain from the N- or C-terminus.

As discussed, the CRISPR nuclease may be engineered to comprise one or more of a nuclear localization sequences (NLS), cell penetrating peptide sequences, and/or affinity tags. In an embodiment, the CRISPR nuclease exhibits increased specificity to a target site compared to the wild-type of the CRISPR nuclease when complexed with the one or more RNA molecules.

In an embodiment, the complex of the CRISPR nuclease and one or more RNA molecules exhibits at least maintained on-target editing activity of the target site and reduced off-target activity compared to the wild-type of the CRISPR nuclease.

In an embodiment, the composition further comprises a recombinant nucleic acid molecule comprising a heterologous promoter operably linked to the nucleotide acid molecule comprising the sequence encoding the CRISPR nuclease.

In an embodiment, the CRISPR nuclease or nucleic acid molecule comprising a sequence encoding the CRISPR nuclease is non-naturally occurring or engineered.

This invention also provides a non-naturally occurring or engineered composition comprising a vector system comprising the nucleic acid molecule comprising a sequence encoding any of the CRISPR nucleases of the invention.

This invention also provides use of any of the compositions of the invention for the treatment of a subject afflicted with a disease associated with a genomic mutation comprising modifying a nucleotide sequence at a target site in the genome of the subject.

This invention provides a method of modifying a nucleotide sequence at a target site in the genome of a mammalian cell comprising introducing into the cell (i) a composition comprising a CRISPR nuclease having at least 95% identity to an amino acid sequence selected from the group consisting of SEQ ID NOs: 1-22 or a nucleic acid molecule comprising a sequence encoding a CRISPR nuclease which sequence has at least 95% identity to a nucleic acid sequence selected from the group consisting of SEQ ID NOs: 23-44 and (ii) a DNA-targeting RNA molecule, or a DNA polynucleotide encoding a DNA-targeting RNA molecule, comprising a nucleotide sequence that is complementary to a sequence in the target DNA.

In some embodiments, the method is performed ex vivo. In some embodiments, the method is performed in vivo. In some embodiments, some steps of the method are performed ex vivo and some steps are performed in vivo. In some embodiments the mammalian cell is a human cell. In an embodiment, the method further comprises introducing into the cell: (iii) an RNA molecule comprising a tracrRNA sequence or a DNA polynucleotide encoding an RNA molecule comprising a tracrRNA sequence.

In an embodiment, the DNA-targeting RNA molecule comprises a crRNA repeat sequence.

In an embodiment, the RNA molecule comprising a tracrRNA sequence is able to bind the DNA-targeting RNA moleculc.

In an embodiment, the DNA-targeting RNA molecule and the RNA molecule comprising a tracrRNA sequence interact to form an RNA complex, and the RNA complex is capable of forming an active complex with the CRISPR nuclease.

In an embodiment, the DNA-targeting RNA molecule and the RNA molecule comprising a nuclease-binding RNA sequence are fused in the form of a single guide RNA molecule that is suitable to form an active complex with the CRISPR nuclease.

In an embodiment, the guide sequence portion comprises a sequence complementary to a protospacer sequence.

In an embodiment, the CRISPR nuclease forms a complex with the DNA-targeting RNA molecule and effects a double strand break in a region that is 3′ or 5′ of a Protospacer Adjacent Motif (PAM). In an embodiment of any of the methods described herein, the method is for treating a subject afflicted with a disease associated with a genomic mutation comprising modifying a nucleotide sequence at a target site in the genome of the subject.

In an embodiment, the method comprises first selecting a subject afflicted with a disease associated with a genomic mutation and obtaining the cell from the subject.

This invention also provides a modified cell or cells obtained by any of the methods described herein. In an embodiment these modified cell or cells are capable of giving rise to progeny cells.

In an embodiment these modified cell or cells are capable of giving rise to progeny cells after engraftment.

This invention also provides a composition comprising these modified cells and a pharmaceutically acceptable carrier. Also provided is an in vitro or ex vivo method of preparing this, comprising mixing the cells with the pharmaceutically acceptable carrier.

### DNA-Targeting RNA Molecules

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence. Accordingly, a molecule comprising a “guide sequence portion” is a type of targeting molecule. Throughout this application, the terms “guide molecule,” “RNA guide molecule,” “guide RNA molecule,” and “gRNA molecule” are synonymous with a molecule comprising a guide sequence portion, and the term “spacer” is synonymous with a “guide sequence portion.

In embodiments of the present invention, the CRISPR nuclease has its greatest cleavage activity when used with an RNA molecule comprising a guide sequence portion having 17, 18, 19 20, 21, 22, 23, 24, 25, 26, 27, 28, 29, or 30 nucleotides.

A single-guide RNA (sgRNA) molecule may be used to direct a CRISPR nuclease to a desired target site. The single-guide RNA comprises a guide sequence portion as well as a scaffold portion. The scaffold portion interacts with a CRISPR nuclease and, together with a guide sequence portion, activates and targets the CRISPR nuclease to a desired target site. A scaffold portion may be further engineered, for example, to have a reduced size.

According to some aspects of the invention, the disclosed methods comprise a method of modifying a nucleotide sequence at a target site in a cell-free system or the genome of a cell comprising introducing into the cell the composition of any one of the embodiments described herein.

In some embodiments, the cell is a eukaryotic cell, preferably a mammalian cell or a plant cell.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, beta thalassemia, sickle cell anemia, cell dysregulation and oncology diseases and disorders, inflammation and immune-related diseases and disorders, metabolic, liver, hypercholesteremia, kidney and protein diseases and disorders, muscular and skeletal diseases and disorders, dermatological diseases and disorders, neurological and neuronal diseases and disorders, pulmonary disease and disorders, corneal disease and disorders, retinal diseases and disorders, and ocular diseases and disorders.

### CRISPR Nuclease Domains

The characteristic targeted nuclease activity of a CRISPR nuclease is imparted by the various functions of its specific domains. In this application the OMNI-XL domains are defined as Domain A, Domain B, Domain C, Domain D, Domain E, Domain F, Domain G, Domain H, Domain I, Domain J, Domain K, Domain L, and Domain M.

In an embodiment Domain A has been identified as amino acids 1-46 of SEQ ID NO: 12, 1-49 of SEQ ID NO: 16, 1-52 of SEQ ID NO: 18, and 1-52 of SEQ ID NO: 19.

In an embodiment Domain B has been identified as amino acids 47-89 of SEQ ID NO: 12, 50-84 of SEQ ID NO: 16, 53-86 of SEQ ID NO: 18, and 53-86 of SEQ ID NO: 19.

In an embodiment Domain C has been identified as amino acids 90-285 of SEQ ID NO: 12, 85-340 of SEQ ID NO: 16, 87-339 of SEQ ID NO: 18, and 87-339 of SEQ ID NO: 19.

In an embodiment Domain D has been identified as amino acids 286-545 of SEQ ID NO: 12, 341-596 of SEQ ID NO: 16, 340-603 of SEQ ID NO: 18, and 340-603 of SEQ ID NO: 19.

In an embodiment Domain E has been identified as amino acids 546-622 of SEQ ID NO: 12, 597-674 of SEQ ID NO: 16, 604-681 of SEQ ID NO: 18, and 604-681 of SEQ ID NO: 19.

In an embodiment Domain F has been identified as amino acids 623-653 of SEQ ID NO: 12, 675-705 of SEQ ID NO: 16, 682-712 of SEQ ID NO: 18, and 682-712 of SEQ ID NO: 19.

In an embodiment Domain G has been identified as amino acids 654-774 of SEQ ID NO: 12, 706-825 of SEQ ID NO: 16, 713-834 of SEQ ID NO: 18, and 713-834 of SEQ ID NO: 19.

In an embodiment Domain H has been identified as amino acids 775-789 of SEQ ID NO: 12, 826-838 of SEQ ID NO: 16, 835-845 of SEQ ID NO: 18, and 835-845 of SEQ ID NO: 19.

In an embodiment Domain I has been identified as amino acids 790-828 of SEQ ID NO: 12, 839-877 of SEQ ID NO: 16, 846-886 of SEQ ID NO: 18, and 846-886 of SEQ ID NO: 19.

In an embodiment Domain J has been identified as amino acids 829-1153 of SEQ ID NO: 12, 878-1179 of SEQ ID NO: 16, 887-1203 of SEQ ID NO: 18, and 887-1203 of SEQ ID NO: 19.

In an embodiment Domain K has been identified as amino acids 1154-1264 of SEQ ID NO: 12, 1180-1278 of SEQ ID NO: 16, 1204-1313 of SEQ ID NO: 18, and 1204-1313 of SEQ ID NO: 19.

In an embodiment Domain L has been identified as amino acids 1265-1360 of SEQ ID NO: 12, 1279-1372 of SEQ ID NO: 16, 1314-1411 of SEQ ID NO: 18, and 1314-1407 of SEQ ID NO: 19.

In an embodiment Domain M has been identified as amino acids 1361-1511 of SEQ ID NO: 12, 1373-1517 of SEQ ID NO: 16, 1412-1565 of SEQ ID NO: 18, and 1408-1566 of SEQ ID NO: 19.

The ranges of each domain may vary slightly based on parameters of the sequence alignment analysis. For example, the start or end of the any one of the above ranges may vary by up to ten amino acids.

The activity of each domain provides aspects of the advantageous features of each nuclease. Description of several CRISPR nuclease domains and their general functions can be found in, inter alia, Mir et al., ACS Chem. Biol. (2019), Palermo et al., Quarterly Reviews of Biophysics (2018), Jiang and Doudna, Annual Review of Biophysics (2017), Nishimasu et al., Cell (2014) and Nishimasu et al., Cell (2015), incorporated herein by reference.

In one aspect of the invention, an amino acid sequence having similarity to an OMNI-XL domain may be utilized in the design and manufacture of a non-naturally occurring peptide, e.g. a CRISPR nuclease, such that the peptide displays the advantageous features of the OMNI-XL domain activity.

In an embodiment, such a peptide, e.g. a CRISPR nuclease, comprises an amino acid sequence that has at least 100%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence of at least one of Domain A, Domain B, Domain C, Domain D, Domain E, Domain F, Domain G, Domain H, Domain I, Domain J, Domain K, Domain L, or Domain M of nuclease. In some embodiments, the identity is to at least one, at least two, at least three, at least four, at least five, at least six, at least seven or at least eight amino acid sequences of Domain A, Domain B, Domain C, Domain D, Domain E, Domain F, Domain G, Domain H, Domain I, Domain J, Domain K, Domain L, or Domain M of the nuclease. Each possibility represents a separate embodiment. In some embodiments the identity is to the amino acid sequence of at least one of Domain A, Domain B, Domain C, Domain D, Domain E, Domain F, Domain G, Domain H, Domain I, Domain J, Domain K, Domain L, or Domain M of the nuclease. In some embodiments the identity is to the amino acid sequence of Domain J of the nuclease. In an embodiment, the peptide exhibits extensive amino acid variability relative to the full length OMNI-XL amino acid sequence outside of the peptide amino acid sequence having at least 100%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence of at least one of Domain A, Domain B, Domain C, Domain D, Domain E, Domain F, Domain G, Domain H, Domain I, Domain J, Domain K, Domain L, or Domain M of the OMNI-XL nuclease. In an embodiment, the peptide comprises an intervening amino acid sequence between two domain sequences. In an embodiment, the intervening amino acid sequence is 1-10, 10-20, 20-40, 40-50 or up to 100 amino acids in length. In an embodiment, the intervening sequence is a linker sequence.

In one aspect of the invention, an amino acid sequence encoding any one of the domains of the OMNI-XL nucleases described herein in the peptide may comprise one or more amino acid substitutions relative to the original OMNI-XL domain sequence. The amino acid substitution may be a conservative substitution, i.e. substitution for an amino acid having similar chemical properties as the original amino acid. For example, a positively charged amino acid may be substituted for an alternate positively charged amino acid, e.g. an arginine residue may be substituted for a lysine residue, or a polar amino acid may be substituted for a different polar amino acid. Conservative substitutions are more tolerable, and the amino acid sequence encoding any one of the domains of the OMNI-XL nuclease may contain as many as 10% of such substitutions. The amino acid substitution may be a radical substitution, i.e. substitution for an amino acid having different chemical properties as the original amino acid. For example, a positively charged amino acid may be substituted for a negatively charged amino acid, e.g. an arginine residue may be substituted for a glutamic acid residue, or a polar amino acid may be substituted for a non-polar amino acid. The amino acid substitution may be a semi-conservative substitution, or the amino acid substitution may be to any other amino acid. The substitution may alter the activity relative to the original OMNI-XL domain function e.g. reduce catalytic nuclease activity.

According to some aspects of the invention, the disclosed compositions comprise a non-naturally occurring composition comprising a CRISPR nuclease, wherein the CRISPR nuclease comprises an amino acid sequence corresponding to the amino acid sequence of at least one of Domain A, Domain B, Domain C, Domain D, Domain E, Domain F, Domain G, Domain H, Domain I, Domain J, Domain K, Domain L, or Domain M of the OMNI-XL nuclease. In some embodiments of the invention, the CRISPR nuclease comprises at least one, at least two, at least three, at least four, or at least five amino acid sequences, wherein each amnio acid sequence corresponds to any one of the amino acid sequences Domain A, Domain B, Domain C, Domain D, Domain E, Domain F, Domain G, Domain H, Domain I, Domain J, Domain K, Domain L, or Domain M of the OMNI-XL nuclease. Accordingly, the CRISPR nuclease may include any combination of amino acid sequences that corresponds to any of Domain A, Domain B, Domain C, Domain D, Domain E, Domain F, Domain G, Domain H, Domain I, Domain J, Domain K, Domain L, or Domain M of the OMNI-XL nuclease. In some embodiments, the amino acid sequence is at least 100-250, 250-500, 500-1000, 1000-1500, 1000-1700, or 1000-2000 amino acids in length.

### Diseases and Therapies

Certain embodiments of the invention target a nuclease to a specific genetic locus associated with a disease or disorder as a form of gene editing, method of treatment, or therapy. For example, to induce editing or knockout of a gene, a novel nuclease disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-XL-12 nuclease to a target site is designed to contain a spacer region complementary to a DNA strand of a DNA double-stranded region that neighbors a OMNI-XL-12 PAM sequence, e.g. “NRNNCCNN” or “NRTNCCRN.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/DISORDERS | GENE(S) |
|---|---|
| Neoplasia | PTEN; ATM; ATR; EGFR; ERBB2; ERBB3; ERBB4; Notch1; |
| | Notch2; Notch3; Notch4; AKT; AKT2; AKT3; HIF; HIF1a; |
| | HIF3a; Met; HRG; Bcl2; PPAR alpha; PPAR gamma; WT1 |
| | (Wilms Tumor); FGF Receptor Family members (5 members: 1, |
| | 2, 3, 4, 5); CDKN2a; APC; RB (retinoblastoma); MEN1; VHL; |
| | BRCA1; BRCA2; AR (Androgen Receptor); TSG101; IGF; IGF |
| | Receptor; Igf1 (4 variants); gf2 (3 variants); Igf 1 Receptor; Igf 2 |
| | Receptor; Bax; Bcl2; caspases family (9 members: 1, 2, 3, 4, 6, |
| | 7, 8, 9, 12); Kras; Apc |
| Age-related Macular | Abcr; Ccl2; Cc2; cp (ceruloplasmin); Timp3; cathepsinD; Vldlr; |
| Degeneration | Ccr2 |
| Schizophrenia | Neuregulin1 (Nrg1); Erb4 (receptor for Neuregulin); |
| | Complexin1 (Cp1x1); Tph1 Tryptophan hydroxylase; Tph2 |
| | Tryptophan hydroxylase 2; Neurexin 1; GSK3; GSK3a; GSK3b |
| Neurological, Neuro | 5-HTT (S1c6a4); COMT; DRD (Drd1a); SLC6A3; DAOA; |
| degenerative, and | DTNBP1; Dao (Dao1) |
| Movement Disorders | |
| Trinucleotide Repeat | HTT (Huntington's Dx); SBMA/SMAX1/AR (Kennedy's Dx); |
| Disorders | FXN/X25 (Friedrich's Ataxia); ATX3 (Machado-Joseph's Dx); |
| | ATXN1 and ATXN2 (spinocerebellar ataxias); DMPK |
| | (myotonic dystrophy); Atrophin-1 and Atn1 (DRPLA Dx); CBP |
| | (Creb-BP - global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); |
| Disorders | PEN-2 |
| Others | Nos1; Parp1; Nat1; Nat2 |
| Prion related disorders | Prp |
| ALS | SOD1; ALS2; STEX; FUS; TARDBP; VEGF (VEGF-a; VEGF- |
| | b; VEGF-c) |
| Addiction | Prkce (alcohol); Drd2; Drd4; ABAT (alcohol); GRIA2; Grm5; |
| | Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, Aquaporin |
| | 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); CTLA4; |
| | Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, PKLR, PK1, NT5C3, |
| diseases and disorders | UMPH1, PSN1, RHAG, RH50A, NRAMP2, SPTB, ALAS2, |
| | ANH1, ASB, ABCB7, ABC7, ASAT); Bare lymphocyte |
| | syndrome (TAPBP, TPSN, TAP2, ABCB3, PSF2, RING11, |
| | MHC2TA, C2TA, RFX5, RFXAP, RFX5), Bleeding disorders |
| | (TBXA2R, P2RX1, P2X1); Factor H and factor H-like 1 (HF1, |
| | CFH, HUS); Factor V and factor VIII (MCFD2); Factor VII |
| | deficiency (F7); Factor X deficiency (F10); Factor XI deficiency |
| | (F11); Factor XII deficiency (F12, HAF); Factor XIIIA |
| | deficiency (F13A1, F13A); Factor XIIIB deficiency (F13B); |
| | Fanconi anemia (FANCA, FACA, FA1, FA, FAA, FAAP95, |
| | FAAP90, FLJ34064, FANCB, FANCC, FACC, BRCA2, |
| | FANCD1, FANCD2, FANCD, FACD, FAD, FANCE, FACE, |
| | FANCF, XRCC9, FANCG, BRIP1, BACH1, FANCJ, PHF9, |
| | FANCL, FANCM, KIAA1596); Hemophagocytic |
| | lymphohistiocytosis disorders (PRF1, HPLH2, UNC13D, |
| | MUNC13-4, HPLH3, HLH3, FHL3); Hemophilia A (F8, F8C, |
| | HEMA); Hemophilia B (F9, HEMB), Hemorrhagic disorders |
| | (PI, ATT, F5); Leukocyde deficiencies and disorders (ITGB2, |
| | CD18, LCAMB, LAD, EIF2B1, EIF2BA, EIF2B2, EIF2B3, |
| | EIF2B5, LVWM, CACH, CLE, EIF2B4); Sickle cell anemia |
| | (HBB); Thalassemia (HBA2, HBB, HBD, LCRB, HBA1) |
| Cell dysregulation and | B-cell non-Hodgkin lymphoma (BCL7A, BCL7); Leukemia |
| oncology diseases and | (TAL1, TCL5, SCL, TAL2, FLT3, NBS1, NBS, ZNFN1A1, |
| disorders | IK1, LYF1, HOXD4, HOX4B, BCR, CML, PHL, ALL, ARNT, |
| | KRAS2, RASK2, GMPS, AF10, ARHGEF12, LARG, |
| | KIAA0382, CALM, CLTH, CEBPA, CEBP, CHIC2, BTL, |
| | FLT3, KIT, PBT, LPP, NPM1, NUP214, D9S46E, CAN, CAIN, |
| | RUNX1, CBFA2, AML1, WHSC1L1, NSD3, FLT3, AF1Q, |
| | NPM1, NUMA1, ZNF145, PLZF, PML, MYL, STAT5B, AF10, |
| | CALM, CLTH, ARL11, ARLTS1, P2RX7, P2X7, BCR, CML, |
| | PHL, ALL, GRAF, NF1, VRNF, WSS, NFNS, PTPN11, |
| | PTP2C, SHP2, NS1, BCL2, CCND1, PRAD1, BCL1, TCRA, |
| | GATA1, GF1, ERYF1, NFE1, ABL1, NQO1, DIA4, NMOR1, |
| | NUP214, D9S46E, CAN, CAIN) |
| Inflammation and immune | AIDS (KIR3DL1, NKAT3, NKB1, AMB11, KIR3DS1, IFNG, |
| related diseases and | CXCL12, SDF1); Autoimmune lymphoproliferative syndrome |
| disorders | (TNFRSF6, APT1, FAS, CD95, ALPS1A); Combined |
| | immunodeficiency, (IL2RG, SCIDX1, SCIDX, IMD4); HIV-1 |
| | (CCL5, SCYA5, D17S136E, TCP228), HIV susceptibility or |
| | infection (IL10, CSIF, CMKBR2, CCR2, CMKBR5, CCCKR5 |
| | (CCR5)); Immunodeficiencies (CD3E, CD3G, AICDA, AID, |
| | HIGM2, TNFRSF5, CD40, UNG, DGU, HIGM4, TNFSF5, |
| | CD40LG, HIGM1, IGM, FOXP3, IPEX, AIID, XPID, PIDX, |
| | TNFRSF14B, TACI); Inflammation (IL-10, IL-1 (IL-1a, IL-1b), |
| | IL-13, IL-17 (IL-17a (CTLA8), IL-17b, IL-17c, IL- 17d, IL- |
| | 17f), II-23, Cx3cr1, ptpn22, TNFa, NOD2/CARD15 for IBD, |
| | IL-6, IL-12 (IL-12a, IL-12b), CTLA4, Cx3cl1); Severe |
| | combined immunodeficiencies (SCIDs)(JAK3, JAKL, |
| | DCLRE1C, ARTEMIS, SCIDA, RAG1, RAG2, ADA, PTPRC, |
| | CD45, LCA, IL7R, CD3D, T3D, IL2RG, SCIDX1, SCIDX, |
| | IMD4) |
| Metabolic, liver, kidney | Amyloid neuropathy (TTR, PALB); Amyloidosis (APOA1, |
| and protein diseases and | APP, AAA, CVAP, AD1, GSN, FGA, LYZ, TTR, PALB); |
| disorders | Cirrhosis (KRT18, KRT8, CIRH1A, NAIC, TEX292, |
| | KIAA1988); Cystic fibrosis (CFTR, ABCC7, CF, MRP7); |
| | Glycogen storage diseases (SLC2A2, GLUT2, G6PC, G6PT, |
| | G6PT1, GAA, LAMP2, LAMPB, AGL, GDE, GBE1, GYS2, |
| | PYGL, PFKM); Hepatic adenoma, 142330 (TCF1, HNF1A, |
| | MODY3), Hepatic failure, early onset, and neurologic disorder |
| | (SCOD1, SCO1), Hepatic lipase deficiency (LIPC), |
| | Hepatoblastoma, cancer and carcinomas (CTNNB1, PDGFRL, |
| | PDGRL, PRLTS, AXIN1, AXIN, CTNNB1, TP53, P53, LFS1, |
| | IGF2R, MPRI, MET, CASP8, MCH5; Medullary cystic kidney |
| | disease (UMOD, HNFJ, FJHN, MCKD2, ADMCKD2); |
| | Phenylketonuria (PAH, PKU1, QDPR, DHPR, PTS); Polycystic |
| | kidney and hepatic disease (FCYT, PKHD1, ARPKD, PKD1, |
| | PKD2, PKD4, PKDTS, PRKCSH, G19P1, PCLD, SEC63) |
| Muscular/Skeletal | Becker muscular dystrophy (DMD, BMD, MYF6), Duchenne |
| diseases and disorders | Muscular Dystrophy (DMD, BMD); Emery-Dreifuss muscular |
| | dystrophy (LMNA, LMN1, EMD2, FPLD, CMD1A, HGPS, |
| | LGMD1B, LMNA, LMN1, EMD2, FPLD, CMD1A); |
| | Facioscapulohumeral muscular dystrophy (FSHMD1A, |
| | FSHD1A); Muscular dystrophy (FKRP, MDC1C, LGMD2I, |
| | LAMA2, LAMM, LARGE, KIAA0609, MDC1D, FCMD, |
| | TTID, MYOT, CAPN3, CANP3, DYSF, LGMD2B, SGCG, |
| | LGMD2C, DMDA1, SCG3, SGCA, ADL, DAG2, LGMD2D, |
| | DMDA2, SGCB, LGMD2E, SGCD, SGD, LGMD2F, CMD1L, |
| | TCAP, LGMD2G, CMD1N, TRIM32, HT2A, LGMD2H, |
| | FKRP, MDC1C, LGMD2I, TTN, CMD1G, TMD, LGMD2J, |
| | POMTI, CAV3, LGMD1C, SEPN1, SELN, RSMD1, PLEC1, |
| | PLTN, EBS1); Osteopetrosis (LRP5, BMND1, LRP7, LR3, |
| | OPPG, VBCH2, CLCN7, CLC7, OPTA2, OSTM1, GL, |
| | TCIRG1, TIRC7, OC116, OPTB1); Muscular atrophy (VAPB, |
| | VAPC, ALS8, SMN1, SMA1, SMA2, SMA3, SMA4, BSCL2, |
| | SPG17, GARS, SMAD1, CMT2D, HEXB, IGHMBP2, |
| | SMUBP2, CATF1, SMARD1) |
| Dermatological diseases | Albinisim (TYR, OCA2, TYRP1, SLC45A2, LYST), |
| and disorders | Ectodermal dysplasias (EDAR, EDARADD, WNT10A), Ehlers- |
| | Danlos syndrome (COL5A1, COL5A2, COL1A1, COL1A2, |
| | COL3A1, TNXB, ADAMTS2, PLOD1, FKBP14), Ichthyosis- |
| | associated disorders (FLG, STS, TGM1, ALOXE3/ALOX12B, |
| | KRT1, KRT10, ABCA12, KRT2, GJB2, TGM1, ABCA12, |
| | CYP4F22, ALOXE3, CERS3, NSHDL, EBP, MBTPS2, GJB2, |
| | SPINK5, AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging syndromes |
| | (POLR3A, PYCR1, LMNA, POLD1, WRN, DMPK) |
| Neurological and Neuronal | ALS (SOD1, ALS2, STEX, FUS, TARDBP, VEGF (VEGF-a, |
| diseases and disorders | VEGF-b, VEGF-c); Alzheimer disease (APP, AAA, CVAP, |
| | AD1, APOE, AD2, PSEN2, AD4, STM2, APBB2, FE65L1, |
| | NOS3, PLAU, URK, ACE, DCP1, ACE1, MPO, PACIP1, |
| | PAXIP1L, PTIP, A2M, BLMH, BMH, PSEN1, AD3); Autism |
| | (Mecp2, BZRAP1, MDGA2, Sema5A, Neurexin 1, GLO1, |
| | MECP2, RTT, PPMX, MRX16, MRX79, NLGN3, NLGN4, |
| | KIAA1260, AUTSX2); Fragile X Syndrome (FMR2, FXR1, |
| | FXR2, mGLUR5); Huntington's disease and disease like |
| | disorders (HD, IT15, PRNP, PRIP, JPH3, JP3, HDL2, TBP, |
| | SCA17); Parkinson disease (NR4A2, NURR1, NOT, TINUR, |
| | SNCAIP, TBP, SCA17, SNCA, NACP, PARK1, PARK4, DJ1, |
| | PARK7, LRRK2, PARK8, PINK1, PARK6, UCHL1, PARK5, |
| | SNCA, NACP, PARK1, PARK4, PRKN, PARK2, PDJ, DBH, |
| | NDUFV2); Rett syndrome (MECP2, RTT, PPMX, MRX16, |
| | MRX79, CDKL5, STK9, MECP2, RTT, PPMX, MRX16, |
| | MRX79, x-Synuclein, DJ-1); Schizophrenia (Neuregulin1 |
| | (Nrg1), Erb4 (receptor for Neuregulin), Complexin1 (Cplx1), |
| | Tph1 Tryptophan hydroxylase, Tph2, Tryptophan hydroxylase 2, |
| | Neurexin 1, GSK3, GSK3a, GSK3b, 5-HTT (Slc6a4), COMT, |
| | DRD (Drd1a), SLC6A3, DAOA, DTNBP1, Dao (Dao1)); |
| | Secretase Related Disorders (APH-1 (alpha and beta), Presenilin |
| | (Psen1), nicastrin, (Ncstn), PEN-2, Nos1, Parp1, Natl, Nat2); |
| | Trinucleotide Repeat Disorders (HTT (Huntington's Dx), |
| | SBMA/SMAX1/AR (Kennedy's Dx), FXN/X25 (Friedrich's |
| | Ataxia), ATX3 (Machado-Joseph's Dx), ATXN1 and ATXN2 |
| | (spinocerebellar ataxias), DMPK (myotonic dystrophy), |
| | Atrophin-1 and Atn1 (DRPLA Dx), CBP (Creb-BP - global |
| | instability), VLDLR (Alzheimer's), Atxn7, Atxn10) |
| Ocular diseases and | Age-related macular degeneration (Abcr, Ccl2, Cc2, cp |
| disorders | (ceruloplasmin), Timp3, cathepsinD, Vldlr, Ccr2); Cataract |
| | (CRYAA, CRYA1, CRYBB2, CRYB2, PITX3, BFSP2, CP49, |
| | CP47, CRYAA, CRYA1, PAX6, AN2, MGDA, CRYBA1, |
| | CRYB1, CRYGC, CRYG3, CCL, LIM2, MP19, CRYGD, |
| | CRYG4, BFSP2, CP49, CP47, HSF4, CTM, HSF4, CTM, MIP, |
| | AQP0, CRYAB, CRYA2, CTPP2, CRYBB1, CRYGD, CRYG4, |
| | CRYBB2, CRYB2, CRYGC, CRYG3, CCL, CRYAA, CRYA1, |
| | GJA8, CX50, CAE1, GJA3, CX46, CZP3, CAE3, CCM1, CAM, |
| | KRIT1); Corneal clouding and dystrophy (APOA1, TGFBI, |
| | CSD2, CDGG1, CSD, BIGH3, CDG2, TACSTD2, TROP2, |
| | M1S1, VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLC1A, JOAG, GPOA, |
| | OPTN, GLC1E, FIP2, HYPL, NRP, CYP1B1, GLC3A, OPA1, |
| | NTG, NPG, CYP1B1, GLC3A); Leber congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular dystrophy |
| | (ELOVL4, ADMD, STGD2, STGD3, RDS, RP7, PRPH2, |
| | PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

It is understood that where a numerical range is recited herein, the present invention contemplates each integer between, and including, the upper and lower limits, unless otherwise stated.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “polynucleotide”, “nucleotide”, “nucleotide sequence”, “nucleic acid” and “oligonucleotide” are used interchangeably. They refer to a polymeric form of nucleotides of any length, either deoxyribonueleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, in Irons, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers, A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate. Each of the RNA sequences described herein may comprise one or more nucleotide analogs.

As used herein, the following nucleotide identifiers are used to represent a referenced nucleotide base(s):

| Nucleotide | |
|---|---|
| reference | Base(s) represented |

| | | | | |
|---|---|---|---|---|
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of a targeting RNA molecule that can form a complex with a CRISPR nuclease with the targeting sequence serving as the targeting portion of the CRISPR complex. When the molecule having the targeting sequence is present contemporaneously with the CRISPR molecule, the RNA molecule is capable of targeting the CRISPR nuclease to the specific target sequence. Each possibility represents a separate embodiment. A targeting RNA molecule can be custom designed to target any desired sequence. The term “targets” as used herein, refers to preferential hybridization of a targeting sequence or a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

In the context of targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is affected by a complex of an RNA molecule and the CRISPR nuclease as a result of hybridization with the target sequence, i.e. on-target hybridization.

As used herein the term “wild type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion) compared to the CRISPR nuclease of any of the CRISPR nucleases indicated in Table 1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

As used herein the term “amino acid” includes natural and/or unnatural or synthetic amino acids, including glycine and both the D or I, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The term “PAM” as used herein refers to a nucleotide sequence of a target DNA located in proximity to the targeted DNA sequence and recognized by the CRISPR nuclease. The PAM sequence may differ depending on the nuclease identity.

The term “mutation disorder” or “mutation disease” as used herein refers to any disorder or disease that is related to dysfunction of a gene caused by a mutation. A dysfunctional gene manifesting as a mutation disorder contains a mutation in at least one of its alleles and is referred to as a “disease-associated gene.” The mutation may be in any portion of the disease-associated gene, for example, in a regulatory, coding, or non-coding portion. The mutation may be any class of mutation, such as a substitution, insertion, or deletion. The mutation of the disease-associated gene may manifest as a disorder or disease according to the mechanism of any type of mutation, such as a recessive, dominant negative, gain-of-function, loss-of-function, or a mutation leading to haploinsufficiency of a gene product.

A skilled artisan will appreciate that embodiments of the present invention disclose RNA molecules capable of complexing with a nuclease, e.g. a CRISPR nuclease, such as to associate with a target genomic DNA sequence of interest next to a protospacer adjacent motif (PAM). The nuclease then mediates cleavage of target DNA to create a double-stranded break within the protospacer.

In embodiments of the present invention, a CRISPR nuclease and a targeting molecule form a CRISPR complex that binds to a target DNA sequence to effect cleavage of the target DNA sequence. A CRISPR nuclease may form a CRISPR complex comprising the CRISPR nuclease and RNA molecule without a further, separate tracrRNA molecule. Alternatively, CRISPR nucleases may form a CRISPR complex between the CRISPR nuclease, an RNA molecule, and a tracrRNA molecule.

The term “protein binding sequence” or “nuclease binding sequence” refers to a sequence capable of binding with a CRISPR nuclease to form a CRISPR complex. A skilled artisan will understand that a tracrRNA capable of binding with a CRISPR nuclease to form a CRISPR complex comprises a protein or nuclease binding sequence.

An “RNA binding portion” of a CRISPR nuclease refers to a portion of the CRISPR nuclease which may bind to an RNA molecule to form a CRISPR complex, e.g. the nuclease binding sequence of a tracrRNA molecule. An “activity portion” or “active portion” of a CRISPR nuclease refers to a portion of the CRISPR nuclease which effects a double strand break in a DNA molecule, for example when in complex with a DNA-targeting RNA molecule.

An RNA molecule may comprise a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a traer mate sequence.

In embodiments of the present invention, the targeting molecule may further comprise the sequence of a tracrRNA molecule. Such embodiments may be designed as a synthetic fusion of the guide portion of the RNA molecule (gRNA or crRNA) and the trans-activating crRNA (tracrRNA), together forming a single guide RNA (sgRNA). (See Jinek et al., Science (2012)). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via base pairing and may be advantageous in certain applications of the invention described herein.

In embodiments of the present invention an RNA molecule may comprise a “nexus” region and/or “hairpin” regions which may further define the structure of the RNA molecule. (See Briner et al., Molecular Cell (2014)).

As used herein, the term “direct repeat sequence” refers to two or more repeats of a specific amino acid sequence of nucleotide sequence.

As used herein, an RNA sequence or molecule capable of “interacting with” or “binding” with a CRISPR nuclease refers to the RNA sequence or molecules ability to form a CRISPR complex with the CRISPR nuclease.

As used herein, the term “operably linked” refers to a relationship (i.e. fusion, hybridization) between two sequences or molecules permitting them to function in their intended manner. In embodiments of the present invention, when an RNA molecule is operably linked to a promoter, both the RNA molecule and the promotor are permitted to function in their intended manner.

As used herein, the term “heterologous promoter” refers to a promoter that does not naturally occur together with the molecule or pathway being promoted.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

### Nuclear Localization Sequences

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known. Non-limiting examples of NLSs include an NLS sequence derived from: the SV40 virus large T-antigen, nucleoplasmin, c-myc, the hRNPAI M9 NLS, the IBB domain from importin-alpha, myoma T protein, human p53, mouse c-abl IV, influenza vims NS1, Hepatitis virus delta antigen, mouse Mx1 protein, human poly(ADP-ribose) polymerase, and the steroid hormone receptors (human) glucocorticoid.

### Delivery

The CRISPR nuclease or CRISPR compositions described herein may be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention. The CRISPR nucleases and/or polynucleotides encoding same described herein, and optionally additional proteins (e.g., ZFPs, TALENs, transcription factors, restriction enzymes) and/or nucleotide molecules such as guide RNA may be delivered to a target cell by any suitable means. The target cell may be any type of cell e.g., eukaryotic or prokaryotic, in any environment e.g., isolated or not, maintained in culture, in vitro, ex vivo, in vivo or in planta.

In some embodiments, the composition to be delivered includes mRNA of the nuclease and RNA of the guide. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. In some embodiments, the composition to be delivered includes mRNA of the nuclease, DNA-targeting RNA and the tracrRNA. In some embodiments, the composition to be delivered includes mRNA of the nuclease, DNA-targeting RNA and the tracrRNA and a donor template. In some embodiments, the composition to be delivered includes the CRISPR nuclease DNA-targeting RNA and the tracrRNA. In some embodiments, the composition to be delivered includes the CRISPR nuclease, DNA-targeting RNA and the tracrRNA and a donor template for gene editing via, for example, homology directed repair.

Any suitable viral vector system may be used to deliver RNA compositions. Conventional viral and non-viral based gene transfer methods can be used to introduce nucleic acids and/or CRISPR nuclease in cells (e.g., mammalian cells, plant cells, etc.) and target tissues. Such methods can also be used to administer nucleic acids encoding and/or CRISPR nuclease protein to cells in vitro. In certain embodiments, nucleic acids and/or CRISPR nuclease are administered for in vivo or ex vivo gene therapy uses. Non-viral vector delivery systems include naked nucleic acid, and nucleic acid complexed with a delivery vehicle such as a liposome or poloxamer. For a review of gene therapy procedures, scc Anderson, Science (1992); Nabel and Felgner, TIBTECH (1993); Mitani and Caskey, TIBTECH (1993); Dillon, TIBTECH (1993); Miller, Nature (1992); Van Brunt, Biotechnology (1988); Vigne et al., Restorative Neurology and Neuroscience 8:35-36 (1995); Kremer and Perricaudet, British Medical Bulletin (1995); Haddada et al., Current Topics in Microbiology and Immunology (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid: nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Non-viral vectors, such as transposon-based systems e.g., recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam.TM., Lipofectin.TM. and Lipofectamine.TM. RNAiMAX). Cationic and neutral lipids that are suitable for efficient receptor-recognition lipofection of polynucleotides include those disclosed in PCT International Publication Nos. WO/1991/017424 and WO/1991/016024.

Delivery can be to cells (ex vivo administration) or target tissues (in vivo administration).

The preparation of lipid: nucleic acid complexes, including targeted liposomes such as immunolipid complexes, is well known to one of skill in the art (scc, e.g., Crystal, Science (1995); Blaese et al., Cancer Gene Ther. (1995); Behr et al., Bioconjugate Chem. (1994); Remy et al., Bioconjugate Chem. (1994); Gao and Huang, Gene Therapy (1995); Ahmad and Allen, Cancer Res., (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al., Nature Biotechnology (2009)).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, recombinant retroviral, lentivirus, adenoviral, adeno-associated, vaccinia and herpes simplex virus vectors for gene transfer. However, an RNA virus is preferred for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997). Packaging cells are used to form virus particles that are capable of infecting a host cell. Such cells include 293 cells, which package adenovirus, AAV, and psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. In some embodiments, delivery of mRNA in vivo and ex vivo, and RNPs delivery may be utilized.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney, “Culture of Animal Cells, A Manual of Basic Technique and Specialized Applications (6th edition, 2010)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOKISV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in-vitro or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., J. Exp. Med. (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (as a non-limiting example see Inaba et al., J. Exp. Med. (1992)). Stem cells that have been modified may also be used in some embodiments.

Notably, any one of the CRISPR nucleases described herein may be suitable for genome editing in post-mitotic cells or any cell which is not actively dividing, e.g., arrested cells. Examples of post-mitotic cells which may be edited using a CRISPR nuclease of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### DNA Repair by Homologous Recombination

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double strand break in a gene prior to repair and (2) a donor RNA template for repair, the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by recombinant viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al.,

“Molecular Cloning: A laboratory Manual” (1989); Ausubel, R. M. (Ed.), “Current Protocols in Molecular Biology” Volumes I-III (1994); Ausubel ct al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells-A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization-A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

CRISPR repeat (crRNA), trans-activating RNA (tracrRNA), and nuclease polypeptide (OMNI) were predicted from different metagenomic databases of sequences of environmental samples.

### Construction of OMNI Nuclease Polypeptides

For construction of novel nuclease polypeptides (OMNI), the open reading frame of several identified OMNIs were codon optimized for human cell line expression. The ORF was cloned into the bacterial expression plasmid pET9a and into the mammalian expression plasmid pmOMNI (Table 4).

Prediction and Construction of sgRNA

For each OMNI the single guide RNA (sgRNA) was predicted by detection of the CRISPR repeat array sequence and a tracrRNA in the respective bacterial genome. The native pre-mature crRNA and tracrRNA sequences were connected in silico with a tetra-loop ‘gaaa’ sequence and the secondary structure elements of the duplex were predicted using an RNA secondary structure prediction tool.

The predicted secondary structures of the full duplex RNA elements (crRNA-tracrRNA chimera) were used for identification of possible tracrRNA sequences for the design of a sgRNA. In order to overcome potential transcriptional and structural constraints and to assess the plasticity of the sgRNA scaffold in the human cellular environmental context, small changes in the nucleotide sequence of the possible sgRNA were made in some cases (Table 2, referred to as “V2” in the guide's table). Finally, up to two versions of possible designed scaffolds were synthesized for each OMNI and connected downstream to a 22nt universal unique spacer sequence (T2, SEQ ID NO: 391) and cloned into a bacterial expressing plasmid under an inducible T7 promoter combined with a U6 promoter for mammalian expression (pShuttleGuide, Table 4).

(SEQ ID NO: 391)
T2-GGAAGAGCAGAGCCTTGGTCTC

In-vitro Depletion assay by TXTL

Depletion of PAM sequences in vitro was followed as described by Maxwell et al, Methods. 2018. Briefly, linear DNA expressing the OMNI nucleases and an sgRNA under T7 promoter were added to a cell-free transcription-translation in vitro system (TXTL mix, Arbor Bioscience) together with a linear construct expressing T7 polymerase. RNA expression and protein translation by the TXTL mix result in the formation of a ribonucleoprotein (RNP) complex. Since linear DNA was used, Chi6 DNA sequences were added to the TXTL reaction mix to inhibit the exonuclease activity of RecBCD, thereby protecting the linear DNA from degradation. The sgRNA spacer is designed to target a library of plasmids containing the target protospacer (pbPOS T2 library, Table 4) flanked by an 8N randomized set of potential PAM sequences. Depletion of PAM sequences from the library was measured by high-throughput sequencing using PCR to add the necessary adapters and indices to both the cleaved library and to a control library expressing a non-targeting gRNA. Following deep sequencing, the in vitro activity was confirmed by the fraction of the depleted sequences having the same PAM sequence relative to their occurrence in the control, indicating functional DNA cleavage by the OMNI nuclease (Table 3).

Activity in human cells on endogenous genomic targets

OMNIs were also assayed for their ability to promote editing on specific genomic locations in human cells. To this end, per each OMNI, the corresponding OMNI-P2A-mCherry expression vector (pmOMNI, Table 4) was transfected into HeLa cells together with an sgRNA designed to target a specific location in the human genome (pShuttle Guide-Table 4, and spacer sequence-Table 5). At 72 hours, cells were harvested. Half of the harvested cells were used for quantification of transfection efficiency by FACS using mCherry fluorescence as a marker. The other half of the harvested cells were lysed, and their genomic DNA was used to PCR amplify the corresponding putative genomic targets. Amplicons were subjected to next-generation sequencing (NGS) and the resulting sequences were used calculate the percentage of editing events in each target site. Short Insertions or deletions (indels) around the cut site are the typical outcome of repair of DNA ends following nuclease induced DNA cleavage. The calculation of percent editing was therefore deduced from the fraction of indels containing sequences within each amplicon.

Genomic activity of each ONMI was assessed using a panel of several unique sgRNA each designed to target a different genomic location. The results of these experiments are summarized in Table 5. As can be seen in the table (column 6, “Max Activity Mean”), all OMNIs exhibit significant editing levels in human cells compared to the negative control (not shown).

TABLE 1
OMNI CRISPR nuclease sequences

| | | SEQ ID NO | Nickase | Nickase | |
|---|---|---|---|---|---|
| | SEQ ID NO | of DNA | having | having | Dead nuclease |
| | of OMNI | sequence | inactivated | inactivated | having inactivated |
| “OMNI” | Amino Acid | encoding | RuvC | HNH | RuvC and HNH |
| Name | Sequence | OMNI | domain | domain | domains |
| OMNI-XL-1 | 1 | 23 | D8 or E650 | E735 or | (D8 or E650 or |
| | | | or H1174 or | H736 or | H1174 or D1177) |
| | | | D1177 | N759 | and (E735 or H736 |
| | | | | | or N759) |
| OMNI-XL-2 | 2 | 24 | D8 or E647 | E732 or | (D8 or E647 or |
| | | | or H1171 or | H733 or | H1171 or D1174) |
| | | | D1174 | N756 | and (E732 or H733 |
| | | | | | or N756) |
| OMNI-XL-3 | 3 | 25 | D8 or E648 | E733 or | (D8 or E648 or |
| | | | or H1175 or | H734 or | H1175 or D1178) |
| | | | D1178 | N757 | and (E733 or H734 |
| | | | | | or N757) |
| OMNI-XL-4 | 4 | 26 | D8 or E669 | E754 or | (D8 or E669 or |
| | | | or H1190 or | H755 or | H1190 or D1193) |
| | | | D1193 | N778 | and (E754 or H755 |
| | | | | | or N778) |
| OMNI-XL-5 | 5 | 27 | D8 or E676 | E757 or | (D8 or E676 or |
| | | | or H1192 or | H758 or | H1192 or D1195) |
| | | | D1195 | N781 | and (E757 or H758 |
| | | | | | or N781) |
| OMNI-XL-6 | 6 | 28 | D8 or E676 | E757 or | (D8 or E676 or |
| | | | or H1192 or | H758 or | H1192 or D1195) |
| | | | D1195 | N781 | and (E757 or 1758 |
| | | | | | or N781) |
| OMNI-XL-7 | 7 | 29 | D8 or E669 | E750 or | (D8 or E669 or |
| | | | or H1185 or | H751 or | H1185 or D1188) |
| | | | D1188 | N774 | and (E750 or H751 |
| | | | | | or N774) |
| OMNI-XL-8 | 8 | 30 | D8 or E647 | E732 or | (D8 or E647 or |
| | | | or H1171 or | H733 or | H1171 or D1174) |
| | | | D1174 | N756 | and (E732 or H733 |
| | | | | | or N756) |
| OMNI-XL-9 | 9 | 31 | D8 or E669 | E754 or | (D8 or E669 or |
| | | | or H1189 or | H755 or | H1189 or D1192) |
| | | | D1192 | N778 | and (E754 or H755 |
| | | | | | or N778) |
| OMNI-XL-10 | 10 | 32 | D9 or E671 | E756 or | (D9 or E671 or |
| | | | or H1186 or | H757 or | H1186 or D1189) |
| | | | D1189 | N780 | and (E756 or H757 |
| | | | | | or N780) |
| OMNI-XL-11 | 11 | 33 | D7 or E617 | E702 or | (D7 or E617 or |
| | | | or H1162 or | H703 or | H1162 or D1165) |
| | | | D1165 | N726 | and (E702 or H703 |
| | | | | | or N726) |
| OMNI-XL-12 | 12 | 34 (or SEQ | D7 or E617 | E703 or | (D7 or E617 or |
| | | ID NO: 392, | or H1163 or | H704 or | H1163 or D1166) |
| | | which is a | D1166 | N727 | and (E703 or H704 |
| | | human | | | or N727) |
| | | optimized | | | |
| | | version | | | |
| | | starting at the | | | |
| | | second amino | | | |
| | | acid) | | | |
| OMNI-XL-13 | 13 | 35 | D11 or | E761 or | (D11 or E674 or |
| | | | E674 or | H762 or | H1211 or D1214) |
| | | | H1211 or | N785 | and (E761 or H762 |
| | | | D1214 | | or N785) |
| OMNI-XL-14 | 14 | 36 | D8 or E625 | E710 or | (D8 or E625 or |
| | | | or H1147 or | H711 or | H1147 or D1150) |
| | | | D1150 | N734 | and (E710 or H711 |
| | | | | | or N734) |
| OMNI-XL-15 | 15 | 37 | D8 or E638 | E723 or | (D8 or E638 or |
| | | | or II1153 or | II724 or | HI1153 or D1156) |
| | | | D1156 | N747 | and (E723 or H724 |
| | | | | | or N747) |
| OMNI-XL-16 | 16 | 38 (or SEQ | D8 or E669 | E754 or | (D8 or E669 or |
| | | ID NO: 393, | or H1189 or | H755 or | H1189 or D1192) |
| | | which is a | D1192 | N778 | and (E754 or H755 |
| | | human | | | or N778) |
| | | optimized | | | |
| | | version | | | |
| | | starting at the | | | |
| | | second amino | | | |
| | | acid) | | | |
| OMNI-XL-17 | 17 | 39 | D8 or E649 | E734 or | (D8 or E649 or |
| | | | or H1173 or | H735 or | H1173 or D1176) |
| | | | D1176 | N758 | and (E734 or H735 |
| | | | | | or N758) |
| OMNI-XL-18 | 18 | 40 (or SEQ | D11 or | E763 or | (D11 or E676 or |
| | | ID NO: 394, | E676 or | H764 or | H1213 or D1216) |
| | | which is a | H1213 or | N787 | and (E763 or H764 |
| | | human | D1216 | | or N787) |
| | | optimized | | | |
| | | version | | | |
| | | starting at the | | | |
| | | second amino | | | |
| | | acid) | | | |
| OMNI-XL-19 | 19 (or SEQ ID | 41 | D11 or | E763 or | (D11 or E676 or |
| | NO: 395, | | E676 or | H764 or | H1213 or D1216) |
| | which is a | | H1213 or | N787 | and E763 or H764 |
| | human | | D1216 | | or N787) |
| | optimized | | | | |
| | version | | | | |
| | starting at the | | | | |
| | second amino | | | | |
| | acid) | | | | |
| OMNI-XL-20 | 20 | 42 | D8 or E638 | E723 or | (D8 or E638 or |
| | | | or H1153 or | H724 or | H1153 or D1156) |
| | | | D1156 | N747 | and (E723 or H724 |
| | | | | | or N747) |
| OMNI-XL-21 | 21 | 43 | D8 or E668 | E753 or | (D8 or E668 or |
| | | | or H1185 or | H754 or | H1185 or D1188) |
| | | | D1188 | N777 | and (E753 or H754 |
| | | | | | or N777) |
| OMNI-XL-22 | 22 | 44 | D8 or E651 | E736 or | (D8 or E651 or |
| | | | or H1172 or | H737 or | H1172 or D1175) |
| | | | D1175 | N760 | and (E736 or H737 |
| | | | | | or N760) |

Table 1. OMNI nuclease sequences: Table 1 lists the OMNI name, its corresponding nuclease protein sequence, its DNA sequence, its human optimized DNA sequence, alternative positions to be substituted to generate a nickase having an inactivated RuvC domain, alternative positions to be substituted to generate a nickase having an inactivated HNH domain, and alternative positions to be substituted to generate a catalytically dead nuclease having inactivated RuvC and HNH domains. Substitution to any other amino acid is permissible for each of the amino acid positions indicated in columns 5-7, except a substitution of glutamic acid (E) to aspartic acid (D) in the HNH domain, in order to achieve inactivation.

TABLE 2
OMNI Guide RNA and Scaffold RNA Sequences

| | | OMNI-XL-1 |
|---|---|---|
| sgRNA | sgRNA V1 | GUUGUGGUUUGAUUAAAGAgaaaUCUUUAAUCUUAUCACAAUAAG |
| Version | | GCUAUAUGCCGUAGAUGAAAAUCUUUAGUCCCGCUUCGGUGGGA |
| | | CUUUUUUUUU (SEQ ID NO: 49) |
| crRNA: tracr | crRNA | GUUGUGGUUUGAUUAAAGA (SEQ ID NO: 50) |
| RNA duplex | (Repeat) | |
| V1 | Partial | GUUGUGGUUUGAUUA (SEQ ID NO: 51) |
| | crRNA 1 | |
| | Partial | GUUGUGGUUUGA (SEQ ID NO: 52) |
| | crRNA 2 | |
| | Partial | GUUGUGGUUU (SEQ ID NO: 53) |
| | crRNA 3 | |
| | tracrRNA | UCUUUAAUCUUAUCACAAUA (SEQ ID NO: 54) |
| | (Antirepeat) | |
| | Partial | UAAUCUUAUCACAAUA (SEQ ID NO: 55) |
| | tracrRNA 1 | |
| | Partial | UCUUAUCACAAUA (SEQ ID NO: 56) |
| | tracrRNA 2 | |
| | Partial | UUAUCACAAUA (SEQ ID NO: 57) |
| | tracrRNA 3 | |
| tracrRNA | tracrRNA | AGGCUAUAUGCCGU (SEQ ID NO: 58) |
| sequences | Portion 1- | |
| | Full | |
| | tracrRNA | AGGCUAUAUGCC (SEQ ID NO: 59) |
| | Portion 1- | |
| | Partial | |
| | tracrRNA | AGAUGAAAAUCUUU (SEQ ID NO: 60) |
| | Portion 2- | |
| | Full | |
| | tracrRNA | AGAUGAAAAUCU (SEQ ID NO: 61) |
| | Portion 2- | |
| | Partial | |
| | tracrRNA | AGUCCCGCUUCGGUGGGACUUUUUUUUU (SEQ ID NO: 62) |
| | Portion 3- | |
| | Full | |
| | tracrRNA | AGUCCCGCUUCGGUGGGACU (SEQ ID NO: 63) |
| | Portion 3- | |
| | Partial | |
| | tracrRNA | AGUCCCGCUUCGGUGGGAC (SEQ ID NO: 64) |
| | Portion 3- | |
| | Without | |
| | polyT | |
| | | OMNI-XL-2 |
| sgRNA | sgRNA V1 | GUUGUGGUUUGAUUAAAGAgaaaUCUUUAAUCUUAUCACAAUAAG |
| Version | | GCUAUAUGCCGUAGAUGAAAAUCUUUAGUCCUGCUUCGGUAGG |
| | | ACUUUUUUUU (SEQ ID NO: 65) |
| crRNA: tracr | crRNA | GUUGUGGUUUGAUUAAAGA (SEQ ID NO: 66) |
| RNA duplex | (Repeat) | |
| V1 | Partial | GUUGUGGUUUGAUUA (SEQ ID NO: 67) |
| | crRNA 1 | |
| | Partial | GUUGUGGUUUGA (SEQ ID NO: 68) |
| | crRNA 2 | |
| | Partial | GUUGUGGUUU (SEQ ID NO: 69) |
| | crRNA 3 | |
| | tracrRNA | UCUUUAAUCUUAUCACAAUA (SEQ ID NO: 70) |
| | (Antirepeat) | |
| | Partial | UAAUCUUAUCACAAUA (SEQ ID NO: 71) |
| | tracrRNA 1 | |
| | Partial | UCUUAUCACAAUA (SEQ ID NO: 72) |
| | tracrRNA 2 | |
| | Partial | UUAUCACAAUA (SEQ ID NO: 73) |
| | tracrRNA 3 | |
| tracrRNA | tracrRNA | AGGCUAUAUGCCGU (SEQ ID NO: 74) |
| sequences | Portion 1- | |
| | Full | |
| | tracrRNA | AGGCUAUAUGCC (SEQ ID NO: 75) |
| | Portion 1- | |
| | Partial | |
| | tracrRNA | AGAUGAAAAUCUUU (SEQ ID NO: 76) |
| | Portion 2- | |
| | Full | |
| | tracrRNA | AGAUGAAAAUCU (SEQ ID NO: 77) |
| | Portion 2- | |
| | Partial | |
| | tracrRNA | AGUCCUGCUUCGGUAGGACUUUUUUUU (SEQ ID NO: 78) |
| | Portion 3- | |
| | Full | |
| | tracrRNA | AGUCCUGCUUCGGUAGGACU (SEQ ID NO: 79) |
| | Portion 3- | |
| | Partial | |
| | tracrRNA | AGUCCUGCUUCGGUAGGAC (SEQ ID NO: 80) |
| | Portion 3- | |
| | Without | |
| | polyT | |
| | | OMNI-XL-3 |
| sgRNA | sgRNA V1 | GUUGUGGUUUGAUAACAAAgaaaUUUGUAUCUUAUCACAAUAAGG |
| Version | | CUAUAUGCCGAAGUUCAAAAGAACUAUGCCUCUGCUUCGGCAGA |
| | | GGCUUUUUU (SEQ ID NO: 81) |
| crRNA: tracr | crRNA | GUUGUGGUUUGAUAACAAA (SEQ ID NO: 82) |
| RNA duplex | (Repeat) | |
| V1 | Partial | GUUGUGGUUUGAUAA (SEQ ID NO: 83) |
| | crRNA 1 | |
| | Partial | GUUGUGGUUUGA (SEQ ID NO: 84) |
| | crRNA 2 | |
| | Partial | GUUGUGGUUU (SEQ ID NO: 85) |
| | crRNA 3 | |
| | tracrRNA | UUUGUAUCUUAUCACAAUA (SEQ ID NO: 86) |
| | (Antirepeat) | |
| | Partial | UAUCUUAUCACAAUA (SEQ ID NO: 87) |
| | tracrRNA 1 | |
| | Partial | UCUUAUCACAAUA (SEQ ID NO: 88) |
| | tracrRNA 2 | |
| | Partial | UUAUCACAAUA (SEQ ID NO: 89) |
| | tracrRNA 3 | |
| tracrRNA | tracrRNA | AGGCUAUAUGCCGA (SEQ ID NO: 90) |
| sequences | Portion 1- | |
| | Full | |
| | tracrRNA | AGGCUAUAUGCC (SEQ ID NO: 91) |
| | Portion 1- | |
| | Partial | |
| | tracrRNA | AGUUCAAAAGAACUAU (SEQ ID NO: 92) |
| | Portion 2- | |
| | Full | |
| | tracrRNA | AGUUCAAAAGAACU (SEQ ID NO: 93) |
| | Portion 2- | |
| | Partial | |
| | tracrRNA | GCCUCUGCUUCGGCAGAGGCUUUUUU (SEQ ID NO: 94) |
| | Portion 3- | |
| | Full | |
| | tracrRNA | GCCUCUGCUUCGGCAGAGGC (SEQ ID NO: 95) |
| | Portion 3- | |
| | Partial | |
| | tracrRNA | GCCUCUGCUUCGGCAGAGGC (SEQ ID NO: 96) |
| | Portion 3- | |
| | Without | |
| | polyT | |
| | | OMNI-XL-4 |
| sgRNA | sgRNA V1 | GUUGUGGUUUGAUUAAAGAgaaaUCUUUAAUCUUAUCACAAUAAG |
| Version | | GCUAUAUGCCGUAGAUGUAAAAUCUUUAGUCCUGCUUCGGUGG |
| | | GACUUUUUUUU (SEQ ID NO: 97) |
| crRNA: tracr | crRNA | GUUGUGGUUUGAUUAAAGA (SEQ ID NO: 98) |
| RNA duplex | (Repeat) | |
| V1 | Partial | GUUGUGGUUUGAUUA (SEQ ID NO: 99) |
| | crRNA 1 | |
| | Partial | GUUGUGGUUUGA (SEQ ID NO: 100) |
| | crRNA 2 | |
| | Partial | GUUGUGGUUU (SEQ ID NO: 101) |
| | crRNA 3 | |
| | tracrRNA | UCUUUAAUCUUAUCACAAUA (SEQ ID NO: 102) |
| | (Antirepeat) | |
| | Partial | UAAUCUUAUCACAAUA (SEQ ID NO: 103) |
| | tracrRNA 1 | |
| | Partial | UCUUAUCACAAUA (SEQ ID NO: 104) |
| | tracrRNA 2 | |
| | Partial | UUAUCACAAUA (SEQ ID NO: 105) |
| | tracrRNA 3 | |
| tracrRNA | tracrRNA | AGGCUAUAUGCCGU (SEQ ID NO: 106) |
| sequences | Portion 1- | |
| | Full | |
| | tracrRNA | AGGCUAUAUGCC (SEQ ID NO: 107) |
| | Portion 1- | |
| | Partial | |
| | tracrRNA | AGAUGUAAAAUCUUU (SEQ ID NO: 108) |
| | Portion 2- | |
| | Full | |
| | tracrRNA | AGAUGUAAAAUCU (SEQ ID NO: 109) |
| | Portion 2- | |
| | Partial | |
| | tracrRNA | AGUCCUGCUUCGGUGGGACUUUUUUUU (SEQ ID NO: 110) |
| | Portion 3- | |
| | Full | |
| | tracrRNA | AGUCCUGCUUCGGUGGGACU (SEQ ID NO: 111) |
| | Portion 3- | |
| | Partial | |
| | tracrRNA | AGUCCUGCUUCGGUGGGAC (SEQ ID NO: 112) |
| | Portion 3- | |
| | Without | |
| | polyT | |
| | | OMNI-XL-5 |
| sgRNA | sgRNA V1 | GUUGUGGUUUGAUUAAAGAgaaaUCUUUAAUCUUAUCACAAUAAG |
| Version | | GCUAUAUGCCGUAGAUGAAAAUCUUUAGUCCUGCUUCGGUGGG |
| | | ACUUUUUUU (SEQ ID NO: 113) |
| crRNA: tracr | crRNA | GUUGUGGUUUGAUUAAAGA (SEQ ID NO: 114) |
| RNA duplex | (Repeat) | |
| V1 | Partial | GUUGUGGUUUGAUUA (SEQ ID NO: 115) |
| | crRNA 1 | |
| | Partial | GUUGUGGUUUGA (SEQ ID NO: 116) |
| | crRNA 2 | |
| | Partial | GUUGUGGUUU (SEQ ID NO: 117) |
| | crRNA 3 | |
| | tracrRNA | UCUUUAAUCUUAUCACAAUA (SEQ ID NO: 118) |
| | (Antirepeat) | |
| | Partial | UAAUCUUAUCACAAUA (SEQ ID NO: 119) |
| | tracrRNA 1 | |
| | Partial | UCUUAUCACAAUA (SEQ ID NO: 120) |
| | tracrRNA 2 | |
| | Partial | UUAUCACAAUA (SEQ ID NO: 121) |
| | tracrRNA 3 | |
| tracrRNA | tracrRNA | AGGCUAUAUGCCGU (SEQ ID NO: 122) |
| sequences | Portion 1- | |
| | Full | |
| | tracrRNA | AGGCUAUAUGCC (SEQ ID NO: 123) |
| | Portion 1- | |
| | Partial | |
| | tracrRNA | AGAUGAAAAUCUUU (SEQ ID NO: 124) |
| | Portion 2- | |
| | Full | |
| | tracrRNA | AGAUGAAAAUCU (SEQ ID NO: 125) |
| | Portion 2- | |
| | Partial | |
| | tracrRNA | AGUCCUGCUUCGGUGGGACUUUUUUU (SEQ ID NO: 126) |
| | Portion 3- | |
| | Full | |
| | tracrRNA | AGUCCUGCUUCGGUGGGACU (SEQ ID NO: 127) |
| | Portion 3- | |
| | Partial | |
| | tracrRNA | AGUCCUGCUUCGGUGGGAC (SEQ ID NO: 128) |
| | Portion 3- | |
| | Without | |
| | polyT | |
| | | OMNI-XL-6 |
| sgRNA | sgRNA V1 | GUUGUGGUUUGAUUAAAGAgaaaUCUUUAAUCUUAUCACAAUAAG |
| Version | | GCUAUAUGCCGUAGAUGUAAAAUCUUUAGUCCUGCUUCGGUGG |
| | | GACUUUUUU (SEQ ID NO: 129) |
| crRNA: tracr | crRNA | GUUGUGGUUUGAUUAAAGA (SEQ ID NO: 130) |
| RNA duplex | (Repeat) | |
| V1 | Partial | GUUGUGGUUUGAUUA (SEQ ID NO: 131) |
| | crRNA 1 | |
| | Partial | GUUGUGGUUUGA (SEQ ID NO: 132) |
| | crRNA 2 | |
| | Partial | GUUGUGGUUU (SEQ ID NO: 133) |
| | crRNA 3 | |
| | tracrRNA | UCUUUAAUCUUAUCACAAUA (SEQ ID NO: 134) |
| | (Antirepeat) | |
| | Partial | UAAUCUUAUCACAAUA (SEQ ID NO: 135) |
| | tracrRNA 1 | |
| | Partial | UCUUAUCACAAUA (SEQ ID NO: 136) |
| | tracrRNA 2 | |
| | Partial | UUAUCACAAUA (SEQ ID NO: 137) |
| | tracrRNA 3 | |
| tracrRNA | tracrRNA | AGGCUAUAUGCCGU (SEQ ID NO: 138) |
| sequences | Portion 1- | |
| | Full | |
| | tracrRNA | AGGCUAUAUGCC (SEQ ID NO: 139) |
| | Portion 1- | |
| | Partial | |
| | tracrRNA | AGAUGUAAAAUCUUU (SEQ ID NO: 140) |
| | Portion 2- | |
| | Full | |
| | tracrRNA | AGAUGUAAAAUCU (SEQ ID NO: 141) |
| | Portion 2- | |
| | Partial | |
| | tracrRNA | AGUCCUGCUUCGGUGGGACUUUUUU (SEQ ID NO: 142) |
| | Portion 3- | |
| | Full | |
| | tracrRNA | AGUCCUGCUUCGGUGGGACU (SEQ ID NO: 143) |
| | Portion 3- | |
| | Partial | |
| | tracrRNA | AGUCCUGCUUCGGUGGGAC (SEQ ID NO: 144) |
| | Portion 3- | |
| | Without | |
| | polyT | |
| | | OMNI-XL-7 |
| sgRNA | sgRNA V1 | GUUGUGGUUUGAUUAAAGAgaaaUCUUUAAUCUUAUCACAAUAAG |
| Version | | GCUAUAUGCCGUAGAUGAAAAUCUUUAGUCCUGCUUCGGUGGG |
| | | ACUUUUUCUUUUUUUUU (SEQ ID NO: 145) |
| crRNA: tracr | crRNA | GUUGUGGUUUGAUUAAAGA (SEQ ID NO: 146) |
| RNA duplex | (Repeat) | |
| V1 | Partial | GUUGUGGUUUGAUUA (SEQ ID NO: 147) |
| | crRNA 1 | |
| | Partial | GUUGUGGUUUGA (SEQ ID NO: 148) |
| | crRNA 2 | |
| | Partial | GUUGUGGUUU (SEQ ID NO: 149) |
| | crRNA 3 | |
| | tracrRNA | UCUUUAAUCUUAUCACAAUA (SEQ ID NO: 150) |
| | (Antirepeat) | |
| | Partial | UAAUCUUAUCACAAUA (SEQ ID NO: 151) |
| | tracrRNA 1 | |
| | Partial | UCUUAUCACAAUA (SEQ ID NO: 152) |
| | tracrRNA 2 | |
| | Partial | UUAUCACAAUA (SEQ ID NO: 153) |
| | tracrRNA 3 | |
| tracrRNA | tracrRNA | AGGCUAUAUGCCGU (SEQ ID NO: 154) |
| sequences | Portion 1- | |
| | Full | |
| | tracrRNA | AGGCUAUAUGCC (SEQ ID NO: 155) |
| | Portion 1- | |
| | Partial | |
| | tracrRNA | AGAUGAAAAUCUUU (SEQ ID NO: 156) |
| | Portion 2- | |
| | Full | |
| | tracrRNA | AGAUGAAAAUCU (SEQ ID NO: 157) |
| | Portion 2- | |
| | Partial | |
| | tracrRNA | AGUCCUGCUUCGGUGGGACUUUUUCUUUUUUUUU (SEQ ID NO: |
| | Portion 3- | 158) |
| | Full | |
| | tracrRNA | AGUCCUGCUUCGGUGGGACU (SEQ ID NO: 159) |
| | Portion 3- | |
| | Partial | |
| | tracrRNA | AGUCCUGCUUCGGUGGGACUUUUUC (SEQ ID NO: 160) |
| | Portion 3- | |
| | Without | |
| | polyT | |
| sgRNA | sgRNA V2 | GUUGUGGUUUGAUUAAAGAgaaaUCUUUAAUCUUAUCACAAUAAG |
| Version | | GCUAUAUGCCGUAGAUGAAAAUCUUUAGUCCUGCUUCGGUGGG |
| | | ACUUCUUCUUUUUUUUU (SEQ ID NO: 161) |
| | tracrRNA | AGUCCUGCUUCGGUGGGACUUCUUCUUUUUUUUU (SEQ ID NO: |
| | Portion 3- | 162) |
| | Full | |
| | tracrRNA | AGUCCUGCUUCGGUGGGACUUCUUC (SEQ ID NO: 163) |
| | Portion 3- | |
| | Without | |
| | polyT | |
| | | OMNI-XL-8 |
| sgRNA | sgRNA V1 | GUUGUGGUUUGAUUAAAGAgaaaUCUUUAAUCUUAUCACAAUAAG |
| Version | | GCUAUAUGCCGUAGAUGAAAAUCUUUAGUCCUGCUUCGGUAGG |
| | | ACUUUUUUU (SEQ ID NO: 164) |
| crRNA: tracr | crRNA | GUUGUGGUUUGAUUAAAGA (SEQ ID NO: 165) |
| RNA duplex | (Repeat) | |
| V1 | Partial | GUUGUGGUUUGAUUA (SEQ ID NO: 166) |
| | crRNA 1 | |
| | Partial | GUUGUGGUUUGA (SEQ ID NO: 167) |
| | crRNA 2 | |
| | Partial | GUUGUGGUUU (SEQ ID NO: 168) |
| | crRNA 3 | |
| | tracrRNA | UCUUUAAUCUUAUCACAAUA (SEQ ID NO: 169) |
| | (Antirepeat) | |
| | Partial | UAAUCUUAUCACAAUA (SEQ ID NO: 170) |
| | tracrRNA 1 | |
| | Partial | UCUUAUCACAAUA (SEQ ID NO: 171) |
| | tracrRNA 2 | |
| | Partial | UUAUCACAAUA (SEQ ID NO: 172) |
| | tracrRNA 3 | |
| tracrRNA | tracrRNA | AGGCUAUAUGCCGU (SEQ ID NO: 173) |
| sequences | Portion 1- | |
| | Full | |
| | tracrRNA | AGGCUAUAUGCC (SEQ ID NO: 174) |
| | Portion 1- | |
| | Partial | |
| | tracrRNA | AGAUGAAAAUCUUU (SEQ ID NO: 175) |
| | Portion 2- | |
| | Full | |
| | tracrRNA | AGAUGAAAAUCU (SEQ ID NO: 176) |
| | Portion 2- | |
| | Partial | |
| | tracrRNA | AGUCCUGCUUCGGUAGGACUUUUUUU (SEQ ID NO: 177) |
| | Portion 3- | |
| | Full | |
| | tracrRNA | AGUCCUGCUUCGGUAGGACU (SEQ ID NO: 178) |
| | Portion 3- | |
| | Partial | |
| | tracrRNA | AGUCCUGCUUCGGUAGGAC (SEQ ID NO: 179) |
| | Portion 3- | |
| | Without | |
| | polyT | |
| | | OMNI-XL-9 |
| sgRNA | sgRNA V1 | GUUGUGGUUUGAUUAAAGAgaaaUCUUUAAUCUUAUCACAAUAAG |
| Version | | GCUAUAUGCCGUAGAUGAAAAUCUUUAGUCCUGCUUCGGUGGG |
| | | ACUUUUUUU (SEQ ID NO: 180) |
| crRNA: tracr | crRNA | GUUGUGGUUUGAUUAAAGA (SEQ ID NO: 181) |
| RNA duplex | (Repeat) | |
| V1 | Partial | GUUGUGGUUUGAUUA (SEQ ID NO: 182) |
| | crRNA 1 | |
| | Partial | GUUGUGGUUUGA (SEQ ID NO: 183) |
| | crRNA 2 | |
| | Partial | GUUGUGGUUU (SEQ ID NO: 184) |
| | crRNA 3 | |
| | tracrRNA | UCUUUAAUCUUAUCACAAUA (SEQ ID NO: 185) |
| | (Antirepeat) | |
| | Partial | UAAUCUUAUCACAAUA (SEQ ID NO: 186) |
| | tracrRNA 1 | |
| | Partial | UCUUAUCACAAUA (SEQ ID NO: 187) |
| | tracrRNA 2 | |
| | Partial | UUAUCACAAUA (SEQ ID NO: 188) |
| | tracrRNA 3 | |
| tracrRNA | tracrRNA | AGGCUAUAUGCCGU (SEQ ID NO: 189) |
| sequences | Portion 1- | |
| | Full | |
| | tracrRNA | AGGCUAUAUGCC (SEQ ID NO: 190) |
| | Portion 1- | |
| | Partial | |
| | tracrRNA | AGAUGAAAAUCUUU (SEQ ID NO: 191) |
| | Portion 2- | |
| | Full | |
| | tracrRNA | AGAUGAAAAUCU (SEQ ID NO: 192) |
| | Portion 2- | |
| | Partial | |
| | tracrRNA | AGUCCUGCUUCGGUGGGACUUUUUUU (SEQ ID NO: 193) |
| | Portion 3- | |
| | Full | |
| | tracrRNA | AGUCCUGCUUCGGUGGGACU (SEQ ID NO: 194) |
| | Portion 3- | |
| | Partial | |
| | tracrRNA | AGUCCUGCUUCGGUGGGAC (SEQ ID NO: 195) |
| | Portion 3- | |
| | Without | |
| | polyT | |
| | | OMNI-XL-10 |
| sgRNA | sgRNA V1 | GUUGUGGAUUGACAGUUCgaaaGAACUGUCAUUUCACAAUAAGGC |
| Version | | UAUAUGCCGAAGGUCUCUGACCUAUUACCCCGCUUCGGCGGGGU |
| | | UUCUUUUUUU (SEQ ID NO: 196) |
| crRNA: tracr | crRNA | GUUGUGGAUUGACAGUUC (SEQ ID NO: 197) |
| RNA duplex | (Repeat) | |
| V1 | Partial | GUUGUGGAUUGACAG (SEQ ID NO: 198) |
| | crRNA 1 | |
| | Partial | GUUGUGGAUUGA (SEQ ID NO: 199) |
| | crRNA 2 | |
| | Partial | GUUGUGGAUU (SEQ ID NO: 200) |
| | crRNA 3 | |
| | tracrRNA | GAACUGUCAUUUCACAAUA (SEQ ID NO: 201) |
| | (Antirepeat) | |
| | Partial | CUGUCAUUUCACAAUA (SEQ ID NO: 202) |
| | tracrRNA 1 | |
| | Partial | UCAUUUCACAAUA (SEQ ID NO: 203) |
| | tracrRNA 2 | |
| | Partial | AUUUCACAAUA (SEQ ID NO: 204) |
| | tracrRNA 3 | |
| tracrRNA | tracrRNA | AGGCUAUAUGCCGA (SEQ ID NO: 205) |
| sequences | Portion 1- | |
| | Full | |
| | tracrRNA | AGGCUAUAUGCC (SEQ ID NO: 206) |
| | Portion 1- | |
| | Partial | |
| | tracrRNA | AGGUCUCUGACCUAUU (SEQ ID NO: 207) |
| | Portion 2- | |
| | Full | |
| | tracrRNA | AGGUCUCUGACCU (SEQ ID NO: 208) |
| | Portion 2- | |
| | Partial | |
| | tracrRNA | ACCCCGCUUCGGCGGGGUUUUUUUUUU (SEQ ID NO: 209) |
| | Portion 3- | |
| | Full | |
| | tracrRNA | ACCCCGCUUCGGCGGGGU (SEQ ID NO: 210) |
| | Portion 3- | |
| | Partial | |
| | tracrRNA | ACCCCGCUUCGGCGGGGUUUC (SEQ ID NO: 211) |
| | Portion 3- | |
| | Without | |
| | polyT | |
| | | OMNI-XL-11 |
| sgRNA | sgRNA V1 | GUUGUGGAUUGAUUACUUUgaaaAAAGUAAUCUUUUCAUAACAAG |
| Version | | GCUAUAUGCCGAAGUUAUUACGGCUCCACUUCGGUGGAGCCUUU |
| | | UUU (SEQ ID NO: 212) |
| crRNA: tracr | crRNA | GUUGUGGAUUGAUUACUUU (SEQ ID NO: 213) |
| RNA duplex | (Repeat) | |
| V1 | Partial | GUUGUGGAUUGAUUA (SEQ ID NO: 214) |
| | crRNA 1 | |
| | Partial | GUUGUGGAUUGA (SEQ ID NO: 215) |
| | crRNA 2 | |
| | Partial | GUUGUGGAUU (SEQ ID NO: 216) |
| | crRNA 3 | |
| | tracrRNA | AAAGUAAUCUUUUCAUAACA (SEQ ID NO: 217) |
| | (Antirepeat) | |
| | Partial | UAAUCUUUUCAUAACA (SEQ ID NO: 218) |
| | tracrRNA 1 | |
| | Partial | UCUUUUCAUAACA (SEQ ID NO: 219) |
| | tracrRNA 2 | |
| | Partial | UUUUCAUAACA (SEQ ID NO: 220) |
| | tracrRNA 3 | |
| tracrRNA | tracrRNA | AGGCUAUAUGCCGAAGUUAUUAC (SEQ ID NO: 221) |
| sequences | Portion 1- | |
| | Full | |
| | tracrRNA | AGGCUAUAUGCC (SEQ ID NO: 222) |
| | Portion 1- | |
| | Partial | |
| | tracrRNA | GGCUCCACUUCGGUGGAGCCUUUUUU (SEQ ID NO: 223) |
| | Portion 2- | |
| | Full | |
| | tracrRNA | GGCUCCACUUCGGUGGAGCC (SEQ ID NO: 224) |
| | Portion 2- | |
| | Partial | |
| | tracrRNA | GGCUCCACUUCGGUGGAGCC (SEQ ID NO: 225) |
| | Portion 2- | |
| | Without | |
| | polyT | |
| sgRNA | sgRNA V2 | GUUGUGGAUUGAUUACUUUgaaaAAAGUAAUCUCUUCAUAACAAG |
| Version | | GCUAUAUGCCGAAGUUAUUACGGCUCCACUUCGGUGGAGCCUUU |
| | | UUU (SEQ ID NO: 226) |
| crRNA: tracr | tracrRNA | AAAGUAAUCUCUUCAUAACA (SEQ ID NO: 227) |
| RNA duplex | (Antirepeat) | |
| V2 | Partial | UAAUCUCUUCAUAACA (SEQ ID NO: 228) |
| | traerRNA 1 | |
| | Partial | UCUCUUCAUAACA (SEQ ID NO: 229) |
| | tracrRNA 2 | |
| | Partial | UCUUCAUAACA (SEQ ID NO: 230) |
| | tracrRNA 3 | |
| | | OMNI-XL-12 |
| sgRNA | sgRNA V1 | GUUGUGGAUUGAUUACUUUGAAAAAAGUAAUCUUUUCAUAACA |
| Version | | AGGCUAUAUGCCGAAGUUAUUACGGCUCCACUUCGGUGGAGCCU |
| | | UUUUU (SEQ ID NO: 231) |
| crRNA: tracr | crRNA | GUUGUGGAUUGAUUACUUU (SEQ ID NO: 232) |
| RNA duplex | (Repeat) | |
| V1 | Partial | GUUGUGGAUUGAUUA (SEQ ID NO: 233) |
| | crRNA 1 | |
| | Partial | GUUGUGGAUUGA (SEQ ID NO: 234) |
| | crRNA 2 | |
| | Partial | GUUGUGGAUU (SEQ ID NO: 235) |
| | crRNA 3 | |
| | tracrRNA | AAAGUAAUCUUUUCAUAACA (SEQ ID NO: 236) |
| | (Antirepeat) | |
| | Partial | UAAUCUUUUCAUAACA (SEQ ID NO: 237) |
| | tracrRNA 1 | |
| | Partial | UCUUUUCAUAACA (SEQ ID NO: 238) |
| | tracrRNA 2 | |
| | Partial | UUUUCAUAACA (SEQ ID NO: 239) |
| | tracrRNA 3 | |
| tracrRNA | tracrRNA | AGGCUAUAUGCCGAAGUUAUUAC (SEQ ID NO: 240) |
| sequences | Portion 1- | |
| | Full | |
| | tracrRNA | AGGCUAUAUGCC (SEQ ID NO: 241) |
| | Portion 1- | |
| | Partial | |
| | tracrRNA | GGCUCCACUUCGGUGGAGCCUUUUUU (SEQ ID NO: 242) |
| | Portion 2- | |
| | Full | |
| | tracrRNA | GGCUCCACUUCGGUGGAGCC (SEQ ID NO: 243) |
| | Portion 2- | |
| | Partial | |
| | tracrRNA | GGCUCCACUUCGGUGGAGCC (SEQ ID NO: 244) |
| | Portion 2- | |
| | Without | |
| | polyT | |
| sgRNA | sgRNA V2 | GUUGUGGAUUGAUUACUUUGAAAAAAGUAAUCUCUUCAUAACA |
| Version | | AGGCUAUAUGCCGAAGUUAUUACGGCUCCACUUCGGUGGAGCCU |
| | | UUUUU (SEQ ID NO: 245) |
| crRNA: tracr | tracrRNA | AAAGUAAUCUCUUCAUAACA (SEQ ID NO: 246) |
| RNA duplex | (Antirepeat) | |
| V2 | Partial | UAAUCUCUUCAUAACA (SEQ ID NO: 247) |
| | tracrRNA 1 | |
| | Partial | UCUCUUCAUAACA (SEQ ID NO: 248) |
| | tracrRNA 2 | |
| | Partial | UCUUCAUAACA (SEQ ID NO: 249) |
| | tracrRNA 3 | |
| | | OMNI-XL-13 |
| sgRNA | sgRNA V1 | GUUGUGGAUUGAUCAGUUUgaaaAAACGAUCUUUUCACAACAAGG |
| Version | | CUAUAUGCCGUAGUUGAUUCUACGCUCCCGCUUCGGCGGGAGCU |
| | | UUUUUUU (SEQ ID NO: 250) |
| crRNA: tracr | crRNA | GUUGUGGAUUGAUCAGUUU (SEQ ID NO: 251) |
| RNA duplex | (Repeat) | |
| V1 | Partial | GUUGUGGAUUGAUCA (SEQ ID NO: 252) |
| | crRNA 1 | |
| | Partial | GUUGUGGAUUGA (SEQ ID NO: 253) |
| | crRNA 2 | |
| | Partial | GUUGUGGAUU (SEQ ID NO: 254) |
| | crRNA 3 | |
| | tracrRNA | AAACGAUCUUUUCACAACA (SEQ ID NO: 255) |
| | (Antirepeat) | |
| | Partial | GAUCUUUUCACAACA (SEQ ID NO: 256) |
| | tracrRNA 1 | |
| | Partial | UCUUUUCACAACA (SEQ ID NO: 257) |
| | tracrRNA 2 | |
| | Partial | UUUUCACAACA (SEQ ID NO: 258) |
| | tracrRNA 3 | |
| tracrRNA | tracrRNA | AGGCUAUAUGCC (SEQ ID NO: 259) |
| sequences | Portion 1- | |
| | Full | |
| | tracrRNA | AGGCUAUAUGCC (SEQ ID NO: 260) |
| | Portion 1- | |
| | Partial | |
| | tracrRNA | GUAGUUGAUUCUAC (SEQ ID NO: 261) |
| | Portion 2- | |
| | Full | |
| | tracrRNA | GUAGUUGAUUCUAC (SEQ ID NO: 262) |
| | Portion 2- | |
| | Partial | |
| | tracrRNA | GCUCCCGCUUCGGCGGGAGCUUUUUUUU (SEQ ID NO: 263) |
| | Portion 3- | |
| | Full | |
| | tracrRNA | GCUCCCGCUUCGGCGGGAGC (SEQ ID NO: 264) |
| | Portion 3- | |
| | Partial | |
| | tracrRNA | GCUCCCGCUUCGGCGGGAGC (SEQ ID NO: 265) |
| | Portion 3- | |
| | Without | |
| | polyT | |
| sgRNA | sgRNA V2 | GUUGUGGAUUGAUCAGUUUgaaaAAACGAUCUCUUCACAACAAGG |
| Version | | CUAUAUGCCGUAGUUGAUUCUACGCUCCCGCUUCGGCGGGAGCU |
| | | UUUUUUU (SEQ ID NO: 266) |
| crRNA: tracr | tracrRNA | AAACGAUCUCUUCACAACA (SEQ ID NO: 267) |
| RNA duplex | (Antirepeat) | |
| V2 | Partial | GAUCUCUUCACAACA (SEQ ID NO: 268) |
| | tracrRNA 1 | |
| | Partial | UCUCUUCACAACA (SEQ ID NO: 269) |
| | tracrRNA 2 | |
| | Partial | UCUUCACAACA (SEQ ID NO: 270) |
| | tracrRNA 3 | |
| | | OMNI-XL-14 |
| sgRNA | sgRNA V1 | GUUGCGGUUUGAUCUUUGAgaaaUCAAAGAUCUUAUCGCAAUAAG |
| Version | | GCUAUAUGCCGAAGGUCGUUUCGACCUAUAUCUCCGCUUAGGCG |
| | | GAGUUUUUUUU (SEQ ID NO: 271) |
| crRNA: tracr | crRNA | GUUGCGGUUUGAUCUUUGA (SEQ ID NO: 272) |
| RNA duplex | (Repeat) | |
| V1 | Partial | GUUGCGGUUUGAUCU (SEQ ID NO: 273) |
| | crRNA 1 | |
| | Partial | GUUGCGGUUUGA (SEQ ID NO: 274) |
| | crRNA 2 | |
| | Partial | GUUGCGGUUU (SEQ ID NO: 275) |
| | crRNA 3 | |
| | tracrRNA | UCAAAGAUCUUAUCGCAAUA (SEQ ID NO: 276) |
| | (Antirepeat) | |
| | Partial | AGAUCUUAUCGCAAUA (SEQ ID NO: 277) |
| | tracrRNA 1 | |
| | Partial | UCUUAUCGCAAUA (SEQ ID NO: 278) |
| | tracrRNA 2 | |
| | Partial | UUAUCGCAAUA (SEQ ID NO: 279) |
| | tracrRNA 3 | |
| tracrRNA | tracrRNA | AGGCUAUAUGCCGA (SEQ ID NO: 280) |
| sequences | Portion 1- | |
| | Full | |
| | tracrRNA | AGGCUAUAUGCC (SEQ ID NO: 281) |
| | Portion 1- | |
| | Partial | |
| | tracrRNA | AGGUCGUUUCGACCUAUAU (SEQ ID NO: 282) |
| | Portion 2- | |
| | Full | |
| | tracrRNA | AGGUCGUUUCGACCU (SEQ ID NO: 283) |
| | Portion 2- | |
| | Partial | |
| | tracrRNA | CUCCGCUUAGGCGGAGUUUUUUUU (SEQ ID NO: 284) |
| | Portion 3- | |
| | Full | |
| | tracrRNA | CUCCGCUUAGGCGGAG (SEQ ID NO: 285) |
| | Portion 3- | |
| | Partial | |
| | tracrRNA | CUCCGCUUAGGCGGAG (SEQ ID NO: 286) |
| | Portion 3- | |
| | Without | |
| | polyT | |
| | | OMNI-XL-15 |
| sgRNA | sgRNA V1 | GCUGUGAUUUGAUGUAAgaaaUUACAUCUUAUCACAGCAAGGCUA |
| Version | | UAUGCCGAAGGAUGUAAUCCUAUACUCCCGCUUCGGUGGGAGUU |
| | | UUUU (SEQ ID NO: 287) |
| crRNA: tracr | crRNA | GCUGUGAUUUGAUGUAA (SEQ ID NO: 288) |
| RNA duplex | (Repeat) | |
| V1 | Partial | GCUGUGAUUUGAUGU (SEQ ID NO: 289) |
| | crRNA 1 | |
| | Partial | GCUGUGAUUUGA (SEQ ID NO: 290) |
| | crRNA 2 | |
| | Partial | GCUGUGAUUU (SEQ ID NO: 291) |
| | crRNA 3 | |
| | tracrRNA | UUACAUCUUAUCACAGCA (SEQ ID NO: 292) |
| | (Antirepeat) | |
| | Partial | ACAUCUUAUCACAGCA (SEQ ID NO: 293) |
| | tracrRNA 1 | |
| | Partial | UCUUAUCACAGCA (SEQ ID NO: 294) |
| | tracrRNA 2 | |
| | Partial | UUAUCACAGCA (SEQ ID NO: 295) |
| | tracrRNA 3 | |
| tracrRNA | tracrRNA | AGGCUAUAUGCCGA (SEQ ID NO: 296) |
| sequences | Portion 1- | |
| | Full | |
| | tracrRNA | AGGCUAUAUGCC (SEQ ID NO: 297) |
| | Portion 1- | |
| | Partial | |
| | tracrRNA | AGGAUGUAAUCCUAU (SEQ ID NO: 298) |
| | Portion 2- | |
| | Full | |
| | tracrRNA | AGGAUGUAAUCCU (SEQ ID NO: 299) |
| | Portion 2- | |
| | Partial | |
| | tracrRNA | ACUCCCGCUUCGGUGGGAGUUUUUU (SEQ ID NO: 300) |
| | Portion 3- | |
| | Full | |
| | tracrRNA | ACUCCCGCUUCGGUGGGAGU (SEQ ID NO: 301) |
| | Portion 3- | |
| | Partial | |
| | tracrRNA | ACUCCCGCUUCGGUGGGAG (SEQ ID NO: 302) |
| | Portion 3- | |
| | Without | |
| | polyT | |
| | | OMNI-XL-16 |
| sgRNA | sgRNA V1 | GUUGUGGUUUGAUGUAAUUGAAAAAAUUACAUCUUAUCACAAU |
| Version | | AAGGCUAUAUGCCGAAGGAUAAAAUGUCCUAUACUCCUGCUUCG |
| | | GUAGGAGUUUUUUU (SEQ ID NO: 303) |
| crRNA: tracr | crRNA | GUUGUGGUUUGAUGUAAUU (SEQ ID NO: 304) |
| RNA duplex | (Repeat) | |
| V1 | Partial | GUUGUGGUUUGAUGU (SEQ ID NO: 305) |
| | crRNA 1 | |
| | Partial | GUUGUGGUUUGA (SEQ ID NO: 306) |
| | crRNA 2 | |
| | Partial | GUUGUGGUUU (SEQ ID NO: 307) |
| | crRNA 3 | |
| | tracrRNA | AAAUUACAUCUUAUCACAAUA (SEQ ID NO: 308) |
| | (Antirepeat) | |
| | Partial | ACAUCUUAUCACAAUA (SEQ ID NO: 309) |
| | tracrRNA 1 | |
| | Partial | UCUUAUCACAAUA (SEQ ID NO: 310) |
| | tracrRNA 2 | |
| | Partial | UUAUCACAAUA (SEQ ID NO: 311) |
| | tracrRNA 3 | |
| tracrRNA | tracrRNA | AGGCUAUAUGCCGA (SEQ ID NO: 312) |
| sequences | Portion 1- | |
| | Full | |
| | tracrRNA | AGGCUAUAUGCC (SEQ ID NO: 313) |
| | Portion 1- | |
| | Partial | |
| | tracrRNA | AGGAUAAAAUGUCCUAU (SEQ ID NO: 314) |
| | Portion 2- | |
| | Full | |
| | tracrRNA | AGGAUAAAAUGUCCU (SEQ ID NO: 315) |
| | Portion 2- | |
| | Partial | |
| | tracrRNA | ACUCCUGCUUCGGUAGGAGUUUUUUU (SEQ ID NO: 316) |
| | Portion 3- | |
| | Full | |
| | tracrRNA | ACUCCUGCUUCGGUAGGAGU (SEQ ID NO: 317) |
| | Portion 3- | |
| | Partial | |
| | tracrRNA | ACUCCUGCUUCGGUAGGAG (SEQ ID NO: 318) |
| | Portion 3- | |
| | Without | |
| | polyT | |
| | | OMNI-XL-17 |
| sgRNA | sgRNA V1 | GUUGUGGUUUGAUUAAAGAgaaaUCUUUAAUCUUAUCACAAUAAG |
| Version | | GCUAUAUGCCGUAGGUAAAAAACCUUGAGUCCCGCUUCGGUGGG |
| | | ACUUGUUUUUUUU (SEQ ID NO: 319) |
| crRNA: tracr | crRNA | GUUGUGGUUUGAUUAAAGA (SEQ ID NO: 320) |
| RNA duplex | (Repeat) | |
| V1 | Partial | GUUGUGGUUUGAUUA (SEQ ID NO: 321) |
| | crRNA 1 | |
| | Partial | GUUGUGGUUUGA (SEQ ID NO: 322) |
| | crRNA 2 | |
| | Partial | GUUGUGGUUU (SEQ ID NO: 323) |
| | crRNA 3 | |
| | tracrRNA | UCUUUAAUCUUAUCACAAUA (SEQ ID NO: 324) |
| | (Antirepeat) | |
| | Partial | UAAUCUUAUCACAAUA (SEQ ID NO: 325) |
| | tracrRNA 1 | |
| | Partial | UCUUAUCACAAUA (SEQ ID NO: X326) |
| | tracrRNA 2 | |
| | Partial | UUAUCACAAUA (SEQ ID NO: 327) |
| | tracrRNA 3 | |
| tracrRNA | tracrRNA | AGGCUAUAUGCCGUAGGU (SEQ ID NO: 328) |
| sequences | Portion 1- | |
| | Full | |
| | tracrRNA | AGGCUAUAUGCC (SEQ ID NO: 329) |
| | Portion 1- | |
| | Partial | |
| | tracrRNA | AAAAAACCUUGAGUCCCGCUUCGGUGGGACUUGUUUUUUUU |
| | Portion 2- | (SEQ ID NO: 330) |
| | Full | |
| | tracrRNA | AAAAAACCUUGAGUCCCGCUUCGGUGGGACUUGUUUUUU (SEQ |
| | Portion 2- | ID NO: 331) |
| | Partial | |
| | tracrRNA | AAAAAACCUUGAGUCCCGCUUCGGUGGGACUUG (SEQ ID NO: 332) |
| | Portion 2- | |
| | Without | |
| | polyT | |
| | | OMNI-XL-18 |
| sgRNA | sgRNA V1 | GUUGUGGAUUGAUCAGUUUGAAAAAACGAUCUUUUCACAACAA |
| Version | | GGCUAUAUGCCGUAGUUGAUUCUACGCUCCCGCUUCGGCGGGAG |
| | | CUUUUUUU (SEQ ID NO: 333) |
| crRNA: tracr | crRNA | GUUGUGGAUUGAUCAGUUU (SEQ ID NO: 334) |
| RNA duplex | (Repeat) | |
| V1 | Partial | GUUGUGGAUUGAUCA (SEQ ID NO: 335) |
| | crRNA 1 | |
| | Partial | GUUGUGGAUUGA (SEQ ID NO: 336) |
| | crRNA 2 | |
| | Partial | GUUGUGGAUU (SEQ ID NO: 337) |
| | crRNA 3 | |
| | tracrRNA | AAACGAUCUUUUCACAACA (SEQ ID NO: 338) |
| | (Antirepeat) | |
| | Partial | GAUCUUUUCACAACA (SEQ ID NO: 339) |
| | tracrRNA 1 | |
| | Partial | UCUUUUCACAACA (SEQ ID NO: 340) |
| | tracrRNA 2 | |
| | Partial | UUUUCACAACA (SEQ ID NO: 341) |
| | tracrRNA 3 | |
| tracrRNA | tracrRNA | AGGCUAUAUGCC (SEQ ID NO: 342) |
| sequences | Portion 1- | |
| | Full | |
| | tracrRNA | AGGCUAUAUGCC (SEQ ID NO: 343) |
| | Portion 1- | |
| | Partial | |
| | tracrRNA | GUAGUUGAUUCUAC (SEQ ID NO: 344) |
| | Portion 2- | |
| | Full | |
| | tracrRNA | GUAGUUGAUUCUAC (SEQ ID NO: 345) |
| | Portion 2- | |
| | Partial | |
| | tracrRNA | GCUCCCGCUUCGGCGGGAGCUUUUUUU (SEQ ID NO: 346) |
| | Portion 3- | |
| | Full | |
| | tracrRNA | GCUCCCGCUUCGGCGGGAGC (SEQ ID NO: 347) |
| | Portion 3- | |
| | Partial | |
| | tracrRNA | GCUCCCGCUUCGGCGGGAGC (SEQ ID NO: 348) |
| | Portion 3- | |
| | Without | |
| | polyT | |
| sgRNA | sgRNA V2 | GUUGUGGAUUGAUCAGUUUGAAAAAACGAUCUCUUCACAACAA |
| Version | | GGCUAUAUGCCGUAGUUGAUUCUACGCUCCCGCUUCGGCGGGAG |
| | | CUUUUUUU (SEQ ID NO: 349) |
| crRNA: tracr | tracrRNA | AAACGAUCUCUUCACAACA (SEQ ID NO: 350) |
| RNA duplex | (Antirepeat) | |
| V2 | Partial | GAUCUCUUCACAACA (SEQ ID NO: 351) |
| | traerRNA 1 | |
| | Partial | UCUCUUCACAACA (SEQ ID NO: 352) |
| | tracrRNA 2 | |
| | Partial | UCUUCACAACA (SEQ ID NO: 353) |
| | tracrRNA 3 | |
| | | OMNI-XL-19 |
| sgRNA | sgRNA V1 | GUUGUGGAUUGAUCAGUUUgaaaAAACGAUCUUUUCACAACAAGG |
| Version | | CUAUAUGCCGUAGUUGAUUCUACGCUCCCGCUUCGGCGGGAGCU |
| | | UUUUUUU (SEQ ID NO: 354) |
| crRNA: tracr | crRNA | GUUGUGGAUUGAUCAGUUU (SEQ ID NO: 355) |
| RNA duplex | (Repeat) | |
| V1 | Partial | GUUGUGGAUUGAUCA (SEQ ID NO: 356) |
| | crRNA 1 | |
| | Partial | GUUGUGGAUUGA (SEQ ID NO: 357) |
| | crRNA 2 | |
| | Partial | GUUGUGGAUU (SEQ ID NO: 358) |
| | crRNA 3 | |
| | tracrRNA | AAACGAUCUUUUCACAACA (SEQ ID NO: 359) |
| | (Antirepeat) | |
| | Partial | GAUCUUUUCACAACA (SEQ ID NO: 360) |
| | tracrRNA 1 | |
| | Partial | UCUUUUCACAACA (SEQ ID NO: 361) |
| | tracrRNA 2 | |
| | Partial | UUUUCACAACA (SEQ ID NO: 362) |
| | tracrRNA 3 | |
| tracrRNA | tracrRNA | AGGCUAUAUGCC (SEQ ID NO: 363) |
| sequences | Portion 1- | |
| | Full | |
| | tracrRNA | AGGCUAUAUGCC (SEQ ID NO: 364) |
| | Portion 1- | |
| | Partial | |
| | tracrRNA | GUAGUUGAUUCUAC (SEQ ID NO: 365) |
| | Portion 2- | |
| | Full | |
| | tracrRNA | GUAGUUGAUUCUAC (SEQ ID NO: 366) |
| | Portion 2- | |
| | Partial | |
| | tracrRNA | GCUCCCGCUUCGGCGGGAGCUUUUUUUU (SEQ ID NO: 367) |
| | Portion 3- | |
| | Full | |
| | tracrRNA | GCUCCCGCUUCGGCGGGAGC (SEQ ID NO: 368) |
| | Portion 3- | |
| | Partial | |
| | tracrRNA | GCUCCCGCUUCGGCGGGAGC (SEQ ID NO: 369) |
| | Portion 3- | |
| | Without | |
| | polyT | |

TABLE 3
OMNI PAM Sequences showing activity for each tested sgRNA

| | TXTL Depletion | | | |
|---|---|---|---|---|
| | | | Activity | |
| | | | (1-Depletion score), | |
| | | | per respective sgRNA | |
| Name | PAM Relaxed | PAM Strict | listed in right col. | sgRNA |
| OMNI-XL-12 | NRNNCCNN | NRTNCCRN | 0.98, 0.97 | V1, V2 |
| OMNI-XL-16 | NARNACVN | NARNACVN | 0.64 | V1 |
| OMNI-XL-18 | NGYCCNNN | NGYCCNNN | 0.97, 0.98 | V1, V2 |
| OMNI-XL-19 | NRGNRCNN | NRGNRCNN | 0.94 | V1 |

TABLE 4
Plasmids and Constructs

| Plasmid | Purpose | Elements | Example |
|---|---|---|---|
| pET9a | Expressing OMNI | T7 promoter HA Tag-Linker- | pETa-OMNI-XL-12 (SEQ ID NO: 370) |
| | polypeptide in the | OMNI ORF (Human optimized) - | |
| | bacterial system | SV40 NLS-8XHisTag -T7 | |
| | | terminator | |
| pShuttle | Expressing OMNI | U6 promoter - T7promoter - | pShuttle Guide-T2-OMNI-XL-12 V1 (SEQ ID NO: 371) |
| Guide T2 | sgRNA in the bacterial | T2 spacer sgRNA scaffold - | |
| | and human cell system | T7 terminator | |
| pbPOS T2 | Bacterial/TXTL | T2 protospacer - 8N PAM | pbPOS T2 library (SEQ ID NO: 372) |
| library | depletion assay | library - chloramphenicol | |
| | | acetyltransferase | |
| pmOMNI | Expressing OMNI | CMV promoter - Kozak - SV40 | pmOMNI-OMNI-XL-12 (SEQ ID NO: 373) |
| | polypeptide in the | NLS - OMNI ORF (human | |
| | human cell system | optimized) - HA - SV40 NLS - | |
| | | P2A - mCherry - bGH poly(A) | |
| | | signal | |

*Depletion score - Average of the ratios from 2 most depleted sites

TABLE 5
Nuclease activity in endogenous context in mammalian cells

| | | | | | Max |
|---|---|---|---|---|---|
| | Gene | Corresponding | | | Activity |
| Nuclease | target | spacer name | Spacer Sequence | PAM | Mean |
| OMNI-XL-12 | CXCR4 | CXCR4_s154_ | GCCUUGCAUAGGAAGU | AGTACCAG | 10.1 |
| | | 22 bp_II | UCCCAA (SEQ ID NO: | | |
| | | | 382) | | |
| | PDCD1 | PDCD1 s146 | GUCUGGUUGCUGGGGC | GGTACCAG | 36.6 |
| | | 22 bp_II | UCAUGC (SEQ ID NO: | | |
| | | | 383) | | |
| OMNI-XL-16 | B2M | B2M_s52_ | CGUGAGUAAACCUGAA | GAGTACCT | 19.8 |
| | | 22 bp_II | UCUUUG (SEQ ID NO: | | |
| | | | 384) | | |
| | CXCR4 | CXCR4_s100_ | UGCCUUGCAUAGGAAG | AAGTACCA | 11.1 |
| | | 22 bp_II | UUCCCA (SEQ ID NO: | | |
| | | | 385) | | |
| | TRAC | TRAC_s195_ | AUCCUCUUGUCCCACAG | CAGAACCC | 33.6 |
| | | 22 bp_II | AUAUC (SEQ ID NO: 386) | | |
| OMNI-XL-18 | CXCR4 | CXCR4_s195_ | AGAGACCCACACACCGG | CGCCCGCT | 16.6 |
| | | 22 bp_II | AGGAG (SEQ ID NO: 387) | | |
| | TRAC | TRAC_s199_ | UCCAGAAGACACCUUC | AGCCCAGG | 12.2 |
| | | 22 bp_II | UUCCCC (SEQ ID NO: | | |
| | | | 388) | | |
| OMNI-XL-19 | CXCR4 | CXCR4_s25_ | UGGCAAGAGACCCACA | AGGAGCGC | 81.28 |
| | | 22 bp_II | CACCGG (SEQ ID NO: | | |
| | | | 389) | | |
| | ELANE | ELANE_g143_ | GCCUGUUGCUGCAGUC | GGGAGCGG | 34.0 |
| | | 22 bp_II | CGGGCU (SEQ ID NO: | | |
| | | | 390) | | |

Table 5. Nuclease activity in endogenous context in mammalian cells: OMNI nucleases were expressed in mammalian cell system (HeLa) by DNA transfection together with an sgRNA expressing plasmid. Cell lysates were used for site specific genomic DNA amplification and NGS. The percentage of indels was measured and analyzed to determine the editing level. Each sgRNA is composed of the tracrRNA (see Table 2) and the spacer detailed here. The spacer 3′ genomic sequence contains the expected PAM relevant for each OMNI nuclease. All tests were done in triplicates. OMNI nuclease only (no guide) transfected cells served as a negative control.

### REFERENCES

Ahmad and Allen (1992) “Antibody-mediated Specific Binging and Cytotoxicity of Lipsome-entrapped Doxorubicin to Lung Cancer Cells in Vitro”, Cancer Research 52:4817-20.
Anderson (1992) “Human gene therapy”, Science 256:808-13.
Basha et al. (2011) “Influence of Cationic Lipid Composition on Gene Silencing Properties of Lipid Nanoparticle Formulations of siRNA in Antigen-Presenting Cells”, Mol. Ther. 19 (12): 2186-200.
Behr (1994) “Gene transfer with synthetic cationic amphiphiles: Prospects for gene therapy”, Bioconjuage Chem 5:382-89.
Blaese et al. (1995) “Vectors in cancer therapy: how will they deliver”, Cancer Gene Ther. 2:291-97.
Blaese et al. (1995) “T lympocyte-directed gene therapy for ADA-SCID: initial trial results after 4 years”, Science 270 (5235): 475-80.
Briner et al. (2014) “Guide RNA functional modules direct Cas9 activity and orthognality”, Molecular Cell 56:333-39.
Buchschacher and Panganiban (1992) “Human immunodeficiency virus vectors for inducible expression of foreign genes”, J. Virol. 66:2731-39.
Burstein et al. (2017) “New CRISPR-Cas systems from uncultivated microbes”, Nature 542:237-41.
Canver et al., (2015) “BCL11A enhancer dissection by Cas9-mediated in situ saturating mutagenesis”, Nature Vol. 527, Pgs. 192-214.
Chang and Wilson (1987) “Modification of DNA ends can decrease end-joining relative to homologous recombination in mammalian cells”, Proc. Natl. Acad. Sci. USA 84:4959-4963.
Charlesworth et al. (2019) “Identification of preexisting adaptive immunity to Cas9 proteins in humans”, Nature Medicine, 25 (2), 249.
Chung et al. (2006) “Agrobacterium is not alone: gene transfer to plants by viruses and other bacteria”, Trends Plant Sci. 11 (1): 1-4.
Coelho et al. (2013) “Safety and efficacy of RNAi therapy for transthyretin amyloidosis” N. Engl. J. Med. 369, 819-829.
Crystal (1995) “Transfer of genes to humans: early lessons and obstacles to success”, Science 270 (5235): 404-10.
Dillon (1993) “Regulation gene expression in gene therapy” Trends in Biotechnology 11 (5): 167-173.
Dranoff et al. (1997) “A phase I study of vaccination with autologous, irradiated melanoma cells engineered to secrete human granulocyte macrophage colony stimulating factor”, Hum. Gene Ther. 8 (1): 111-23.
Dunbar et al. (1995) “Retrovirally marked CD34-enriched peripheral blood and bone marrow cells contribute to long-term engraftment after autologous transplantation”, Blood 85:3048-57.
Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
Gao and Huang (1995) “Cationic liposome-mediated gene transfer” Gene Ther. 2 (10): 710-22.
Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
Han et al. (1995) “Ligand-directed retro-viral targeting of human breast cancer cells”, Proc. Natl. Acad. Sci. USA 92 (21): 9747-51.
Humbert et al., (2019) “Therapeutically relevant engraftment of a CRISPR-Cas9-edited HSC-enriched population with HbF reactivation in nonhuman primates”, Sci. Trans. Med., Vol. 11, Pgs. 1-13.
Inaba et al. (1992) “Generation of large numbers of dendritic cells from mouse bone marrow cultures supplemented with granulocyte/macrophage colony-stimulating factor”, J Exp Med. 176 (6): 1693-702.
Jinek et al. (2012) “A programmable dual-RNA-guided DNA endonuclease in adaptive bacterial immunity”, Science 337 (6096): 816-21.
Johan et al. (1992) “GLVR1, a receptor for gibbon ape leukemia virus, is homologous to a phosphate permease of Neurospora crassa and is expressed at high levels in the brain and thymus”, J Virol 66 (3): 1635-40.
Judge et al. (2006) “Design of noninflammatory synthetic siRNA mediating potent gene silencing in vivo”, Mol Ther. 13 (3): 494-505.
Kohn et al. (1995) “Engraftment of gene-modified umbilical cord blood cells in neonates with adnosine deaminase deficiency”, Nature Medicine 1:1017-23.
Kremer and Perricaudet (1995) “Adenovirus and adeno-associated virus mediated gene transfer”, Br. Med. Bull. 51 (1): 31-44.
Macdiarmid et al. (2009) “Sequential treatment of drug-resistant tumors with targeted minicells containing siRNA or a cytotoxic drug”, Nat Biotehcnol. 27 (7): 643-51.
Malech et al. (1997) “Prolonged production of NADPH oxidase-corrected granulocyes after gene therapy of chronic granulomatous disease”, PNAS 94 (22): 12133-38.
Maxwell et al. (2018) “A detailed cell-free transcription-translation-based assay to decipher CRISPR protospacer adjacent motifs”, Methods 14348-57
Miller et al. (1991) “Construction and properties of retrovirus packaging cells based on gibbon ape leukemia virus”, J Virol. 65 (5): 2220-24.
Miller (1992) “Human gene therapy comes of age”, Nature 357:455-60.
Mitani and Caskey (1993) “Delivering therapeutic genes-matching approach and application”, Trends in Biotechnology 11 (5): 162-66.
Nabel and Felgner (1993) “Direct gene transfer for immunotherapy and immunization”, Trends in Biotechnology 11 (5): 211-15.
Nehls et al. (1996) “Two genetically separable steps in the differentiation of thymic epithelium” Science 272:886-889.
Remy et al. (1994) “Gene Transfer with a Series of Lipphilic DNA-Binding Molecules”, Bioconjugate Chem. 5 (6): 647-54.
Sentmanat et al. (2018) “A Survey of Validation Strategies for CRISPR-Cas9 Editing”, Scientific Reports 8:888, doi: 10.1038/s41598-018-19441-8.
Sommerfelt et al. (1990) “Localization of the receptor gene for type D simian retroviruses on human chromosome 19”, J. Virol. 64 (12): 6214-20.
Van Brunt (1988) “Molecular framing: transgenic animals as bioactors” Biotechnology 6:1149-54.
Vigne et al. (1995) “Third-generation adenovectors for gene therapy”, Restorative Neurology and Neuroscience 8 (1,2): 35-36.
Wagner et al. (2019) “High prevalence of Streptococcus pyogenes Cas9-reactive T cells within the adult human population” Nature Medicine, 25 (2), 242
Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
Yu et al. (1994) “Progress towards gene therapy for HIV infection”, Gene Ther. 1 (1): 13-26.
Zetsche et al. (2015) “Cpf1 is a single RNA-guided endonuclease of a class 2 CRIPSR-Cas system” Cell 163 (3): 759-71.
Zuris et al. (2015) “Cationic lipid-mediated delivery of proteins enables efficient protein based genome editing in vitro and in vivo” Nat Biotechnol. 33 (1): 73-80.

What is claimed is:

1. A non-naturally occurring composition comprising a CRISPR nuclease comprising a sequence having at least 90% identity to the amino acid sequence selected from the group consisting of SEQ ID NOs: 12, 16, 18, 19, 1-11, 13-15, 17, and 20-22, or a nucleic acid molecule comprising a sequence encoding the CRISPR nuclease, wherein the CRISPR nuclease further comprises one or more nuclear localization sequences (NLSs).

2. The composition of claim 1, further comprising one or more RNA molecules, or a DNA polynucleotide encoding any one of the one or more RNA molecules, wherein the one or more RNA molecules and the CRISPR nuclease do not naturally occur together and the one or more RNA molecules are capable of forming a complex with the CRISPR nuclease and/or target the complex to a target site.

3. The composition of claim 2, wherein the CRISPR nuclease comprises a sequence having at least 90% identity to the amino acid sequence set forth in SEQ ID NO: 1, and at least one RNA molecule comprises a sequence selected from the group consisting of SEQ ID NOs:
49-64.

4. The composition of claim 3, wherein the CRISPR nuclease comprises a sequence having at least 90% identity to the amino acid sequence set forth in SEQ ID NO: 1 and at least one RNA molecule is a CRISPR RNA (crRNA) molecule comprising a guide sequence portion and a sequence selected from the group consisting of SEQ ID NOs: 50-53.

5. The composition of claim 4, further comprising a transactivating CRISPR RNA (tracrRNA) molecule comprising a sequence set forth in the group consisting of SEQ ID NOs: 54-64.

6. The composition of claim 2, wherein the CRISPR nuclease comprises a sequence having at least 90% identity to the amino acid sequence set forth in SEQ ID NO: 1 and at least one RNA molecule is a single-guide RNA (sgRNA) molecule comprising a guide sequence portion and a sequence selected from the group consisting of SEQ ID NOs: 49-64.

7. The composition of claim 2, wherein the CRISPR nuclease comprises a sequence having at least 90% identity to the amino acid sequence set forth in SEQ ID NO: 2, and at least one RNA molecule comprises a sequence selected from the group consisting of SEQ ID NOs: 65-80.

8. The composition of claim 7, wherein the CRISPR nuclease comprises a sequence having at least 90% identity to the amino acid sequence set forth in SEQ ID NO: 2 and at least one RNA molecule is a CRISPR RNA (crRNA) molecule comprising a guide sequence portion and a sequence selected from the group consisting of SEQ ID NOs: 66-69.

9. The composition of claim 8, further comprising a transactivating CRISPR RNA (tracrRNA) molecule comprising a sequence set forth in the group consisting of SEQ ID NOs: 70-80.

10. The composition of claim 2, wherein the CRISPR nuclease comprises a sequence having at least 90% identity to the amino acid sequence set forth in SEQ ID NO: 2 and at least one RNA molecule is a single-guide RNA (sgRNA) molecule comprising a guide sequence portion and a sequence selected from the group consisting of SEQ ID NOs: 65-80.

11. The composition of claim 2, wherein the CRISPR nuclease comprises a sequence having at least 90% identity to the amino acid sequence set forth in SEQ ID NO: 3, and at least one RNA molecule comprises a sequence selected from the group consisting of SEQ ID NOs: 81-96.

12. The composition of claim 11, wherein the CRISPR nuclease comprises a sequence having at least 90% identity to the amino acid sequence set forth in SEQ ID NO: 3 and at least one RNA molecule is a CRISPR RNA (crRNA) molecule comprising a guide sequence portion and a sequence selected from the group consisting of SEQ ID NOs: 82-85.

13. The composition of claim 12, further comprising a transactivating CRISPR RNA (tracrRNA) molecule comprising a sequence set forth in the group consisting of SEQ ID NOs: 86-96.

14. The composition of claim 2, wherein the CRISPR nuclease comprises a sequence having at least 90% identity to the amino acid sequence set forth in SEQ ID NO: 3 and at least one RNA molecule is a single-guide RNA (sgRNA) molecule comprising a guide sequence portion and a sequence selected from the group consisting of SEQ ID NOs: 81-96.

15. The composition of claim 2, wherein the CRISPR nuclease comprises a sequence having at least 90% identity to the amino acid sequence set forth in SEQ ID NO: 4, and at least one RNA molecule comprises a sequence selected from the group consisting of SEQ ID NOs: 97-112.

16. The composition of claim 15, wherein the CRISPR nuclease comprises a sequence having at least 90% identity to the amino acid sequence set forth in SEQ ID NO: 4 and at least one RNA molecule is a CRISPR RNA (crRNA) molecule comprising a guide sequence portion and a sequence selected from the group consisting of SEQ ID NOs: 98-101.

17. The composition of claim 16, further comprising a transactivating CRISPR RNA (tracrRNA) molecule comprising a sequence set forth in the group consisting of SEQ ID NOs: 102-112.

18. The composition of claim 2, wherein the CRISPR nuclease comprises a sequence having at least 90% identity to the amino acid sequence set forth in SEQ ID NO: 4 and at least one RNA molecule is a single-guide RNA (sgRNA) molecule comprising a guide sequence portion and a sequence selected from the group consisting of SEQ ID NOs: 97-112.

19. The composition of claim 2, wherein the CRISPR nuclease comprises a sequence having at least 90% identity to the amino acid sequence set forth in SEQ ID NO: 5, and at least one RNA molecule comprises a sequence selected from the group consisting of SEQ ID NOs: 113-128.

20. The composition of claim 19, wherein the CRISPR nuclease comprises a sequence having at least 90% identity to the amino acid sequence set forth in SEQ ID NO: 5 and at least one RNA molecule is a CRISPR RNA (crRNA) molecule comprising a guide sequence portion and a sequence selected from the group consisting of SEQ ID NOs: 114-117.

21. The composition of claim 20, further comprising a transactivating CRISPR RNA (tracrRNA) molecule comprising a sequence set forth in the group consisting of SEQ ID NOs: 118-128.

22. The composition of claim 2, wherein the CRISPR nuclease comprises a sequence having at least 90% identity to the amino acid sequence set forth in SEQ ID NO: 5 and at least one RNA molecule is a single-guide RNA (sgRNA) molecule comprising a guide sequence portion and a sequence selected from the group consisting of SEQ ID NOs: 113-128.

23. The composition of claim 2, wherein the CRISPR nuclease comprises a sequence having at least 90% identity to the amino acid sequence set forth in SEQ ID NO: 6, and at least one RNA molecule comprises a sequence selected from the group consisting of SEQ ID NOs: 129-144.

24. The composition of claim 23, wherein the CRISPR nuclease comprises a sequence having at least 90% identity to the amino acid sequence set forth in SEQ ID NO: 6 and at least one RNA molecule is a CRISPR RNA (crRNA) molecule comprising a guide sequence portion and a sequence selected from the group consisting of SEQ ID NOs: 130-133.

25. The composition of claim 24, further comprising a transactivating CRISPR RNA (tracrRNA) molecule comprising a sequence set forth in the group consisting of SEQ ID NOs: 134-144.

26. The composition of claim 2, wherein the CRISPR nuclease comprises a sequence having at least 90% identity to the amino acid sequence set forth in SEQ ID NO: 6 and at least one RNA molecule is a single-guide RNA (sgRNA) molecule comprising a guide sequence portion and a sequence selected from the group consisting of SEQ ID NOs: 129-144.

27. The composition of claim 2, wherein the CRISPR nuclease comprises a sequence having at least 90% identity to the amino acid sequence set forth in SEQ ID NO: 7, and at least one RNA molecule comprises a sequence selected from the group consisting of SEQ ID NOs: 145-163.

28. The composition of claim 27, wherein the CRISPR nuclease comprises a sequence having at least 90% identity to the amino acid sequence set forth in SEQ ID NO: 7 and at least one RNA molecule is a CRISPR RNA (crRNA) molecule comprising a guide sequence portion and a sequence selected from the group consisting of SEQ ID NOs: 146-149.

29. The composition of claim 28, further comprising a transactivating CRISPR RNA (tracrRNA) molecule comprising a sequence set forth in the group consisting of SEQ ID NOs: 150-160, 162, and 163.

30. The composition of claim 2, wherein the CRISPR nuclease comprises a sequence having at least 90% identity to the amino acid sequence set forth in SEQ ID NO: 7 and at least one RNA molecule is a single-guide RNA (sgRNA) molecule comprising a guide sequence portion and a sequence selected from the group consisting of SEQ ID NOs: 145-163.

31. The composition of claim 2, wherein the CRISPR nuclease comprises a sequence having at least 90% identity to the amino acid sequence set forth in SEQ ID NO: 8, and at least one RNA molecule comprises a sequence selected from the group consisting of SEQ ID NOs: 164-179.

32. The composition of claim 31, wherein the CRISPR nuclease comprises a sequence having at least 90% identity to the amino acid sequence set forth in SEQ ID NO: 8 and at least one RNA molecule is a CRISPR RNA (crRNA) molecule comprising a guide sequence portion and a sequence selected from the group consisting of SEQ ID NOs: 165-168.

33. The composition of claim 32, further comprising a transactivating CRISPR RNA (tracrRNA) molecule comprising a sequence set forth in the group consisting of SEQ ID NOs: 169-179.

34. The composition of claim 2, wherein the CRISPR nuclease comprises a sequence having at least 90% identity to the amino acid sequence set forth in SEQ ID NO: 8 and at least one RNA molecule is a single-guide RNA (sgRNA) molecule comprising a guide sequence portion and a sequence selected from the group consisting of SEQ ID NOs: 164-179.

35. The composition of claim 2, wherein the CRISPR nuclease comprises a sequence having at least 90% identity to the amino acid sequence set forth in SEQ ID NO: 9, and at least one RNA molecule comprises a sequence selected from the group consisting of SEQ ID NOs: 180-195.

36. The composition of claim 35, wherein the CRISPR nuclease comprises a sequence having at least 90% identity to the amino acid sequence set forth in SEQ ID NO: 9 and at least one RNA molecule is a CRISPR RNA (crRNA) molecule comprising a guide sequence portion and a sequence selected from the group consisting of SEQ ID NOs: 181-184.

37. The composition of claim 36, further comprising a transactivating CRISPR RNA (tracrRNA) molecule comprising a sequence set forth in the group consisting of SEQ ID NOs: 185-195.

38. The composition of claim 2, wherein the CRISPR nuclease comprises a sequence having at least 90% identity to the amino acid sequence set forth in SEQ ID NO: 9 and at least one RNA molecule is a single-guide RNA (sgRNA) molecule comprising a guide sequence portion and a sequence selected from the group consisting of SEQ ID NOs: 180-195.

39. The composition of claim 2, wherein the CRISPR nuclease comprises a sequence having at least 90% identity to the amino acid sequence set forth in SEQ ID NO: 10, and at least one RNA molecule comprises a sequence selected from the group consisting of SEQ ID NOs: 196-211.

40. The composition of claim 39, wherein the CRISPR nuclease comprises a sequence having at least 90% identity to the amino acid sequence set forth in SEQ ID NO: 10 and at least one RNA molecule is a CRISPR RNA (crRNA) molecule comprising a guide sequence portion and a sequence selected from the group consisting of SEQ ID NOs: 197-200.

41. The composition of claim 40, further comprising a transactivating CRISPR RNA (tracrRNA) molecule comprising a sequence set forth in the group consisting of SEQ ID NOs: 201-211.

42. The composition of claim 2, wherein the CRISPR nuclease comprises a sequence having at least 90% identity to the amino acid sequence set forth in SEQ ID NO: 10 and at least one RNA molecule is a single-guide RNA (sgRNA) molecule comprising a guide sequence portion and a sequence selected from the group consisting of SEQ ID NOs: 196-211.

43. The composition of claim 2, wherein the CRISPR nuclease comprises a sequence having at least 90% identity to the amino acid sequence set forth in SEQ ID NO: 11, and at least one RNA molecule comprises a sequence selected from the group consisting of SEQ ID NOs: 212-230.

44. The composition of claim 43, wherein the CRISPR nuclease comprises a sequence having at least 90% identity to the amino acid sequence set forth in SEQ ID NO: 11 and at least one RNA molecule is a CRISPR RNA (crRNA) molecule comprising a guide sequence portion and a sequence selected from the group consisting of SEQ ID NOs: 213-216.

45. The composition of claim 44, further comprising a transactivating CRISPR RNA (tracrRNA) molecule comprising a sequence set forth in the group consisting of SEQ ID NOs: 217-225 and 227-230.

46. The composition of claim 2, wherein the CRISPR nuclease comprises a sequence having at least 90% identity to the amino acid sequence set forth in SEQ ID NO: 11 and at least one RNA molecule is a single-guide RNA (sgRNA) molecule comprising a guide sequence portion and a sequence selected from the group consisting of SEQ ID NOs: 212-230.

47. The composition of claim 2, wherein the CRISPR nuclease comprises a sequence having at least 90% identity to the amino acid sequence set forth in SEQ ID NO: 12, and at least one RNA molecule comprises a sequence selected from the group consisting of SEQ ID NOs: 231-249.

48. The composition of claim 47, wherein the CRISPR nuclease comprises a sequence having at least 90% identity to the amino acid sequence set forth in SEQ ID NO: 12 and at least one RNA molecule is a CRISPR RNA (crRNA) molecule comprising a guide sequence portion and a sequence selected from the group consisting of SEQ ID NOs: 232-235.

49. The composition of claim 48, further comprising a transactivating CRISPR RNA (tracrRNA) molecule comprising a sequence set forth in the group consisting of SEQ ID NOs: 236-244 and 246-249.

50. The composition of claim 2, wherein the CRISPR nuclease comprises a sequence having at least 90% identity to the amino acid sequence set forth in SEQ ID NO: 12 and at least one RNA molecule is a single-guide RNA (sgRNA) molecule comprising a guide sequence portion and a sequence selected from the group consisting of SEQ ID NOs: 231-249.

51. The composition of claim 2, wherein the CRISPR nuclease comprises a sequence having at least 90% identity to the amino acid sequence set forth in SEQ ID NO: 13, and at least one RNA molecule comprises a sequence selected from the group consisting of SEQ ID NOs: 250-270.

52. The composition of claim 51, wherein the CRISPR nuclease comprises a sequence having at least 90% identity to the amino acid sequence set forth in SEQ ID NO: 13 and at least one RNA molecule is a CRISPR RNA (crRNA) molecule comprising a guide sequence portion and a sequence selected from the group consisting of SEQ ID NOs: 251-254.

53. The composition of claim 52, further comprising a transactivating CRISPR RNA (tracrRNA) molecule comprising a sequence set forth in the group consisting of SEQ ID NOs: 255-265 and 267-270.

54. The composition of claim 2, wherein the CRISPR nuclease comprises a sequence having at least 90% identity to the amino acid sequence set forth in SEQ ID NO: 13 and at least one RNA molecule is a single-guide RNA (sgRNA) molecule comprising a guide sequence portion and a sequence selected from the group consisting of SEQ ID NOs: 250-270.

55. The composition of claim 2, wherein the CRISPR nuclease comprises a sequence having at least 90% identity to the amino acid sequence set forth in SEQ ID NO: 14, and at least one RNA molecule comprises a sequence selected from the group consisting of SEQ ID NOs: 271-286.

56. The composition of claim 55, wherein the CRISPR nuclease comprises a sequence having at least 90% identity to the amino acid sequence set forth in SEQ ID NO: 14 and at least one RNA molecule is a CRISPR RNA (crRNA) molecule comprising a guide sequence portion and a sequence selected from the group consisting of SEQ ID NOs: 272-275.

57. The composition of claim 56, further comprising a transactivating CRISPR RNA (tracrRNA) molecule comprising a sequence set forth in the group consisting of SEQ ID NOs: 276-286.

58. The composition of claim 2, wherein the CRISPR nuclease comprises a sequence having at least 90% identity to the amino acid sequence set forth in SEQ ID NO: 14 and at least one RNA molecule is a single-guide RNA (sgRNA) molecule comprising a guide sequence portion and a sequence selected from the group consisting of SEQ ID NOs: 271-286.

59. The composition of claim 2, wherein the CRISPR nuclease comprises a sequence having at least 90% identity to the amino acid sequence set forth in SEQ ID NO: 15, and at least one RNA molecule comprises a sequence selected from the group consisting of SEQ ID NOs: 287-302.

60. The composition of claim 59, wherein the CRISPR nuclease comprises a sequence having at least 90% identity to the amino acid sequence set forth in SEQ ID NO: 15 and at least one RNA molecule is a CRISPR RNA (crRNA) molecule comprising a guide sequence portion and a sequence selected from the group consisting of SEQ ID NOs: 288-291.

61. The composition of claim 60, further comprising a transactivating CRISPR RNA (tracrRNA) molecule comprising a sequence set forth in the group consisting of SEQ ID NOs: 292-302.

62. The composition of claim 2, wherein the CRISPR nuclease comprises a sequence having at least 90% identity to the amino acid sequence set forth in SEQ ID NO: 15 and at least one RNA molecule is a single-guide RNA (sgRNA) molecule comprising a guide sequence portion and a sequence selected from the group consisting of SEQ ID NOs: 287-302.

63. The composition of claim 2, wherein the CRISPR nuclease comprises a sequence having at least 90% identity to the amino acid sequence set forth in SEQ ID NO: 16, and at least one RNA molecule comprises a sequence selected from the group consisting of SEQ ID NOs: 303-318.

64. The composition of claim 63, wherein the CRISPR nuclease comprises a sequence having at least 90% identity to the amino acid sequence set forth in SEQ ID NO: 16 and at least one RNA molecule is a CRISPR RNA (crRNA) molecule comprising a guide sequence portion and a sequence selected from the group consisting of SEQ ID NOs: 304-307.

65. The composition of claim 64, further comprising a transactivating CRISPR RNA (tracrRNA) molecule comprising a sequence set forth in the group consisting of SEQ ID NOs: 308-318.

66. The composition of claim 2, wherein the CRISPR nuclease comprises a sequence having at least 90% identity to the amino acid sequence set forth in SEQ ID NO: 16 and at least one RNA molecule is a single-guide RNA (sgRNA) molecule comprising a guide sequence portion and a sequence selected from the group consisting of SEQ ID NOs: 303-318.

67. The composition of claim 2, wherein the CRISPR nuclease comprises a sequence having at least 90% identity to the amino acid sequence set forth in SEQ ID NO: 17, and at least one RNA molecule comprises a sequence selected from the group consisting of SEQ ID NOs: 319-332.

68. The composition of claim 67, wherein the CRISPR nuclease comprises a sequence having at least 90% identity to the amino acid sequence set forth in SEQ ID NO: 17 and at least one RNA molecule is a CRISPR RNA (crRNA) molecule comprising a guide sequence portion and a sequence selected from the group consisting of SEQ ID NOs: 320-323.

69. The composition of claim 68, further comprising a transactivating CRISPR RNA (tracrRNA) molecule comprising a sequence set forth in the group consisting of SEQ ID NOs: 324-332.

70. The composition of claim 2, wherein the CRISPR nuclease comprises a sequence having at least 90% identity to the amino acid sequence set forth in SEQ ID NO: 17 and at least one RNA molecule is a single-guide RNA (sgRNA) molecule comprising a guide sequence portion and a sequence selected from the group consisting of SEQ ID NOs: 319-332.

71. The composition of claim 2, wherein the CRISPR nuclease comprises a sequence having at least 90% identity to the amino acid sequence set forth in SEQ ID NO: 18, and at least one RNA molecule comprises a sequence selected from the group consisting of SEQ ID NOs: 333-353.

72. The composition of claim 71, wherein the CRISPR nuclease comprises a sequence having at least 90% identity to the amino acid sequence set forth in SEQ ID NO: 18 and at least one RNA molecule is a CRISPR RNA (crRNA) molecule comprising a guide sequence portion and a sequence selected from the group consisting of SEQ ID NOs: 334-337.

73. The composition of claim 72, further comprising a transactivating CRISPR RNA (tracrRNA) molecule comprising a sequence set forth in the group consisting of SEQ ID NOs: 338-348 and 350-353.

74. The composition of claim 2, wherein the CRISPR nuclease comprises a sequence having at least 90% identity to the amino acid sequence set forth in SEQ ID NO: 18 and at least one RNA molecule is a single-guide RNA (sgRNA) molecule comprising a guide sequence portion and a sequence selected from the group consisting of SEQ ID NOs: 333-353.

75. The composition of claim 2, wherein the CRISPR nuclease comprises a sequence having at least 90% identity to the amino acid sequence set forth in SEQ ID NO: 19, and at least one RNA molecule comprises a sequence selected from the group consisting of SEQ ID NOs: 354-369.

76. The composition of claim 75, wherein the CRISPR nuclease comprises a sequence having at least 90% identity to the amino acid sequence set forth in SEQ ID NO: 19 and at least one RNA molecule is a CRISPR RNA (crRNA) molecule comprising a guide sequence portion and a sequence selected from the group consisting of SEQ ID NOs: 355-358.

77. The composition of claim 76, further comprising a transactivating CRISPR RNA (tracrRNA) molecule comprising a sequence set forth in the group consisting of SEQ ID NOs: 359-369.

78. The composition of claim 2, wherein the CRISPR nuclease comprises a sequence having at least 90% identity to the amino acid sequence set forth in SEQ ID NO: 19 and at least one RNA molecule is a single-guide RNA (sgRNA) molecule comprising a guide sequence portion and a sequence selected from the group consisting of SEQ ID NOs: 354-369.

79. The composition of any one of claims 1-78, wherein the amino acid sequence of the CRISPR nuclease comprises a conserved insert consisting of 343 consensus positions, wherein each consensus position may be either an amino acid or a gap such that the full conserved insert sequence is provided by linking together amino acids and removing gaps,
wherein consensus position 1 of the conserved insert region is an asparagine (N) residue;
wherein consensus position 4 of the conserved insert region is a leucine (L) residue;
wherein consensus position 45 of the conserved insert region is a lysine (K) residue;
wherein consensus position 50 of the conserved insert region is a proline (P) residue;
wherein consensus position 61 of the conserved insert region is a valine (V) residue;
wherein consensus position 63 of the conserved insert region is a valine (V) residue;
wherein consensus position 87 of the conserved insert region is a leucine (L) residue;
wherein consensus position 94 of the conserved insert region is a glycine (G) residue;
wherein consensus position 162 of the conserved insert region is a glycine (G) residue;
wherein consensus position 164 of the conserved insert region is a tyrosine (Y) residue;
wherein consensus position 165 of the conserved insert region is a tryptophan (W) residue;
wherein consensus position 184 of the conserved insert region is a arginine (R) residue;
wherein consensus position 197 of the conserved insert region is a glycine (G) residue;
wherein consensus position 204 of the conserved insert region is a phenylalanine (F) residue;
wherein consensus position 232 of the conserved insert region is a leucine (L) residue;
wherein consensus position 241 of the conserved insert region is a phenylalanine (F) residue;
wherein consensus position 243 of the conserved insert region is a lysine (K) residue;
wherein consensus position 249 of the conserved insert region is a proline (P) residue;
wherein consensus position 258 of the conserved insert region is a leucine (L) residue;
wherein consensus position 273 of the conserved insert region is an aspartic acid (D) residue;
wherein consensus position 280 of the conserved insert region is a glutamine (Q) residue;
wherein consensus position 291 of the conserved insert region is a tyrosine (Y) residue;
wherein consensus position 316 of the conserved insert region is a proline (P) residue;
wherein consensus position 327 of the conserved insert region is a glycine (G) residue; and/or
wherein consensus position 343 of the conserved insert region is a proline (P) residue.

80. The composition of any one of claims 1-78, wherein the amino acid sequence of the CRISPR nuclease comprises a conserved insert consisting of 343 consensus positions, wherein each consensus position may be either an amino acid or a gap such that the full conserved insert sequence is provided by linking together amino acids and removing gaps,
wherein consensus position 1 of the conserved insert region is an asparagine (N) residue;
wherein consensus position 2 of the conserved insert region is a lysine (K), asparagine (N), or serine(S) residue;
wherein consensus position 3 of the conserved insert region is an isoleucine (I), methionine (M), or valine (V) residue;
wherein consensus position 4 of the conserved insert region is an isoleucine (I), leucine (L), or valine (V) residue;
wherein consensus position 8 of the conserved insert region is a proline (P), serine(S), or tyrosine (Y) residue;
wherein consensus position 19 of the conserved insert region is a cysteine (C), isoleucine (I), or leucine (L) residue, or a gap;
wherein consensus position 29 of the conserved insert region is an isoleucine (I), valine (V), tryptophan (W), or tyrosine (Y) residue;
wherein consensus position 50 of the conserved insert region is a proline (P) residue;
wherein consensus position 56 of the conserved insert region is an aspartic acid (D), glutamic acid (E), or glutamine (Q) residue;
wherein consensus position 65 of the conserved insert region is a glutamic acid (E), glycine (G), or asparagine (N) residue, or a gap;
wherein consensus position 66 of the conserved insert region is a glycine (G), histidine (H), lysine (K), asparagine (N), or threonine (T) residue;
wherein consensus position 76 of the conserved insert region is a glutamic acid (E) or arginine (R) residue, or a gap;
wherein consensus position 77 of the conserved insert region is a histidine (H) or leucine (L) residue, or a gap;
wherein consensus position 87 of the conserved insert region is a glutamic acid (E), leucine (L), or arginine (R) residue;
wherein consensus position 89 of the conserved insert region is a glycine (G), leucine (L), or glutamine (Q) residue, or a gap;
wherein consensus position 90 of the conserved insert region is an aspartic acid (D), lysine (K), asparagine (N), or serine(S) residue, or a gap;
wherein consensus position 91 of the conserved insert region is a glycine (G), asparagine (N), or tyrosine (Y) residue, or a gap;
wherein consensus position 92 of the conserved insert region is a glutamic acid (E), phenylalanine (F), or valine (V) residue, or a gap;
wherein consensus position 94 of the conserved insert region is a glycine (G) residue;
wherein consensus position 96 of the conserved insert region is an alanine (A), histidine (H), or tyrosine (Y) residue;
wherein consensus position 100 of the conserved insert region is a leucine (L), valine (V), or tyrosine (Y) residue;
wherein consensus position 105 of the conserved insert region is a glycine (G), lysine (K), or asparagine (N) residue, or a gap;
wherein consensus position 113 of the conserved insert region is a phenylalanine (F), histidine (H), lysine (K), or tyrosine (Y) residue;
wherein consensus position 117 of the conserved insert region is an alanine (A) or proline (P) residue;
wherein consensus position 125 of the conserved insert region is an isoleucine (I), arginine (R), or valine (V) residue;
wherein consensus position 128 of the conserved insert region is an alanine (A), glycine (G), threonine (T), or valine (V) residue;
wherein consensus position 132 of the conserved insert region is a leucine (L) residue, or a gap;
wherein consensus position 133 of the conserved insert region is an asparagine (N) residue, or a gap;
wherein consensus position 134 of the conserved insert region is a lysine (K) or arginine (R) residue, or a gap;
wherein consensus position 135 of the conserved insert region is an aspartic acid (D) residue, or a gap;
wherein consensus position 136 of the conserved insert region is a glycine (G) or serine(S) residue, or a gap;
wherein consensus position 140 of the conserved insert region is a phenylalanine (F) or leucine (L) residue;
wherein consensus position 147 of the conserved insert region is a phenylalanine (F) residue or a gap;
wherein consensus position 156 of the conserved insert region is a aspartic acid (D), arginine (R), or serine(S) residue, or a gap;
wherein consensus position 162 of the conserved insert region is a glycine (G) or serine(S) residue;
wherein consensus position 164 of the conserved insert region is a cysteine (C) or tyrosine (Y) residue;
wherein consensus position 165 of the conserved insert region is a lysine (K) or tryptophan (W) residue;
wherein consensus position 176 of the conserved insert region is a valine (V) or tryptophan (W) residue, or a gap;
wherein consensus position 186 of the conserved insert region is an alanine (A), lysine (K), or arginine (R) residue, or a gap;
wherein consensus position 187 of the conserved insert region is a threonine (T) residue or a gap;
wherein consensus position 195 of the conserved insert region is a leucine (L) or valine (V) residue;
wherein consensus position 197 of the conserved insert region is a glycine (G) residue;
wherein consensus position 199 of the conserved insert region is an isoleucine (I), leucine (L), threonine (T), or valine (V) residue;
wherein consensus position 204 of the conserved insert region is a phenylalanine (F) residue;
wherein consensus position 208 of the conserved insert region is a proline (P) residue or a gap;
wherein consensus position 209 of the conserved insert region is an aspartic acid (D) residue or a gap;
wherein consensus position 211 of the conserved insert region is a phenylalanine (F) or tyrosine (Y) residue;
wherein consensus position 215 of the conserved insert region is a glutamic acid (E) or valine (V) residue, or a gap;
wherein consensus position 216 of the conserved insert region is an aspartic acid (D) or glutamic acid (E) residue, or a gap;
wherein consensus position 217 of the conserved insert region is an asparagine (N) or serine(S) residue, or a gap;
wherein consensus position 218 of the conserved insert region is a serine(S) residue or a gap;
wherein consensus position 219 of the conserved insert region is a valine (V) residue or a gap;
wherein consensus position 225 of the conserved insert region is an aspartic acid (D), histidine (H), or proline (P) residue;
wherein consensus position 226 of the conserved insert region is a glycine (G) or arginine (R) residue;
wherein consensus position 235 of the conserved insert region is an aspartic acid (D), glutamic acid (E), phenylalanine (F), methionine (M), or asparagine (N) residue;
wherein consensus position 241 of the conserved insert region is a phenylalanine (F), isoleucine (I), or tyrosine (Y) residue;
wherein consensus position 249 of the conserved insert region is a glycine (G), proline (P), or serine(S) residue;
wherein consensus position 260 of the conserved insert region is an alanine (A) or glycine (G) residue;
wherein consensus position 268 of the conserved insert region is an alanine (A), phenylalanine (F), or leucine (L) residue;
wherein consensus position 273 of the conserved insert region is an aspartic acid (D) or asparagine (N) residue;
wherein consensus position 274 of the conserved insert region is a glycine (G) residue, or a gap;
wherein consensus position 288 of the conserved insert region is a glycine (G), serine(S), or threonine (T) residue;
wherein consensus position 289 of the conserved insert region is a lysine (K), glutamine (Q), or arginine (R) residue;
wherein consensus position 290 of the conserved insert region is a histidine (H) or tyrosine (Y) residue;
wherein consensus position 303 of the conserved insert region is a glutamic acid (E) or lysine (K) residue, or a gap;
wherein consensus position 304 of the conserved insert region is a glutamic acid (E) residue, or a gap;
wherein consensus position 305 of the conserved insert region is a glutamine (Q) or arginine (R) residue, or a gap;
wherein consensus position 306 of the conserved insert region is a valine (V) residue, or a gap;
wherein consensus position 307 of the conserved insert region is a threonine (T) residue, or a gap;
wherein consensus position 316 of the conserved insert region is a proline (P) or glutamine (Q) residue;
wherein consensus position 326 of the conserved insert region is an aspartic acid (D) or glutamic acid (E) residue;
wherein consensus position 327 of the conserved insert region is a glycine (G) residue;
wherein consensus position 331 of the conserved insert region is an alanine (A) or leucine (L) residue, or a gap;
wherein consensus position 333 of the conserved insert region is an aspartic acid (D) or asparagine (N) residue;
wherein consensus position 338 of the conserved insert region is a glutamic acid (E), leucine (L), or arginine (R) residue;
wherein consensus position 339 of the conserved insert region is an alanine (A), isoleucine (I), or tyrosine (Y) residue;
wherein consensus position 341 of the conserved insert region is a phenylalanine (F) or leucine (L) residue; and/or
wherein consensus position 343 of the conserved insert region is a proline (P) or serine(S) residue.

81. The composition of any one of claims 1-80, wherein the CRISPR nuclease amino acid sequence is other than SEQ ID NOs: 45-48.

82. The composition of any one of claims 1-81, wherein the CRISPR nuclease is a nickase having an inactivated RuvC domain created by an amino acid substitution at a position provided for the CRISPR nuclease in column 5 of Table 1.

83. The composition of any one of claims 1-81, wherein the CRISPR nuclease is a nickase having an inactivated HNH domain created by an amino acid substitution at a position provided for the CRISPR nuclease in column 6 of Table 1.

84. The composition of any one of claims 1-81, wherein the CRISPR nuclease is a catalytically dead nuclease having an inactivated RuvC domain and an inactivated HNH domain created by substitutions at the positions provided for the CRISPR nuclease in column 7 of Table 1.

85. A method of modifying a nucleotide sequence at a DNA target site in a cell-free system or the genome of a cell comprising introducing into the cell the composition of any one of claims 2-23.

86. The method of claim 85, wherein the CRISPR nuclease effects a DNA break in a DNA strand adjacent to a protospacer adjacent motif (PAM) sequence provided for the CRISPR nuclease in columns 2-3 of Table 3, and/or effects a DNA break in a DNA strand adjacent to a sequence that is complementary to the PAM sequence.

87. The method of claim 85, wherein the CRISPR nuclease is a nickase having an inactivated RuvC domain created by an amino acid substitution at a position provided for the CRISPR nuclease in column 5 of Table 1, and effects a DNA break in a DNA strand adjacent to a sequence that is complementary to the PAM sequence.

88. The method of claim 85, wherein the CRISPR nuclease is a nickase having an inactivated HNH domain created by an amino acid substitution at a position provided for the CRISPR nuclease in column 6 of Table 1, and effects a DNA break in a DNA strand adjacent to the PAM sequence.

89. The method of any one of claims 85-88, wherein the cell is a eukaryotic cell or a prokaryotic cell.

90. The method of claim 89, wherein the cell is a mammalian cell.

91. The method of claim 90, wherein the cell is a human cell.
